(19) United States
(12) Patent Application Publication
(10) Pub. No.: US 2021/0284621 A1
(43) Pub. Date: Sep. 16, 2021

(54) CANNABINOIDS AND USES THEREOF

(71) Applicant: Corbus Pharmaceuticals, Inc., Norwood, MA (US)

(72) Inventors: Kristos Adrian MOSHOS, Watertown, MA (US); Clifton D. LEIGH, Somerville, MA (US); Yuhua George ZHANG, Auburndale, MA (US); Mark TEPPER, Newton, MA (US); Hongfeng DENG, Acton, MA (US)

(21) Appl. No.: 17/058,980

(22) PCT Filed: May 31, 2019
(86) PCT No.: PCT/US19/34965

§ 371 Date: Nov. 25, 2020

Related U.S. Application Data
(60) Provisional application No. 62/678,689, filed on May 31, 2018.

(51) Int. Cl. C07D 311/78; C07D 405/12; C07D 407/12; C07D 413/06

(52) U.S. Cl. CPC C07D 311/78; C07D 413/06; C07D 407/12; C07D 405/12

(57) ABSTRACT

The invention relates to cannabinoid compounds, pharmaceutical compositions including one or more cannabinoid compounds, and the use of pharmaceutical compositions including one or more cannabinoid compounds for the treatment of a disease or condition (e.g., a fibrotic disease or an inflammatory disease) in a subject in need thereof.

### BACKGROUND OF THE INVENTION

Cannabinoids are a class of chemicals found in Cannabis sativa L (Cannabis) and related derivatives that have been shown to exhibit various pharmacologic activities. Tetrahydrocannabinol (THC) is the major psychoactive cannabinoid of cannabis. In addition to mood-altering effects, THC has been reported to exhibit other activities, some of which may have therapeutic value. The potential therapeutic value of THC has led to a search for related compounds which minimize the psychoactive effects, while retaining the activities of potential medicinal value.

Cannabinoids in current therapeutic use, such as nabilone, activate both the cannabinoid type 1 receptor (CB₁) and the cannabinoid type 2 receptor (CB₂). Selective CB₂ activation may provide some of the therapeutic effects of cannabinoids, such as their immuno-modulatory properties, without the psychoactive effects of CB₁ activation. Therefore, cannabinoid CB₂ receptors represent an attractive target for drug development.

(6aR,10aR)-1-hydroxy-6,6-dimethyl-3-(2-methyl-2-octanyl)-6a,7,10,10a-tetrahydro-6H-benzo[c]chromene-9-carboxylic acid (also known as ajulemic acid, AJA, JBT-101, resunab, anabasum, or lenabasum) has been investigated for its potential therapeutic benefits in a number of diseases, including fibrotic diseases and inflammatory diseases, for which there is a need for new therapies with improved safety and efficacy profiles. Ajulemic acid has been shown to exhibit receptor selectivity for CB₂ over CB₁.

There is a continued need for the development of cannabinoids with improved potency and selectivity for the CB₂ receptor.

### SUMMARY OF THE INVENTION

The invention relates to cannabinoid compounds, pharmaceutical compositions including one or more cannabinoid compounds, and the use of pharmaceutical compositions including one or more cannabinoid compounds for the treatment of a disease or condition (e.g., a fibrotic disease or an inflammatory disease) in a subject in need thereof. In particular, the invention features compounds sharing structural features with (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid). In some embodiments, the invention features compounds which are agonists of the CB₂ receptor. In preferred embodiments of the invention, the invention features compounds that have increased affinity for the CB₂ receptor (e.g., increased affinity for the CB₂ receptor compared to ajulemic acid), increased selectivity for the CB₂ receptor (e.g., increased selectivity for the CB₂ receptor over the CB₁ receptor compared to ajulemic acid), or both increased affinity and increased selectivity for the CB₂ receptor. In some embodiments, the invention features compounds with an increased safety or efficacy profile in the treatment of a disease or condition (e.g., a fibrotic disease or an inflammatory disease), as compared to other cannabinoids, such as ajulemic acid. In some embodiments, the invention features compounds having improved pharmacokinetic properties or improved stability (e.g., improved pharmacokinetic properties or improved stability as compared to ajulemic acid).

In a first aspect, the invention features a compound described by formula (I):

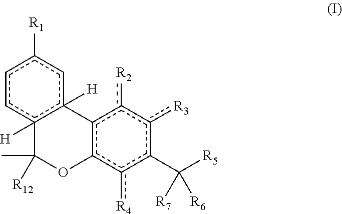

wherein each dashed line is optionally a double bond; R₁ is optionally substituted carboxyl, optionally substituted amide, optionally substituted thioester, optionally substituted thioamide, optionally substituted sulfonamide, an optionally substituted alkyl, or cyano; R₂ is H, O, Cl, F, NH₂, hydroxyl, or optionally substituted alkoxy; R₃ and R₄ are each independently H, O, Cl, or F; R₅ is optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, or optionally substituted C1-C20 alkoxy; R₆ and R₇ are each independently H, —CH₃, —CF₃, or —CH₂OH; and R₁₂ is —CH₃ or —CH₂OH, or a pharmaceutically acceptable salt thereof; and wherein the compound described by formula (I) is not ajulemic acid (AJA):

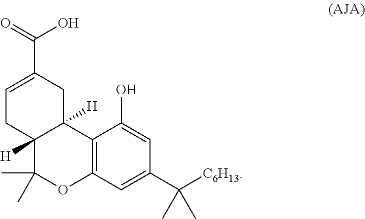

In some embodiments, the compound is described by formula (II):

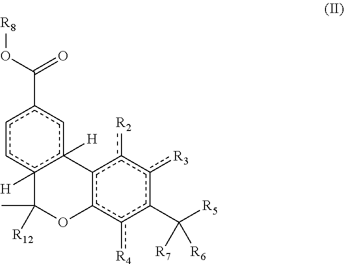

wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino, or a pharmaceutically acceptable salt thereof.

In some embodiments, the compound is described by any one of formulas (II-1), (II-2), (II-3), (II-4), (II-5), (II-6), (II-7), or (II-8):

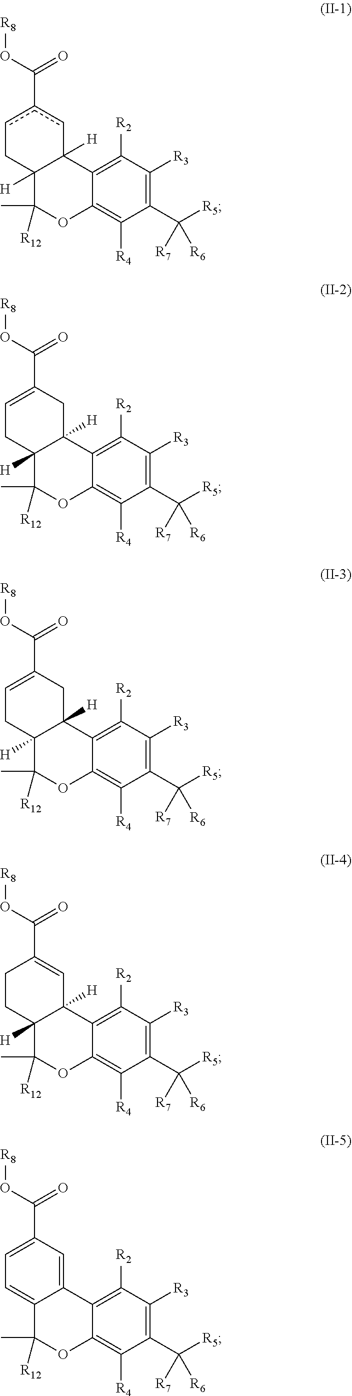

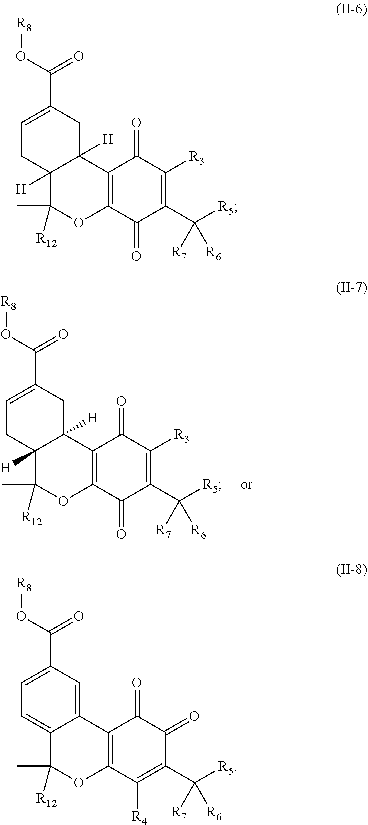

In some embodiments, the compound is described by formula (III):

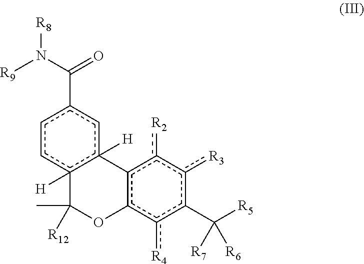

wherein R₈ and R₉ are each independently H, OH, optionally substituted amine, optionally substituted C1-C20 alkyl (e.g., —CH₃), optionally substituted C1-C20 alkenyl, optionally substituted C1-C20 alkynyl, optionally substituted C1-C20 alkoxy, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or R₈ and R₉ form an optionally substituted C3-C20 heterocyclyl; or a pharmaceutically acceptable salt thereof.

In some embodiments, the compound is described by any one of formulas (III-1), (III-2), (III-3), (III-4), (III-5), (III-6), (III-7), or (III-8):

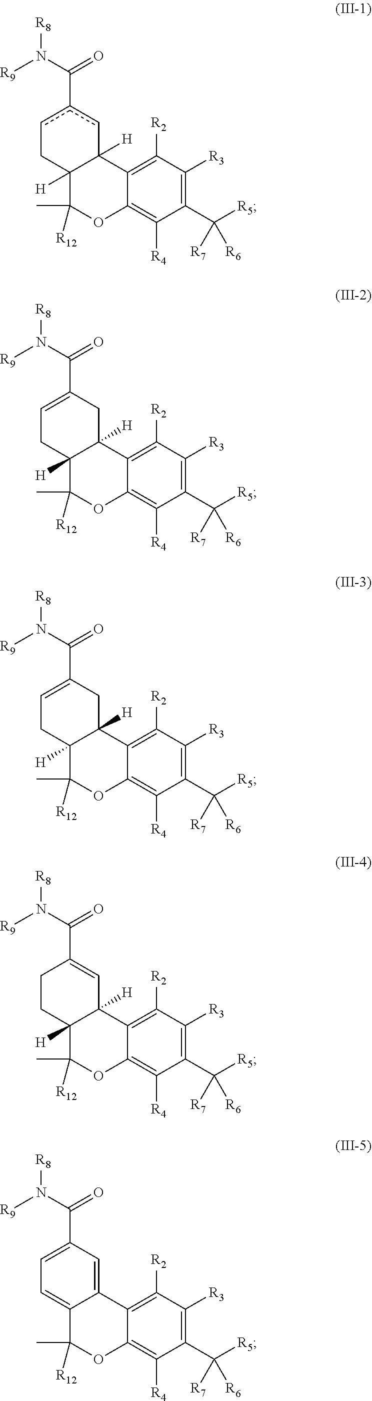

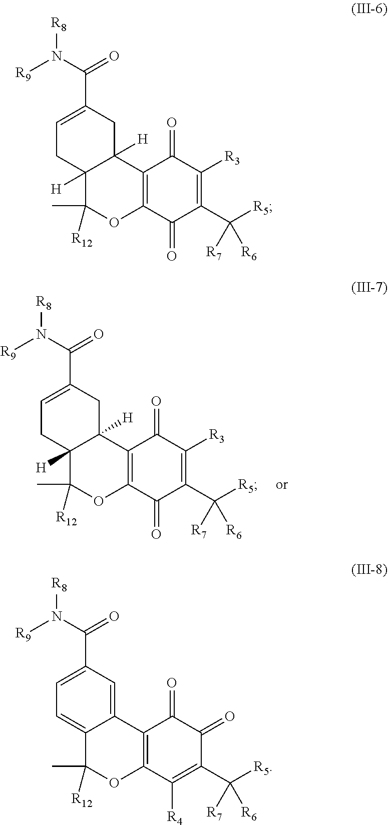

In some embodiments, the compound is described by formula (IV):

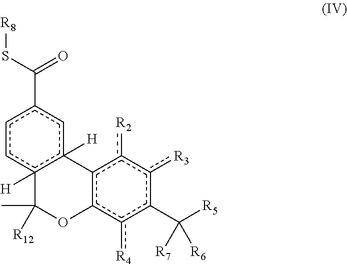

wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino, or a pharmaceutically acceptable salt thereof.

In some embodiments, the compound is described by any one of formulas (IV-1), (IV-2), (IV-3), (IV-4), (IV-5), (IV-6), (IV-7), or (IV-8):

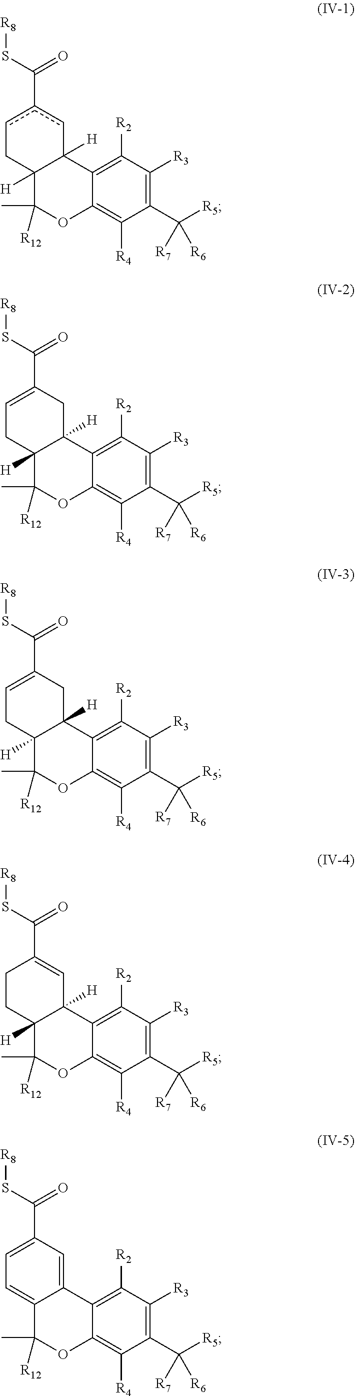

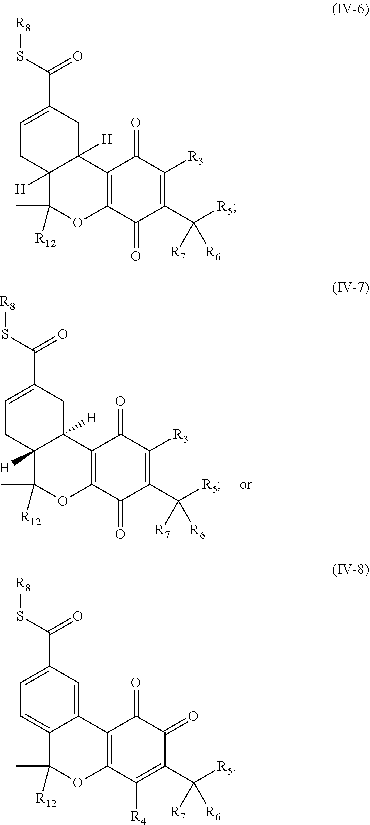

In some embodiments, the compound is described by formula (V):

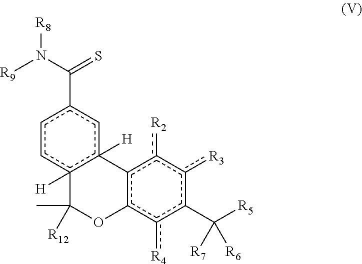

wherein R₈ is H, OH, optionally substituted amine, optionally substituted C1-C20 alkyl, optionally substituted C1-C20 alkenyl, optionally substituted C1-C20 alkynyl, optionally substituted C1-C20 alkoxy, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino, or a pharmaceutically acceptable salt thereof.

In some embodiments, the compound is described by any one of formulas (V-1), (V-2), (V-3), (V-4), (V-5), (V-6), (V-7), or (V-8):

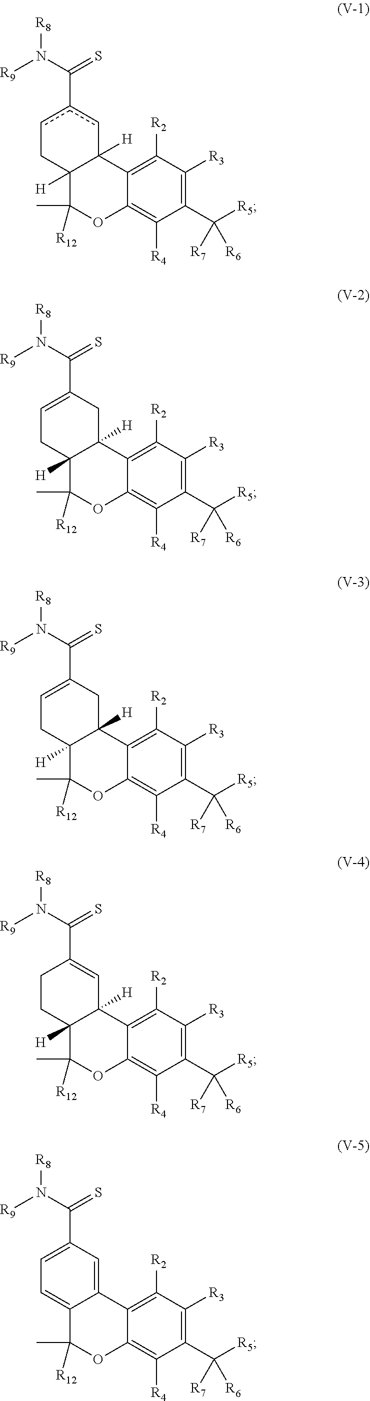

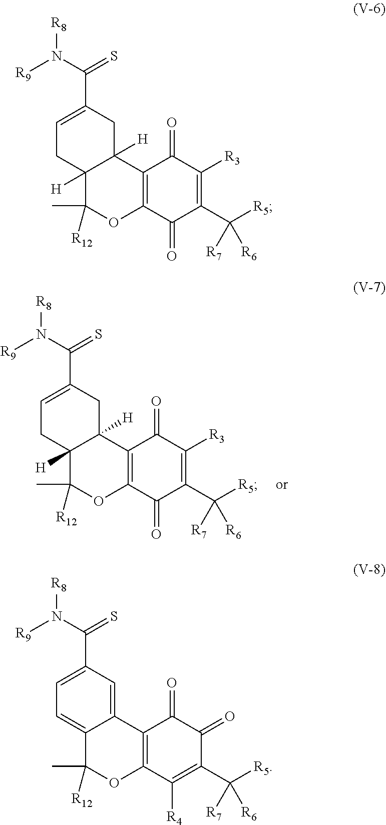

In some embodiments, the compound is described by formula (VI):

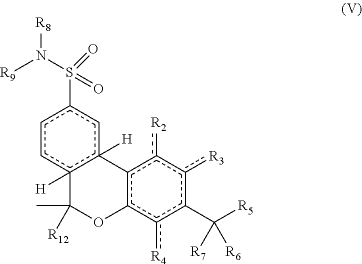

wherein R₈ is H, OH, optionally substituted amine, optionally substituted C1-C20 alkyl, optionally substituted C1-C20 alkenyl, optionally substituted C1-C20 alkynyl, optionally substituted C1-C20 alkoxy, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino, or a pharmaceutically acceptable salt thereof.

In some embodiments, the compound is described by any one of formulas (VI-1), (VI-2), (VI-3), (VI-4), (VI-5), (VI-6), (VI-7), or (VI-8):

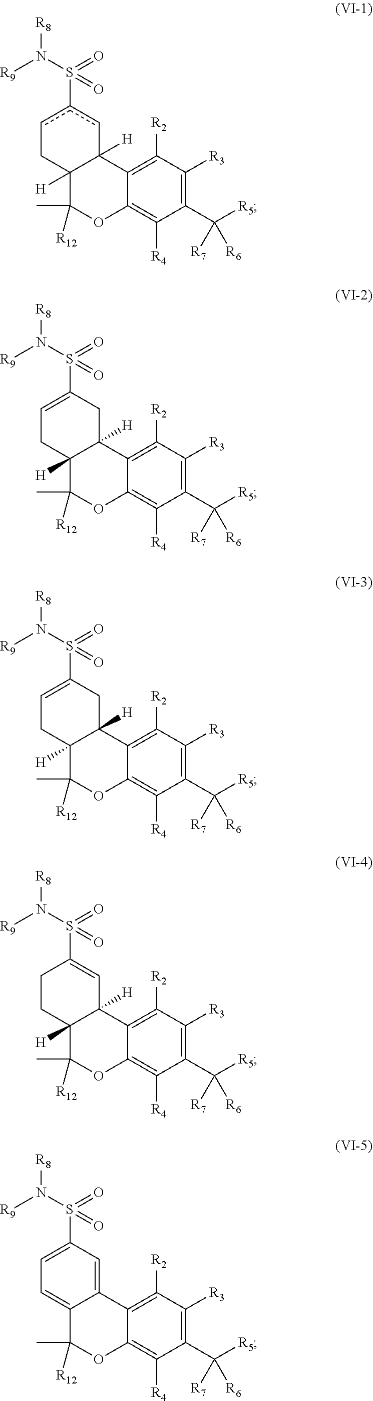

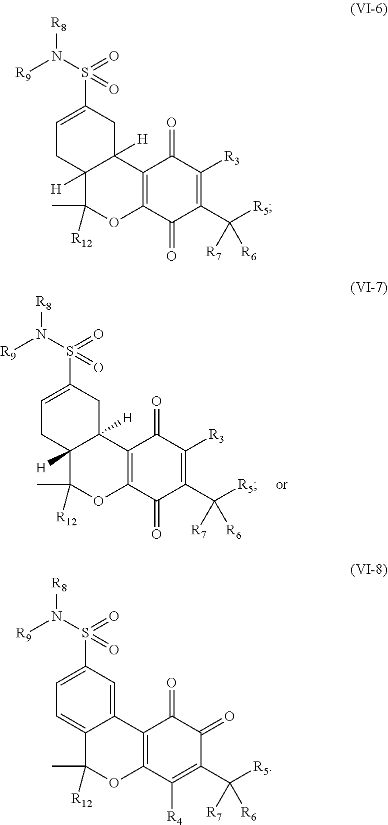

In some embodiments, the compound is described by formula (VII):

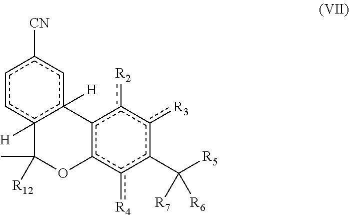

or a pharmaceutically acceptable salt thereof.

In some embodiments, the compound is described by any one of formulas (VII-1), (VII-2), (VII-3), (VII-4), (VII-5), (VII-6), (VII-7), or (VII-8):

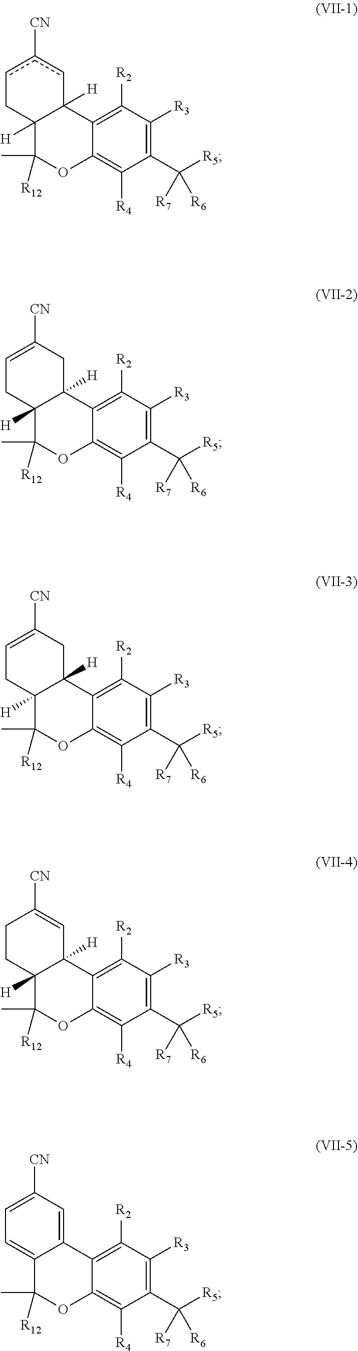

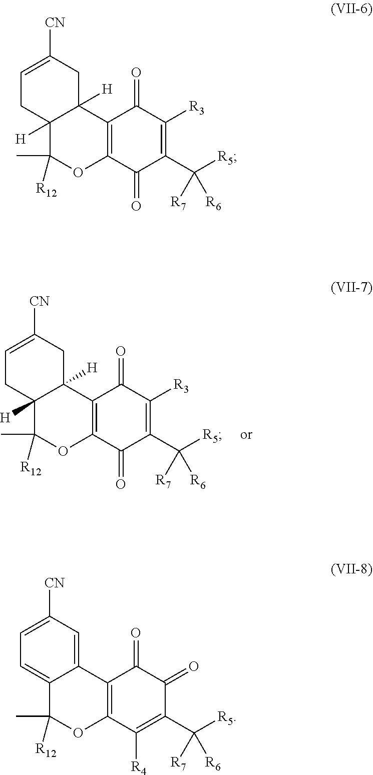

In some embodiments, the compound is described by formula (VIII):

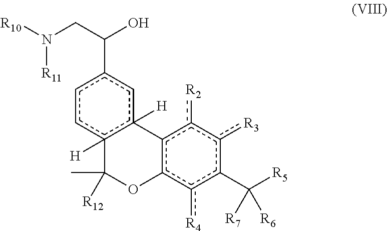

wherein R₁₀ and R₁₁ are each independently H, OH, optionally substituted amine, optionally substituted C1-C20 alkyl, optionally substituted C1-C20 alkenyl, optionally substituted C1-C20 alkynyl, optionally substituted C1-C20 alkoxy, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or wherein R₁₀ and R₁₁ form an optionally substituted C3-C20 heterocyclyl, or a pharmaceutically acceptable salt thereof.

In some embodiments, the compound is described by any one of formulas (VIII-1), (VIII-2), (VIII-3), (VIII-4), (VIII-5), (VIII-6), (VIII-7), or (VIII-8):

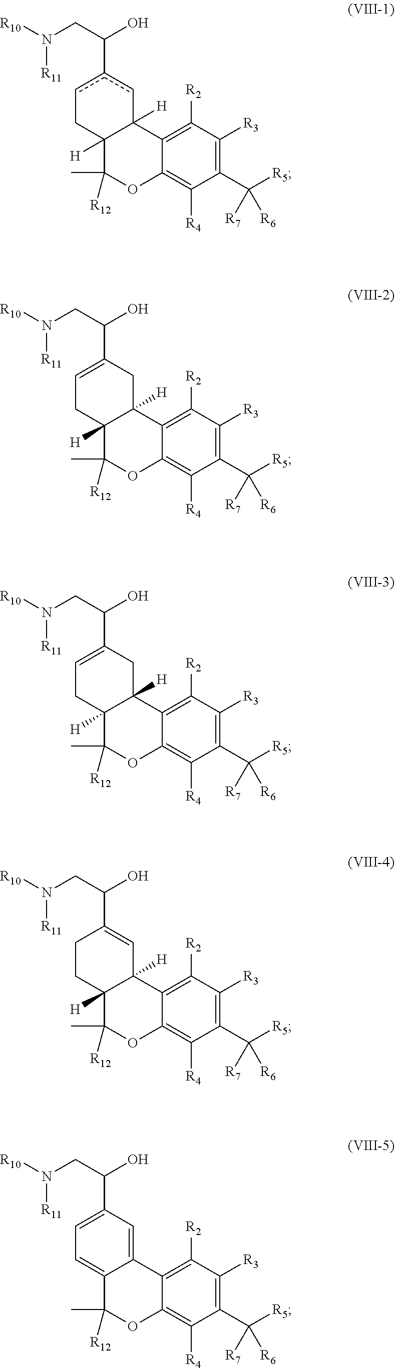

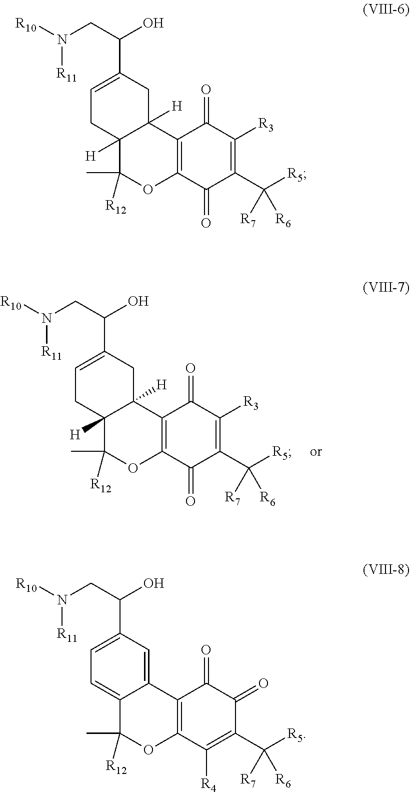

In some embodiments, R₂ (e.g., R₂ of any one of formulas II-1, II-2, II-3, II-4, II-5, III-1, III-2, III-3, III-4, III-5, III IV-1, IV-2, IV-3, IV-4, IV-5, V-1, V-2, V-3, V-4, V-5, VI-1, VI-2, VI-3, VI-4, VI-5, VII-1, VII-2, VII-3, VII-4, VII-5, VIII-1, VIII-2, VIII-3, VIII-4, or VIII-5) is H, O, Cl, F, NH₂, or methoxy.

In some embodiments, R₃ (e.g., R₃ of any one of formulas II-1, II-2, II-3, II-4, II-5, II-6, II-7, II-1, III-2, III-3, III-4, III-5, III-6, III-7, IV-1, IV-2, IV-3, IV-4, IV-5, IV-6, IV-7, V-1, V-2, V-3, V-4, V-5, V-6, V-7, VI-1, VI-2, VI-3, VI-4, VI-5, VI-6, VI-7, VII-1, VII-2, VII-3, VII-4, VII-5, VII-6, VII-7, VIII-1, VIII-2, VIII-3, VIII-4, VIII-5, VIII-6, or VIII-7) and R₄ (e.g., R₄ of any one of formulas I-1, II-2, II-3, II-4, II-5, II-8, II-1, III-2, III-3, III-4, III-5, III-8, IV-1, IV-2, IV-3, IV-4, IV-5, IV-8, V-1, V-2, V-3, V-4, V-5, V-8, VI-1, VI-2, VI-3, VI-4, VI-5, VI-8, VII-1, VII-2, VII-3, VII-4, VII-5, VII-8, VIII-1, VIII-2, VIII-3, VIII-4, VIII-5, or VIII-8) are each independently H, O, Cl, or F.

In some embodiments, R₅ (e.g., R₅ of any one of formulas II-1, II-2, II-3, II-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V- 4, V-5, V-6, V-7, V-8, VI-1, VI-2, VI-3, VI-4, VI-5, VI-6, VI-7, VI-8, VII-1, VII-2, VII-3, VII-4, VII-5, VII-6, VII-7, VII-8, VIII-1, VIII-2, VIII-3, VIII-4, VIII-5, VIII-6, VIII-7, or VIII-8) is selected from:

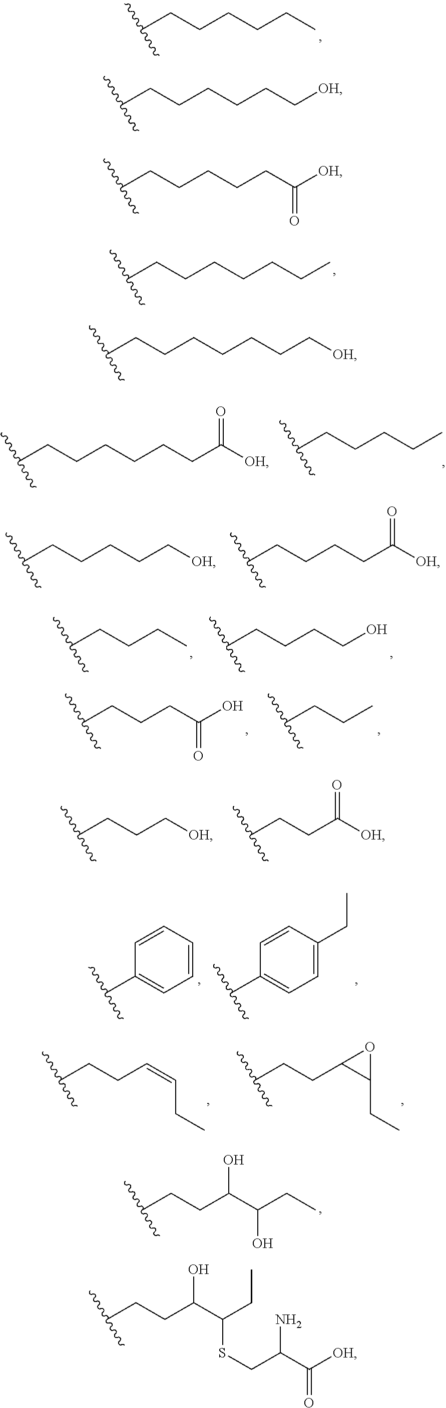

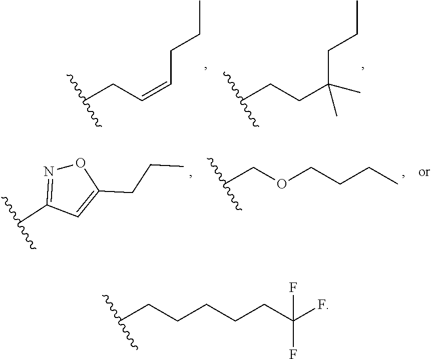

In some embodiments, R₆ (e.g., R₆ of any one of formulas II-1, II-2, II-3, II-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V- 4, V-5, V-6, V-7, V-8, VI-1, VI-2, VI-3, VI-4, VI-5, VI-6, VI-7, VI-8, VII-1, VII-2, VII-3, VII-4, VII-5, VII-6, VII-7, VII-8, VIII-1, VIII-2, VIII-3, VIII-4, VIII-5, VIII-6, VIII-7, or VIII-8) and R₇ (e.g., R₇ of any one of formulas II-1, II-2, II-3, II-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V-4, V-5, V-6, V-7, V-8, VI-1, VI-2, VI-3, VI-4, VI-5, VI-6, VI-7, VI-8, VII-1, VII-2, VII-3, VII-4, VII-5, VII-6, VII-7, VII-8, VIII-1, VIII-2, VIII-3, VIII-4, VIII-5, VIII-6, VIII-7, or VIII-8) are each independently selected from H, —CH₃, —CF₃, or —CH₂OH.

In some embodiments, R₈ (e.g., R₈ of any one of formulas II-1, II-2, II-3, II-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V- 4, V-5, V-6, V-7, V-8, VI-1, VI-2, VI-3, VI-4, VI-5, VI-6, VI-7, or VI-8) is selected from:

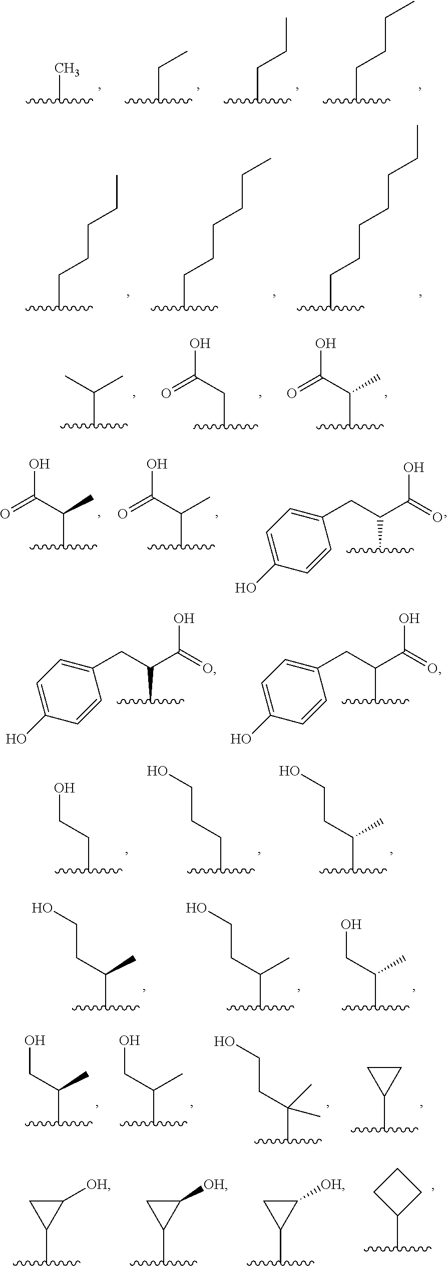

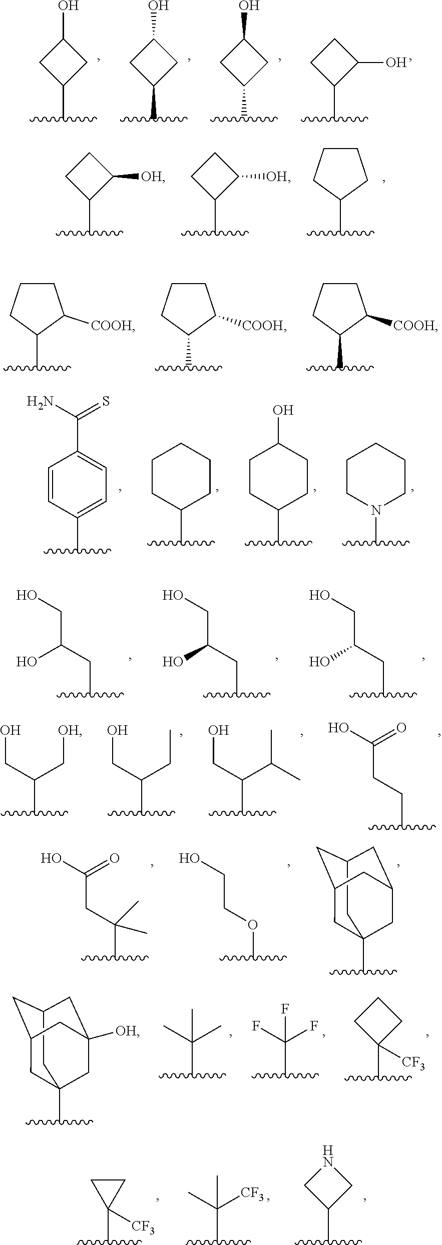

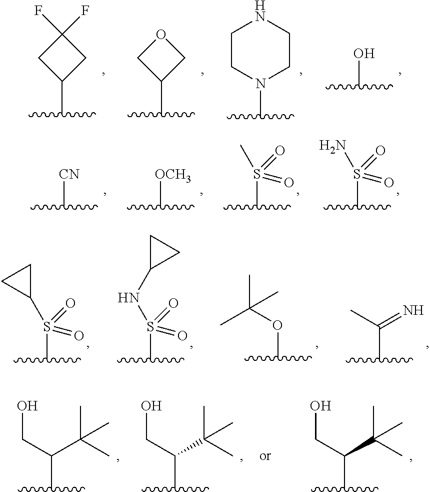

In some embodiments, R₉ (e.g., R₉ of any one of formulas III-1, III-2, III-3, III-4, III-5, III-6, III-7, III-8, V-1, V-2, V-3, V-4. V-5, C-6, V-7, V-8, VI-1, VI-2, VI-3, VI-4, VI-5, VI-6, VI-7, or VI-8) is H or C1-C4 alkyl (e.g., H or CH₃).

In some embodiments, R₈ and R₉ (e.g., R₈ and R₉ of any one of formulas III-1, III-2, III-3, III-4, III-5, III-6, III-7, III-8, V-1, V-2, V-3, V-4. V-5, C-6, V-7, V-8, VI-1, VI-2, VI-3, VI-4, VI-5, VI-6, VI-7, or VI-8) form an optionally substituted C3-C20 heterocyclyl. In some embodiments the optionally substituted C3-C20 heterocyclyl is selected from:

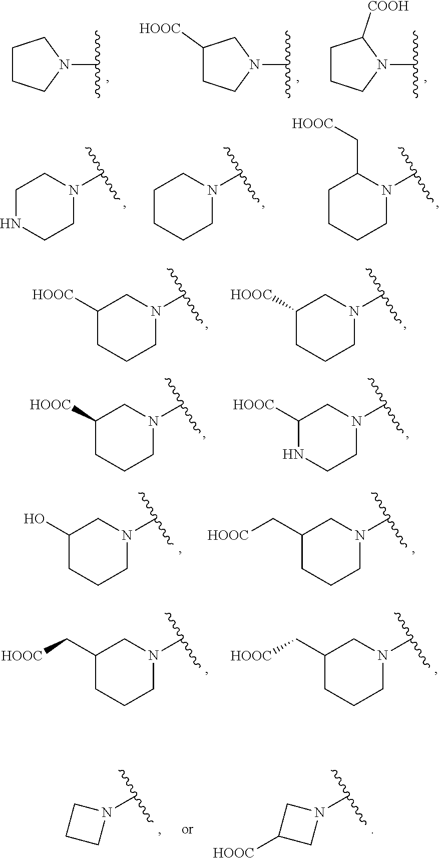

In some embodiments, each of R₁₀ and R₁₁ (e.g., each of R₁₀ or R₁₁ of any one of formulas VIII-1, VIII-2, VIII-3, VIII-4, VIII-5, VIII-6, VIII-7, or VIII-8) are independently selected from H or any one of:

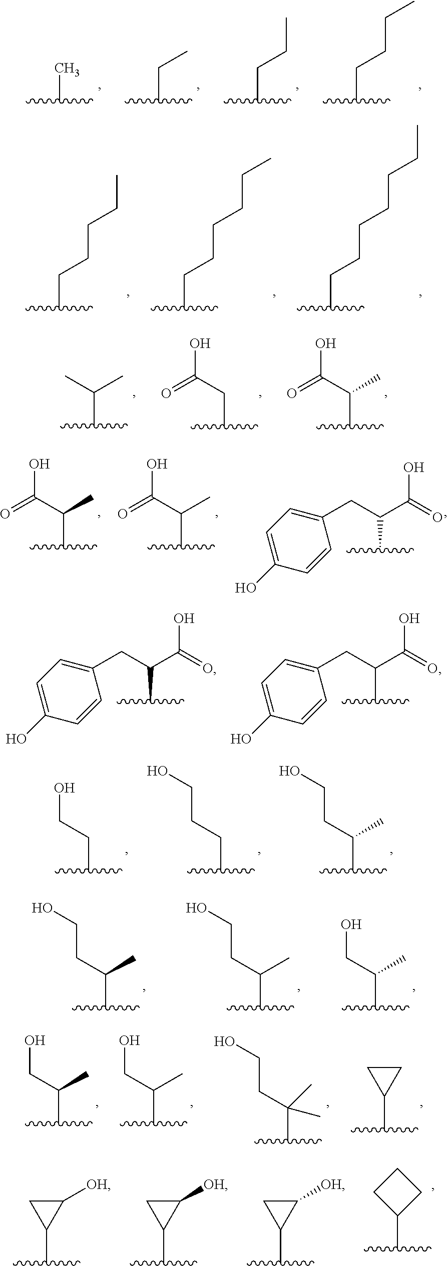

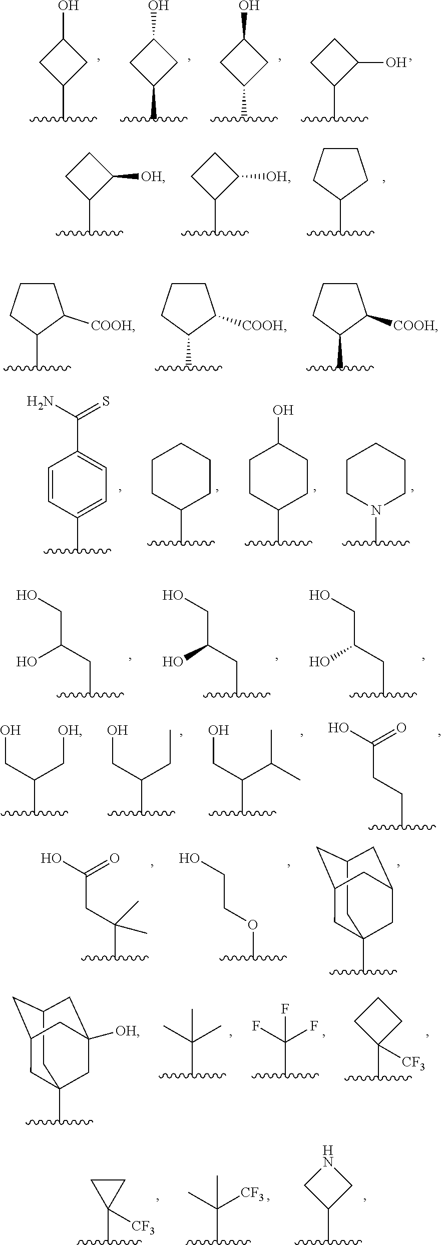

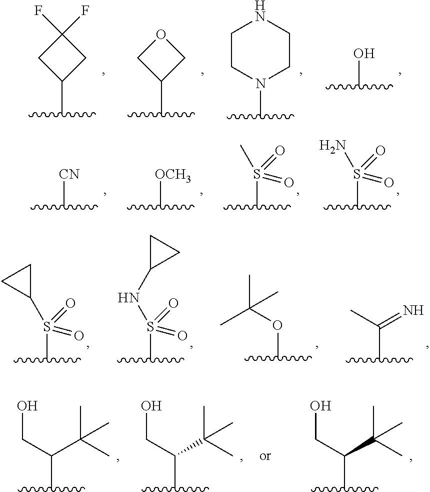

In some embodiments, R₁ (e.g., R₁ of any one of formulas III-1, III-2, III-3, III-4, III-5, III-6, III-7, III-8, V-1, V-2, V-3, V-4. V-5, C-6, V-7, V-8, VI-1, VI-2, VI-3, VI-4, VI-5, VI-6, VI-7, or VI-8) is H or C1-C4 alkyl (e.g., H or CH₃).

In some embodiments, R₁₀ and R₁₁ (e.g., R₁₀ and R₁₁ of any one of formulas VIII-1, VIII-2, VIII-3, VIII-4, VIII-5, VIII-6, VIII-7, or VIII-8) form an optionally substituted C3-C20 heterocyclyl. In some embodiments, R₁₀ and R₁₁ form an optionally substituted C3-C20 heterocyclyl selected from:

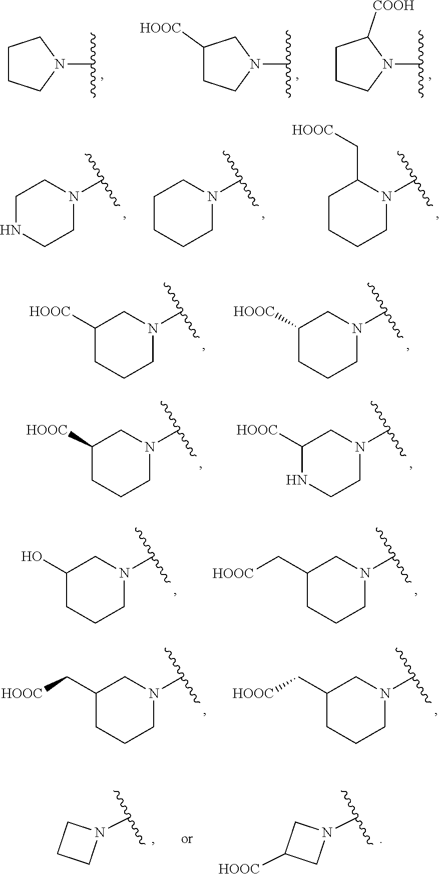

In some embodiments, R₁₂ (e.g., R₁₂ of any one of formulas II-1, II-2, II-3, II-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V-4, V-5, V-6, V-7, V-8, VI-1, VI-2, VI-3, VI-4, VI-5, VI-6, VI-7, VI-8, VII-1, VII-2, VII-3, VII-4, VII-5, VII-6, VII-7, VII-8, VIII-1, VIII-2, VIII-3, VIII-4, VIII-5, VIII-6, VIII-7, or VIII-8) is selected from —CH₃ or —CH₂OH.

In some embodiments, the compound is a compound of Table 1 (e.g., any one of compounds 1-144):

TABLE 1

| | |
|---|---|
| 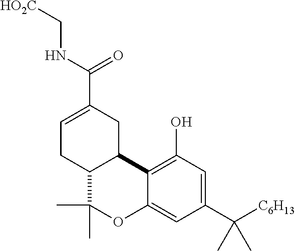 | Compound 1 |
| 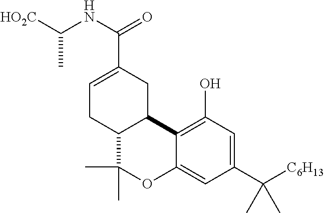 | Compound 2 |
| 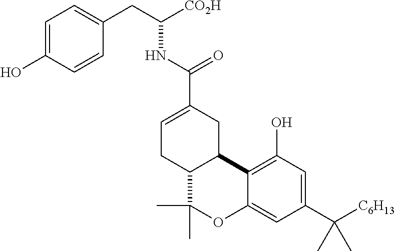 | Compound 3 |
| 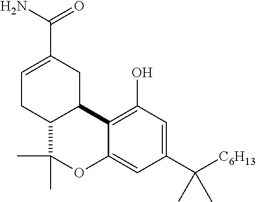 | Compound 4 |
| 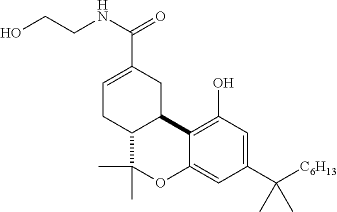 | Compound 5 |
| 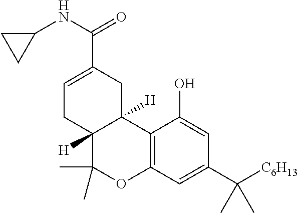 | Compound 6 |
| 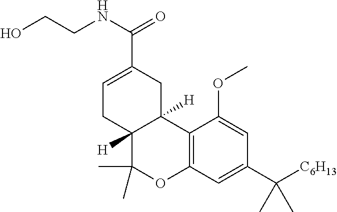 | Compound 7 |
| 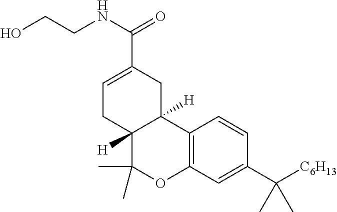 | Compound 8 |
| 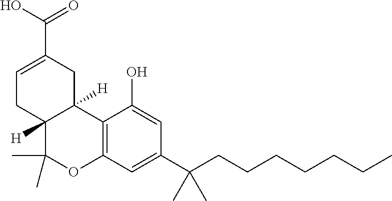 [SMILES: CCCCCCCC(C)(C)c1cc(O)c2c(c1)OC(C)(C)[C@@H]1CC=C(C(=O)O)C[C@@H]21] | Compound 9 |
| 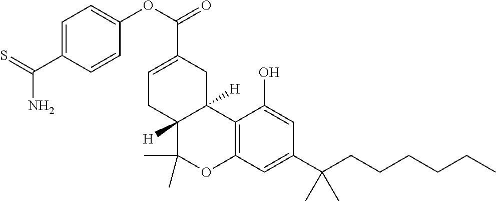 [SMILES: CCCCCCC(C)(C)c1cc(O)c2c(c1)OC(C)(C)[C@@H]1CC=C(C(=O)Oc3ccc(C(N)=S)cc3)C[C@@H]21] | Compound 10 |
| 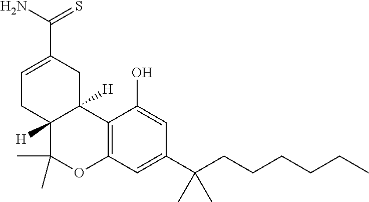 [SMILES: CCCCCCC(C)(C)c1cc(O)c2c(c1)OC(C)(C)[C@@H]1CC=C(C(N)=S)C[C@@H]21] | Compound 11 |
| 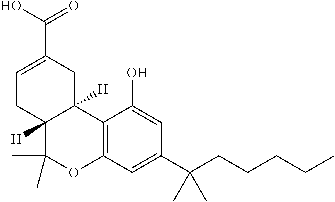 [SMILES: CCCCCC(C)(C)c1cc(O)c2c(c1)OC(C)(C)[C@@H]1CC=C(C(=O)O)C[C@@H]21] | Compound 12 |
| 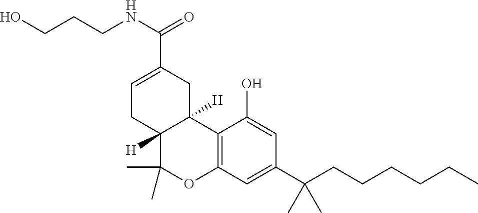 [SMILES: CCCCCCC(C)(C)c1cc(O)c2c(c1)OC(C)(C)[C@@H]1CC=C(C(=O)NCCCO)C[C@@H]21] | Compound 13 |
| 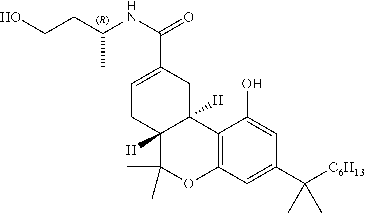 | Compound 14 |
| 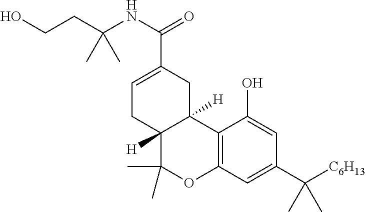 | Compound 15 |
| 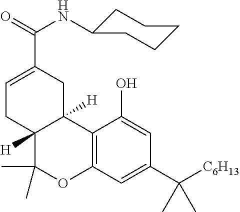 | Compound 16 |
| 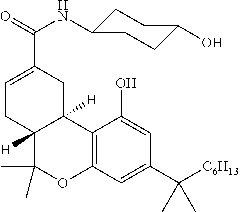 | Compound 17 |
| 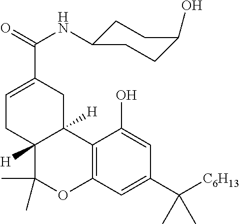 | Compound 18 |
| 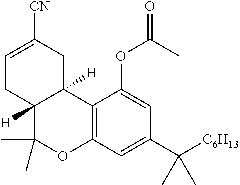 | Compound 19 |
| 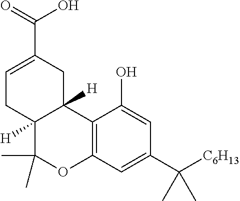 | Compound 20 |
| 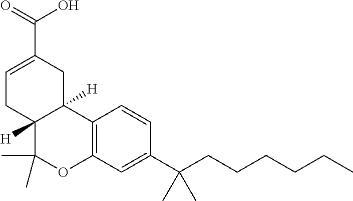 [SMILES: CCCCCCC(C)(C)c1ccc2c(c1)OC(C)(C)[C@@H]1CC=C(C(=O)O)C[C@@H]21] | Compound 21 |
| 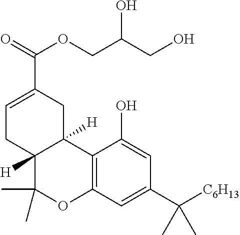 | Compound 22 |
| 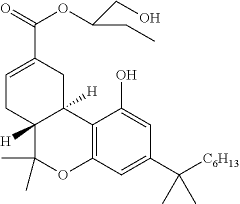 | Compound 23 |
| 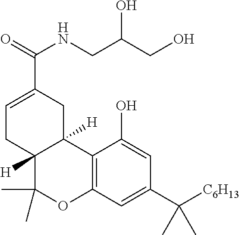 | Compound 24 |
| 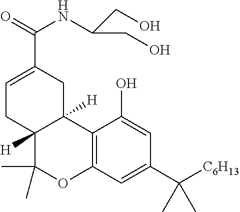 | Compound 25 |
| 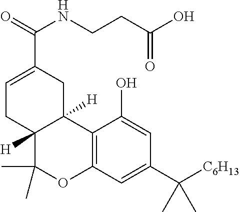 | Compound 26 |
| 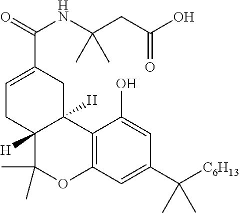 | Compound 27 |
| 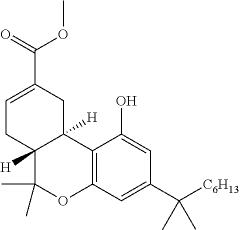 | Compound 28 |
| 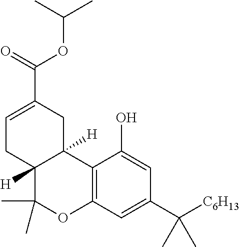 | Compound 29 |
| 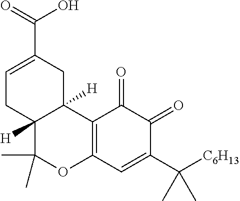 | Compound 30 |
| 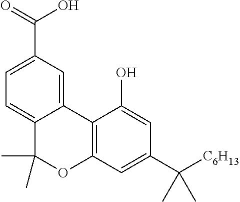 | Compound 31 |
| 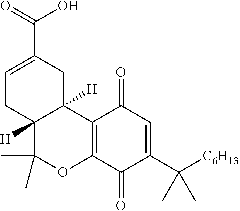 | Compound 32 |
| 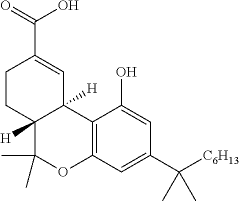 | Compound 33 |
| 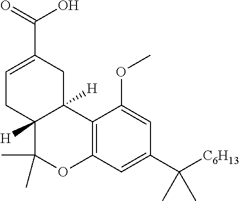 | Compound 34 |
| 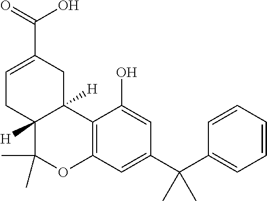 [SMILES: CC(C)(c1ccccc1)c1cc(O)c2c(c1)OC(C)(C)[C@@H]1CC=C(C(=O)O)C[C@@H]21] | Compound 35 |
| 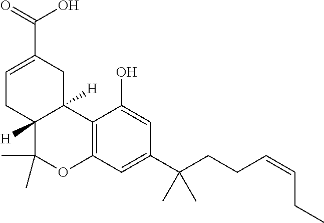 [SMILES: CC/C=C\CCC(C)(C)c1cc(O)c2c(c1)OC(C)(C)[C@@H]1CC=C(C(=O)O)C[C@@H]21] | Compound 36 |
| 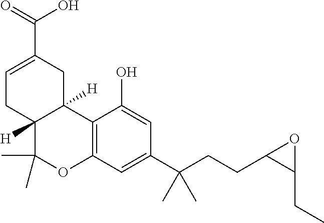 [SMILES: CCC1OC1CCC(C)(C)c1cc(O)c2c(c1)OC(C)(C)[C@@H]1CC=C(C(=O)O)C[C@@H]21] | Compound 37 |
| 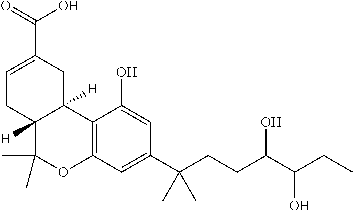 [SMILES: CCC(O)C(O)CCC(C)(C)c1cc(O)c2c(c1)OC(C)(C)[C@@H]1CC=C(C(=O)O)C[C@@H]21] | Compound 38 |
| 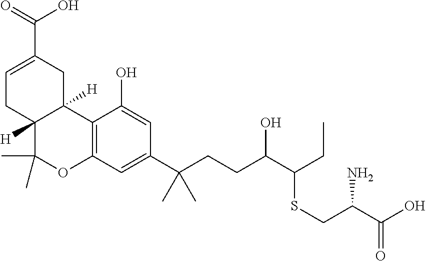 [SMILES: CCC(SC[C@H](N)C(=O)O)C(O)CCC(C)(C)c1cc(O)c2c(c1)OC(C)(C)[C@@H]1CC=C(C(=O)O)C[C@@H]21] | Compound 39 |
| 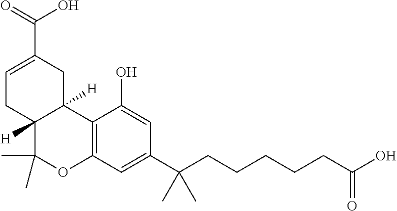 [SMILES: CC(C)(CCCCCC(=O)O)c1cc(O)c2c(c1)OC(C)(C)[C@@H]1CC=C(C(=O)O)C[C@@H]21] | Compound 40 |
| 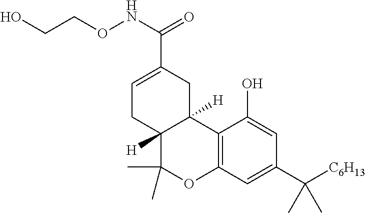 | Compound 41 |
| 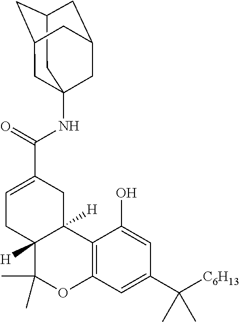 | Compound 42 |
| 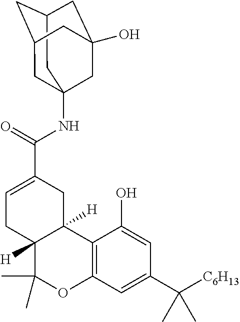 | Compound 43 |
| 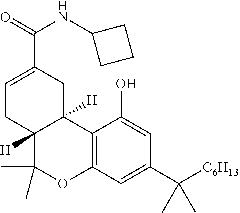 | Compound 44 |
| 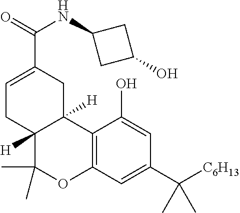 | Compound 45 |
| 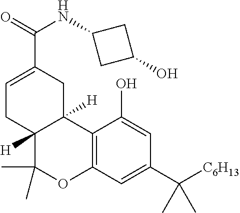 | Compound 46 |
| 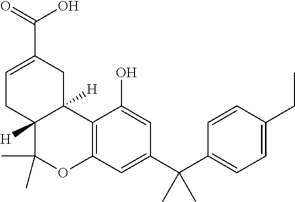 [SMILES: CCc1ccc(C(C)(C)c2cc(O)c3c(c2)OC(C)(C)[C@@H]2CC=C(C(=O)O)C[C@@H]32)cc1] | Compound 47 |
| 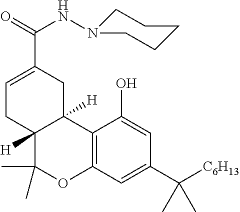 | Compound 48 |
| 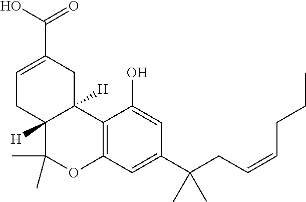 [SMILES: CCC/C=C\CC(C)(C)c1cc(O)c2c(c1)OC(C)(C)[C@@H]1CC=C(C(=O)O)C[C@@H]21] | Compound 49 |
| 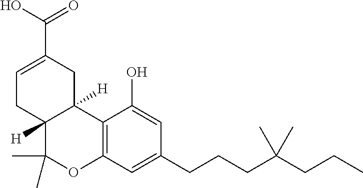 [SMILES: CCCC(C)(C)CCCc1cc(O)c2c(c1)OC(C)(C)[C@@H]1CC=C(C(=O)O)C[C@@H]21] | Compound 50 |
| 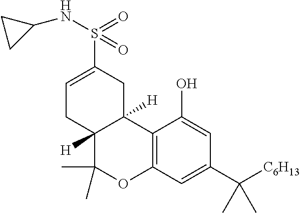 | Compound 51 |
| 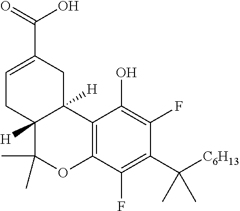 | Compound 52 |
| 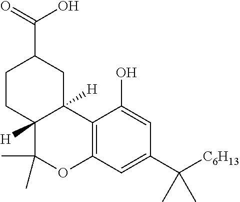 | Compound 53 |
| 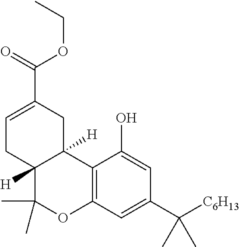 | Compound 54 |
| 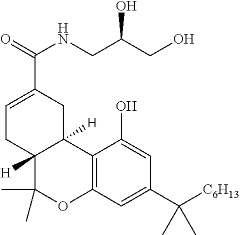 | Compound 55 |
| 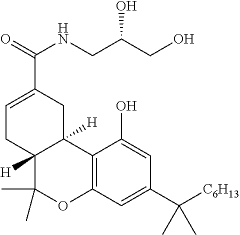 | Compound 56 |
| 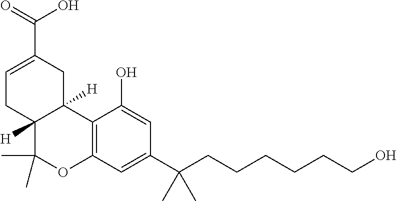 [SMILES: CC(C)(CCCCCCO)c1cc(O)c2c(c1)OC(C)(C)[C@@H]1CC=C(C(=O)O)C[C@@H]21] | Compound 57 |
| 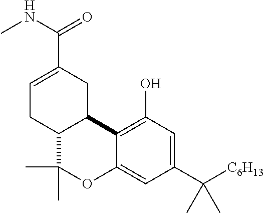 | Compound 58 |
| 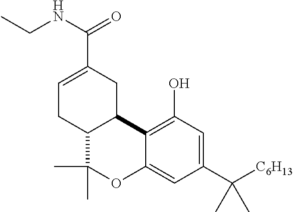 | Compound 59 |
| 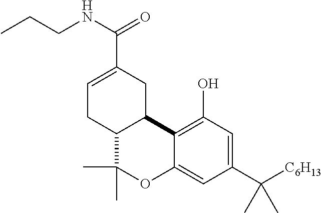 | Compound 60 |
| 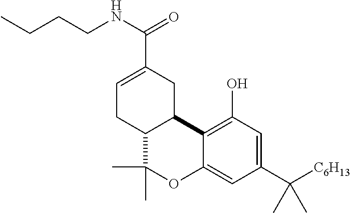 | Compound 61 |
| 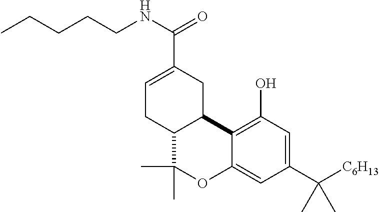 | Compound 62 |
| 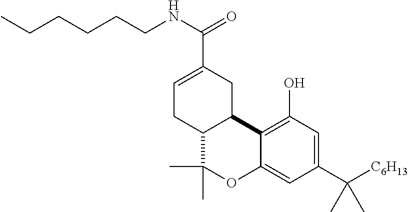 | Compound 63 |
| 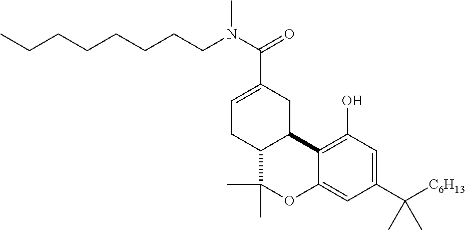 | Compound 64 |
| 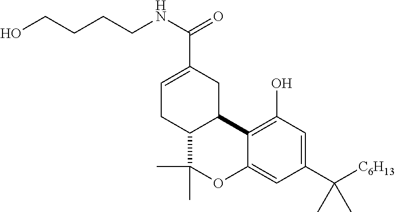 | Compound 65 |
| 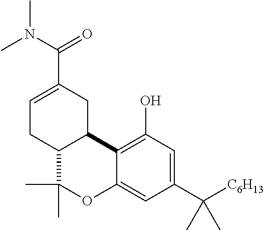 | Compound 66 |
| 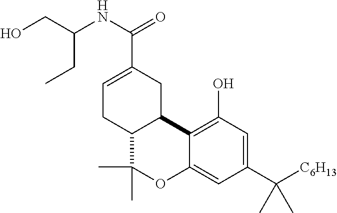 | Compound 67 |
| 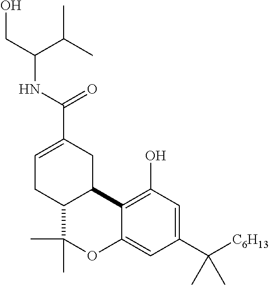 | Compound 68 |
| 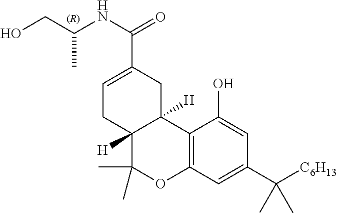 | Compound 69 |
| 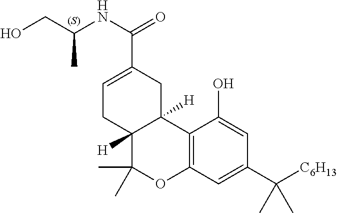 | Compound 70 |
| 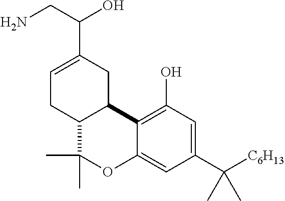 | Compound 71 |
| 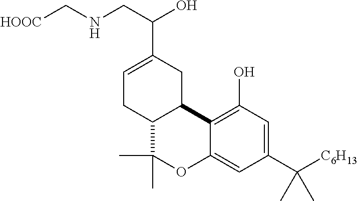 | Compound 72 |
| 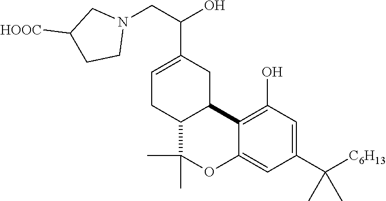 | Compound 73 |
| 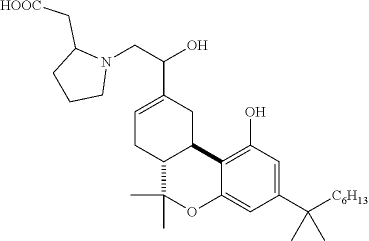 | Compound 74 |
| 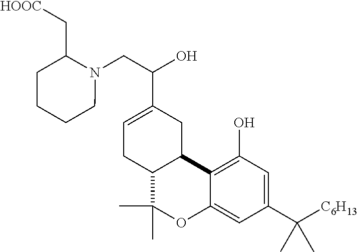 | Compound 75 |
| 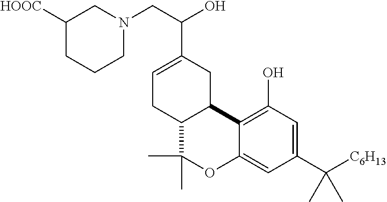 | Compound 76 |
| 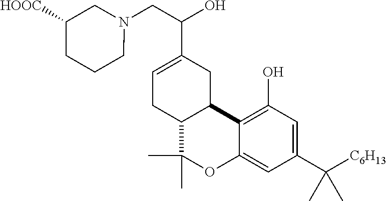 | Compound 77 |
| 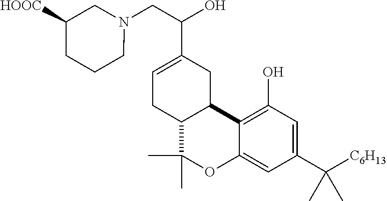 | Compound 78 |
| 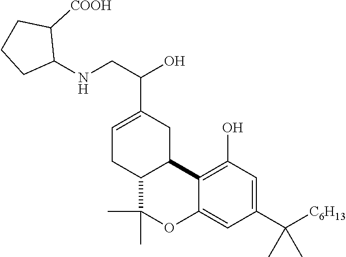 | Compound 79 |
| 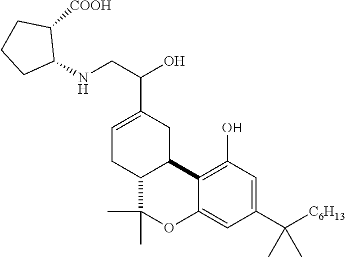 | Compound 80 |
| 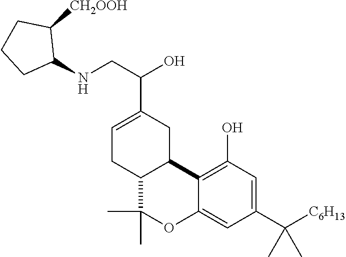 | Compound 81 |
| 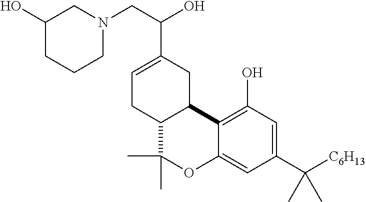 | Compound 82 |
| 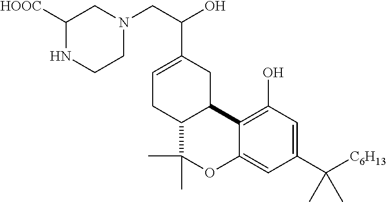 | Compound 83 |
| 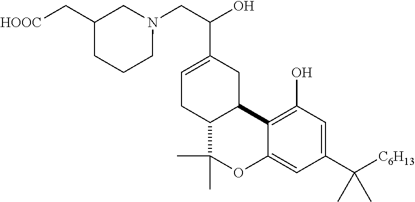 | Compound 84 |
| 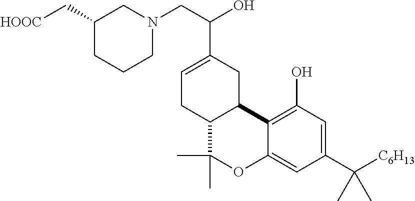 | Compound 85 |
| 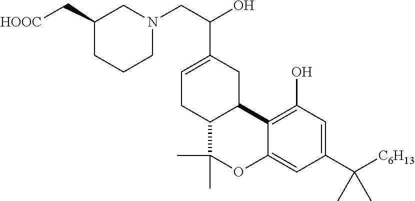 | Compound 86 |
| 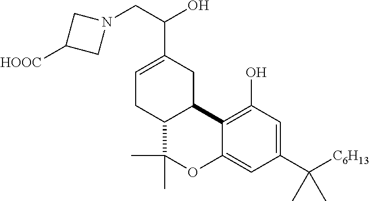 | Compound 87 |
| 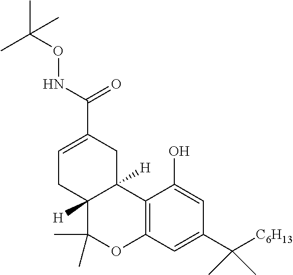 | Compound 88 |
| 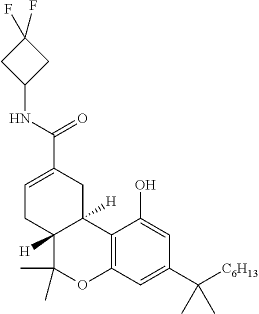 | Compound 89 |
| 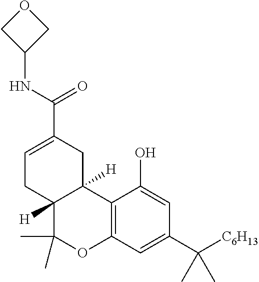 | Compound 90 |
| 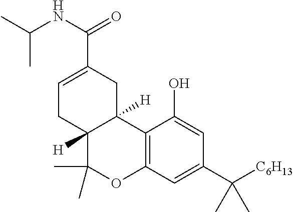 | Compound 91 |
| 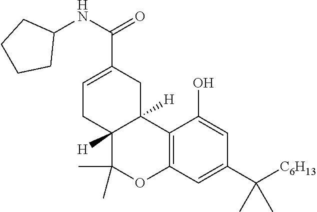 | Compound 92 |
| 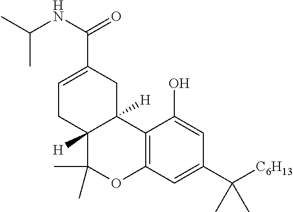 | Compound 93 |
| 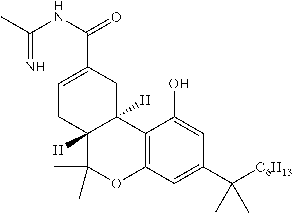 | Compound 94 |
| 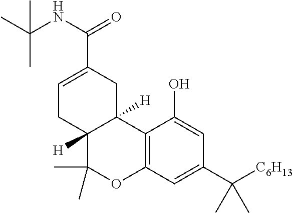 | Compound 95 |
| 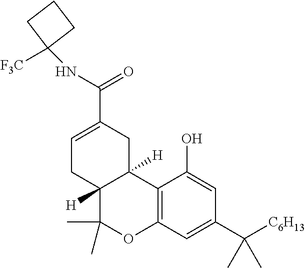 | Compound 96 |
| 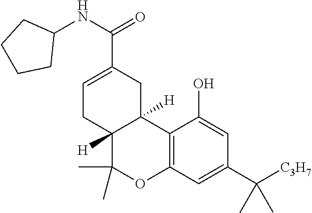 | Compound 97 |
| 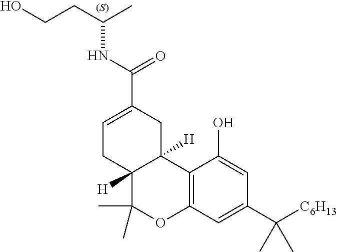 | Compound 98 |
| 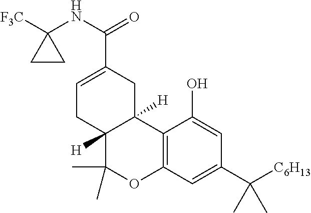 | Compound 99 |
| 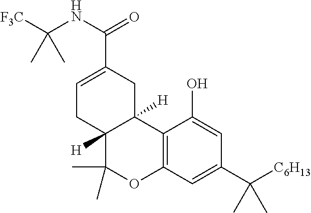 | Compound 100 |
| 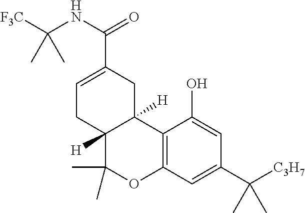 | Compound 101 |
| 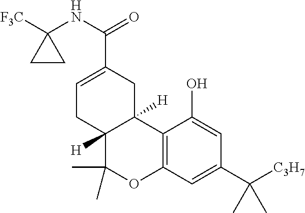 | Compound 102 |
| 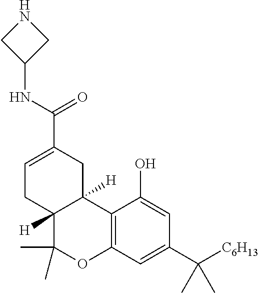 | Compound 103 |
| 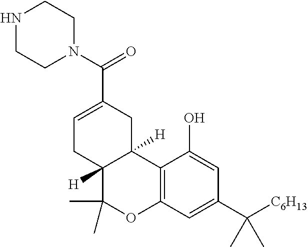 | Compound 104 |
| 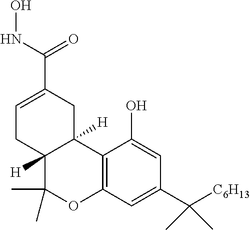 | Compound 105 |
| 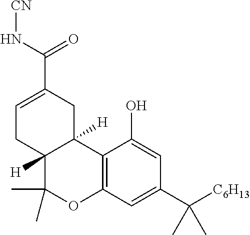 | Compound 106 |
| 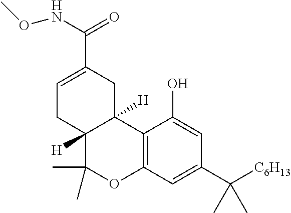 | Compound 107 |
| 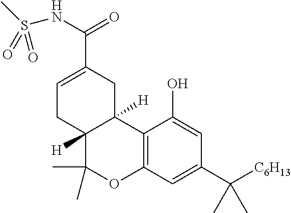 | Compound 108 |
| 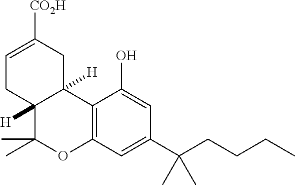 [SMILES: CCCCC(C)(C)c1cc(O)c2c(c1)OC(C)(C)[C@@H]1CC=C(C(=O)O)C[C@@H]21] | Compound 109 |
| 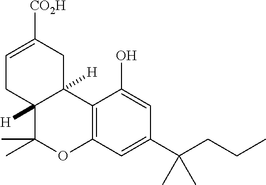 [SMILES: CCCC(C)(C)c1cc(O)c2c(c1)OC(C)(C)[C@@H]1CC=C(C(=O)O)C[C@@H]21] | Compound 110 |
| 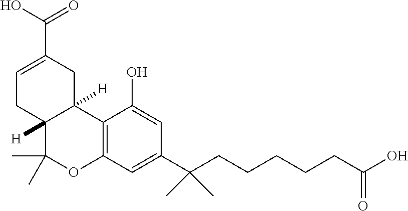 [SMILES: CC(C)(CCCCCC(=O)O)c1cc(O)c2c(c1)OC(C)(C)[C@@H]1CC=C(C(=O)O)C[C@@H]21] | Compound 111 |
| 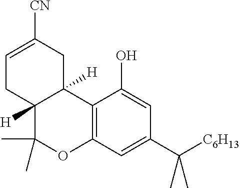 | Compound 112 |
| 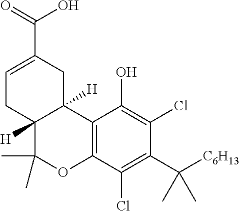 | Compound 113 |
| 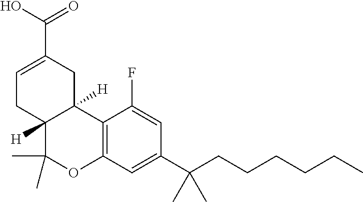 [SMILES: CCCCCCC(C)(C)c1cc(F)c2c(c1)OC(C)(C)[C@@H]1CC=C(C(=O)O)C[C@@H]21] | Compound 114 |
| 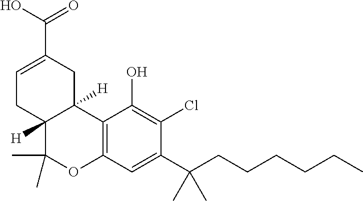 [SMILES: CCCCCCC(C)(C)c1cc2c(c(O)c1Cl)[C@@H]1CC(C(=O)O)=CC[C@H]1C(C)(C)O2] | Compound 115 |
| 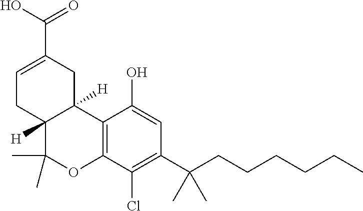 [SMILES: CCCCCCC(C)(C)c1cc(O)c2c(c1Cl)OC(C)(C)[C@@H]1CC=C(C(=O)O)C[C@@H]21] | Compound 116 |
| 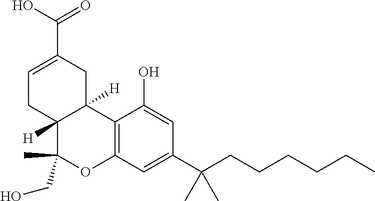 [SMILES: CCCCCCC(C)(C)c1cc(O)c2c(c1)O[C@](C)(CO)[C@@H]1CC=C(C(=O)O)C[C@@H]21] | Compound 117 |
| 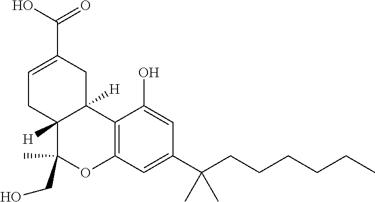 [SMILES: CCCCCCC(C)(C)c1cc(O)c2c(c1)O[C@@](C)(CO)[C@@H]1CC=C(C(=O)O)C[C@@H]21] | Compound 118 |
| 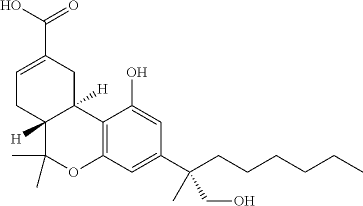 [SMILES: CCCCCC[C@](C)(CO)c1cc(O)c2c(c1)OC(C)(C)[C@@H]1CC=C(C(=O)O)C[C@@H]21] | Compound 119 |
| 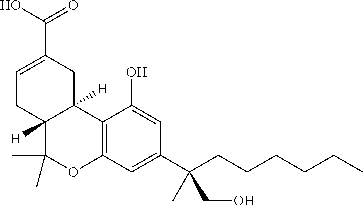 [SMILES: CCCCCC[C@@](C)(CO)c1cc(O)c2c(c1)OC(C)(C)[C@@H]1CC=C(C(=O)O)C[C@@H]21] | Compound 120 |
| 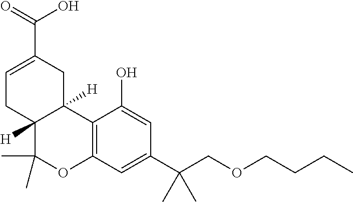 [SMILES: CCCCOCC(C)(C)c1cc(O)c2c(c1)OC(C)(C)[C@@H]1CC=C(C(=O)O)C[C@@H]21] | Compound 121 |
| 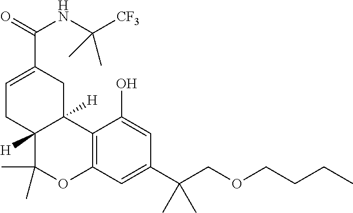 [SMILES: CCCCOCC(C)(C)c1cc(O)c2c(c1)OC(C)(C)[C@@H]1CC=C(C(=O)NC(C)(C)C(F)(F)F)C[C@@H]21] | Compound 122 |
| 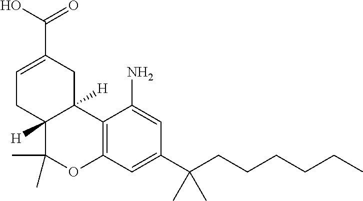 [SMILES: CCCCCCC(C)(C)c1cc(N)c2c(c1)OC(C)(C)[C@@H]1CC=C(C(=O)O)C[C@@H]21] | Compound 123 |
| 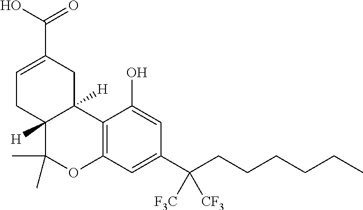 [SMILES: CCCCCCC(c1cc(O)c2c(c1)OC(C)(C)[C@@H]1CC=C(C(=O)O)C[C@@H]21)(C(F)(F)F)C(F)(F)F] | Compound 124 |
| 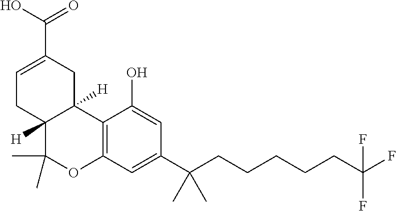 [SMILES: CC(C)(CCCCCC(F)(F)F)c1cc(O)c2c(c1)OC(C)(C)[C@@H]1CC=C(C(=O)O)C[C@@H]21] | Compound 125 |
| 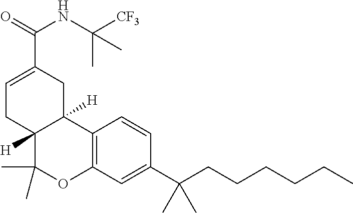 [SMILES: CCCCCCC(C)(C)c1ccc2c(c1)OC(C)(C)[C@@H]1CC=C(C(=O)NC(C)(C)C(F)(F)F)C[C@@H]21] | Compound 126 |
| 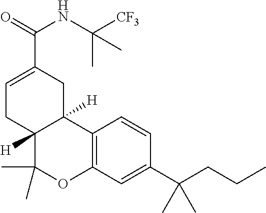 [SMILES: CCCC(C)(C)c1ccc2c(c1)OC(C)(C)[C@@H]1CC=C(C(=O)NC(C)(C)C(F)(F)F)C[C@@H]21] | Compound 127 |
| 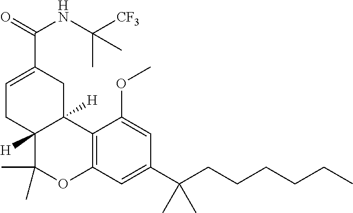 [SMILES: CCCCCCC(C)(C)c1cc(OC)c2c(c1)OC(C)(C)[C@@H]1CC=C(C(=O)NC(C)(C)C(F)(F)F)C[C@@H]21] | Compound 128 |
| 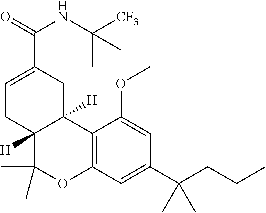 [SMILES: CCCC(C)(C)c1cc(OC)c2c(c1)OC(C)(C)[C@@H]1CC=C(C(=O)NC(C)(C)C(F)(F)F)C[C@@H]21] | Compound 129 |
| 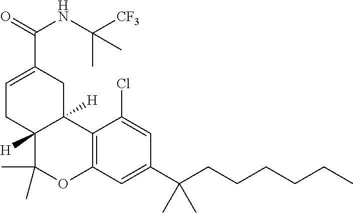 [SMILES: CCCCCCC(C)(C)c1cc(Cl)c2c(c1)OC(C)(C)[C@@H]1CC=C(C(=O)NC(C)(C)C(F)(F)F)C[C@@H]21] | Compound 130 |
| 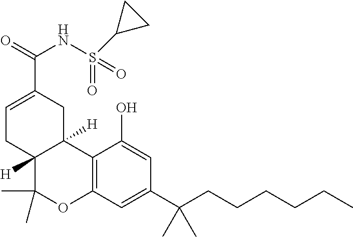 [SMILES: CCCCCCC(C)(C)c1cc(O)c2c(c1)OC(C)(C)[C@@H]1CC=C(C(=O)NS(=O)(=O)C3CC3)C[C@@H]21] | Compound 131 |
| 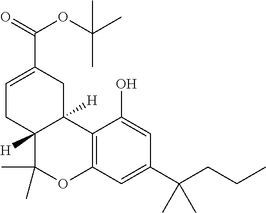 [SMILES: CCCC(C)(C)c1cc(O)c2c(c1)OC(C)(C)[C@@H]1CC=C(C(=O)OC(C)(C)C)C[C@@H]21] | Compound 132 |
| 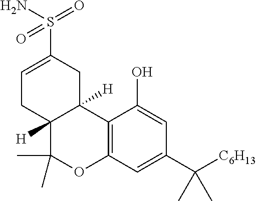 | Compound 133 |
| 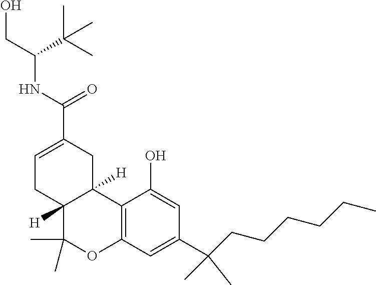 [SMILES: CCCCCCC(C)(C)c1cc(O)c2c(c1)OC(C)(C)[C@@H]1CC=C(C(=O)N[C@H](CO)C(C)(C)C)C[C@@H]21] | Compound 134 |
| 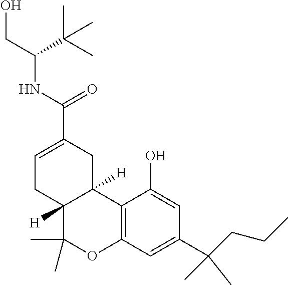 [SMILES: CCCC(C)(C)c1cc(O)c2c(c1)OC(C)(C)[C@@H]1CC=C(C(=O)N[C@H](CO)C(C)(C)C)C[C@@H]21] | Compound 135 |
| 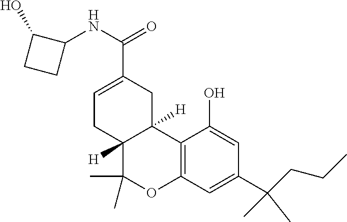 [SMILES: CCCC(C)(C)c1cc(O)c2c(c1)OC(C)(C)[C@@H]1CC=C(C(=O)NC3CC[C@@H]3O)C[C@@H]21] | Compound 136 |
| 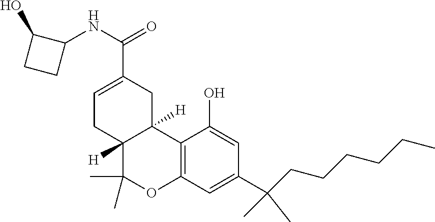 [SMILES: CCCCCCC(C)(C)c1cc(O)c2c(c1)OC(C)(C)[C@@H]1CC=C(C(=O)NC3CC[C@H]3O)C[C@@H]21] | Compound 137 |
| 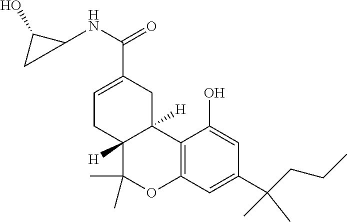 [SMILES: CCCC(C)(C)c1cc(O)c2c(c1)OC(C)(C)[C@@H]1CC=C(C(=O)NC3C[C@@H]3O)C[C@@H]21] | Compound 138 |
| 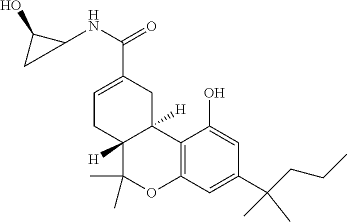 [SMILES: CCCC(C)(C)c1cc(O)c2c(c1)OC(C)(C)[C@@H]1CC=C(C(=O)NC3C[C@H]3O)C[C@@H]21] | Compound 139 |
| 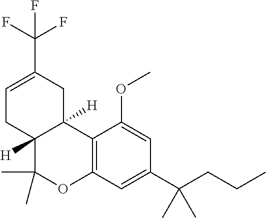 [SMILES: CCCC(C)(C)c1cc(OC)c2c(c1)OC(C)(C)[C@@H]1CC=C(C(F)(F)F)C[C@@H]21] | Compound 140 |
| 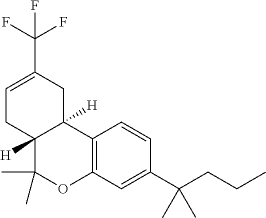 [SMILES: CCCC(C)(C)c1ccc2c(c1)OC(C)(C)[C@@H]1CC=C(C(F)(F)F)C[C@@H]21] | Compound 141 |
| 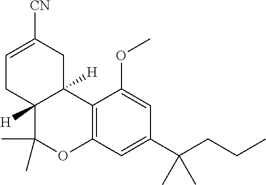 [SMILES: CCCC(C)(C)c1cc(OC)c2c(c1)OC(C)(C)[C@@H]1CC=C(C#N)C[C@@H]21] | Compound 142 |
| 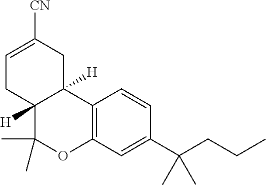 [SMILES: CCCC(C)(C)c1ccc2c(c1)OC(C)(C)[C@@H]1CC=C(C#N)C[C@@H]21] | Compound 143 |
| 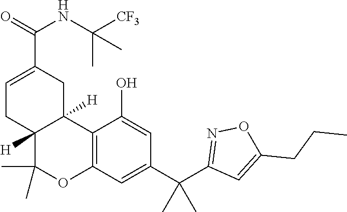 [SMILES: CCCc1cc(C(C)(C)c2cc(O)c3c(c2)OC(C)(C)[C@@H]2CC=C(C(=O)NC(C)(C)C(F)(F)F)C[C@@H]32)no1] | Compound 144 |

In another aspect, the invention provides a pharmaceutical composition including a compound of the invention (e.g., a compound of any one of formulas (I)-(VIII) or any one of compounds 1-144), or a salt thereof, and a pharmaceutically acceptable excipient.

In another aspect, the invention provides a method of treating an inflammatory disease in a subject in need thereof. The method includes administering to the subject a pharmaceutical composition including a compound of the invention (e.g., a compound of any one of formulas (I)-(VIII) or any one of compounds 1-144), or a salt thereof, and a pharmaceutically acceptable excipient, in an amount sufficient to treat the condition.

In some embodiments, the inflammatory disease is selected from the group consisting of dermatomyositis, systemic lupus erythematosus, acquired immune deficiency syndrome (AIDS), multiple sclerosis, rheumatoid arthritis, psoriasis, diabetes, cancer, asthma, atopic dermatitis, an autoimmune thyroid disorder, ulcerative colitis, Crohn's disease, stroke, ischemia, a neurodegenerative disease (e.g., Alzheimer's disease and Parkinson's disease), amyotrophic lateral sclerosis (ALS), chronic traumatic encephalopathy (CTE), chronic inflammatory demyelinating polyneuropathy, an autoimmune inner ear disease, uveitis, iritis, and peritonitis. In another aspect, the invention provides a method of treating a fibrotic disease in a subject in need thereof. The method includes administering to the subject the pharmaceutical composition including a compound of the invention (e.g., a compound of any one of formulas (I)-(VIII) or any one of compounds 1-144), or a salt thereof, and a pharmaceutically acceptable excipient, in an amount sufficient to treat the condition.

In some embodiments, the fibrotic disease is selected from the group consisting of cystic fibrosis, scleroderma (e.g., systemic sclerosis, localized scleroderma, or sine scleroderma), liver cirrhosis, interstitial pulmonary fibrosis, idiopathic pulmonary fibrosis, Dupuytren's contracture, keloids, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, and organ fibrosis (e.g., dermal fibrosis, lung fibrosis, liver fibrosis, kidney fibrosis, or heart fibrosis).

In some embodiments, the compound has increased affinity for the CB₂ receptor compared to affinity for the CB₁ receptor. In some embodiments, the compound has 10%, 20%, 30% 40%, 50%, 60% 70%, 80%, 90%, 100%, 200%, 300%, 400%, 500%, 600%, 700%, 800%, 900%, or 1000% or more greater affinity for the CB₂ receptor compared to the CB₁ receptor. In some embodiments, the compound has 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, or 50-fold or more greater affinity for the CB₂ receptor compared to the CB₁ receptor. In some embodiments, the compound has greater CB₂ receptor selectivity compared to the CB₂ receptor selectivity of ajulemic acid.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “treat” or “treatment” includes administration of a compound to a subject, e.g., by any route, e.g., orally, topically, by inhalation, by ex-vivo contact with one or more cells of the subject. The compound can be administered alone or in combination with one or more additional compounds. Treatments may be sequential, with the present compound being administered before or after the administration of other agents. Alternatively, compounds may be administered concurrently.

The subject, e.g., a patient, can be one having a disorder (e.g., a disease or condition described herein), a symptom of a disorder, or a predisposition toward a disorder. Treatment is not limited to curing or complete healing, but can result in one or more of alleviating, relieving, altering, partially remedying, ameliorating, improving or affecting the disorder, reducing one or more symptoms of the disorder or the predisposition toward the disorder. In an embodiment the treatment (at least partially) alleviates or relieves symptoms related to a fibrotic disease. In an embodiment the treatment (at least partially) alleviates or relieves symptoms related to an inflammatory disease. In one embodiment, the treatment reduces at least one symptom of the disorder or delays onset of at least one symptom of the disorder.

The effect is beyond what is seen in the absence of treatment.

The terms “therapeutically effective amount” or “amount sufficient to treat” as used interchangeably herein, refer to an amount, e.g., pharmaceutical dose, effective in inducing a desired effect in a subject or in treating a subject having a condition or disorder described herein (e.g., a fibrotic disease of an inflammatory disease). It is also to be understood herein that a “therapeutically effective amount” may be interpreted as an amount giving a desired therapeutic and/or preventative effect, taken in one or more doses or in any dosage or route, and/or taken alone or in combination with other therapeutic agents.

The term “subject,” as used herein, can be a human, non-human primate, or other mammal, such as but not limited to dog, cat, horse, cow, pig, turkey, goat, fish, monkey, chicken, rat, mouse, and sheep.

The term “pharmaceutical composition” refers to the combination of an active agent with an excipient, inert or active, making the composition especially suitable for diagnostic or therapeutic use in vivo or ex vivo. A “pharmaceutically acceptable excipient,” after being administered to or upon a subject, does not cause undesirable physiological effects. The excipient in the pharmaceutical composition must be “acceptable” also in the sense that it is compatible with the active ingredient. The excipient may also be capable of stabilizing the active ingredient. One or more solubilizing agents can be utilized as pharmaceutical excipients for delivery of an active compound. Examples of pharmaceutically acceptable excipients include, but are not limited to, biocompatible vehicles, adjuvants, additives, and diluents to achieve a composition usable as a dosage form. Examples of other excipients include colloidal silicon oxide, magnesium stearate, cellulose, and sodium lauryl sulfate.

As used herein, the term “carrier” refers to a diluent, adjuvant, excipient, or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, sodium stearate, glycerol monostearate, talc, sodium chloride, glycerol, propylene glycol, water, and ethanol. The present compositions, if desired, can also contain minor amounts of wetting or emulsifying agents, or pH buffering agents.

As used herein, the term “pharmaceutically acceptable salt” represents a salt of a compound of the invention (e.g., a compound of any one of formulas (I)-(VIII) or any one of compounds 1-144) that is within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of a compound described herein or separately by reacting the free base group with a suitable organic acid.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C2-C20, C2-C18, C2 C16, C2-C14, C2-C12, C2-C10, C2-C8, C2-C6, or C2-C4). Examples include, but are not limited to, methyl, ethyl, isobutyl, sec-butyl, tert-butyl, 2-propenyl, and 3-butynyl.

The term “alkoxy,” as used herein, represents a chemical substituent of formula —OR, where R is a C₁₋₂₀ alkyl group (e.g., C₁₋₆ or C₁₋₁₀ alkyl), unless otherwise specified. Exemplary alkoxy groups include methoxy, ethoxy, propoxy (e.g., n-propoxy and isopropoxy), t-butoxy, and the like. In some embodiments, the alkyl group can be further substituted with 1, 2, 3, or 4 substituent groups as defined herein (e.g., hydroxy or alkoxy).

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C5-C6, C5-C7, C5-C8, C5-C9, C5-C10, C5-C11, C5-C12, C5-C13, C5-C14, or C5-C15 aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroaryl). The inclusion of a heteroatom permits inclusion of 5 membered rings to be considered aromatic as well as 6 membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are possible, a group such as phthalimido is also considered heteroaryl.

In some embodiments, the aryl or heteroaryl group is a 5- or 6-membered aromatic ring system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “heterocyclyl,” as used herein represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined herein.

The term “cycloalkyl,” as used herein, represents a monovalent saturated or unsaturated nonaromatic cyclic alkyl group. A cycloalkyl may have, e.g., three to twenty carbons (e.g., a C3-C7, C3-C8, C3-C9, C3-C10, C3-C11, C3-C12, C3-C14, C3-C16, C3-C18, or C3-C20 cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C4-C7, C4-C8, C4-C9, C4-C10, C4-C11, C4-C12, C4-C14, C4-C16, C4-C18, or C4-C20 cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C8-C9, C8-C10, C8-C11, C8-C12, C8-C14, C8-C16, C8-C18, or C8-C20 cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantyl. The term “cycloalkyl” also includes bicyclic, tricyclic, and tetracyclic fused ring structures, e.g., decalin and spiro-cyclic compounds.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C6-C35 alkaryl (e.g., C6-C16, C6-C14, C6-C12, C6-C10, C6-C9, C6-C8, C7, or C6 alkaryl), in which the number of carbons indicates the total number of carbons in both the aryl portion and the alkylene, alkenylene, or alkynylene portion of the alkaryl. Examples of alkaryls include, but are not limited to, (C1-C8)alkylene(C6-C12)aryl, (C2-C8)alkenylene(C6-C12)aryl, or (C2 C8)alkynylene(C6-C12)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “carboxyl,” as used herein, represents a —COOH group. An optionally substituted carboxyl includes, for example, a —COOR group, wherein R is H or any substituent group described herein.

The term “amine,” as used herein, represents an —NH₂ group. An optionally substituted amine includes, for example, a —NHR or a —NR₁R₂ group, wherein R, R₁, and R₂ are each independently H or any substituent group described herein. In some embodiments, R₁ and R₂ form cyclic ring (e.g., a 5- or 6-membered ring), such that —NR₁R₂ is an optionally substituted heterocycle or heteroaryl.

The term “amide,” as used herein, represents a —C(═O)NH₂ group. An optionally substituted amide includes, for example, a —C(═O)NHR or a —C(═O)NR₁R₂ group, wherein R, R₁, and R₂ are each independently H or any substituent group described herein.

The term “imino” as used herein, represents a —C(═NR₁)R₂ group. An optionally substituted imino includes, for example, a —C(═NR₁)R₂ group, wherein each of R₁ and R₂ are independently selected from H or any substituted group described herein.

The term “thioester,” as used herein, represents a —C(═O)SH group. An optionally substituted thioester includes, for example, a —C(═O)SR group, wherein R is H or any substituent group described herein.

The term “thioamide,” as used herein, represents a —C(═S)NH₂ group. An optionally substituted thioamide includes, for example, a —C(═S)NHR or a —C(═S)NR₁R₂ group, wherein R, R₁, and R₂ are each independently H or any substituent group described herein.

The term “sulfonamide,” as used herein, represents a —S(═O)₂NH₂ group. An optionally substituted sulfonamide includes, for example, a —S(═O)₂NHR or a —S(═O)₂NR₁R₂ group, wherein R, R₁, and R₂ are each independently H or any substituent group described herein.

The term “sulfonyl,” as used herein, represents a —S(═O)₂R group. An optionally substituted sulfonyl includes, for example, a S(═O)₂R, wherein R is an H or any substituent group described herein.

The term “cyano,” as used herein, represents a —CN group.

The term “hydroxyl,” as used herein, represents an —OH group.

The term “oxo,” as used herein, refers to a substituent having the structure ═O, where there is a double bond between an atom and an oxygen atom.

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, Br, methyl, ethyl, propyl, butyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members. An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N.

### DETAILED DESCRIPTION OF THE INVENTION

The invention relates to cannabinoid compounds, pharmaceutical compositions including one or more cannabinoid compounds, and the use of pharmaceutical compositions including one or more cannabinoid compounds for the treatment of a disease or condition (e.g., a fibrotic disease or an inflammatory disease) in a subject in need thereof.

### Compounds

The disclosure provides compounds (e.g., a cannabinoid compound, a compound described by any one of formulas (I)-(VIII), or any one of compounds 1-144) useful for the treatment of disease (e.g., a fibrotic disease or an inflammatory disease).

In particular, the invention features compounds sharing structural features with (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid). In some embodiments, the invention features compounds which are agonists of the CB₂ receptor. In preferred embodiments of the invention, the invention features compounds that have increased affinity for the CB₂ receptor (e.g., increased affinity for the CB₂ receptor compared to ajulemic acid), increased selectivity for the CB₂ receptor (e.g., increased selectivity for the CB₂ receptor over the CB₁ receptor compared to ajulemic acid), or both increased affinity and increased selectivity for the CB₂ receptor.

In some embodiments, the invention features compounds with an increased safety or efficacy profile in the treatment of a disease or condition (e.g., a fibrotic disease or an inflammatory disease), as compared to other cannabinoids, such as ajulemic acid. In some embodiments, administration of a compound of the invention to a subject (e.g., a subject having a disease or condition described herein) results in a decrease in treatment-associated adverse events relative to treatment with one or more other cannabinoids (e.g., treatment with an equivalent dose and method of administration of ajulemic acid). In some embodiments, administration of a compound of the invention to a subject (e.g., a subject having a disease or condition described herein) results in a decrease in CB₁-associated adverse events relative to treatment with one or more other cannabinoids (e.g., ajulemic acid). In some embodiments, administration of a compound of the invention to a subject (e.g., a subject having a disease or condition described herein) results in a decrease in the rate of occurrence, severity, or risk of one or more of the following adverse events: dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation. The reduction in adverse events may be a reduction of 10%, 20%, 30%, 40%, 50%, 60%, 70%, 80%, 90%, 95% or more in the occurrence or severity of any one of the above-described adverse events (e.g., compared to a subject or subjects treated with an equivalent dose and method of administration of another cannabinoid, such as ajuelmic acid).

In some embodiments, the invention features compounds having improved pharmacokinetic properties or improved stability (e.g., improved pharmacokinetic properties or improved stability as compared to ajulemic acid).

In some embodiments, a compound of the invention is an ethanolamide or an ethanolamine derivative of ajulemic acid. The carboxy group of ajulemic acid may be substituted with an ethanolamide (e.g., Compound 5) or an ethanolamine (e.g., Compound 71), or an ethanolamide or ethanolamine derivative. In some embodiments, the ethanolamide or ethanolamine derivative is not cleaved by fatty acid amide hydrolase (FAAH). Exemplary ethanolamide and ethanolamine derivatives, in particular ethanolamide and ethanolamine derivatives not cleavable by FAAH, are known to those of skill in the art; see for example: Woodward D F et al. Prostamide (prostaglandin-ethanolamides) and their pharmacology. Bristish Journal of Pharmacology. 153:410-419 (2008); Woodward D F et al. Recent progress in prostaglandin F2a ethanolamide (prostamide F2a) research and therapeutics. Pharmacological Reviews. 65:1135-1147 (2013); and Gilmore J L et al. Discovery and structure-activity relationship (SAR) of a series of ethanolamine-based direct-acting agonists of sphingosine-1-phosphate (S1P1). Journal of Medicinal Chemistry. 59:6248-6264 (2016).

In some embodiments, a compound of the invention is described by any one of formulas (I)-(VIII) (e.g., a compound described by any one of formulas I-1, III-2, III-3, III-4, II-5, II-6, II-7, II-8, III-1, III-2, III-3, III-4, III-5, III-6, III-7, III-8, IV-1, IV-2, IV-3, IV-4, IV-5, IV-6, IV-7, IV-8, V-1, V-2, V-3, V-4, V-5, V-6, V-7, V-8, VI-1, VI-2, VI-3, VI-4, VI-5, VI-6, VI-7, VI-8, VII-1, VII-2, VII-3, VII-4, VII-5, VII-6, VII-7, VII-8, VIII-1, VIII-2, VIII-3, VIII-4, VIII-5, VIII-6, VIII-7, or VIII-8), wherein the compound is not ajulemic acid (AJA):

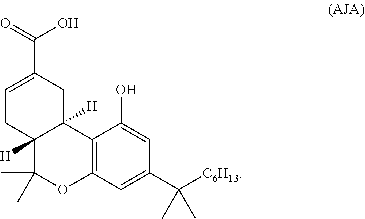

In some embodiments, the compound of the invention is a compound of Table 1 (e.g., a compound selected from any one of compounds 1-144 of Table 1):

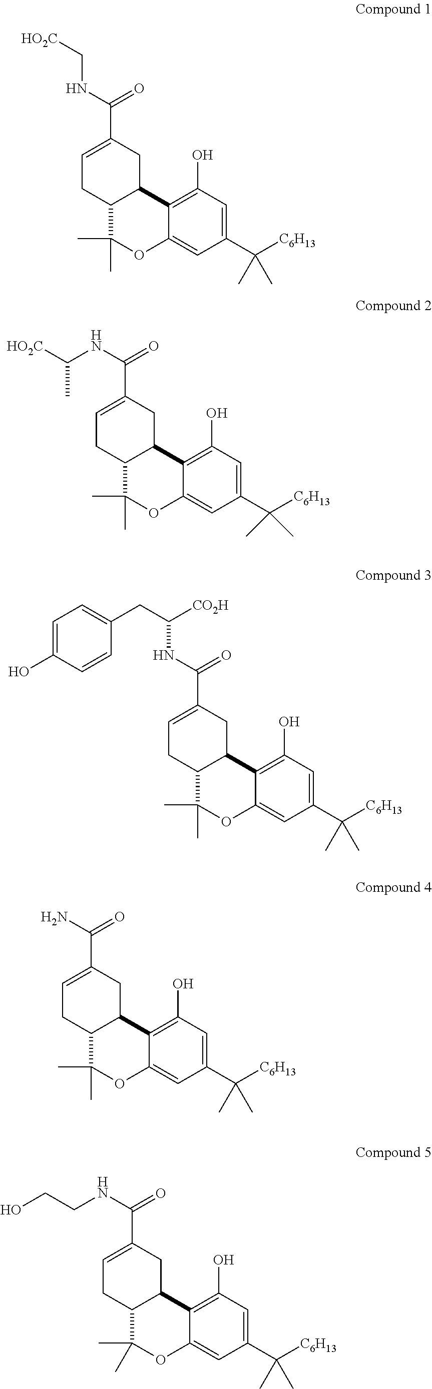

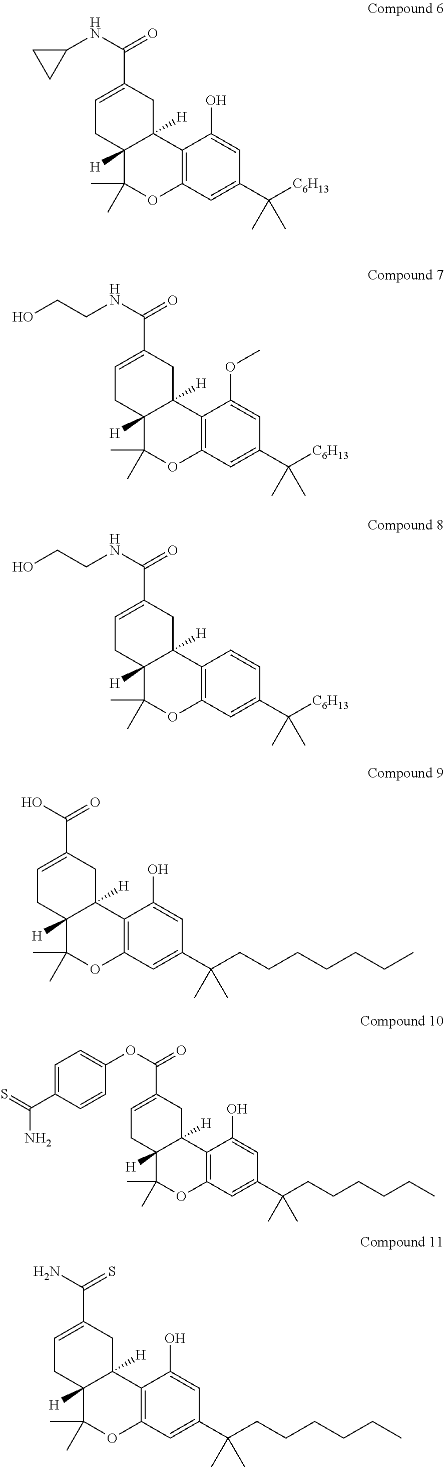

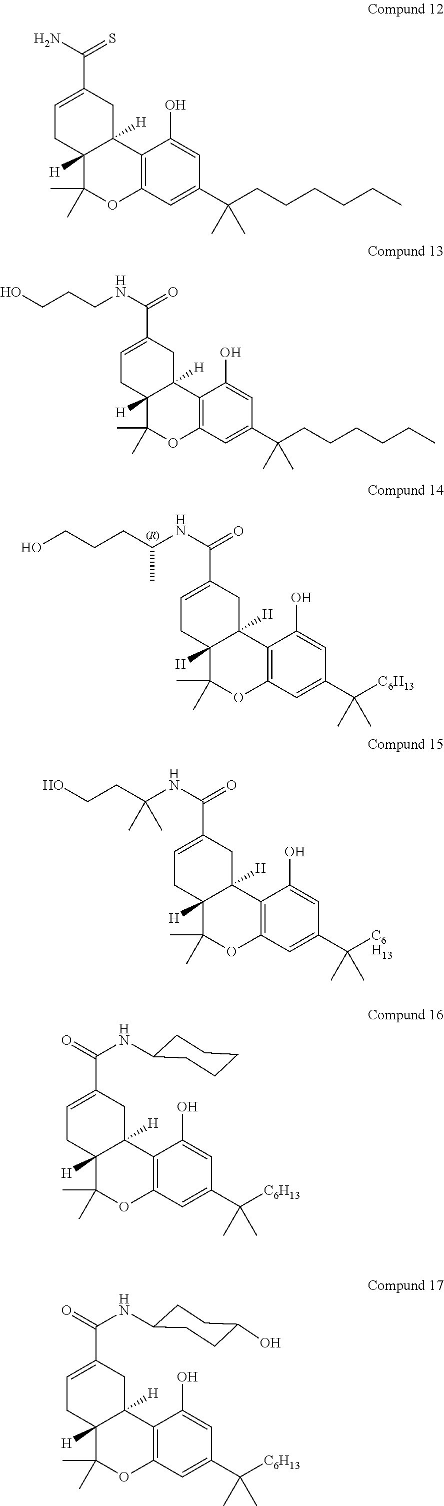

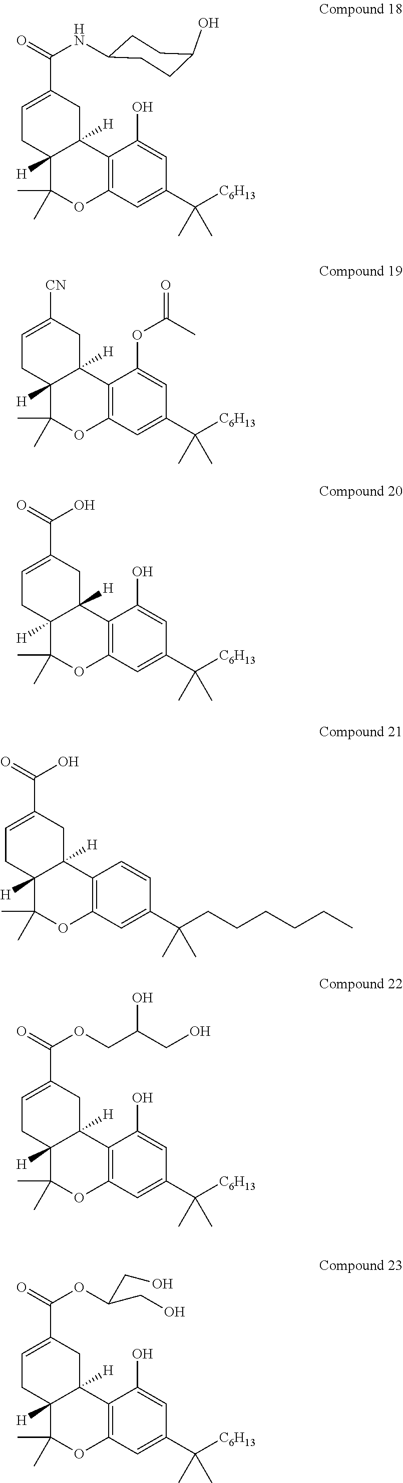

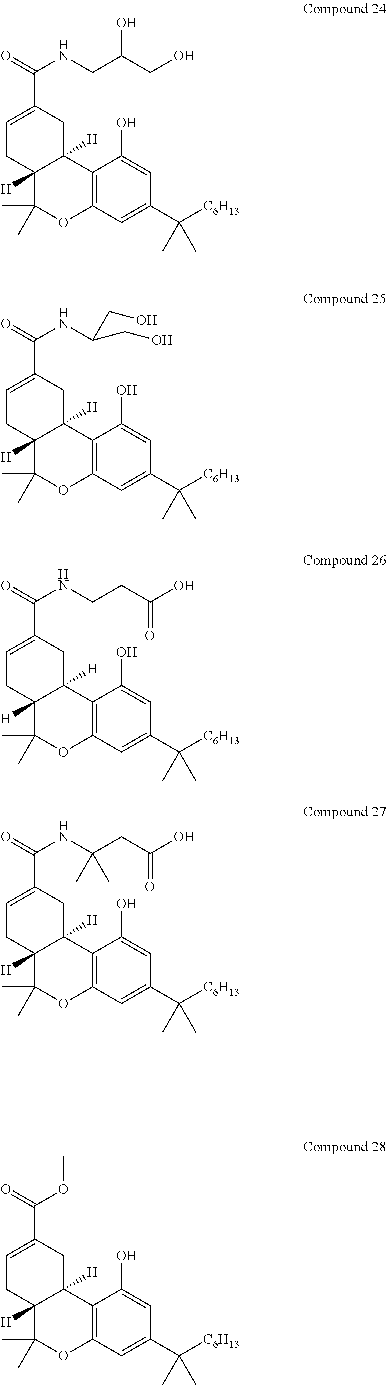

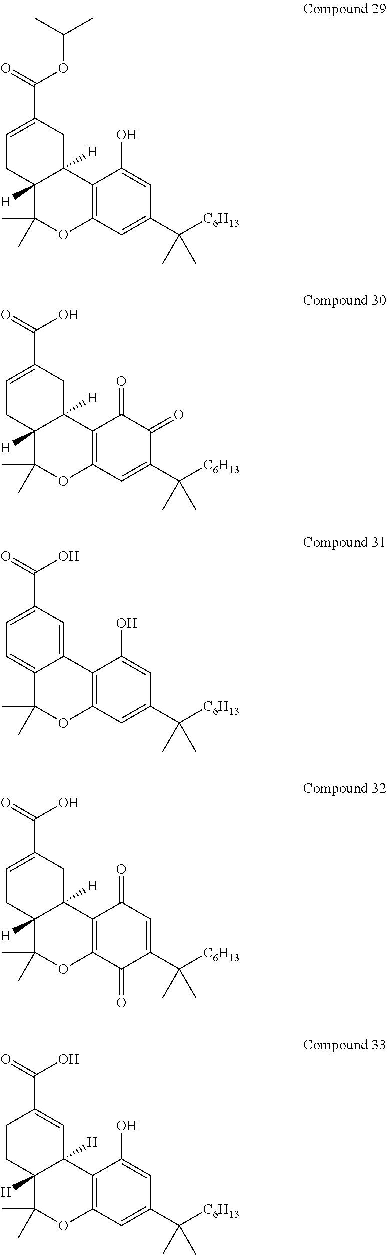

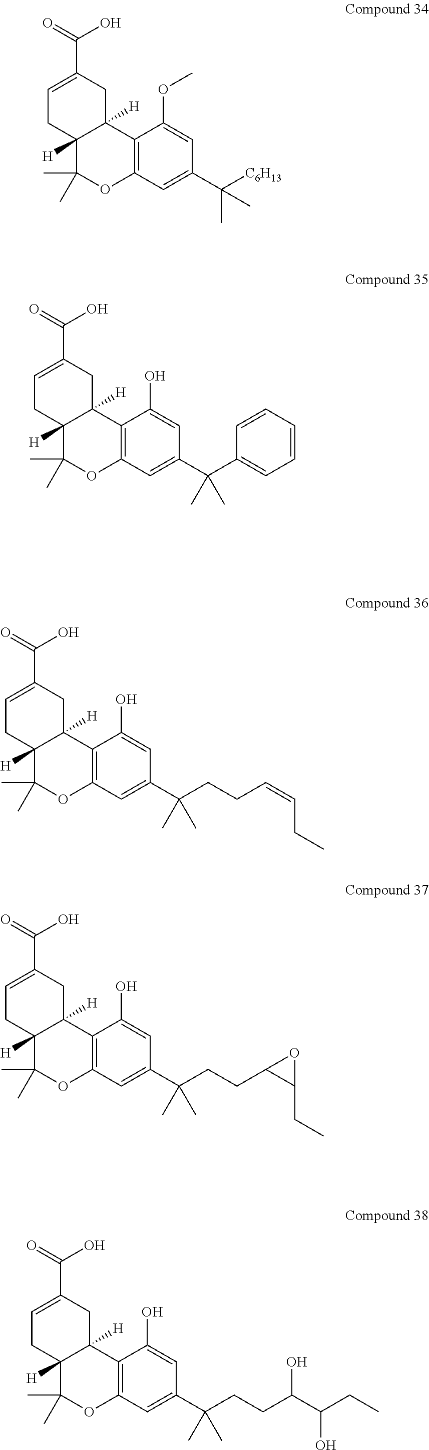

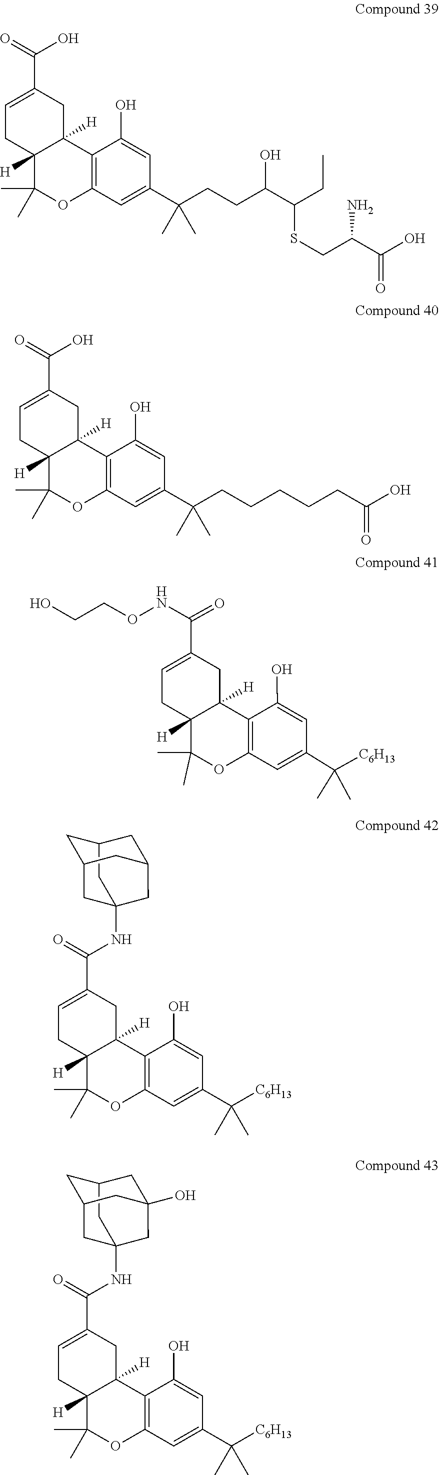

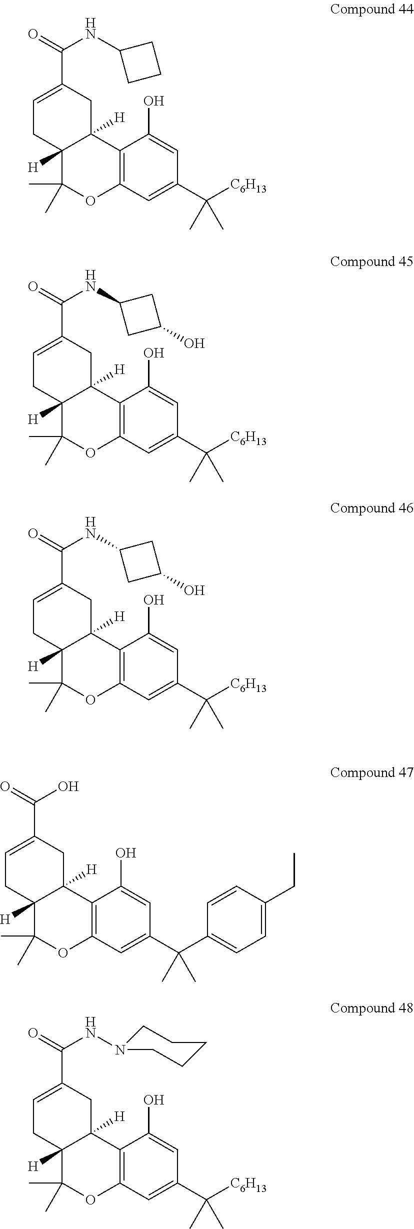

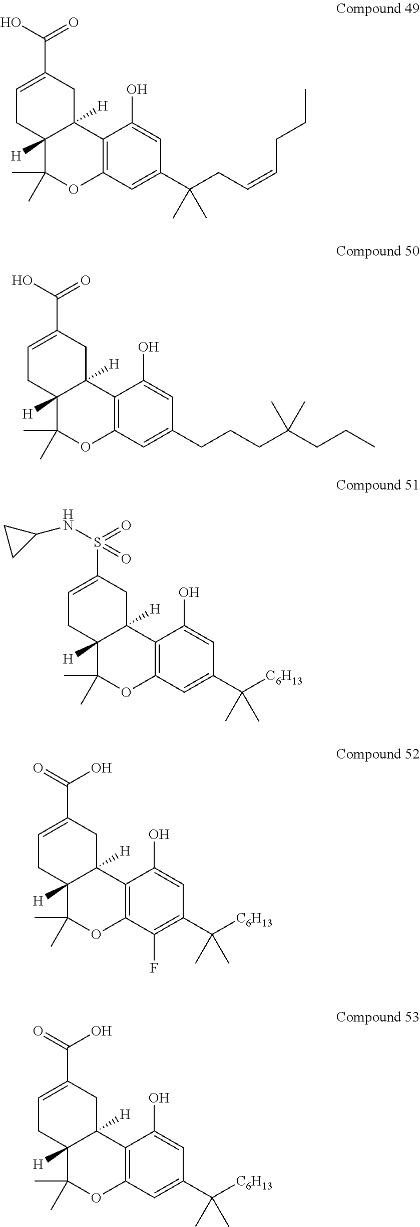

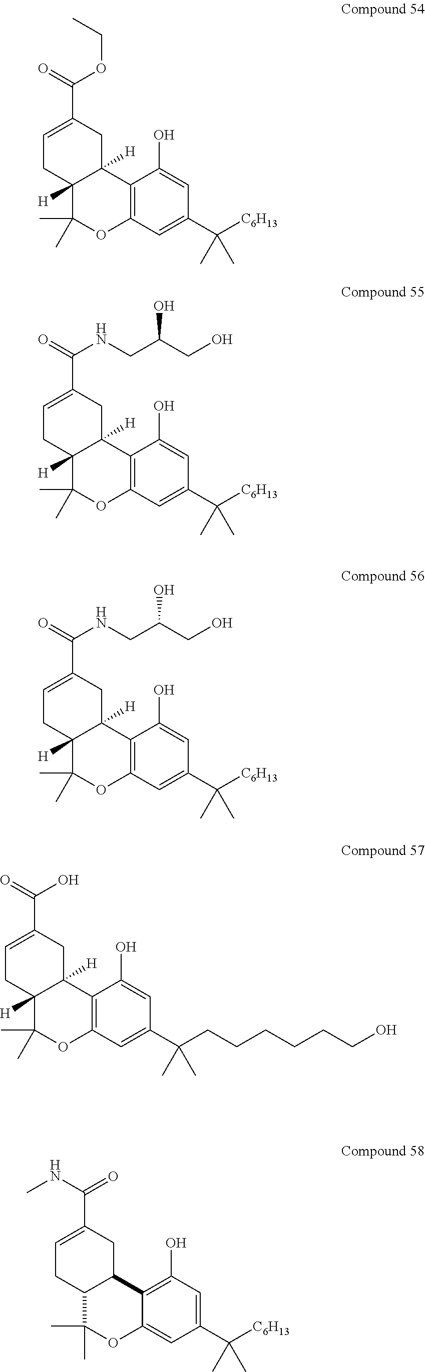

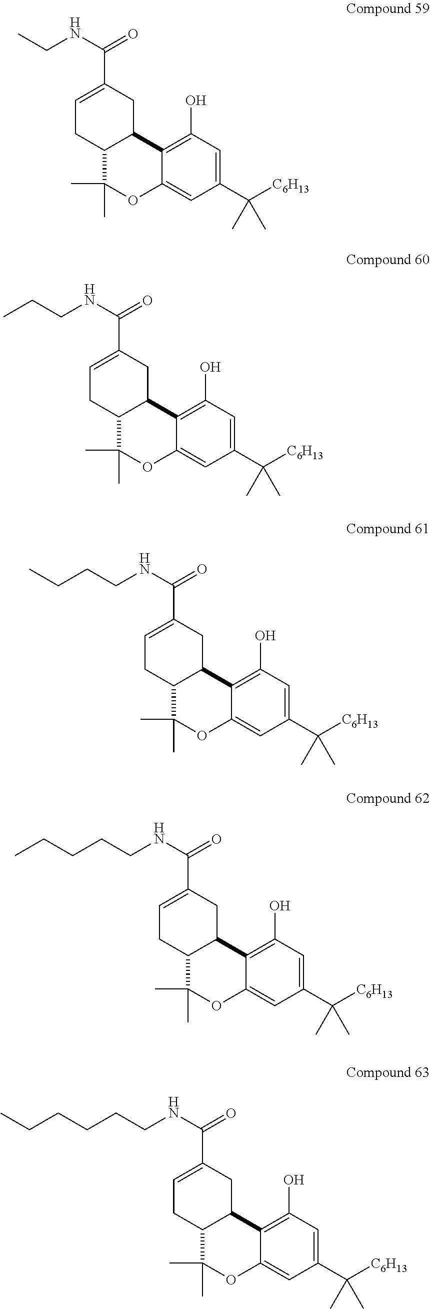

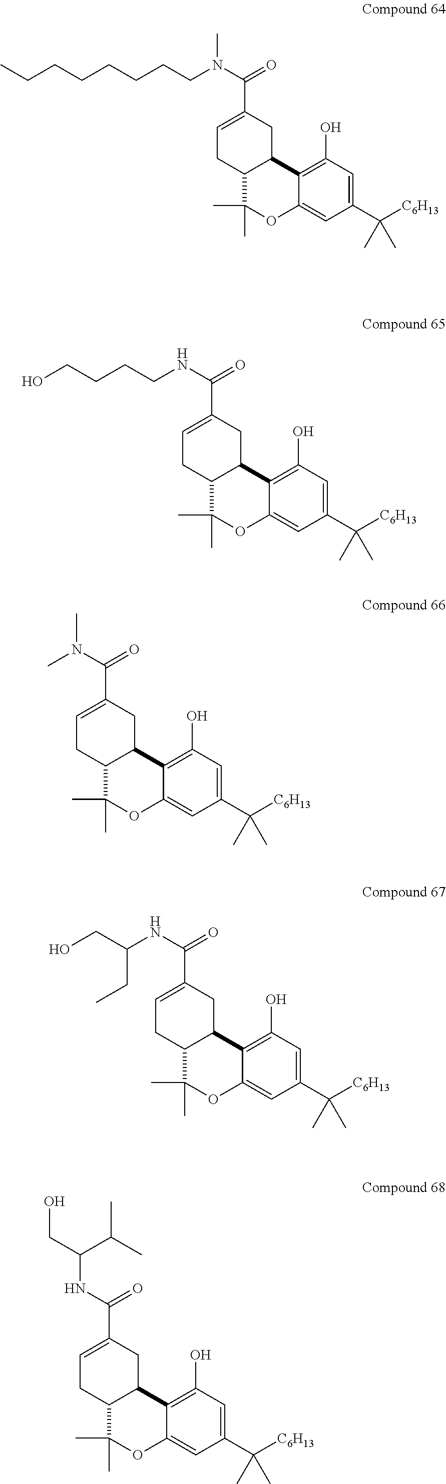

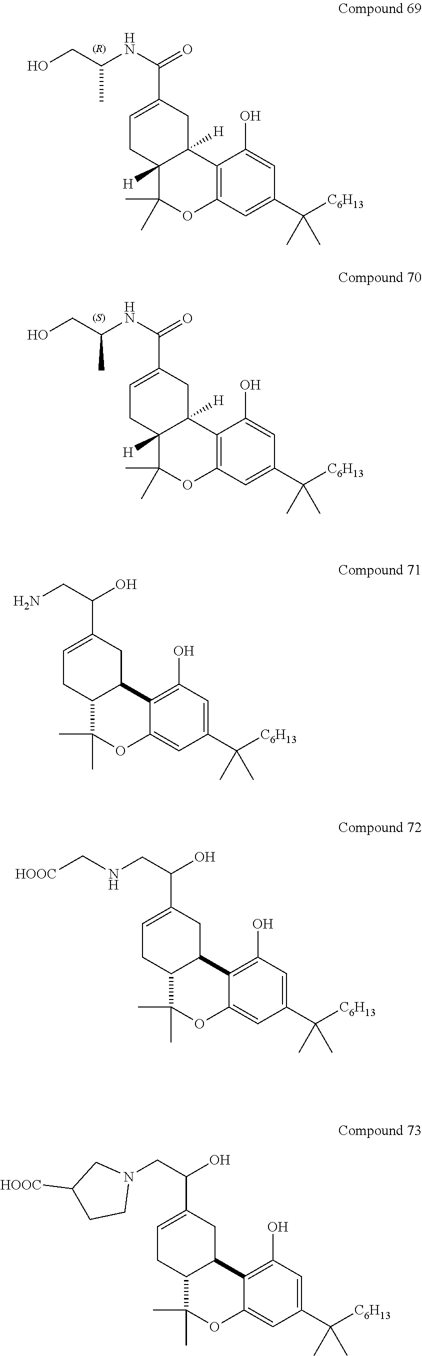

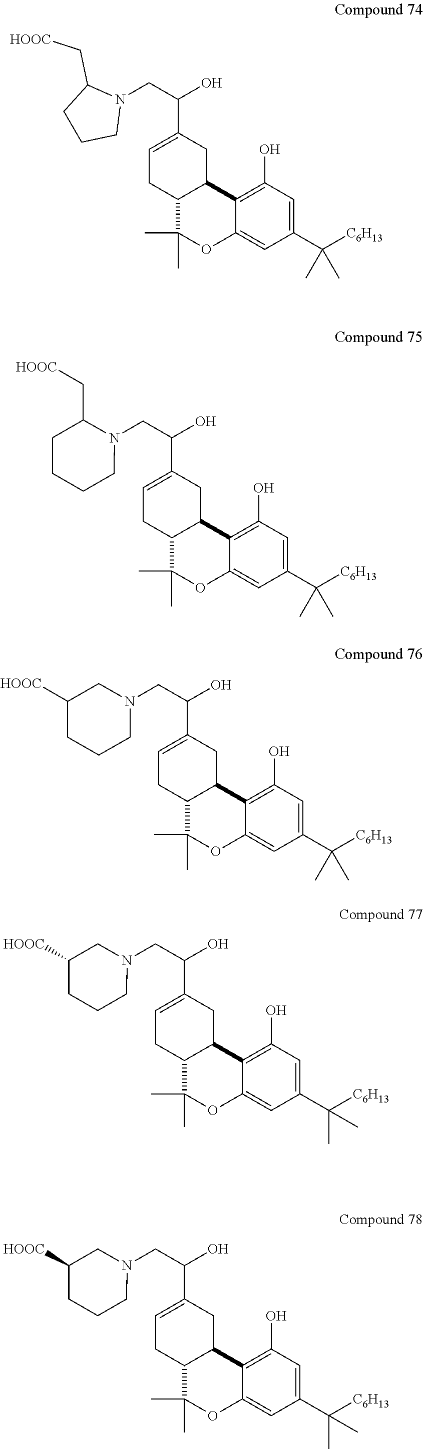

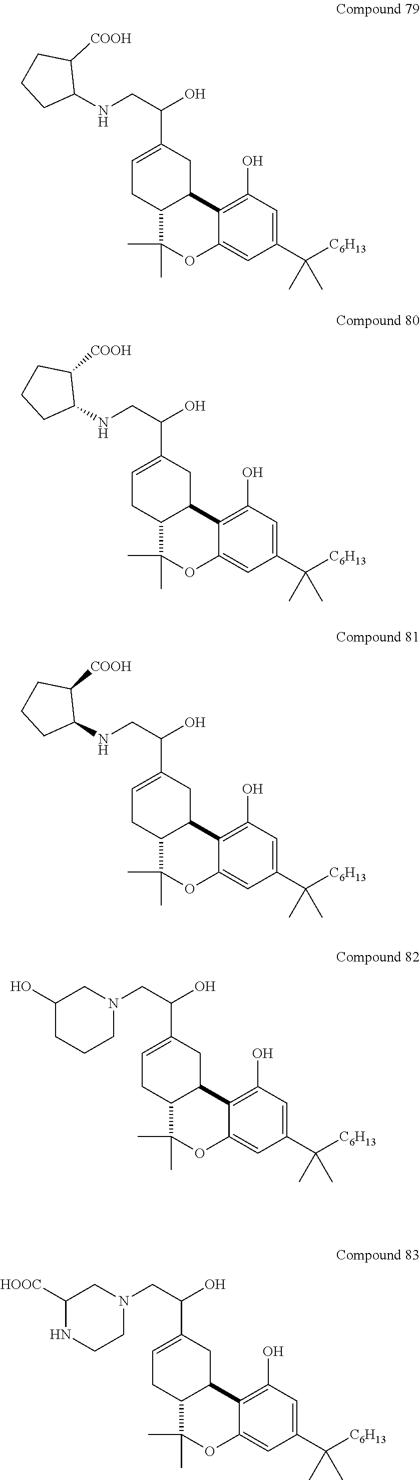

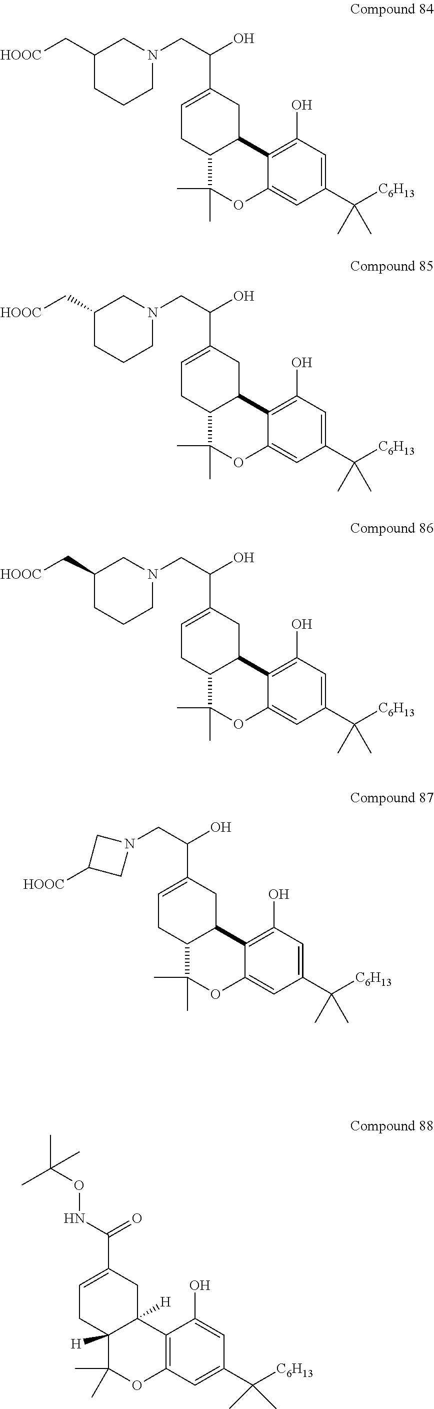

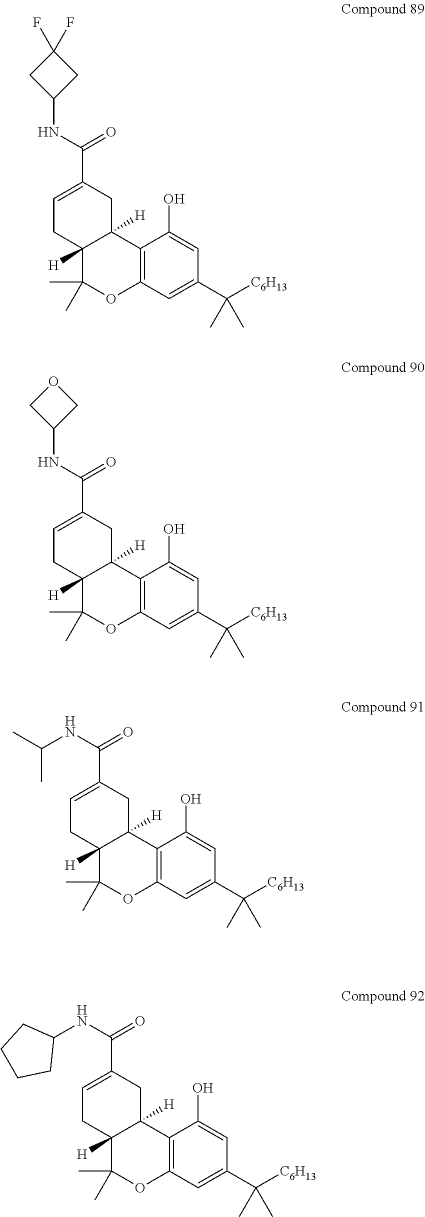

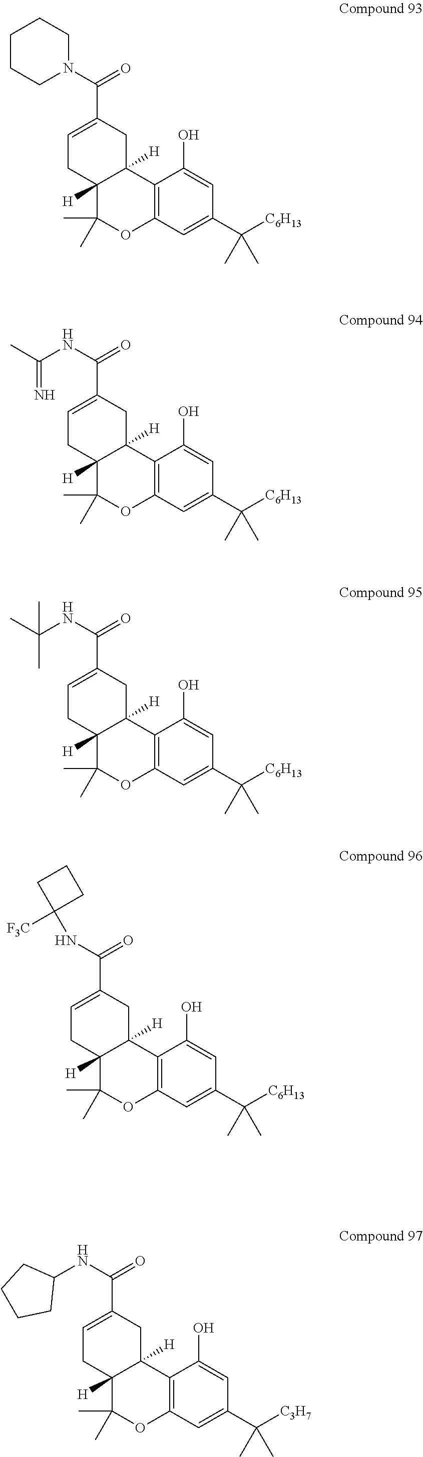

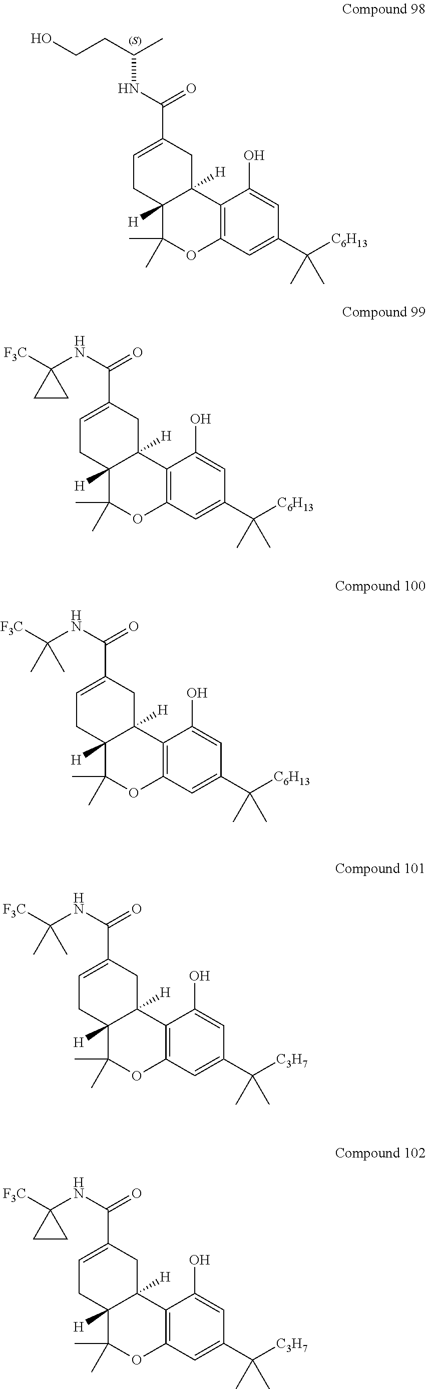

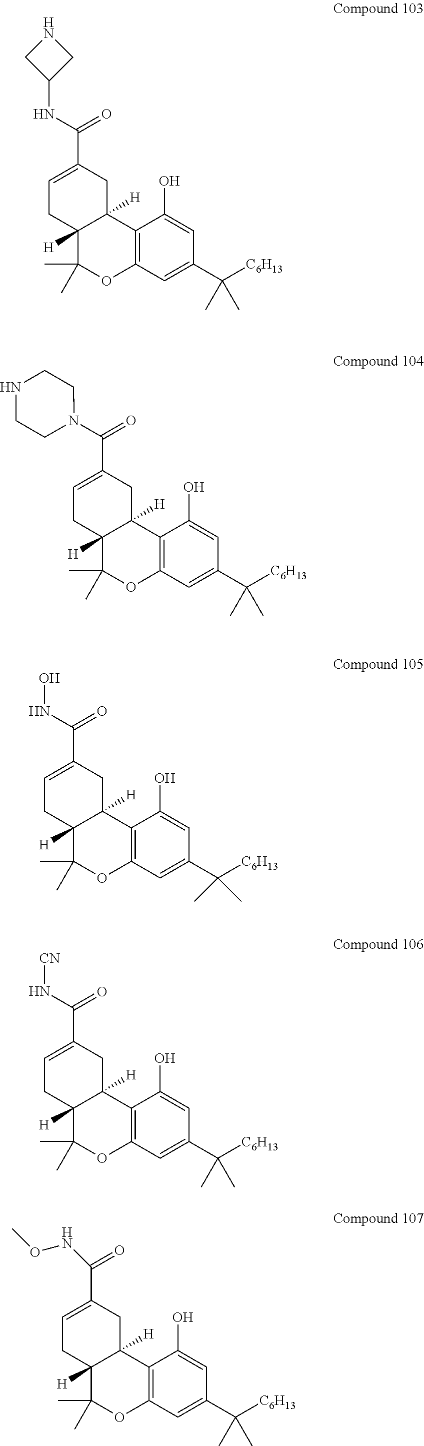

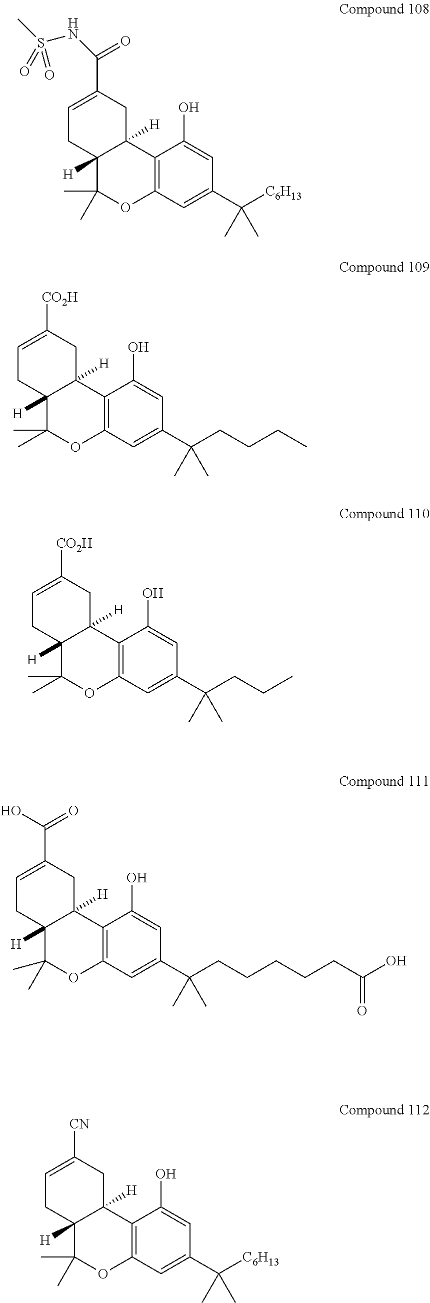

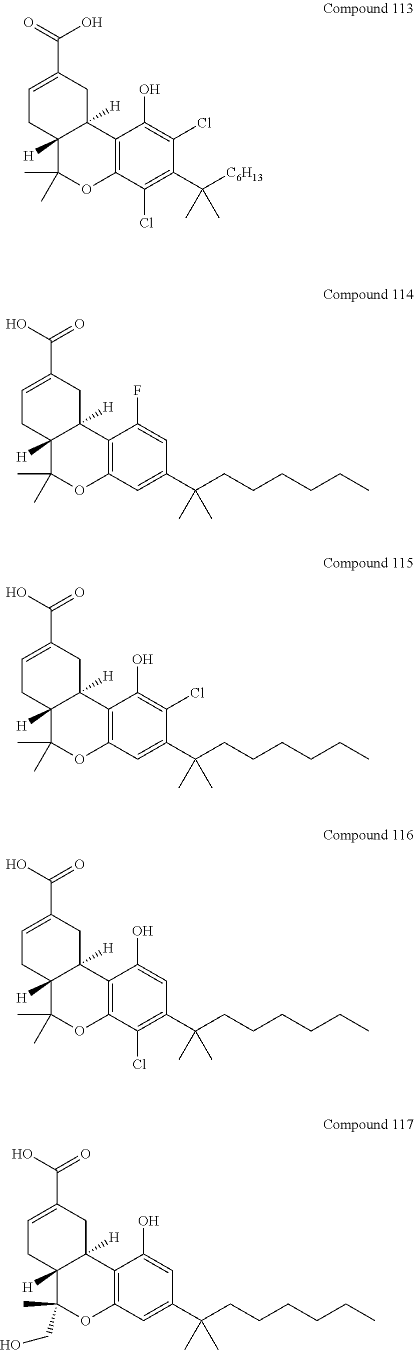

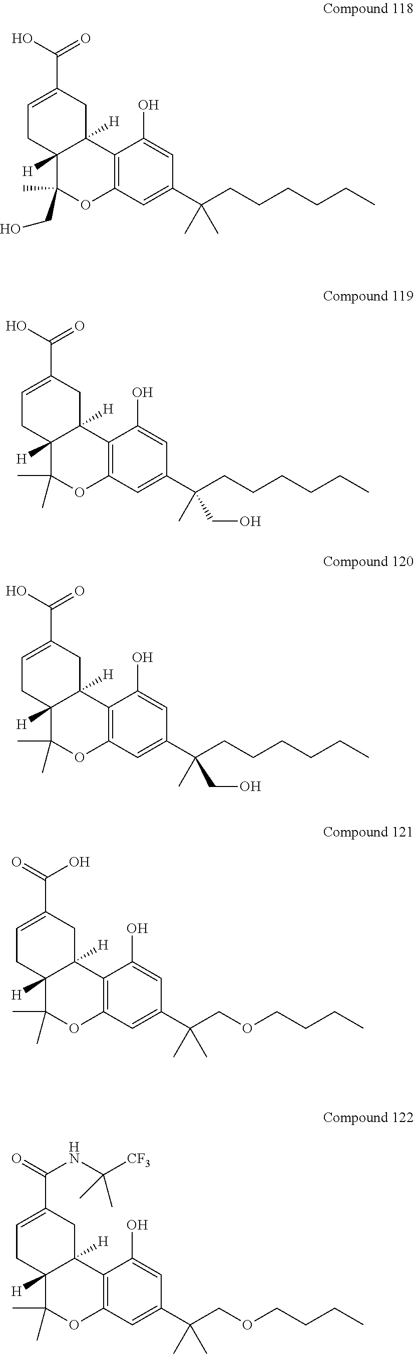

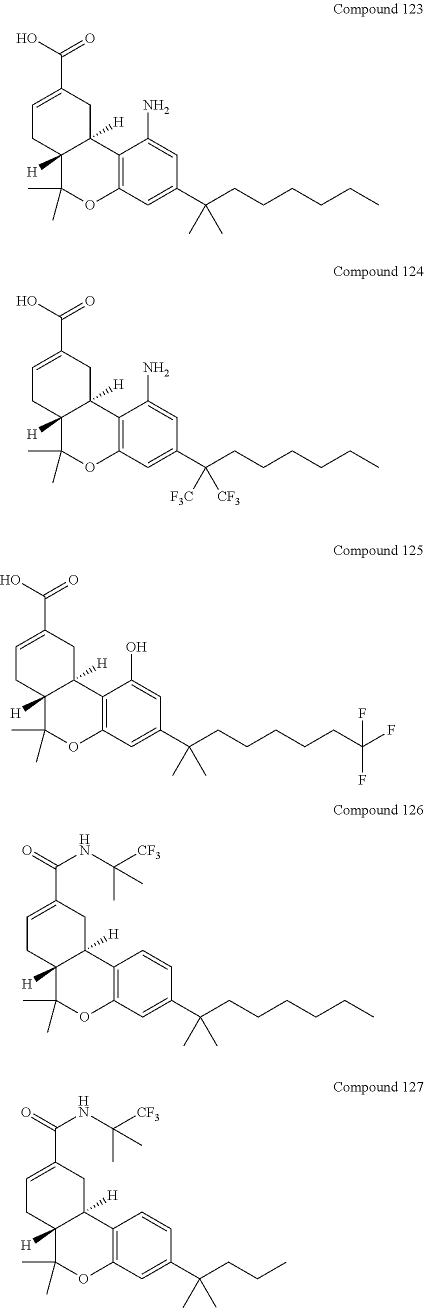

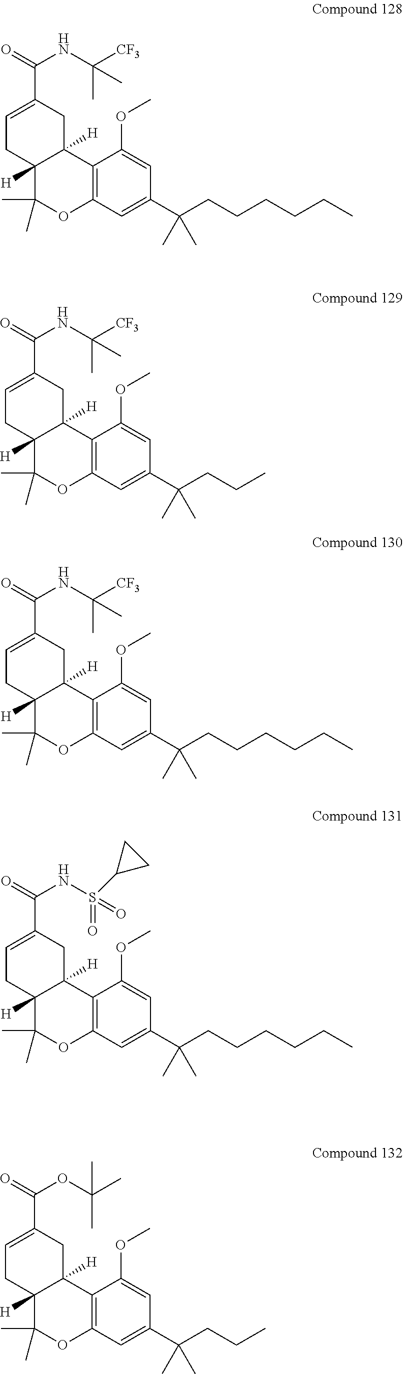

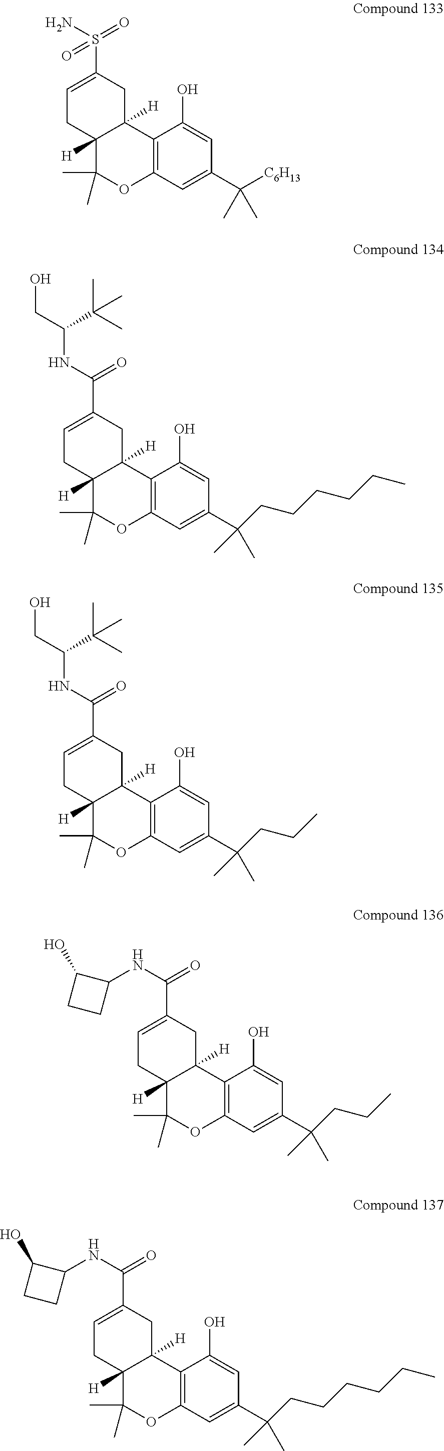

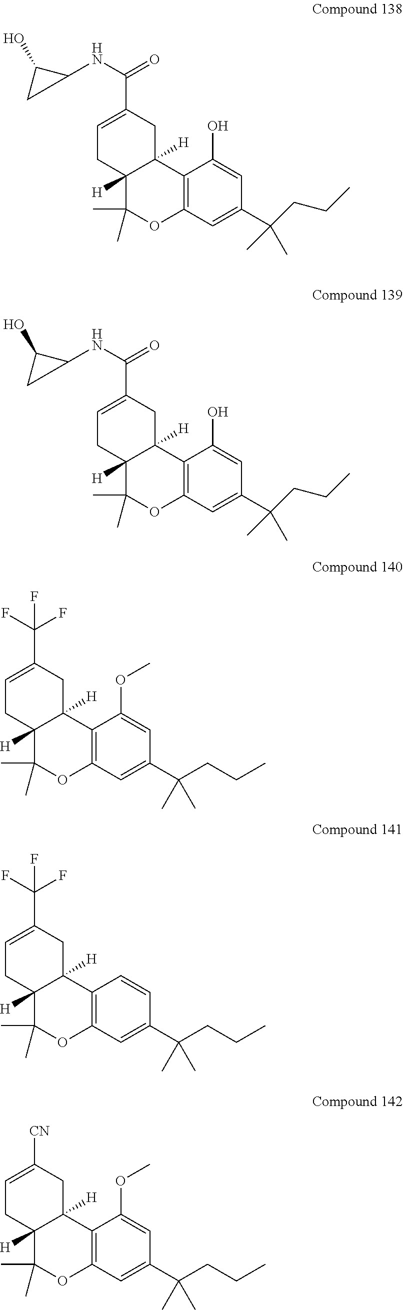

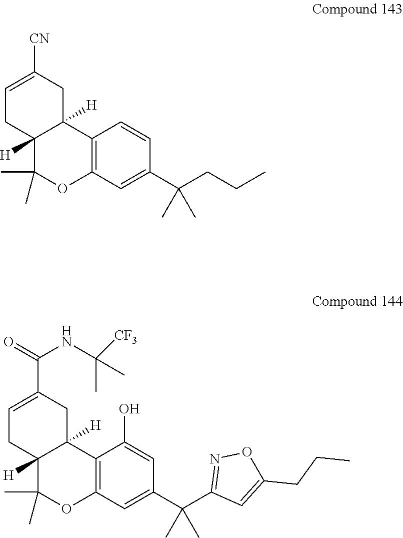

### Pharmaceutical Compositions

Compounds of the invention (e.g., a cannabinoid compound, a compound described by any one of formulas (I)-(VIII), or any one of compounds 1-144) prepared by any of the methods described herein may be formulated as a pharmaceutical composition for the treatment of disease. As described above, the pharmaceutical compositions of the invention additionally include a pharmaceutically acceptable excipient, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The above-described compositions (e.g., compositions including a cannabinoid compound, a compound described by any one of formulas (I)-(VIII), or any one of compounds 1-144), in any of the forms described herein, can be used for treating fibrotic disease, inflammatory disease, or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, buccally, by ophthalmic administration, or by inhalation. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acids, such as, but not limited to, oleic acid and its glyceride derivatives, are useful in the preparation of injectables, as are natural pharmaceutically acceptable oils, such as, but not limited to, olive oil or castor oil, or polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets (e.g. a pressed tablet), emulsions and aqueous suspensions, dispersions, and solutions. In the case of tablets, commonly used excipients include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable excipient suitable for application to the skin. A “cosmetically acceptable” or “dermatologically-acceptable” composition or component refers to a composition or component that is suitable for use in contact with human skin without undue toxicity, incompatibility, instability, or allergic response. The excipient enables an active agent and optional component to be delivered to the skin at an appropriate concentration(s). The excipient thus can act as a diluent, dispersant, solvent, or the like to ensure that the active materials are applied to and distributed evenly over the selected target at an appropriate concentration. The excipient can be solid, semi-solid, or liquid. The excipient can be in the form of a lotion, a cream, or a gel, in particular one that has a sufficient thickness or yield point to prevent the active materials from sedimenting. The excipient can be inert or possess dermatological benefits. It also should be physically and chemically compatible with the active components described herein, and should not unduly impair stability, efficacy, or other use benefits associated with the composition.

### Pharmaceutical Dosage Forms

Various dosage forms of compounds of the invention (e.g., a cannabinoid compound, a compound described by any one of formulas (I)-(VIII), or any one of compounds 1-144) produced by any of the methods described herein can be used for preventing and/or treating a condition (e.g., an inflammatory disease or a fibrotic disease). In some embodiments, the dosage form is an oral dosage form such as a pressed tablet, hard or soft gel capsule, enteric coated tablet, osmotic release capsule, or unique combination of excipients.

In further embodiments, the dosage form includes an additional agent or is provided together with a second dosage form, which includes the additional agent. Exemplary additional agents include an analgesic agent such as an NSAID or opiate, an anti-inflammatory agent or a natural agent such as a triglyceride containing unsaturated fatty acid, or isolated pure fatty acids such as eicosapentaenoic acid (EPA), dihomogamma linolenic acid (DGLA), docosahexaenoic acid (DHA) and others. In additional embodiments, the dosage form includes a capsule wherein the capsule contains a mixture of materials to provide a desired sustained release formulation.

The dosage forms can include a tablet coated with a semipermeable coating. In certain embodiments, the tablet includes two layers, a layer containing a compound of the invention (e.g. a cannabinoid compound, a compound described by any one of formulas (I)-(VIII), or any one of compounds 1-144) and a second layer referred to as a “push” layer. The semi-permeable coating is used to allow a fluid (e.g., water) to enter the tablet and erode a layer or layers. In certain embodiments, this sustained release dosage form further includes a laser hole drilled in the center of the coated tablet. The layer containing the compound of the invention may include a compound of the invention (e.g., a cannabinoid compound, a compound described by any one of formulas (I)-(VIII), or any one of compounds 1-144), a disintegrant, a viscosity enhancing agent, a binding agent, and an osmotic agent. The push layer includes a disintegrant, a binding agent, an osmotic agent, and a viscosity enhancing agent.

The present compositions may be formulated for sustained release (e.g., over a 2 hour period, over a 6 hour period, over a 12 hour period, over a 24 hour period, or over a 48 hour period).

In further embodiments, the dosage form includes a tablet including a biocompatible matrix and a compound of the invention (e.g., a cannabinoid compound, a compound described by any one of formulas (I)-(VIII), or any one of compounds 1-144). The sustained release dosage form may also include a hard-shell capsule containing bio-polymer microspheres that contains the therapeutically active agent. The biocompatible matrix and bio-polymer microspheres each contain pores for drug release and delivery. These pores are formed by mixing the biocompatible matrix of bio-polymer microsphere with a pore forming agent. Each biocompatible matrix or bio-polymer microsphere is made up of a biocompatible polymer or mixture of biocompatible polymers. The matrix and microspheres can be formed by dissolving the biocompatible polymer and active agent (compound described herein) in a solvent and adding a pore-forming agent (e.g., a volatile salt). Evaporation of the solvent and pore forming agent provides a matrix or microsphere containing the active compound. In additional embodiments, the sustained release dosage form includes a tablet, wherein the tablet contains a compound of the invention (e.g., a cannabinoid compound, a compound described by any one of formulas (I)-(VIII), or any one of compounds 1-144) and one or more polymers and wherein the tablet can be prepared by compressing the compounds (e.g., a cannabinoid compound, a compound described by any one of formulas (I)-(VIII), or any one of compounds 1-144) and one or more polymers. In some embodiments, the one or more polymers may include a hygroscopic polymer formulated with the compound (e.g., a cannabinoid compound, a compound described by any one of formulas (I)-(VIII), or any one of compounds 1-144). Upon exposure to moisture, the tablet dissolves and swells. This swelling allows the sustained release dosage form to remain in the upper GI tract. The swelling rate of the polymer mixture can be varied using different grades of polyethylene oxide.

In other embodiments, the sustained release dosage form includes a capsule further including particle cores coated with a suspension of active agent and a binding agent which is subsequently coated with a polymer. The polymer may be a rate-controlling polymer. In general, the delivery rate of the rate-controlling polymer is determined by the rate at which the active agent is dissolved.

In some embodiments, one or more of the therapeutic agents may be formulated with a pharmaceutically acceptable carrier, vehicle or adjuvant. The term “pharmaceutically acceptable carrier, vehicle, or adjuvant” refers to a carrier, vehicle or adjuvant that may be administered to a subject, together with the present compounds, and which does not destroy the pharmacological activity thereof and is nontoxic when administered in doses sufficient to deliver a therapeutic amount of the compound.

Pharmaceutically acceptable carriers, adjuvants and vehicles that may be used in the dosage forms of this invention include, but are not limited to, ion exchangers, alumina, aluminum stearate, lecithin, self-emulsifying drug delivery systems (SEDDS) such as d-E-tocopherol polyethylene-glycol 1000 succinate; surfactants used in pharmaceutical dosage forms such as Tweens or other similar polymeric delivery matrices; serum proteins such as human serum albumin; buffer substances such as phosphates, glycine, sorbic acid, potassium sorbate, partial glyceride mixtures of saturated vegetable fatty acids, water, salts; or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxmethylcellulose, polyacrylates, waxes, polyethylene-polyoxypropylene-block polymers, polyethylene glycol and wool fat. Cyclodextrins such as alpha, beta and gamma-cyclodextrin, or chemically modified derivatives such as hydroxyalkylcyclodextrins, including 2- and 3-hydroxypropyl-beta cyclodextrins, or other solubilized derivatives may also be advantageously used to enhance delivery of compounds of the formulae described herein that can be used in the methods of the invention for preventing and/or treating fibrotic conditions. In certain embodiments, unit dosage formulations are compounded for immediate release, though unit dosage formulations compounded for delayed or prolonged release of one or both agents are also disclosed.

In some embodiments, one or more therapeutic agents (e.g., a cannabinoid compound, a compound described by any one of formulas (I)-(VIII), or any one of compounds 1-144) may be formulated in a single unit dose such that the agents are released from the dosage at different times.

In another embodiment, for example, where one or more of the therapeutic agents is administered once or twice per day, the agent is formulated to provide extended release. For example, the agent is formulated with an enteric coating. In an alternative embodiment, the agent is formulated using a biphasic controlled release delivery system, thereby providing prolonged gastric residence. For example, in some embodiments, the delivery system includes (1) an inner solid particulate phase formed of substantially uniform granules containing a pharmaceutical having a high water solubility, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophilic polymers, one or more hydrophobic polymers and/or one or more hydrophobic materials such as one or more waxes, fatty alcohols and/or fatty acid esters, which may be compressed into tablets or filled into capsules. In some embodiments, the agent is incorporated into polymeric matrices comprised of hydrophilic polymers that swell upon imbibition of water to a size that is large enough to promote retention of the dosage form in the stomach during the fed mode.

One or more therapeutic agents (e.g., a cannabinoid compound, a compound described by any one of formulas (I)-(VIII), or any one of compounds 1-144) may be formulated as a combination of fast-acting and controlled release forms. For example, one or more therapeutic agents (e.g., a cannabinoid compound, a compound described by any one of formulas (I)-(VIII), or any one of compounds 1-144) may be formulated with a single release property. For example, it is not present in a modified release form, e.g., a controlled release form.

The present compositions may be taken just prior to or with each of three meals, each of two major meals, or one meal. In other embodiments, a composition disclosed herein can be administered one or more times daily (e.g., once daily, twice daily, or three times daily) and need not be administered just before or with a meal.

The present compounds or compositions may be administered orally, for example as a component in a dosage form. The dosage forms may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The dosage forms of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase and may be combined with emulsifying and/or suspending agents. If desired, certain sweetening and/or flavoring and/or coloring agents may be added.

Non-limiting examples of capsules include but are not limited to gelatin capsules, HPMC, hard shell, soft shell, or any other suitable capsule for holding a sustained release mixture. The solvents used in the above sustained release dosage forms include, but are not limited to ethyl acetate, triacetin, dimethyl sulfoxide (DIV1S0), propylene carbonate, N-methylpyrrolidone (NMP), ethyl alcohol, benzyl alcohol, glycofurol, alpha-tocopherol, Miglyol 810, isopropyl alcohol, diethyl phthalate, polyethylene glycol 400 (PEG 400), triethyl citrate, and benzyl benzoate.

The viscosity modifiers that may be used in the above pharmaceutical compositions include, but are not limited to caprylic/capric triglyceride (Miglyol 810), isopropyl myristate (IPM), ethyl oleate, triethyl citrate, dimethyl phthalate, benzyl benzoate and various grades of polyethylene oxide. The high viscosity liquid carriers used in the above sustained release dosage forms include, but are not limited to sucrose acetate isobutyrate (SA1B) and cellulose acetate butyrate (CAB) 381-20.

Non-limiting examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof; ethylene vinyl acetate copolymers, polyethylene, copolymers of ethylene, polyolefins including ethylene oxide copolymers (e.g., Engage®, Dupont Dow Elastomers), polyamides, cellulosic materials, polyurethanes, polyether blocked amides, and copolymers (e.g., PEBAX®, cellulosic acetate butyrate and polyvinyl acetate). Non-limiting examples of disintegrants that may be employed in the above sustained release dosage forms include but are not limited to croscarmellose sodium, crospovidone, sodium alginate or similar excipients.

Non-limiting examples of binding agents that may be employed in the above dosage forms include but are not limited to hydroxyalkylcellulose, a hydroxyalkylalkylcellulose, or a polyvinylpyrrolidone.

Non-limiting examples of osmotic agents that may be employed in the above dosage forms include but are not limited to, sorbitol, mannitol, sodium chloride, or other salts. Non-limiting examples of biocompatible polymers employed in the above sustained release dosage forms include but are not limited to poly(hydroxy acids), polyanhydrides, polyorthoesters, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly(vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

Non-limiting examples of hygroscopic polymers that may be employed in the above dosage forms include but are not limited to polyethylene oxide (e.g., Polyox® with MWs from 4,000,000 to 10,000,000), cellulose hydroxymethyl cellulose, hydroxyethyl-cellulose, crosslinked polyacrylic acids and xanthan gum.

Non-limiting examples of rate-controlling polymers the may be employed in the above dosage forms include but are not limited to polymeric acrylate, methacrylate lacquer or mixtures thereof, polymeric acrylate lacquer, methacrylate lacquer, an acrylic resin including a copolymer of acrylic and methacrylic acid esters or an ammonium methacrylate lacquer with a plasticizer.

### Methods of Treatment

In some embodiments of the invention, any of the above-described compositions (e.g., compositions including a cannabinoid compound, a compound described by any one of formulas (I)-(VIII), or any one of compounds 1-144 prepared according to the methods of the invention), including any of the above-described pharmaceutical compositions, may be administered to a subject (e.g., a mammal, such as a human, cat, dog, horse, cow, or pig) having a disease (e.g., a fibrotic disease or an inflammatory disease) in order to treat, prevent, or ameliorate the disease.

Inflammation

A therapeutically effective amount of any of the compositions described herein (e.g. a pharmaceutical composition comprising a compound described by any one of formulas (I)-(VIII), or any one of compounds 1-144) may be used to treat or prevent inflammatory disease.

Inflammatory diseases include, for example, dermatomyositis, systemic lupus erythematosus, acquired immune deficiency syndrome (AIDS), multiple sclerosis, rheumatoid arthritis, psoriasis, diabetes (e.g., Type 1 diabetes), cancer, asthma, atopic dermatitis, autoimmune thyroid disorders, ulcerative colitis, Crohn's disease, stroke, ischemia, and neurodegenerative diseases, (e.g., Alzheimer's disease and Parkinson's disease), amyotrophic lateral sclerosis (ALS), chronic traumatic encephalopathy (CTE), chronic inflammatory demyelinating polyneuropathy, an autoimmune inner ear disease, uveitis, iritis, and peritonitis.

In some embodiments, inflammation can be assayed by measuring the chemotaxis and activation state of inflammatory cells. In some embodiments, inflammation can be measured by examining the production of specific inflammatory mediators such as interleukins, cytokines and eicosanoids. In some embodiments, in vivo inflammation is measured by swelling and edema of a localized tissue or migration of leukocytes. Inflammation may also be measured by organ function such as in the lung or kidneys and by the production of pro-inflammatory factors. Inflammation may also be assessed by other suitable methods. Other methods known to one skilled in the art may also be suitable methods for the assessment of inflammation and may be used to evaluate or score the response of the subject to treatment with one or more therapeutic agents of the invention (e.g., a cannabinoid compound, a compound described by any one of formulas (I)-(VIII), or any one of compounds 1-144).

### Fibrotic Diseases

A therapeutically effective amount of any of the compositions described herein (e.g. a pharmaceutical composition comprising a compound described by any one of formulas (I)-(VIII), or any one of compounds 1-144) may be used to treat or prevent inflammatory disease.

Fibrotic diseases include, for example, scleroderma (e.g., systemic sclerosis, localized scleroderma, sine scleroderma), liver cirrhosis, interstitial pulmonary fibrosis, idiopathic pulmonary fibrosis, Dupuytren's contracture, keloids, cystic fibrosis, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, or organ fibrosis (e.g., dermal fibrosis, lung fibrosis, liver fibrosis, kidney fibrosis, or heart fibrosis).

Non-limiting examples of fibrosis include liver fibrosis, lung fibrosis (e.g., silicosis, asbestosis, idiopathic pulmonary fibrosis), oral fibrosis, endomyocardial fibrosis, retroperitoneal fibrosis, deltoid fibrosis, kidney fibrosis (including diabetic nephropathy), cystic fibrosis, and glomerulosclerosis. Liver fibrosis, for example, occurs as a part of the wound-healing response to chronic liver injury. Fibrosis can occur as a complication of haemochromatosis, Wilson's disease, alcoholism, schistosomiasis, viral hepatitis, bile duct obstruction, exposure to toxins, and metabolic disorders. Endomyocardial fibrosis is an idiopathic disorder that is characterized by the development of restrictive cardiomyopathy. In endomyocardial fibrosis, the underlying process produces patchy fibrosis of the endocardial surface of the heart, leading to reduced compliance and, ultimately, restrictive physiology as the endomyocardial surface becomes more generally involved. Oral submucous fibrosis is a chronic, debilitating disease of the oral cavity characterized by inflammation and progressive fibrosis of the submucosal tissues (lamina propria and deeper connective tissues). The buccal mucosa is the most commonly involved site, but any part of the oral cavity can be involved, even the pharynx. Retroperitoneal fibrosis is characterized by the development of extensive fibrosis throughout the retroperitoneum, typically centered over the anterior surface of the fourth and fifth lumbar vertebrae.

Treatment of fibrosis may be assessed by suitable methods known to one of skill in the art including the improvement, amelioration, or slowing the progression of one or more symptoms associated with the particular fibrotic disease being treated.

Scleroderma

Scleroderma is a disease of the connective tissue characterized by fibrosis of the skin and internal organs. Scleroderma has a spectrum of manifestations and a variety of therapeutic implications. It includes localized scleroderma, systemic sclerosis, scleroderma-like disorders, and sine scleroderma. Systemic sclerosis can be diffuse or limited. Limited systemic sclerosis is also called CREST (calcinosis, Raynaud's esophageal dysfunction, sclerodactyly, telangiectasia). Systemic sclerosis includes: scleroderma lung disease, scleroderma renal crisis, cardiac manifestations, muscular weakness including fatigue or limited CREST, gastrointestinal dysmotility and spasm, and abnormalities in the central, peripheral and autonomic nervous system.

The major symptoms or manifestations of scleroderma, and in particular of systemic sclerosis, are inappropriate excessive collagen synthesis and deposition, endothelial dysfunction, vasospasm, collapse and obliteration of vessels by fibrosis. In terms of diagnosis, an important clinical parameter may be skin thickening proximal to the metacarpophalangeal joints. Raynaud's phenomenon may be a component of scleroderma. Raynaud's may be diagnosed by color changes of the skin upon cold exposure. Ischemia and skin thickening may also be symptoms of Raynaud's disease.

A therapeutically effective amount of any of the compositions described herein (e.g. a cannabinoid compound, a compound described by any one of formulas (I)-(VIII), or any one of compounds 1-144 prepared by any of the methods described herein) may be used to treat or prevent fibrosis. Fibrosis may be assessed by suitable methods known to one of skill in the art.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

General Methods

High Performance Liquid Chromatography (HPLC) Methods

HPLC Method A: ThermoScientific Biobasic-18 column (5 μM, 4.6 mm×150 mm), 10-90% MeCN gradient in H₂O containing 0.1% trifluoroacetic acid, run time=30 min

HPLC Method B: Zorbax RX-C18 (5 μM, 4.6 mm×150 mm), 10-95% MeCN gradient in H₂O containing 0.1% formic acid, run time=23 min

HPLC Method C: YMC pack ODS-AQ C18 column (3 μm, 4.6 mm×50 mm) at 35° C., with a flow rate of 2.6 mL/min. A gradient elution was performed from 5% acetonitrile/95% (water+0.1% formic acid) to 95% acetonitrile/5% (water+0.1% formic acid) in 4.8 min. Acquisition ranges were set to 190-400 nm for the UV-PDA detector and 100-1400 m/z for the MS detector.

HPLC Method D: YMC pack ODS-AQ C18 column (3 μm, 4.6 mm×50 mm) at 35° C., with a flow rate of 2.6 mL/min. A gradient elution was performed using ISET 2V1.0 Emulated Agilent Pump G1312A V1.0 from 5% Acetonitrile/95% (Water+0.1% Formic acid) to 95% Acetonitrile/5% (Water+0.1% Formic acid) in 4.8 min. Acquisition ranges were set to 190-400 nm for the UV-PDA detector and 100-1000 m/z for the TOF-MS detector.

HPLC Method E: Thermo Scientific Accucore aQ C18 column (2.6 μm, 4.6 mm×50 mm) at 35° C., with a flow rate of 2.6 mL/min. A gradient elution was performed from 50% (Water+50 mM NH₄OAc)/50% Acetonitrile to 5% (Water+50 mM NH₄OAc)/95% Acetonitrile in 4.8 min. Acquisition ranges were set to 190-400 nm for the UV-PDA detector and 100-1400 m/z for the MS detector.

Synthesis of Ajulemic Acid

Ajulemic acid may be synthesized as known in the art. Preferably, ajulemic acid is an ultrapure formulation of ajulemic acid including more than 99% ajulemic acid and less than 1% highly-active CB-1 impurities, e.g., HU-210. Ajulemic acid may be synthesized as described in U.S. Patent Publication No. 2015/0141501, which is incorporated herein by reference.

General Procedure for Amide Bond Formation with tBu-Protected Amino Acids

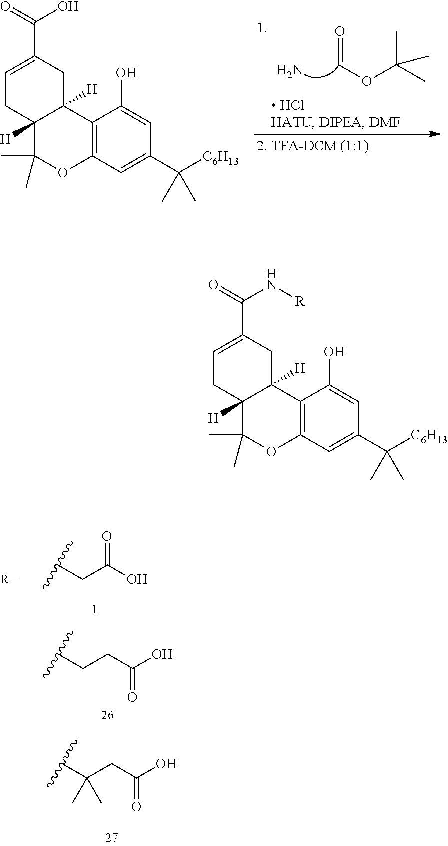

(R,R)-Ajulemic acid (AJA) (1 eq) and 1-[bis(dimethylamino)methylene]-1H-1,2,3-triazolo[4,5-b]pyridinium 3-oxid hexafluorophosphate (HATU) (1 eq) were dissolved in N,N-dimethylformamide (DMF) (130 eq), N,N-diisopropylethylamine (DIPEA) (3.3 eq) was added and the mixture stirred at room temperature for 15 min after which the amine hydrochloride amine (1.1 eq) was added and stirring continued for another 16 h. The reaction mixture was diluted with EtOAc and washed with water, then dried over MgSO₄ and filtered. The solvents were removed by rotary evaporation under reduced pressure and the residue dissolved in dichloromethane (DCM) (217 eq) and trifluoroacetic acid (TFA) (180 eq). The solution was stirred at room temperature for 2 h, after which the volatiles were removed by rotary evaporation under reduced pressure. Purification of the residue by flash chromatography on silica gel eluting with an increasing proportion of EtOAc in hexanes provided the title compound.

General Procedure for Amide Bond Formation with HATU

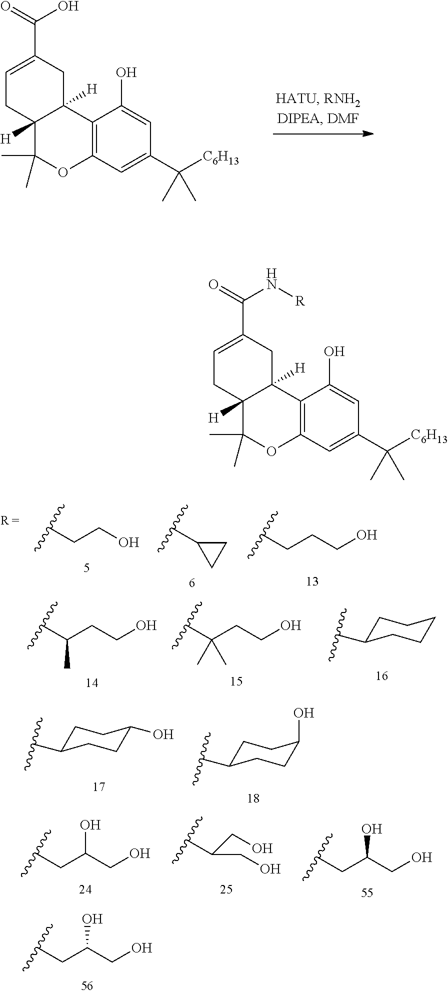

(R,R)-Ajulemic acid (AJA) (1 eq) and 1-[bis(dimethylamino)methylene]-1H-1,2,3-triazolo[4,5-b]pyridinium 3-oxide hexafluorophosphate (HATU) (1.05 eq) were dissolved in N,N-dimethylformamide (DMF) (53 eq), N,N-diisopropylethylamine (DIPEA) (2 eq) was added and the mixture stirred at room temperature for 10 min, after which amine (1.1 eq) was added and stirring continued for 16 h. The reaction mixture was diluted with diethyl ether (Et₂O) or ethyl acetate (EtOAc) and washed with water and brine, then dried over MgSO₄ and filtered. The solvents were removed by rotary evaporation under reduced pressure after which purification of the residue by flash chromatography on silica gel eluting with an increasing proportion of methanol (MeOH) in dichloromethane (DCM) or hexane in ethyl acetate (EtOAc) provided the title compound.

General Procedure for Amide Bond Formation with Me-Protected Amino Acids

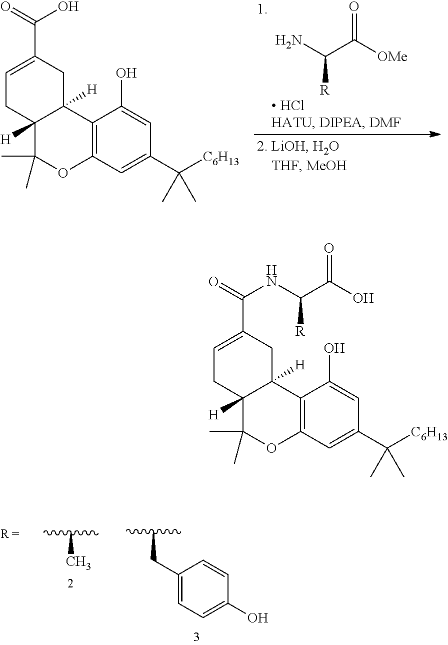

(R,R)-Ajulemic acid (AJA) (1 eq) and 1-[bis(dimethylamino)methylene]-1H-1,2,3-triazolo[4,5-b]pyridinium 3-oxid hexafluorophosphate (HATU) (1 eq) were dissolved in N,N-dimethylformamide (DMF) (130 eq), N,N-diisopropylethylamine (DIPEA) (3.3 eq) was added and the mixture stirred at room temperature for 15 min after which the amine hydrochloride amine (1.1 eq) was added and stirring continued for another 16 h. The reaction mixture was diluted with EtOAc and washed with water, then dried over MgSO₄ and filtered. The solvents were removed by rotary evaporation under reduced pressure and the residue dissolved in a 1:1:1 mixture of water, THE and MeOH. Lithium hydroxide (6 eq) was added and the solution was stirred at 0° C. for 3 h. The reaction mixture was acidified to pH 2 using 1 M HCl, then extracted with EtOAc. The organic layer was washed twice with water, then dried over MgSO₄, after which the volatiles were removed by rotary evaporation under reduced pressure. Purification of the residue by flash chromatography on silica gel eluting with an increasing proportion of EtOAc in hexanes provided the title compound.

General Procedure for Esterification Using Acetyl Chloride

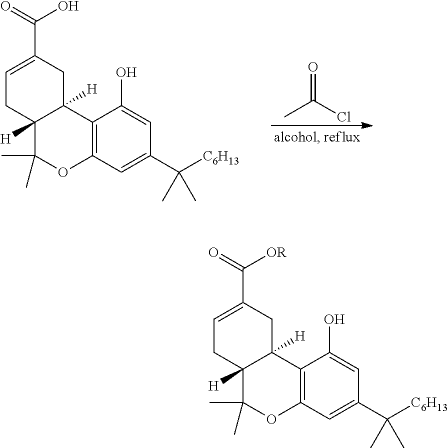

Acetyl chloride (14 eq) was added dropwise to the alcohol solvent (162 eq) at 0° C. The solution was allowed to warm up to room temperature (rt) and stirred for 1 h. (R,R)-Ajulemic acid (AJA) (1 eq) was added to the solution and the mixture was stirred under reflux for 16 h. The solvent was removed by rotary evaporation under reduced pressure after which purification of the residue by flash chromatography on silica gel eluting with an increasing proportion of EtOAc in hexanes provided the desired compound.

### Example 1. Synthesis of ((6aR,10aR)-1-Hydroxy-6,6-dimethyl-3-(2-methyloctan-2-yl)-6a,7,10,10a-tetrahydro-6H-benzo[c]chromene-9-carbonyl)glycine (Compound 1)

Compound 1 was synthesized according to the “general procedure for amide bond formation with tBu-protected amino acids.” Yield 67% (27 mg). ¹H NMR (400 MHz, CD₃OD): δ 0.86 (3H, t, J=6.7 Hz), 1.05-1.10 (4H, m), 1.20-1.22 (12H, m), 1.38 (3H, s), 1.50-1.56 (3H, m), 1.79 (1H, td, J=11.7, 4.5 Hz), 1.89-2.08 (2H, m), 2.38-2.45 (1H, m), 2.66 (1H, td, J=11.1, 4.5 Hz), 3.81-3.86 (1H, m), 3.95 (2H, s), 6.23 (1H, d, J=1.9 Hz), 6.34 (1H, d, J=1.9 Hz), 6.69-6.71 (1H, m). LC-MS (ESI−): 456.2 (M−H)⁻.

### Example 2. Synthesis of 3-((6aR,10aR)-1-Hydroxy-6,6-dimethyl-3-(2-methyloctan-2-yl)-6a,7,10,10a-tetrahydro-6H-benzo[c]chromene-9-carboxamido)propanoic acid (Compound 26)

Compound 26 was synthesized according to the “general procedure for amide bond formation with tBu-protected amino acids.” Yield 76% (274.3 mg). ¹H NMR (400 MHz, CDCl₃): δ 0.84 (3H, t, J=6.8 Hz), 1.03-1.07 (2H, m), 1.10 (3H, s), 1.16-1.25 (12H, m), 1.39 (3H, s), 1.47-1.51 (2H, m), 1.80 (1H, td, J=11.4, 4.3 Hz), 1.93-2.00 (2H, m), 2.33-2.39 (1H, m), 2.61-2.72 (3H, m), 3.51-3.59 (1H, m), 3.66-3.71 (1H, m), 3.73-3.78 (1H, m), 6.34 (1H, s), 6.36 (1H, s), 6.55 (1H, t, J=6.0 Hz), 6.79 (1H, d, J=4.8 Hz). LC-MS (ESI−): 470.3 (M−H)⁻. HPLC RT=18.7 min (HPLC Method A).

### Example 3. Synthesis of 3-((6aR,10aR)-1-Hydroxy-6,6-dimethyl-3-(2-methyloctan-2-yl)-6a,7,10,10a-tetrahydro-6H-benzo[c]chromene-9-carboxamido)-3-methylbutanoic acid (Compound 27)

Compound 27 was synthesized according to the “general procedure for amide bond formation with tBu-protected amino acids.” Yield 83% (180 mg). ¹H NMR (400 MHz, CDCl₃): δ 0.84 (3H, t, J=6.8 Hz), 1.04 (3H, s), 1.06-1.11 (2H, m), 1.16-1.25 (12H, m), 1.38 (3H, s), 1.47 (2H, m), 1.49 (3H, s), 1.52 (3H, s), 1.79 (1H, td, J=11.4, 4.3 Hz), 1.90-2.0 (2H, m), 2.33-2.39 (1H, m), 2.68 (1H, td, J=10.9, 4.5 Hz), 2.78 (1H, d, J=15.1 Hz), 2.97 (1H, d, J=15.1 Hz), 2.33-2.39 (1H, m), 6.28 (1H, d, J=1.7 Hz), 6.34 (1H, s), 6.37 (1H, d, J=1.7 Hz), 6.72 (1H, d, J=4.9 Hz). LC-MS (ESI−): 498.32 (M−H)⁻. HPLC RT=19.9 min (HPLC Method A).

### Example 4. Synthesis of ((6aR,10aR)-1-Hydroxy-6,6-dimethyl-3-(2-methyloctan-2-yl)-6a,7,10,10a-tetrahydro-6H-benzo[c]chromene-9-carbonyl)-D-alanine (Compound 2)

Compound 2 was synthesized according to the “general procedure for amide bond formation with Me-protected amino acids.” Yield 26% (31 mg). ¹H NMR (400 MHz, CDCl₃): δ 0.84 (3H, t, J=6.8 Hz), 1.07-1.11 (6H, m), 1.19-1.22 (13H, m), 1.39 (3H, s), 1.50 (5H, t, J=7.1 Hz), 1.79-1.85 (1H, m), 2.01 (2H, d, J=13.9 Hz), 2.39 (1H, d, J=17.7 Hz), 2.70 (1H, td, J=10.9, 4.5 Hz), 3.79 (1H, d, J=16.6 Hz), 4.64 (1H, t, J=7.1 Hz), 6.29 (1H, d, J=1.7 Hz), 6.37-6.38 (2H, m), 6.81 (1H, d, J=4.9 Hz). LC-MS (ESI−): 470.3 (M−H)⁻.

### Example 5. Synthesis of 3-((6aR,10aR)-1-Hydroxy-6,6-dimethyl-3-(2-methyloctan-2-yl)-6a,7,10,10a-tetrahydro-6H-benzo[c]chromene-9-carbonyl)-D-tyrosine (Compound 3)

Compound 3 was synthesized according to the “general procedure for amide bond formation with Me-protected amino acids.” Yield 2-17% (106 mg). ¹H NMR (400 MHz, CD₃OD): δ 0.86 (3H, t, J=6.7 Hz), 1.06-1.09 (4H, m), 1.20-1.24 (10H, m), 1.37 (3H, s), 1.45-1.56 (3H, m), 1.72-1.79 (1H, m), 1.86-2.04 (2H, m), 2.33-2.41 (1H, m), 2.59-2.66 (1H, m), 2.95 (1H, dd, J=14.0, 8.9 Hz), 3.12-3.17 (1H, m), 3.71-3.77 (1H, m), 4.63 (1H, dd, J=8.9, 4.9 Hz), 6.23 (1H, d, J=1.8 Hz), 6.35 (1H, t, J=1.7 Hz), 6.55-6.57 (1H, m), 6.71 (2H, d, J=8.2 Hz), 7.05 (2H, d, J=8.2 Hz). LC-MS (ESI−): 562.3 (M−H)⁻.

### Example 6. Synthesis of (6aR,10aR)-1-Hydroxy-6,6-dimethyl-3-(2-methyloctan-2-yl)-6a,7,10,10a-tetrahydro-6H-benzo[c]chromene-9-carboxamide (Compound 4)

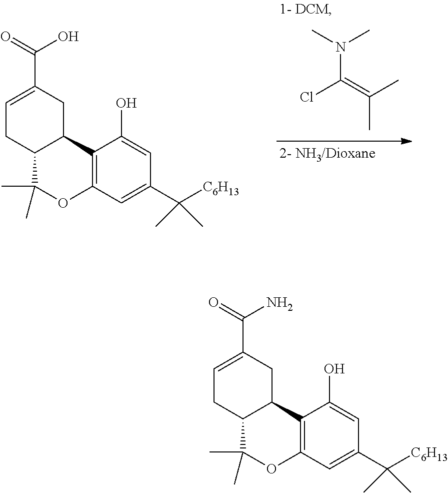

(R,R)-Ajulemic acid (AJA) (194 mg, 0.48 mmol, 1 eq) was dissolved in dichloromethane (DCM) (2 mL), Ghosez's reagent (70 mL, 0.53 mmol, 1.1 eq) was added and the mixture was stirred at room temperature for 1 h. Ammonia in dioxane was added and stirring continued for 16 h. The reaction mixture was diluted with dichloromethane (DCM) and washed with water, 1N HCl and brine, then dried over MgSO₄ and filtered. The solvents were removed by rotary evaporation under reduced pressure after which purification of the residue by flash chromatography on silica gel eluting with an increasing proportion of ethyl acetate (EtOAc) in hexane provided the title compound. ¹H NMR (400 MHz, CDCl₃): δ 0.85 (3H, t, J=6.8 Hz), 1.02-1.09 (2H, m), 1.13 (3H, s), 1.17-1.25 (12H, m), 1.41 (3H, s), 1.48-1.53 (2H, m), 1.80-1.87 (1H, m), 1.96-2.07 (2H, m), 2.36-2.43 (1H, m), 2.72 (1H, td, J=11.0, 4.6 Hz), 3.73-3.80 (1H, m), 6.26 (1H, d, J=1.8 Hz), 6.39 (1H, d, J=1.8 Hz), 6.81 (1H, m). LC-MS (ESI+): 400.3 (M+H)⁺.

HPLC RT=18.2 min (HPLC Method A).

### Example 7. Synthesis of (6aR,10aR)-1-Hydroxy-N-(2-hydroxyethyl)-6,6-dimethyl-3-(2-methyloctan-2-yl)-6a,7,10,10a-tetrahydro-6H-benzo[c]chromene-9-carboxamide (Compound 5)

Compound 4 was synthesized according to the “general procedure for amide bond formation with HATU.” Yield 86% (2.10 g). ¹H NMR (400 MHz, CDCl₃): δ 0.84 (3H, t, J=6.7 Hz), 1.03-1.08 (2H, m), 1.13 (3H, s), 1.17-1.29 (12H, m), 1.40 (3H, s), 1.47-1.52 (2H, m), 1.82 (1H, td, J=11.5, 4.3 Hz), 1.95-2.05 (2H, m), 2.38 (1H, dt, J=17.3, 4.6 Hz), 2.71 (1H, td, J=11.0, 4.6 Hz), 2.95 (1H, t, J=5.1 Hz), 3.47-3.57 (2H, m), 3.73-3.79 (3H, m), 5.76 (1H, s), 6.28 (2H, m), 6.38 (1H, d, J=1.6 Hz), 6.79 (1H, d, J=4.9 Hz). LC-MS (ESI+): 444.3 (M+H)⁺. HPLC RT=17.6 min (HPLC Method B).

### Example 8. Synthesis of (6aR,10aR)—N-Cyclopropyl-1-hydroxy-6,6-dimethyl-3-(2-methyloctan-2-yl)-6a,7,10,10a-tetrahydro-6H-benzo[c]chromene-9-carboxamide (Compound 6)

Compound 6 was synthesized according to the “general procedure for amide bond formation with HATU.” Yield 75% (180 mg). ¹H NMR (400 MHz, CDCl₃): δ 0.50-0.54 (2H, m), 0.78-0.85 (5H, m), 1.03-1.08 (2H, m), 1.12 (3H, s), 1.16-1.21 (12H, m), 1.40 (3H, s), 1.47-1.51 (2H, m), 1.81 (1H, td, J=11.5, 4.3 Hz), 1.92-2.01 (2H, m), 2.33-2.39 (1H, m), 2.65-2.72 (1H, m), 2.76-2.80 (1H, m), 3.69-3.75 (1H, m), 5.51 (1H, s), 5.83 (1H, s), 6.31 (1H, d, J=1.8 Hz), 6.37 (1H, d, J=1.8 Hz), 6.69 (1H, d, J=5.0 Hz). HPLC RT=14.89 min (HPLC Method B).

### Example 9. Synthesis of (6aR,10aR)-1-Hydroxy-N-(3-hydroxypropyl)-6,6-dimethyl-3-(2-methyloctan-2-yl)-6a,7,10,10a-tetrahydro-6H-benzo[c]chromene-9-carboxamide (Compound 13)

Compound 13 was synthesized according to the “general procedure for amide bond formation with HATU.” Yield 72% (900 mg). ¹H NMR (400 MHz, CDCl₃): δ 0.84 (3H, t, J=6.8 Hz), 1.03-1.08 (2H, m), 1.13 (3H, s), 1.16-1.25 (14H, m), 1.40 (3H, s), 1.47-1.51 (2H, m), 1.68-1.74 (2H, m), 1.78-1.86 (1H, m), 1.95-2.03 (2H, m), 2.34-2.41 (1H, m), 2.68-2.75 (1H, m), 3.51 (2H, q, J=5.9 Hz), 3.65 (2H, t, J=5.4 Hz), 3.72-3.78 (1H, m), 6.20-6.23 (1H, m), 6.27 (1H, d, J=1.7 Hz), 6.38 (1H, d, J=1.6 Hz), 6.80 (1H, d, J=4.7 Hz). HPLC RT=13.21 min (HPLC Method B).

### Example 10. Synthesis of (6aR,10aR)-1-Hydroxy-N—((R)-4-hydroxybutan-2-yl)-6,6-dimethyl-3-(2-methyloctan-2-yl)-6a,7,10,10a-tetrahydro-6H-benzo[c]chromene-9-carboxamide (Compound 14)

Compound 14 was synthesized according to the “general procedure for amide bond formation with HATU.” Yield 51% (180 mg). ¹H NMR (400 MHz, CDCl₃): δ 0.84 (3H, t, J=6.8 Hz), 1.03-1.09 (2H, m), 1.13 (3H, s), 1.16-1.21 (12H, m), 1.26 (3H, d, J=6.7 Hz), 1.31-1.38 (1H, m), 1.40 (3H, s), 1.47-1.51 (2H, m), 1.80-1.92 (2H, m), 1.95-2.03 (2H, m), 2.35-2.41 (1H, m), 2.71 (1H, td, J=10.9, 4.6 Hz), 2.81 (1H, s), 3.52-3.64 (2H, m), 3.75 (1H, dd, J=16.1, 4.5 Hz), 4.27-4.33 (1H, m), 5.78 (1H, d, J=8.4 Hz), 5.81 (1H, s), 6.28 (1H, d, J=1.8 Hz), 6.37 (1H, d, J=1.7 Hz), 6.77 (1H, d, J=5.0 Hz). LC-MS (ESI+): 472.3 (M+H)⁺. HPLC RT=12.52 min (HPLC Method B).

### Example 11. Synthesis of (6aR,10aR)-1-Hydroxy-N-(4-hydroxy-2-methylbutan-2-yl)-6,6-dimethyl-3-(2-methyloctan-2-yl)-6a,7,10,10a-tetrahydro-6H-benzo[c]chromene-9-carboxamide (Compound 15)

Compound 15 was synthesized according to the “general procedure for amide bond formation with HATU.” Yield 60% (180 mg). ¹H NMR (400 MHz, CDCl₃): δ 0.84 (3H, t, J=6.9 Hz), 1.03-1.08 (2H, m), 1.11 (3H, s), 1.16-1.26 (12H, m), 1.39 (3H, s), 1.45 (6H, d, J=4.3 Hz), 1.47-1.51 (2H, m), 1.80 (1H, td, J=11.4, 4.2 Hz), 1.89 (2H, t, J=5.7 Hz), 1.90-2.00 (2H, m), 2.36-2.29 (1H, m), 2.67 (1H, td, J=10.9, 4.5 Hz), 3.72-3.78 (1H, m), 3.84 (2H, t, J=5.7 Hz), 6.28 (1H, d, J=1.8 Hz), 6.37 (1H, d, J=1.7 Hz), 6.63 (2H, m). LC-MS (ESI+): 486.3 (M+H)⁺. HPLC RT=14.72 min (HPLC Method B).

### Example 12. Synthesis of (6aR,10aR)—N-Cyclohexyl-1-hydroxy-6,6-dimethyl-3-(2-methyloctan-2-yl)-6a,7,10,10a-tetrahydro-6H-benzo[c]chromene-9-carboxamide (Compound 16)

Compound 16 was synthesized according to the “general procedure for amide bond formation with HATU.” Yield 64% (360 mg). ¹H NMR (400 MHz, CDCl₃): δ 0.84 (3H, t, J=6.8 Hz), 1.01-1.11 (3H, m), 1.12 (3H, s), 1.15-1.24 (14H, m), 1.30-1.38 (2H, m), 1.40 (3H, s), 1.48-1.52 (2H, m), 1.59-1.65 (1H, m), 1.68-1.73 (2H, m), 1.82 (1H, td, J=11.4, 4.2 Hz), 1.92-2.01 (4H, m), 2.31-2.38 (1H, m), 2.67-2.74 (1H, m), 3.78-3.90 (2H, m) 5.58-5.60 (1H, m), 5.84 (1H, s), 6.33 (1H, d, J=1.8 Hz), 6.37 (1H, d, J=1.8 Hz), 6.63 (1H, d, J=4.9 Hz). HPLC RT=22.98 min (HPLC Method B).

### Example 13. Synthesis of (6aR,10aR)-1-Hydroxy-N-(trans-4-hydroxycyclohexyl)-6,6-dimethyl-3-(2-methyloctan-2-yl)-6a,7,10,10a-tetrahydro-6H-benzo[c]chromene-9-carboxamide (Compound 17)

Compound 17 was synthesized according to the “general procedure for amide bond formation with HATU.” Yield 50% (180 mg). ¹H NMR (400 MHz, CDCl₃): δ 0.84 (3H, t, J=6.8 Hz), 1.03-1.07 (2H, m), 1.12 (3H, s), 1.17-1.25 (14H, m), 1.40 (3H, s), 1.41-1.53 (5H, m), 1.81 (1H, td, J=11.5, 4.3 Hz), 1.94-2.09 (6H, m), 2.35 (1H, dt, J=16.5, 4.8 Hz), 2.70 (1H, td, J=10.9, 4.5 Hz), 3.58-3.64 (1H, m), 3.76-3.88 (2H, m), 5.53 (1H, d, J=7.9 Hz), 6.02 (1H, s), 6.33 (1H, d, J=1.8 Hz), 6.36 (1H, d, J=1.7 Hz), 6.63 (1H, d, J=4.9 Hz). LC-MS (ESI+): 498.3 (M+H)⁺. HPLC RT=11.78 min (HPLC Method B).

### Example 14. Synthesis of (6aR,10aR)-1-Hydroxy-N-(cis-4-hydroxycyclohexyl)-6,6-dimethyl-3-(2-methyloctan-2-yl)-6a,7,10,10a-tetrahydro-6H-benzo[c]chromene-9-carboxamide (Compound 18)

Compound 18 was synthesized according to the “general procedure for amide bond formation with HATU.” Yield 43% (180 mg). ¹H NMR (400 MHz, CDCl₃): δ 0.84 (3H, t, J=6.8 Hz), 1.03-1.07 (2H, m), 1.12 (3H, s), 1.16-1.24 (12H, m), 1.40 (3H, s), 1.16-1.51 (2H, m), 1.64-1.75 (8H, m), 1.78-1.85 (1H, m), 1.92-2.01 (2H, m), 2.31-2.39 (1H, m), 2.70 (1H, td, J=10.8, 4.5 Hz), 3.82-3.87 (1H, m), 3.94-3.98 (2H, m), 5.76 (1H, d, J=7.9 Hz), 6.34 (1H, d, J=1.8 Hz), 6.38 (1H, d, J=1.8 Hz), 6.63 (1H, d, J=4.9 Hz). LC-MS (ESI+): 498.3 (M+H)⁺. HPLC RT=11.88 min (HPLC Method B).

### Example 15. Synthesis of (6aR,10aR)-N-(2,3-Dihydroxypropyl)-1-hydroxy-6,6-dimethyl-3-(2-methyloctan-2-yl)-6a,7,10,10a-tetrahydro-6H-benzo[c]chromene-9-carboxamide (Compound 24)

Compound 24 was synthesized according to the “general procedure for amide bond formation with HATU.” Yield 40% (235 mg). ¹H NMR (400 MHz, CDCl₃): δ 0.82 (3H, t, J=6.8 Hz), 1.01-1.06 (2H, m), 1.08 (3H, s), 1.15-1.24 (12H, m), 1.37 (3H, s), 1.45-1.49 (2H, m), 1.78 (1H, td, J=11.4, 4.3 Hz), 1.91-2.02 (2H, m), 2.34 (1H, m), 2.68 (1H, td, J=10.8, 4.5 Hz), 3.25-3.62 (4H, m), 3.73 (1H, m), 3.80-3.87 (1H, m), 6.35 (2H, m), 6.85-6.94 (2H, m). HPLC RT=17.13 min (HPLC Method B).

### Example 16. Synthesis of (6aR,10aR)-N-(1,3-Dihydroxypropan-2-yl)-1-hydroxy-6,6-dimethyl-3-(2-methyloctan-2-yl)-6a,7,10,10a-tetrahydro-6H-benzo[c]chromene-9-carboxamide (Compound 25)

Compound 25 was synthesized according to the “general procedure for amide bond formation with HATU.” Yield 45% (265 mg). ¹H NMR (400 MHz, CDCl₃): δ 0.83 (3H, t, J=6.8 Hz), 1.03-1.07 (2H, m), 1.08 (3H, s), 1.16-1.24 (12H, m), 1.37 (3H, s), 1.46-1.50 (2H, m), 1.77 (1H, td, J=11.4, 4.4 Hz), 1.91-2.02 (2H, m), 2.33 (1H, m), 2.65 (1H, m), 3.71-3.89 (5H, m), 4.03 (1H, m), 6.30 (1H, d, J=1.8 Hz), 6.36 (1H, d, J=1.7 Hz), 6.77 (2H, m). HPLC RT=16.98 min (HPLC Method B).

### Example 17. Synthesis of (6aR,10aR)-N—((R)-2,3-Dihydroxypropyl)-1-hydroxy-6,6-dimethyl-3-(2-methyloctan-2-yl)-6a,7,10,10a-tetrahydro-6H-benzo[c]chromene-9-carboxamide (Compound 55)

Compound 24 was synthesized according to the “general procedure for amide bond formation with HATU.” Yield 41% (147 mg). ¹H NMR (400 MHz, CDCl₃): δ 0.83 (3H, t, J=6.8 Hz), 1.06-1.09 (6H, m), 1.17-1.20 (12H, m), 1.38 (3H, s), 1.46-1.50 (3H, m), 1.66 (1H, s), 1.81 (1H, d, J=12.0 Hz), 1.98 (2H, br s), 2.37 (1H, d, J=16.6 Hz), 2.69 (1H, s), 3.35-3.71 (4H, m), 3.69-3.80 (2H, m), 6.34 (2H, d, J=17.4 Hz), 6.65 (1H, s), 6.85 (1H, s). HPLC RT=17.74 min (HPLC Method A).

### Example 18. Synthesis of (6aR,10aR)-N—((S)-2,3-Dihydroxypropyl)-1-hydroxy-6,6-dimethyl-3-(2-methyloctan-2-yl)-6a,7,10,10a-tetrahydro-6H-benzo[c]chromene-9-carboxamide (Compound 56)

Compound 24 was synthesized according to the “general procedure for amide bond formation with HATU.” Yield 31% (112 mg). ¹H NMR (400 MHz, CDCl₃): δ 0.77 (3H, t, J=6.8 Hz), 1.00-1.04 (4H, m), 1.12-1.15 (10H, m), 1.33 (3H, s), 1.40-1.44 (3H, m), 1.54 (4H, br s), 1.76 (1H, d, J=12.5 Hz), 1.93 (2H, d, J=12.7 Hz), 2.29 (1H, s), 2.64 (1H, d, J=5.0 Hz), 3.30 (1H, d, J=13.7 Hz), 3.48-3.51 (3H, m), 3.65 (2H, d, J=16.0 Hz), 3.77 (1H, s), 6.25 (1H, s), 6.31 (1H, d, J=1.7 Hz), 6.51 (1H, s), 6.80 (1H, s). HPLC RT=17.63 min (HPLC Method A).

### Example 19. Synthesis of (6aR,10aR)-N-(2-Hydroxyethyl)-1-methoxy-6,6-dimethyl-3-(2-methyloctan-2-yl)-6a,7,10,10a-tetrahydro-6H-benzo[c]chromene-9-carboxamide (Compound 7)

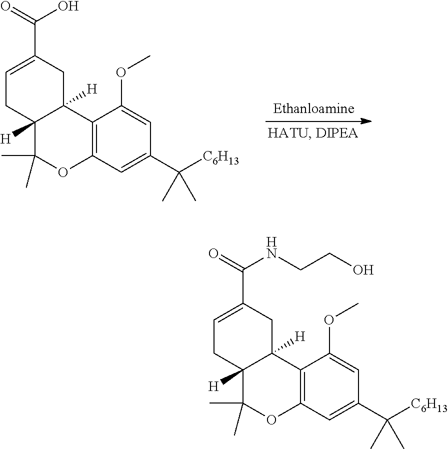

(6aR,10aR)-1-Methoxy-6,6-dimethyl-3-(2-methyloctan-2-yl)-6a,7,10,10a-tetrahydro-6H-benzo[c]chromene-9-carboxylic acid (Compound 34, 276 mg, 0.67 mmol, 1 eq) and 1-[bis(dimethylamino)methylene]-1H-1,2,3-triazolo[4,5-b]pyridinium 3-oxid hexafluorophosphate (HATU) (304 mg, 0.80 mmol, 1.2 eq) were dissolved in N,N-dimethylformamide (DMF) (5 mL), N,N-diisopropylethylamine (DIPEA) (291 μL, 1.66 mmol, 2.5 eq) was added and the mixture stirred at room temperature for 30 min, after which ethanolamine (45 μL, 0.73 mmol, 1.1 eq) was added and stirring continued for 16 h. The reaction mixture was diluted with ethyl acetate (EtOAc) and washed with water, 1N HCl and brine, then dried over MgSO₄ and filtered. The solvents were removed by rotary evaporation under reduced pressure after which purification of the residue by flash chromatography on silica gel eluting with an increasing proportion of ethyl acetate (EtOAc) in hexane provided the title compound. Yield 40% (110 mg). ¹H NMR (400 MHz, CDCl₃): δ 0.85 (3H, t, J=6.8 Hz), 1.04-1.10 (2H, m), 1.11 (3H, s), 1.21-1.25 (12H, m), 1.40 (3H, s), 1.80-1.87 (1H, m), 1.92-2.01 (2H, m), 2.34-2.42 (1H, m), 2.64-2.72 (2H, m), 2.80 (1H, s), 3.48-3.53 (2H, m), 3.60-3.67 (1H, m), 3.77 (2H, q, J=5.0 Hz), 3.82 (3H, s), 3.99-4.05 (1H, m), 6.10-6.15 (1H, m), 6.39 (1H, d, J=1.7 Hz), 6.43 (1H, d, J=1.7 Hz), 6.76-6.78 (1H, m).

### Example 20. Synthesis of (6aR,10aR)-1-Hydroxy-6,6-dimethyl-3-(2-methylnonan-2-yl)-6a,7,10,10a-tetrahydro-6H-benzo[c]chromene-9-carboxylic acid (Compound 9)

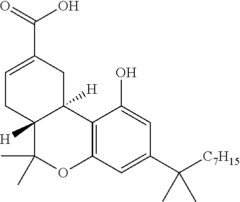

The standard procedure for synthesis of Ajulemic acid (AJA) was followed with replacement of 5-(2-methyloctan-2-yl)benzene-1,3-diol (DMHR) by 5-(2-methylnonan-2-yl)benzene-1,3-diol. ¹H NMR (400 MHz, CDCl₃): δ 0.84-0.87 (3H, m), 1.02-1.09 (3H, m), 1.14 (3H, s), 1.19-1.22 (12H, m), 1.42 (3H, s), 1.46-1.59 (5H, m), 1.81-1.88 (1H, m), 2.40-2.48 (1H, m), 2.64-2.71 (1H, m), 3.79-3.85 (1H, m), 6.23 (1H, d, J=1.8 Hz), 6.40 (1H, d, J=1.8 Hz), 7.13 (1H, m).

### Example 21. Synthesis of 4-Carbamothioylphenyl (6aR,10aR)-1-hydroxy-6,6-dimethyl-3-(2-methyloctan-2-yl)-6a,7,10,10a-tetrahydro-6H-benzo[c]chromene-9-carboxylate (Compound 10)

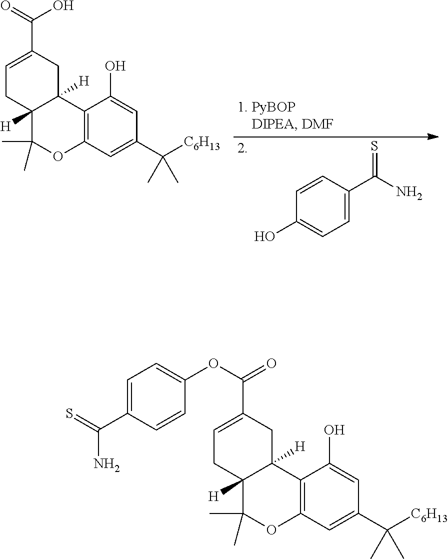

(R,R)-Ajulemic acid (AJA) (50 mg, 0.12 mmol, 1 eq) and 4-hydroxybenzothioamide (19 mg, 0.14 mmol, 1.1 eq) were dissolved in N,N-dimethylformamide (DMF) (3 mL), after which benzotriazol-1-yl-oxytripyrrolidinophosphonium hexafluorophosphate (PyBOP) (72 mg, 0.14 mmol, 1.1 eq) and diisopropylethylamine (43 μL, 0.31 mmol, 2.5 eq) were added to the mixture. The reaction mixture was stirred at room temperature for 16 h, then diluted in dichloromethane (DCM) and washed with water, 1N HCl and brine before drying over MgSO₄ and filtering. The solvents were removed by rotary evaporation under reduced pressure after which purification of the residue by flash chromatography on silica gel eluting with an increasing proportion of ethyl acetate (EtOAc) in hexane provided the title compound. Yield 53% (39 mg). ¹H NMR (400 MHz, CDCl₃): δ 0.83 (3H, t, J=6.6 Hz), 1.02-1.08 (2H, m), 1.15 (3H, s), 1.17-1.25 (12H, m), 1.43 (3H, s), 1.47-1.51 (2H, m), 1.85-1.92 (1H, m), 2.05-2.13 (2H, m), 2.46-2.53 (1H, m), 2.73 (1H, td, J=10.8, 4.4 Hz), 3.89-3.95 (1H, m), 6.28 (1H, s), 6.39 (1H, s), 7.12 (2H, d, J=8.2 Hz), 7.29-7.31 (1H, m), 7.51 (1H, s), 7.71 (1H, s), 7.89 (2H, d, J=8.2 Hz). HPLC RT=22.29 min (HPLC Method B).

### Example 22. Synthesis of (6aR,10aR)-1-Hydroxy-6,6-dimethyl-3-(2-methylheptan-2-yl)-6a,7,10,10a-tetrahydro-6H-benzo[c]chromene-9-carboxylic acid (Compound 12)

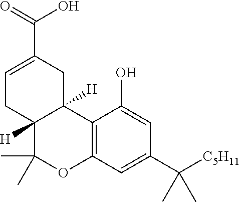

The standard procedure for synthesis of Ajulemic acid (AJA) was followed with replacement of 5-(2-methyloctan-2-yl)benzene-1,3-diol (DMHR) by 5-(2-methylheptan-2-yl)benzene-1,3-diol. ¹H NMR (400 MHz, CDCl₃): δ 0.82 (3H, t, J=7.6 Hz), 1.00-1.22 (2H, m), 1.14 (3H, s), 1.16-1.35 (10H, m), 1.41 (3H, s), 1.45-1.55 (2H, m), 1.80-1.90 (1H, m), 1.94-2.10 (2H, m), 2.38-2.50 (1H, m), 2.66 (1H, dt, J=2.4, 10.4 Hz), 3.82 (1H, d, J=18.8 Hz), 6.24 (1H, s), 6.39 (1H, s), 7.15 (1H, s).

### Example 23. Synthesis of (6aR,10aR)-9-Cyano-6,6-dimethyl-3-(2-methyloctan-2-yl)-6a,7,10,10a-tetrahydro-6H-benzo[c]chromen-1-yl acetate (Compound 19)

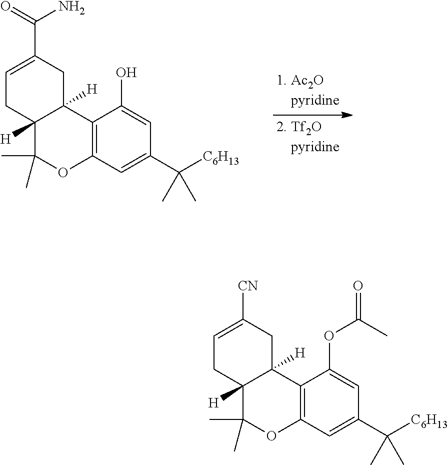

(6aR,10aR)-1-Hydroxy-6,6-dimethyl-3-(2-methyloctan-2-yl)-6a,7,10,10a-tetrahydro-6H-benzo[c]chromene-9-carboxamide (Compound 4, 196 mg, 0.49 mmol, 1 eq) was dissolved in dry pyridine (2 mL), after which acetic anhydride (Ac₂O) (70 μL, 0.74 mmol, 1.5 eq) was added. The reaction mixture was stirred at room temperature for 1 h, then diluted with water and ethyl acetate. The organic layer was washed with 1N HCl and brine, dried over MgSO₄ and filtered. The solvents were removed by rotary evaporation under reduced pressure and the residue was dissolved in pyridine (2 mL). After cooling to 0° C., triflic anhydride (42 μL, 0.54 mmol, 1.1 eq) was added. The reaction mixture was allowed to warm to room temperature and stirred for 1 h, then diluted with water and ethyl acetate. The organic layer was washed with 1N HCl and brine, dried over MgSO₄ and filtered. The solvents were removed by rotary evaporation under reduced pressure after which purification of the residue by flash chromatography on silica gel eluting with an increasing proportion of ethyl acetate (EtOAc) in hexane provided the title compound. Yield 60% (124 mg). ¹H NMR (400 MHz, CDCl₃): δ 0.84 (3H, t, J=6.8 Hz), 1.03-1.08 (2H, m), 1.12 (3H, s), 1.16-1.24 (12H, m), 1.40 (3H, s), 1.49-1.53 (2H, m), 1.83 (1H, td, J=11.6, 4.3 Hz), 1.94-2.04 (1H, m), 2.12-2.20 (1H, m), 2.32 (3H, s), 2.37-2.45 (1H, m), 2.63 (1H, td, J=11.2, 4.7 Hz), 3.08-3.14 (1H, m), 6.54 (1H, d, J=1.9 Hz), 6.67 (1H, m), 6.69 (1H, d, J=1.8 Hz). HPLC RT=25.93 min (HPLC Method B).

### Example 24. Synthesis of (6aS,10aS)-1-Hydroxy-6,6-dimethyl-3-(2-methyloctan-2-yl)-6a,7,10,10a-tetrahydro-6H-benzo[c]chromene-9-carboxylic acid (Compound 20)

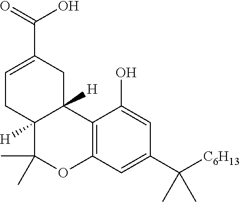

The standard procedure for synthesis of Ajulemic acid (AJA) was followed except that (4S)-PMD was used in place of the standard (4R) isomer. ¹H NMR (400 MHz, CDCl₃): δ 0.85 (3H, t, J=6.8 Hz), 1.00-1.15 (2H, m), 1.16 (3H, s), 1.18-1.32 (12H, m), 1.44 (3H, s), 1.48-1.56 (2H, m), 1.85 (1H, t, J=12 Hz), 1.93-2.108 (2H, m), 2.38-2.46 (1H, m), 2.70 (1H, t, J=11 Hz), 3.80-3.93 (1H, m), 6.26 (1H, s), 6.42 (1H, s), 7.18 (1H, s). Chiral purity: >99% ee (Chiralcel OD, 4.6×250 mm, isocratic 5% EtOH (0.1% TFA) in Heptane, run time: 25 min).

### Example 25. Synthesis of 2,3-Dihydroxypropyl (6aR,10aR)-1-hydroxy-6,6-dimethyl-3-(2-methyloctan-2-yl)-6a,7,10,10a-tetrahydro-6H-benzo[c]chromene-9-carboxylate (Compound 22)

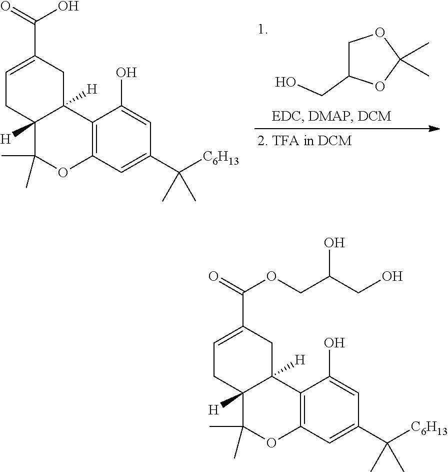

Step 1: (R,R)-Ajulemic acid (AJA) (300 mg, 0.75 mmol, 1 eq) was dissolved in dichloromethane (DCM) (5 mL) and 1-ethyl-3-(3-dimethylaminopropyl)carbodiimide (EDCl) (127.90 mg, 0.82 mmol, 1.1 eq), 4-(dimethylamino)pyridine (DMAP) (45.75 mg, 0.37 mmol, 0.5 eq) and 1,2-isopropylideneglycerol (690 μL, 7.49 mmol, 10 eq) were added sequentially. The reaction mixture was stirred at room temperature (rt) for 16 h, then diluted with EtOAc and washed with an aqueous solution of 1 M HCl before being dried over MgSO₄ and filtered. The solvents were removed by rotary evaporation under reduced pressure and the residue purified by flash chromatography on silica gel eluting with an increasing proportion of EtOAc in hexanes to provide the protected glycerol ester.

Step 2: The intermediate was dissolved in dichloromethane (DCM) (2 mL) and trifluoroacetic acid (TFA) (689 μL, 1.91 mmol, 9 eq). The reaction mixture was stirred at room temperature for 1.5 h after which the volatiles were removed by rotary evaporation under reduced pressure. Purification of the residue by flash chromatography on silica gel eluting with an increasing proportion of EtOAc in hexanes provided the title compound.

Yield: 11% (41 mg). ¹H NMR (400 MHz, CDCl₃): δ 0.85 (3H, t, J=6.8 Hz), 1.03-1.10 (2H, m), 1.13 (3H, s), 1.18-1.25 (12H, m), 1.41 (3H, s), 1.48-1.52 (2H, m), 1.80-1.88 (1H, m), 1.96-2.11 (3H, m), 2.38-2.46 (1H, m), 2.55 (1H, s), 2.64-2.70 (1H, m), 3.61-3.65 (1H, m), 3.70-3.74 (1H, m), 3.78-3.84 (1H, m), 3.97 (1H, s), 4.22-4.33 (2H, m), 4.77 (1H, s), 6.24 (1H, d, J=1.8 Hz), 6.40 (1H, d, J=1.7 Hz), 7.06-7.08 (1H, m). LC-MS (ESI−): 473.27 (M−H)⁻. HPLC RT=19.3 min (HPLC Method A).

### Example 26. Synthesis of Methyl (6aR,10aR)-1-hydroxy-6,6-dimethyl-3-(2-methyloctan-2-yl)-6a,7,10,10a-tetrahydro-6H-benzo[c]chromene-9-carboxylate (Compound 28)

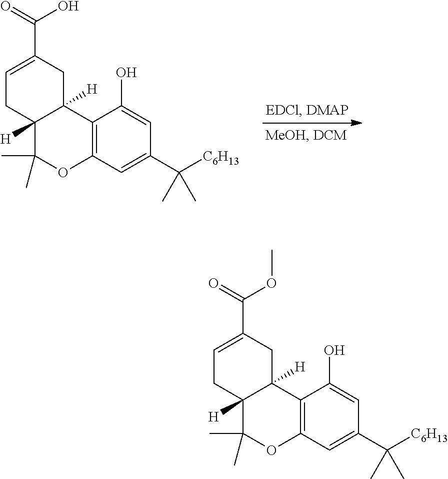

(R,R)-Ajulemic acid (AJA) (200 mg, 0.50 mmol, 1 eq) was dissolved in dichloromethane (DCM) (5 mL). 1-Ethyl-3-(3-dimethylaminopropyl)carbodiimide (EDCI, 85.27 mg, 0.55 mmol, 1.1 eq), 4-(dimethylamino)pyridine (DMAP, 30.5 mg, 0.25 mmol, 0.5 eq) and methanol (MeOH, 203 μL, 4.99 mmol. 10 eq) were added sequentially. The reaction mixture was stirred at room temperature (rt) for 16 h, then diluted with EtOAc and washed with 1 M HCl before being dried over MgSO₄ and filtered. The solvents were removed by rotary evaporation under reduced pressure, after which purification of the residue by flash chromatography on silica gel eluting with an increasing proportion of EtOAc in hexanes provided the title compound.

Yield 46% (99 mg). ¹H NMR (400 MHz, CDCl₃): δ 0.84 (3H, t, J=6.9 Hz), 1.03-1.08 (2H, m), 1.13 (3H, s), 1.16-1.25 (12H, m), 1.41 (3H, s), 1.47-1.52 (2H, m), 1.83 (1H, td, J=11.5, 4.3 Hz), 1.96-2.05 (2H, m), 2.37-2.44 (1H, m), 2.66 (1H, td, J=11.1, 4.5 Hz), 3.74 (3H, s), 3.78-3.84 (1H, m), 4.87 (1H, s), 6.25 (1H, d, J=1.8 Hz), 6.39 (1H, d, J=1.8 Hz), 7.02 (1H, d, J=5.1 Hz). LC-MS (ESI−): 413.32 (M−H)⁻. HPLC RT=22.6 min (HPLC Method A).

### Example 27. Synthesis of Isopropyl (6aR,10aR)-1-hydroxy-6,6-dimethyl-3-(2-methyloctan-2-yl)-6a,7,10,10a-tetrahydro-6H-benzo[c]chromene-9-carboxylate (Compound 29)

Compound 29 was synthesized according to the “general procedure for esterification using acetyl chloride.” Yield 49% (82.5 mg). ¹H NMR (400 MHz, CDCl₃): δ 0.84 (3H, t, J=6.8 Hz), 1.03-1.07 (2H, m), 1.13 (3H, s), 1.16-1.21 (12H, m), 1.27 (6H, t, J=6.7 Hz), 1.40 (3H, s), 1.47-1.52 (2H, m), 1.83 (1H, td, J=11.5, 4.3 Hz), 2.03-1.95 (2H, m), 2.43-2.36 (1H, m), 2.66 (1H, td, J=11.1, 4.5 Hz), 3.81 (1H, dd, J=16.7, 4.4 Hz), 4.97 (1H, s), 5.05-5.12 (1H, m), 6.26 (1H, d, J=1.8 Hz), 6.38 (1H, d, J=1.8 Hz), 6.99 (1H, m). LC-MS (ESI−): 441.47 (M−H)⁻. HPLC RT=23.9 min (HPLC Method A).

### Example 28. Synthesis of Ethyl (6aR,10aR)-1-hydroxy-6,6-dimethyl-3-(2-methyloctan-2-yl)-6a,7,10,10a-tetrahydro-6H-benzo[c]chromene-9-carboxylate (Compound 54)

Compound 54 was synthesized according to the “general procedure for esterification using acetyl chloride.” Yield 81% (357.2 mg). ¹H NMR (400 MHz, CDCl₃): δ 0.84 (3H, t, J=6.8 Hz), 1.03-1.08 (2H, m), 1.13 (3H, s), 1.16-1.25 (12H, m), 1.30 (3H, t, J=7.1 Hz), 1.41 (3H, s), 1.47-1.51 (2H, m), 1.83 (1H, td, J=11.5, 4.3 Hz), 1.96-2.05 (2H, m), 2.37-2.43 (1H, m), 2.66 (1H, td, J=11.1, 4.5 Hz), 3.80-3.86 (1H, m), 4.22 (2H, q, J=7.1 Hz), 5.09 (1H, s), 6.26 (1H, d, J=1.8 Hz), 6.38 (1H, d, J=1.8 Hz), 7.0-7.02 (1H, m). LC-MS (ESI+): 429.26 (M+H)⁺. HPLC RT=23.11 min (HPLC Method A).

### Example 29. Synthesis of (6aR,10aR)-6,6-dimethyl-3-(2-methyloctan-2-yl)-1,2-dioxo-2,6,6a,7,10,10a-hexahydro-1H-benzo[c]chromene-9-carboxylic acid (Compound 30) and (6aR,10aR)-6,6-dimethyl-3-(2-methyloctan-2-yl)-1,4-dioxo-4,6,6a,7,10,10a-hexahydro-1H-benzo[c]chromene-9-carboxylic acid (Compound 32)

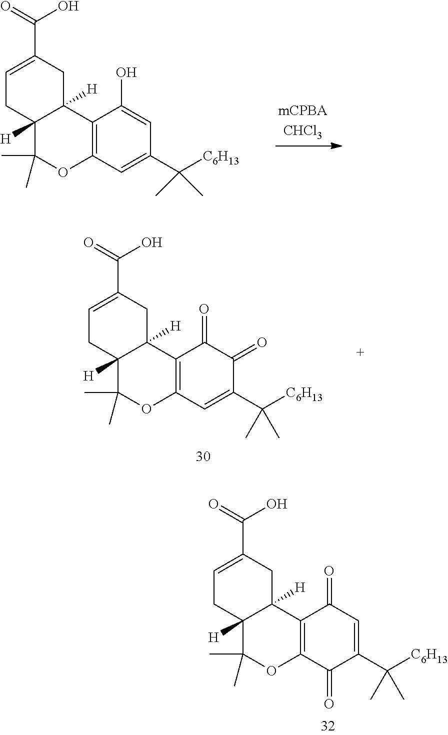

A solution of (R,R)-ajulemic acid (250 mg, 620 μmol) in CHCl₃ (5 mL) was treated with 70% mCPBA (172 mg, 700 μmol, 1.1 equiv) with stirring at rt for 16 h while being protected from light. The reaction mixture was diluted with water and dichloromethane and the organic layer washed with water, dried over MgSO₄, filtered and concentrated in vacuo. Purification of the residue by flash chromatography on silica gel eluting with a gradient of EtOAc in hexanes containing 5% AcOH provided the title compounds.

Compound 30: Yield: 33% (85 mg). ¹H NMR (400 MHz, CDCl₃): δ 0.85 (3H, t, J=6.8 Hz), 1.02 (2H, m), 1.19 (6H, s), 1.22 (3H, s), 1.25-1.19 (6H, m), 1.47 (3H, s), 1.75-1.61 (3H, m), 1.84 (1H, m), 2.01 (1H, m), 2.41 (2H, m), 3.62 (1H, m), 6.46 (1H, s), 7.08 (1H, m). LCMS (ESI+): 415.3 (M+H⁺).

Compound 32: ¹H NMR (400 MHz, CDCl₃): δ 0.85 (3H, t, J=6.8 Hz), 1.03 (2H, m), 1.21 (3H, s), 1.26 (6H, s), 1.30-1.11 (6H, m), 1.54 (3H, s), 1.76-1.62 (3H, m), 1.90 (1H, m), 2.04 (1H, m), 2.35 (2H, m), 3.56 (1H, m), 6.41 (1H, s), 7.12 (1H, m). LCMS (ESI+): 415.3 (M+H⁺).

### Example 30: Synthesis of 1-hydroxy-6,6-dimethyl-3-(2-methyloctan-2-yl)-6H-benzo[c]chromene-9-carboxylic acid (Compound 31)

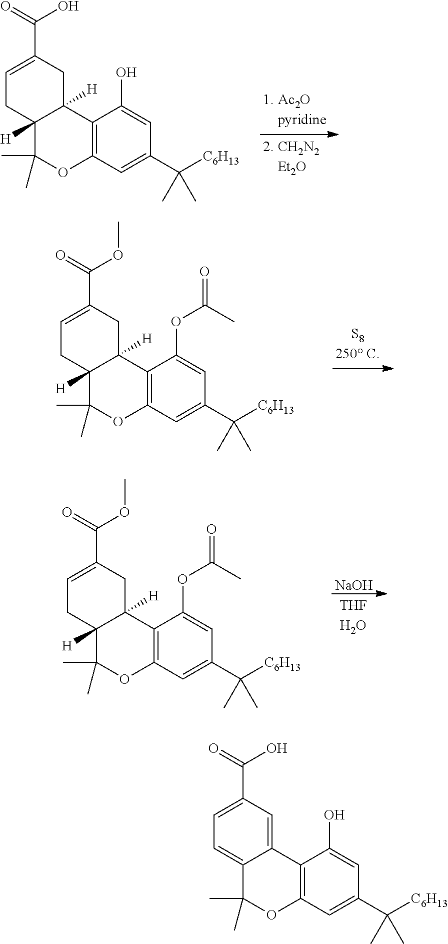

To a solution of (R,R)-Ajulemic acid (3.44 g, 8.59 mmol, 1.0 eq) in dry pyridine (25 mL) was added Ac₂O dropwise at rt. The resulting mixture was stirred for 2h at rt. Upon completion of the reaction, water was added and the product was extracted with EtOAc (×3). The combined organic layers were washed with 1N HCl, then brine, dried over MgSO₄ and concentrated in vacuum. The residue was purified by silica gel chromatography (eluting with a gradient of EtOAc in hexanes) to afford 3.4 g of the acetylated intermediate in 89% yield.

To a solution of the acetylated intermediate (3.4 g, 7.69 mmol, 1.0 eq) in dry diethyl ether (20 mL) was added CH₂N₂ (0.5 M in solution in Et₂O, 20 mL, 10 mmol, 1.3 eq) dropwise. The resulting mixture was stirred for 2h at rt, after which the excess diazomethane was quenched with AcOH (0.132 mL, 2.31 mmol, 0.3 eq) and the solvents were evaporated under vacuum. The resulting mixture was purified by silica gel chromatography (eluting with a gradient of EtOAc in hexanes) to afford 3.06 g of the methyl ester as an orange oil in 87% yield.

A reaction vial was loaded with the methyl ester (3.06 g, 6.70 mmol, 1.0 eq) and S₈ (430 mg, 13.4 mmol, 2.0 eq). The resulting mixture was heated neat to 250° C. overnight, then allowed to cool to rt. The mixture was dissolved in dichloromethane and purified by silica gel chromatography (eluting with a gradient of EtOAc in hexanes) to afford 2.7 g of the aromatized intermediate in 90% yield.

The aromatized intermediate (2.4 g, 5.3 mmol, 1.0) was dissolved in THF (10 mL), after which 50% aqueous NaOH (w/w, 6 mL) was added. The resulting mixture was heated to 50° C. for 4h. After cooling to rt, the reaction mixture was acidified with 6M HCl and extracted with EtOAc (×3). The combined organic layers were washed with brine, dried over MgSO₄ and concentrated in vacuum. The residue was purified by silica gel chromatography (eluting with a gradient of EtOAc in hexanes) to afford 2 g of the title compound.

Yield: 95% (2 g). ¹H NMR (400 MHz, CDCl₃): δ 0.83 (3H, t, J=6.8 Hz), 1.02-1.08 (2H, m), 1.14-1.30 (6H, m), 1.24 (6H, s), 1.50-1.56 (2H, m), 1.65 (6H, s), 6.43 (1H, s), 6.58 (1H, s), 7.34 (1H, d, J=8.0 Hz), 7.99 (1H, dd, J=1.2 Hz, 8.0 Hz), 9.2 (1H, s). LCMS (ESI+): 397.2 (M+H+).

### Example 31. Synthesis of (6aR,10aR)-1-Hydroxy-6,6-dimethyl-3-(2-methyloctan-2-yl)-6a,7,8,10a-tetrahydro-6H-benzo[c]chromene-9-carboxylic acid (Compound 33)

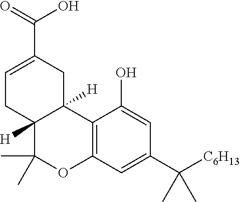

The standard procedure for synthesis of Ajulemic acid (AJA) was followed except that temperatures in the first step (comprised of coupling between DMHR and PMD, cyclization and acetylation) were held at or below 40° C. ¹H NMR (400 MHz, CDCl₃): δ 0.85 (3H, t, J=6.8 Hz), 0.95-1.10 (2H, m), 1.14 (3H, s), 1.10-1.35 (12H, m), 1.46 (3H, s), 1.35-1.60 (4H, m), 1.75 (1H, t, J=12 Hz), 1.93-2.11 (1H, m), 2.35-2.52 (1H, m), 2.52-2.68 (1H, m), 3.40 (1H, d, J=9 Hz), 6.27 (1H, s), 6.40 (1H, s), 8.15 (1H, s).

### Example 32. Synthesis of (6aR,10aR)-1-methoxy-6,6-dimethyl-3-(2-methyloctan-2-yl)-6a,7,10,10a-tetrahydro-6H-benzo[c]chromene-9-carboxylic acid (Compound 34)

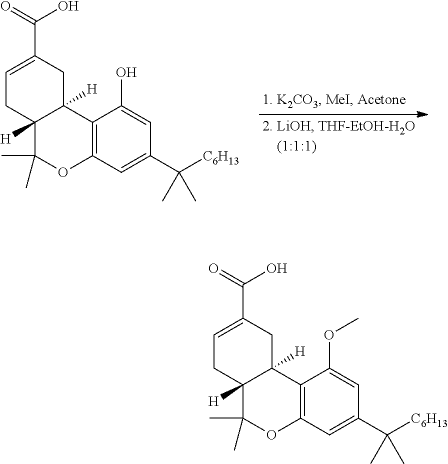

(R,R)-Ajulemic acid (AJA) (300 mg, 0.75 mmol, 1 eq) was dissolved in acetone (8 mL), and potassium carbonate (828 mg, 5.99 mmol, 8 eq) was added, followed by iodomethane (1 mL, 14.98 mmol, 20 eq). The reaction was stirred at 60° C. for 16 h in a closed vessel. The reaction mixture was concentrated by rotary evaporation under reduced pressure and the residue dissolved in ether and water. The organic layer was dried over MgSO₄, filtered and concentrated by rotary evaporation. The residue was dissolved in THF-MeOH—H₂O (1:1:1) (6 mL). Lithium hydroxide (LiOH) (108 mg, 4.49 mmol, 6 eq) was added and the mixture stirred at 50° C. for 3 h before cooling to 0° C. and acidifying it to pH 2 using 1N HCl. The mixture was extracted with EtOAc and the organic layer washed twice with H₂O before being dried over MgSO₄, filtered and concentrated to provide the title compound,

Yield: 87% (282 mg). ¹H NMR (400 MHz, CDCl₃): δ 0.85 (3H, t, J=6.8 Hz), 1.04-1.12 (2H, m), 1.12 (3H, s), 1.17-1.22 (6H, m), 1.24 (6H, s), 1.41 (3H, s), 1.51-1.56 (2H, m), 1.84 (1H, td, J=11.6, 4.4 Hz), 1.90-2.07 (2H, m), 2.43 (1H, m), 2.64 (1H, td, J=11.1, 4.5 Hz), 3.72-3.77 (1H, m), 3.82 (3H, s), 6.39 (1H, d, J=1.7 Hz), 6.43 (1H, d, J=1.7 Hz), 7.13-7.15 (1H, m). LC-MS (ESI+): 415.23 (M+H)⁺. HPLC RT=23 min (HPLC Method A).

### Example 33. Synthesis of (6aR,10aR)-1-Hydroxy-6,6-dimethyl-3-(2-phenylpropan-2-yl)-6a,7,10,10a-tetrahydro-6H-benzo[c]chromene-9-carboxylic acid (Compound 35)

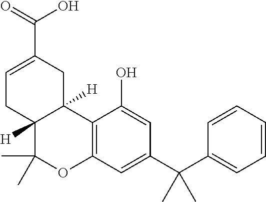 [SMILES: CC(C)(c1ccccc1)c1cc(O)c2c(c1)OC(C)(C)[C@@H]1CC=C(C(=O)O)C[C@@H]21]

The standard procedure for synthesis of Ajulemic acid (AJA) was followed with replacement of 5-(2-methyloctan-2-yl)benzene-1,3-diol (DMHR) by 5-(2-phenylpropan-2-yl)benzene-1,3-diol.

¹H NMR (400 MHz, CDCl₃): δ 1.14 (3H, s), 1.40 (3H, s), 1.60 (6H, d, J=3.3 Hz), 1.76-1.88 (2H, m), 1.92-2.07 (3H, m), 2.39-2.46 (1H, m), 2.61-2.68 (1H, m), 3.76-3.82 (1H, m), 6.00 (1H, s), 6.42 (1H, s), 7.10-7.18 (2H, m), 7.23-7.25 (3H, m). LC-MS (ESI+): 393.2 (M+H)⁺. HPLC RT=16.8 min (HPLC Method A).

### Example 34. Synthesis of (6aR,10aR)-N-(tert-butoxy)-1-hydroxy-6,6-dimethyl-3-(2-methyloctan-2-yl)-6a,7,10,10a-tetrahydro-6H-benzo[c]chromene-9-carboxamide (Compound 88)

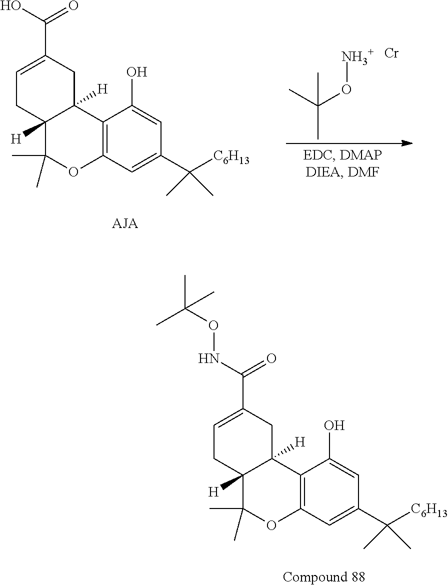

Compound 88 was synthesized according to the “general procedure for amide bond formation with HATU.” Yield 35.2% (166 mg). ¹H NMR (300 MHz, CDCl₃) δ 10.28 (s, 1H), 9.24 (s, 1H), 6.41 (s, 1H), 6.31 (s, 1H), 6.13 (s, 1H), 3.67 (d, J=17.25 Hz, 1H), 2.47 (t, J=7.98 Hz, 1H), 2.28 (d, J=16.9 Hz, 1H), 1.94 (t, J=15 Hz, 1H), 1.80-1.57 (m, 2H), 1.50-1-41 (m, H) 1.31 (s, 3H), 1.16 (s, 21H), 1.02 (s, 5H), 0.81 (s 3H). LC-MS (ESI+): 472.3 (M+H⁺); HPLC RT: 4.35 min. (HPLC Method C).

### Example 35. Synthesis of (6aR,10aR)-1-hydroxy-6,6-dimethyl-3-(2-methyloctan-2-yl)-N-(piperidin-1-yl)-6a,7,10,10a-tetrahydro-6H-benzo[c]chromene-9-carboxamide (Compound 48)

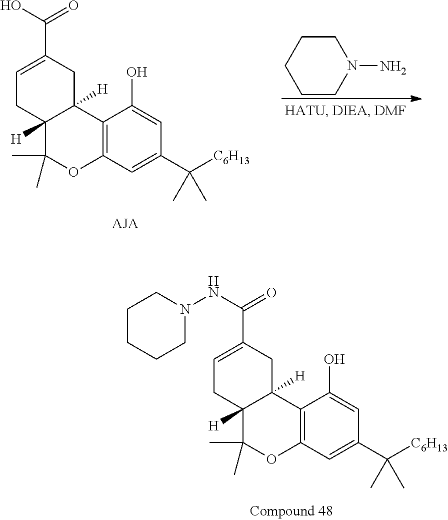

Compound 48 was synthesized according to the “general procedure for amide bond formation with HATU.” Yield 32.6% (157 mg). ¹H NMR (300 MHz, CDCl₃) δ 9.22 (s, 1H), 8.64 (s, 1H), 6.38 (s, 1H), 6.30 (s, 1H), 6.12 (s, 1H), 3.63 (d, J=16.5 Hz, 1H) 2.70 (s, 4H), 2.45 (t, J=3.4 Hz, 1H), 2.27 (d, J=14.2 Hz, 1H), 1.91 (t, J=15.6 Hz, 1H), 1.79-1-40 (m, 7H), 1.30 (s, 5H), 1.14 (s, 13H), 1.01 (s, 5H), 0.81 (s, 3H). LC-MS (ESI-TOF+): 483.5130 (M+H⁺); HPLC RT: 4.467 min. (HPLC Method D).

### Example 36. (6aR,10aR)-1-hydroxy-N-((1R,3R)-3-hydroxycyclobutyl)-6,6-dimethyl-3-(2-methyloctan-2-yl)-6a,7,10,10a-tetrahydro-6H-benzo[c]chromene-9-carboxamide (Compound 45)

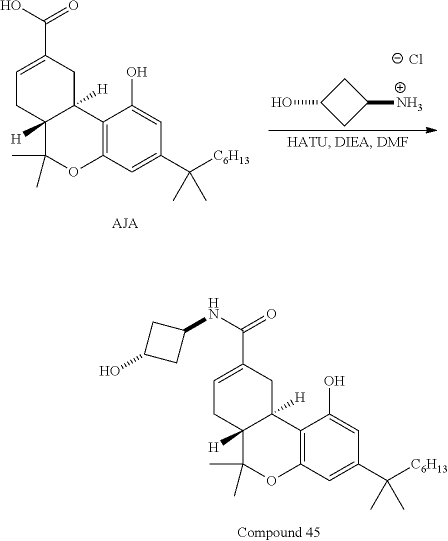

Compound 45 was synthesized according to the “general procedure for amide bond formation with HATU.” Yield 63.9% (300 mg). ¹H NMR (300 MHz, CDCl₃) δ 9.12 (s, 1H), 7.86 (d, J=6.33 Hz, 1H), 6.47 (s, 1H), 6.31 (s, 1H), 6.13 (s, 1H), 4.94 (s, J=5.01 Hz, 1H), 4.24 (d, J=4.74 Hz, 2H), 3.65 (d, J=17.1 Hz, 1H), 2.44 (t, J=12.27 Hz, 1H), 2.35-1-86 (m, 6H), 1.79-1-59 (m, 2H), 1.47-1.40 (m, 2H, 1.31 (s, 3H), 1.14 (s, 12H), 1.01 (s, 5H), 0.81 (s, 3H). LC-MS (ESI+): 470.3 (M+H⁺); HPLC RT: 3.99 min. (HPLC Method C).

### Example 37. (6aR,10aR)-1-hydroxy-N-((1 S,3S)-3-hydroxycyclobutyl)-6,6-dimethyl-3-(2-methyloctan-2-yl)-6a,7,10,10a-tetrahydro-6H-benzo[c]chromene-9-carboxamide (Compound 46)

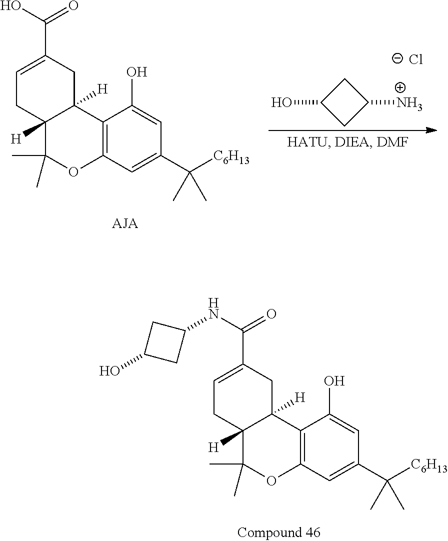

Compound 46 was synthesized according to the “general procedure for amide bond formation with HATU.” Yield 54.4% (179 mg). ¹H NMR (300 MHz, DMSO-d₆) δ 9.20 (s, 1H); 7.82 (d, J=6.72 Hz, 1H), 6.48 (s, 1H), 6.31 (s, 1H), 6.13 (s, 1H), 5.01 (d, J=4.77 Hz, 1H), 3.84-3.59 (m, 3H), 2.43 (bs, 2H), 2.27 (d, J=16.4 Hz, 1H), 2.02-1.57 (m, 5H), 1.45 (bs, 2H), 1.31 (s, 3H), 1.14 (s, 13H), 1.01 (s, 5H), 0.81 (s, 3H). LC-MS (ESI+): 470.3 (M+H⁺); HPLC RT: 3.98 min. (HPLC Method C).

### Example 38. (6aR,10aR)-N-(adamantan-1-yl)-1-hydroxy-6,6-dimethyl-3-(2-methyloctan-2-yl)-6a,7,10,10a-tetrahydro-6H-benzo[c]chromene-9-carboxamide (Compound 42)

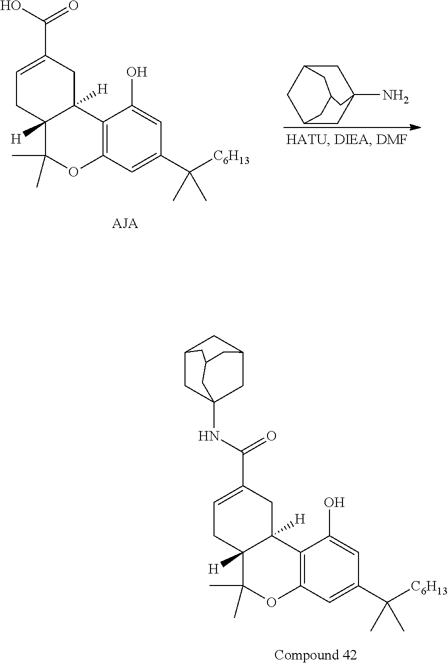

Compound 42 was synthesized according to the “general procedure for amide bond formation with HATU.” Yield 35.6% (190 mg). ¹H NMR (300 MHz, CDCl₃) δ 9.21 (s, 1H), 6.82 (s, 1H), 6.37 (s, 1H), 6.32 (s, 1H), 6.13 (s, 1H), 3.63 (d, J=17.34 Hz, 1H), 2.44 (t, J=10.5 Hz, 1H), 2.26 (d, J=13.47 Hz, 1H), 1.99 (d, J=11.97 Hz, 10H), 1.76 (d, J=11.8 Hz, 1H), 1.46 (bs, 2H), 1.31 (s, 3H), 1.16 (d, J=8.31 Hz, 12H), 1.02 (s, 5H), 0.83 (s, 3H). LC-MS (ESI+): 534.3 (M+H⁺); HPLC RT: 4.97 min. (HPLC Method C).

### Example 39. (6aR,10aR)-1-hydroxy-N-(3-hydroxyadamantan-1-yl)-6,6-dimethyl-3-(2-methyloctan-2-yl)-6a,7,10,10a-tetrahydro-6H-benzo[c]chromene-9-carboxamide (Compound 43)

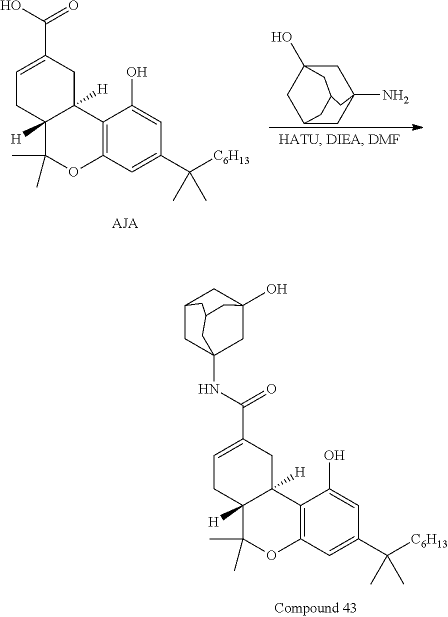

Compound 43 was synthesized according to the “general procedure for amide bond formation with HATU.” Yield 21.8% (120 mg). ¹H NMR (300 MHz, DMSO-d₆) δ 9.21 (s, 1H), 6.91 (s, 1H), 6.36 (s, 1H), 6.31 (s, 1H), 6.12 (s, 1H), 4.45 (s, 1H), 3.61 (d, J=17.61 Hz, 1H), 2.43 (t, J=10.32 Hz, 1H), 2.25 (d, J=16.98 Hz, 1H), 2.11 (s, 2H), 1.84 (s, 7H), 1.77-1.57 (m, 2H) 1.51 (s, 4H), 1.44 (s, 4H), 1.30 (s, 3H), 1.15 (d, J=10.35 Hz, 12H), 1.01 (s, 5H), 0.81 (t, J=6.15 Hz, 3H). LC-MS (ESI+): 550.3 (M+H⁺); HPLC RT: 4.39 min. (HPLC Method C).

### Example 40. (6aR,10aR)-N-(3,3-difluorocyclobutyl)-1-hydroxy-6,6-dimethyl-3-(2-methyloctan-2-yl)-6a,7,10,10a-tetrahydro-6H-benzo[c]chromene-9-carboxamide (Compound 89)

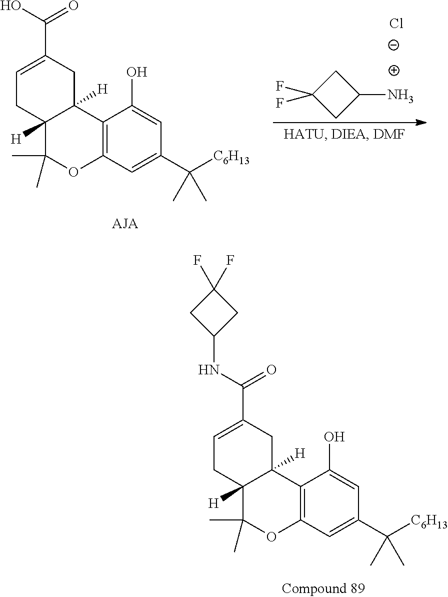

Compound 89 was synthesized according to the “general procedure for amide bond formation with HATU.” Yield 40.5% (198 mg). 1H NMR (300 MHz, CDCl₃) δ 6.68 (d, J=4.77 Hz, 1H), 6.35 (dd, J=11.76-1.44 Hz, 2H), 6.00 (s, 1H), 5.97 (d, J=6.66 Hz, 1H), 4.34 (m, 1H), 3.81 (dd, J=14.16-4.79 Hz, 1H), 3.11-2.94 (m, 2H), 2.70 (dt, J=10.87-4.41 Hz, 1H), 2.58-2.33 (m, 3H), 2.05-1.92 (m, 2H), 1.82 (dt, J=11.63-3.93 Hz, 1H), 1.52-1.46 (m, 2H), 1.40 (s, 3H), 1.20 (s, 12H), 1.12 (s, 3H), 1.06 (bs, 2H), 0.84 (t, J=6.97 Hz, 3H). LC-MS (ESI+): 489.3 (M+H⁺); R.T.: 5.316 min. (HPLC Method D).

### Example 41. (6aR,10aR)-1-hydroxy-6,6-dimethyl-3-(2-methyloctan-2-yl)-N-(oxetan-3-yl)-6a,7,10,10a-tetrahydro-6H-benzo[c]chromene-9-carboxamide (Compound 90)

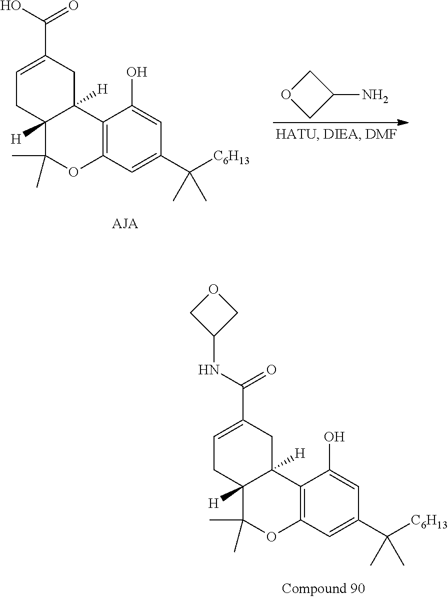

Compound 90 was synthesized according to the “general procedure for amide bond formation with HATU.” Yield 28.4% (97 mg). ¹H NMR (300 MHz, CDCl₃) 36.76 (d, J=4.47 Hz, 1H) 6.37 (s, 1H), 6.27 (d, J=8.94 Hz, 2H), 5.84 (s, 1H), 5.11 (m, 1H), 4.96 (q, J=5.88 Hz, 2H), 3.80 (dd, J=15.02-3.12 Hz, 1H), 2.71 (dt, J=10.56-4.38 Hz, 1H) 2.43-2.33 (m, 1H), 2.07-1.93, (m, 2H), 1.82 (dt, J=11.14-3.75 Hz, 1H), 1.61 (s, 1H), 1.52-1.46 (m, 2H), 1.40 (s, 3H), 1.24 (s, 1H), 1.20 (s, 11H), 1.12 (s, 3H), 1.06 (bs, 2H), 0.84 (t, J=6.93 Hz, 3H). LC-MS (ESI+): 456.2 (M+H⁺); R.T.: 4.409 min. (HPLC Method C).

### Example 42. (6aR,10aR)-1-hydroxy-N-isopropyl-6,6-dimethyl-3-(2-methyloctan-2-yl)-6a,7,10,10a-tetrahydro-6H-benzo[c]chromene-9-carboxamide (Compound 91)

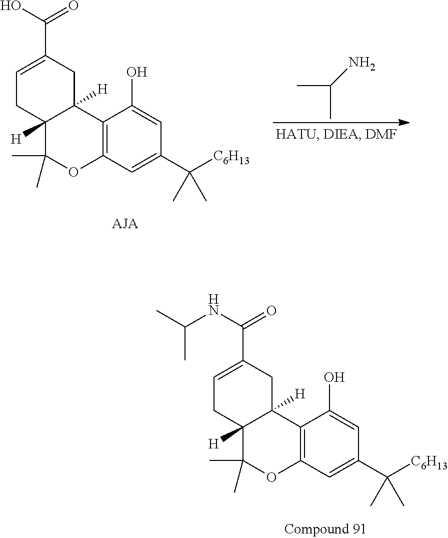

Compound 91 was synthesized according to the “general procedure for amide bond formation with HATU.” Yield 47.5% (157 mg). ¹H NMR (300 MHz, CDCl₃), δ 9.21 (s, 1H), 7.46 (d, J=7.77 Hz, 1H), 6.45 (s, 1H), 6.31 (d, J=1.53 Hz, 1H) 6.13 (d, J=1.50 Hz, 1H), 3.92 (m, 1H), 3.65 (dd, J=17.74-2.86 Hz, 1H), 2.44 (dt, J=10.94-4.37 Hz, 1H), 2.28 (d, J=19.11 Hz, 1H), 1.93 (t, J=16.2 Hz, 1H), 1.76 (d, J=14.91 Hz, 1H) 1.62 (dt, J=11.49-4.41 Hz, 1H), 1.48-1.43 (m, 2H), 1.31 (s, 3H), 1.16 (s, 5H), 1.14 (s, 6H), 1.06 (dd, J=6.38-2.76 Hz, 7H), 1.02 (s, 5H), 0.81 (t, J=6.81 Hz, 3H). LC-MS (ESI+): 442.3 (M+H⁺); R.T.: 4.459 min. (HPLC Method C).

### Example 43. (6aR,10aR)-N-cyclopentyl-1-hydroxy-6,6-dimethyl-3-(2-methyloctan-2-yl)-6a,7,10,10a-tetrahydro-6H-benzo[c]chromene-9-carboxamide (Compound 92)

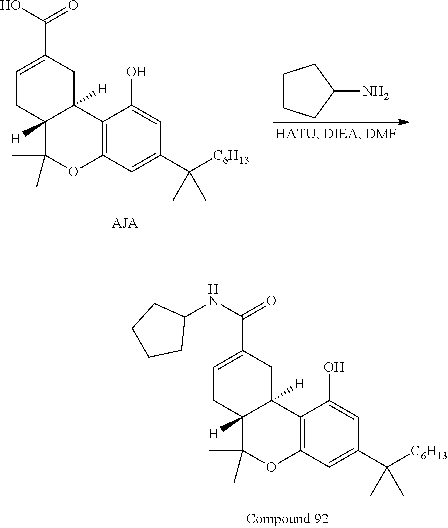

Compound 92 was synthesized according to the “general procedure for amide bond formation with HATU.” Yield 22.7% (106 mg). ¹H NMR (300 MHz, CDCl₃) δ 6.61 (s, 1H), 6.57 (d, J=4.68 Hz, 1H), 6.41 (d, J=1.44 Hz, 1H), 6.34 (d, J=1.50 Hz, 1H), 5.71 (d, J=7.26 Hz, 1H), 4.30 (m, 1H), 3.87 (dd, J=14.91-4.02. 1H), 2.70 (dt, J=10.74-4.44 Hz, 1H), 2.34 (m, 1H), 2.09-1.893 (m, 4H), 1.81 (dt, J=12.07-3.78 Hz, 1H), 1.72-1.56 (m, 5H), 1.52-1.44 (m, 2H), 1.39 (s, 4H), 1.19 (s, 12H), 1.11 (s, 3H), 1.06 (bs, 2H), 0.83 (t, J=6.99 Hz, 3H). LC-MS (ESI+): 468.3 (M+H⁺); R.T.: 5.501 min. (HPLC Method D).

### Example 44. ((6aR,10aR)-1-hydroxy-6,6-dimethyl-3-(2-methyloctan-2-yl)-6a,7,10,10a-tetrahydro-6H-benzo[c]chromen-9-yl)(piperidin-1-yl)methanone (Compound 93)

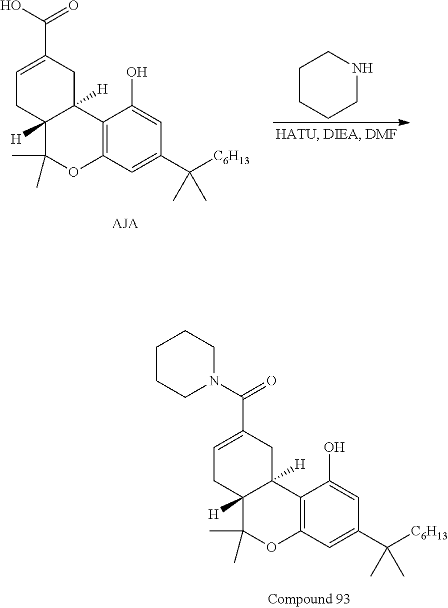

Compound 93 was synthesized according to the “general procedure for amide bond formation with HATU.” Yield 28.2% (99 mg). ¹H NMR (300 MHz, CDCl₃) δ 6.81 (s, 1H), 6.37 (d, J=1.61, 1H), 6.32 (d, J=1.61 Hz, 1H), 5.79 (d, J=4.72 Hz, 1H), 3.76 (dd, J=17.32-4.72 Hz, 1H), 3.51 (bs, 4H), 2.77 (m, 1H), 2.32-2.22 (m, 1H), 2.05-1.81 (m, 2H), 1.68-1.46 (m, 8H), 1.38 (s, 3H), 1.25 (s, 2H), 1.19 (s, 11H), 1.08 (s, 4H), 0.84 (t, J=6.975 Hz, 3H). LC-MS (ESI+): 468.3 (M+H⁺); R.T.: 4.507 min. (HPLC Method D).

### Example 45. (6aR,10aR)-1-hydroxy-N-(1-iminoethyl)-6,6-dimethyl-3-(2-methyloctan-2-yl)-6a,7,10,10a-tetrahydro-6H-benzo[c]chromene-9-carboxamide (Compound 94)

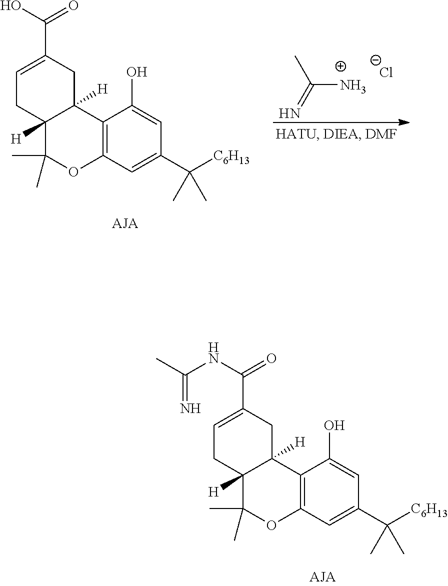

Compound 94 was synthesized according to the “general procedure for amide bond formation with HATU.” Yield 1.3% (7.0 mg). ¹H NMR (300 MHz, DMSO-d₆) δ 8.13 (s, 0.3H) 6.82 (dd, J=22.56-4.83 Hz, 0.5H) 6.39-6.34 (m, 1H), 6-26 (s, 1H), 5.63 (bs, 1H), 3.95-3-71 (m, 1H), 3.64 (s, 1H), 2.74-2.61 (m, 1H), 2.52 (s, 1H), 2.49-2.28 (m, 1H), 2-16 (s, 1H), 2-08-1.97 (m, 2H), 1.87-1.78 (dt, J=11.4-4.23 Hz, 1H), 1.52-1.45 (m, 2H), 1.40 (d, J=4.89 Hz, 3H), 1.25 (s, 6H), 1.20 (m, 9H), 1.28 (d, J=1.68 Hz, 3H), 0.84 (m, 4H). LC-MS (ESI+): 441.3130 (M+H⁺); R.T.: 2.073 min. (HPLC Method D).

### Example 46. (6aR,10aR)-N-(tert-butyl)-1-hydroxy-6,6-dimethyl-3-(2-methyloctan-2-yl)-6a,7,10,10a-tetrahydro-6H-benzo[c]chromene-9-carboxamide (Compound 95)

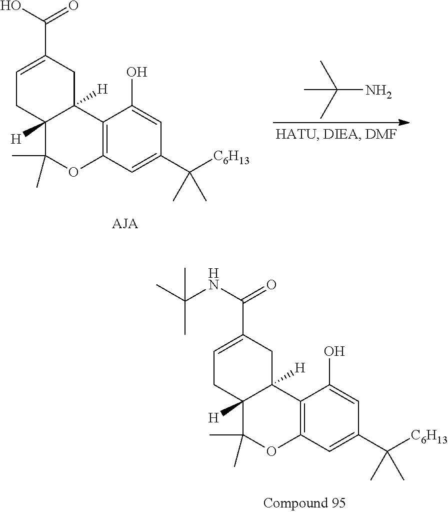

Compound 95 was synthesized according to the “general procedure for amide bond formation with HATU.” Yield 28.2% (99 mg). ¹H NMR (300 MHz, DMSO-d₆) δ 9.2 (s, 1H), 6.97 (s, 1H), 6.38 (s, 1H), 6.31 (d, J=1.47 Hz, 1H), 6.13 (d, J=1.47 Hz, 1H), 3.62 (d, J=14.70 Hz, 1H), 2.43 (dt, J=11.1-4.32 Hz. 1H), 2.30-2.20 (m, 1H), 1.91 (t, J=15.72 Hz, 1H), 1.72 (t, J=14.82 Hz, 1H), 1.61 (dt, J=11.40-4.41 Hz, 1H), 1.48-1.43 (m, 2H), 1.31 (s, 3H), 1.27 (s, 9H), 1.16 (s, 5H), 1.14 (s, 7H), 1.01 (s, 4H), 0.81 (m, 4H). LC-MS (ESI+): 456.3 (M+H⁺); R.T.: 4.632 min. (HPLC Method C).

### Example 47. (6aR,10aR)-1-hydroxy-6,6-dimethyl-3-(2-methyloctan-2-yl)-N-(1-(trifluoromethyl)cyclobutyl)-6a,7,10,10a-tetrahydro-6H-benzo[c]chromene-9-carboxamide (Compound 96)

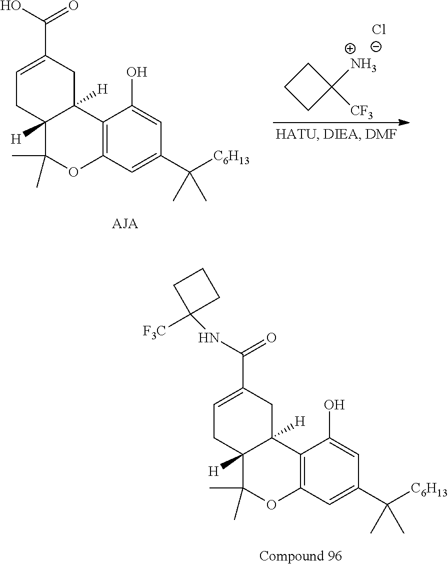

Compound 96 was synthesized according to the “general procedure for amide bond formation with HATU.” Yield 36.9% (96 mg). ¹H NMR (400 MHz, CDCl₃) δ 6.69 (d, J=5.32 Hz, 1H), 6.37 (d, J=1.68 Hz, 1H), 6.27 (d, J=1.72 Hz, 1H), 5.76 (s, 1H), 5.42 (s, 1H), 3.79 (dd, J=15.20-3.88 Hz, 1H), 2.70 (dt, J=10.64-4.56 Hz, 1H), 2.57 (t, J=8.61 Hz, 4H), 2.41-2.33 (m, 1H), 2.06-1.94 (m, 4H), 1.82 (dt, J=11.48-4.20 Hz, 1H), 1.53-1.47 (m, 2H), 1.40 (t, 3H), 1.27-1.15 (m, 12H), 1.12 (s, 3H), 1.04 (bs, 2H), 0.84 (t, J=7.04 Hz, 3H). LC-MS (ESI+): 522.3 (M+H⁺); R.T.: 4.948 min. (HPLC Method C).

### Example 48. (6aR,10aR)-N-cyclopentyl-1-hydroxy-6,6-dimethyl-3-(2-methylpentan-2-yl)-6a,7,10,10a-tetrahydro-6H-benzo[c]chromene-9-carboxamide (Compound 97)

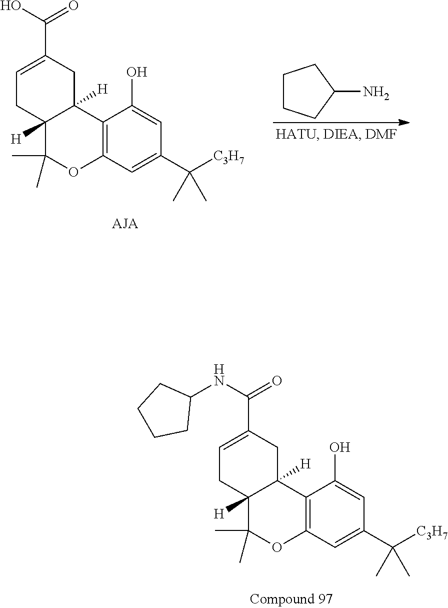

Compound 97 was synthesized according to the “general procedure for amide bond formation with HATU.” Yield 38.9% (23 mg). ¹H NMR (400 MHz, DMSO-d₆) δ 9.22 (s, 1H), 7.54 (d, J=7.32 Hz, 1H), 6.44 (m, 1H), 6.31 (d, J=1.84 Hz, 1H), 6.13 (d, J=1.80 Hz, 1H), 4.05 (m, 1H), 3.65 (dd, J=17.56-2.16 Hz, 1H), 2.44 (dt, J=11.24-4.48 Hz, 1H), 2.27 (m, 1H), 1.93 (m, 1H), 1.77 (m, 3H), 1.63 (m, 3H), 1.51-1.36 (m, 6H), 1.31 (s, 3H), 1.23 (s, 1H), 1.14 (s, 6H), 1.02 (s, 5H), 0.78 (t, J=7.44 Hz, 3H). LC-MS (ESI+): 426.2 (M+H⁺); R.T.: 4.368 min. (HPLC Method C).

### Example 49. (6aR,10aR)-1-hydroxy-N—((S)-4-hydroxybutan-2-yl)-6,6-dimethyl-3-(2-methyloctan-2-yl)-6a,7,10,10a-tetrahydro-6H-benzo[c]chromene-9-carboxamide (Compound 98)

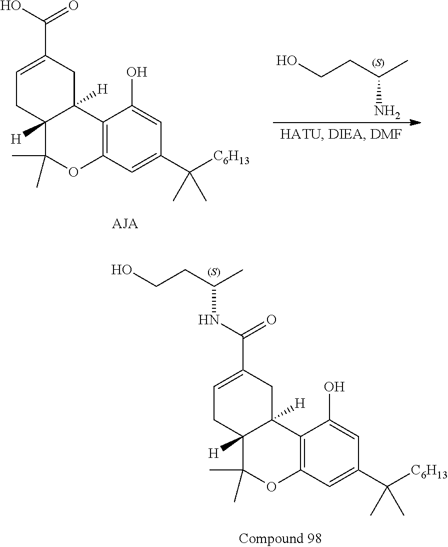

Compound 98 was synthesized according to the “general procedure for amide bond formation with HATU.” Yield 60.7% (286 mg). ¹H NMR (400 MHz, DMSO-d₆) δ 9.22 (s, 1H), 7.46 (d, J=8.24 Hz, 1H), 6.46 (m, 1H), 6.31 (d, J=1.72 Hz, 1H), 6.13 (d, J=1.72 Hz, 1H), 4.38 (t, J=5.12 Hz, 1H), 3.94 (m, 1H), 3.65 (d, J=15.40 Hz, 1H), 3.39 (q, J=5.60 Hz, 2H), 2.45 (dt, J=11.20-4.36 Hz, 1H), 2.32-2.24 (m, 1H), 1.98-1.88 (m, 1H), 1.78-1.69 (m, 1H), 1.67-1.51 (m, 3H), 1.48-1.43 (m, 2H), 1.31 (s, 3H), 1.21-1.14 (m, 13H), 1.06-1.01 (m, 8H), 0.81 (m, 4H). LC-MS (ESI+): 472.3 (M+H⁺); R.T.: 4.635 min. (HPLC Method C).

### Example 50. (6aR,10aR)-1-hydroxy-N-(1-trifluoromethyl-cyclopropyl)-6,6-dimethyl-3-(2-methyloctan-2-yl)-6a,7,10,10a-tetrahydro-6H-benzo[c]chromene-9-carboxamide (Compound 99)

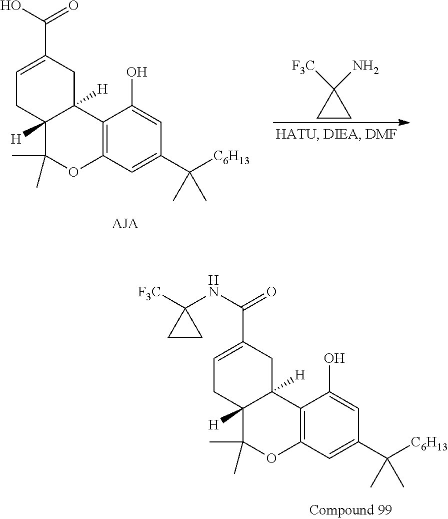

Compound 99 was synthesized according to the “general procedure for amide bond formation with HATU,” with the modification that the reaction was heated at 100° C. under microwave conditions. Yield 13.7% (52 mg). ¹H NMR (300 MHz, CDCl₃) 6.68 (d, J=4.77 Hz, 1H), 6.35 (s, 1H), 6.33 (s, 1H), 6.22 (s, 1H), 6.08 (s, 1H), 3.82 (dd, J=14.91-4.32 Hz, 1H), 2.69 (dt, J=10.74-4.41 Hz, 1H), 2.36 (dt, J=15.6-4.44 Hz, 1H), 2.0 (d, J=10.98 Hz, 1H), 1.95 (d, J=15.21 Hz, 1H), 1.80 (dt, J=10.35-3.9 Hz, 1H), 1.52-1.40 (m, 2H), 1.39 (s, 3H), 1.34 (s, 2H), 1.19 (s, 15H), 1.11 (s, 3H), 1.05 (bs, 2H), 0.83 (t, J=6.99 Hz, 3H). LC-MS (ESI+): 508.3 (M+H⁺); R.T.: 4.519 min. (HPLC Method C).

### Example 51. (6aR,10aR)-1-hydroxy-6,6-dimethyl-3-(2-methyloctan-2-yl)-N-(1,1,1-trifluoro-2-methylpropan-2-yl)-6a,7,10,10a-tetrahydro-6H-benzo[c]chromene-9-carboxamide (Compound 100)

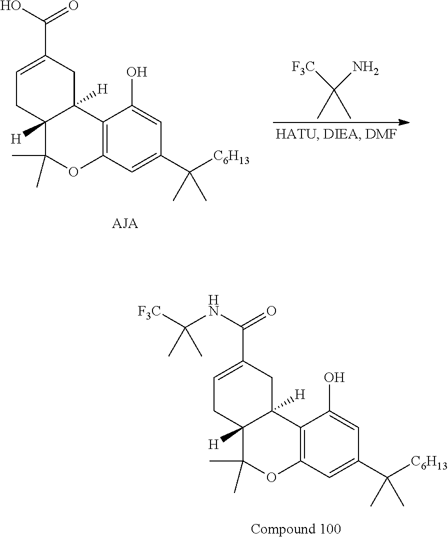

Compound 100 was synthesized according to the “general procedure for amide bond formation with HATU,” with the modification that the reaction was heated at 100° C. under microwave conditions. Yield 30.2% (192 mg). ¹H NMR (300 MHz, CDCl₃) δ 6.56 (d, J=4.98 Hz, 1H), 6.35 (d, J=1.53 Hz, 1H), 6.31 (d, J=1.59 Hz, 1H), 6.23 (s, 1H), 5.75 (s, 1H), 3.86 (dd, J=14.95-4.26 Hz, 1H), 2.69 (dt, J=11.14-4.50 Hz, 1H) 2.40-2.32 (m, 1H), 2.02-1.90 (m, 2H), 1.80 (dt, J=11.52-3.99 Hz, 1H), 1.64 (s, 6H), 1.59 (s, 1H) 1.51-1.46 (m, 2H), 1.39 (s, 3H), 1.19 (s, 15H), 1.11 (s, 3H), 1.06 (bs, 2H) 0.84 (t, J=6.96 Hz, 3H). LC-MS (ESI+): 510.3 (M+H⁺); R.T.: 4.664 min. (HPLC Method C).

### Example 52. (6aR,10aR)-1-hydroxy-6,6-dimethyl-3-(2-methylpentan-2-yl)-N-(1-(trifluoromethyl)cyclopropyl)-6a,7,10,10a-tetrahydro-6H-benzo[c]chromene-9-carboxamide (Compound 101)

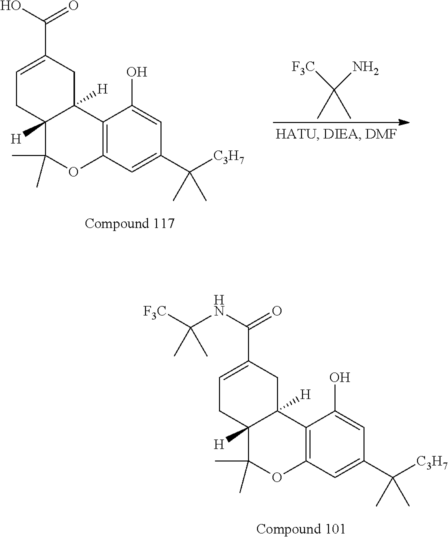

Compound 101 was synthesized according to the “general procedure for amide bond formation with HATU,” with the modification that the reaction was heated at 100° C. under microwave conditions. Yield 13.9% 38 mg). ¹H NMR (400 MHz, CDCl₃) δ 9.22 (s, 1H), 7.44 (s, 1H), 6.42 (m, 1H), 6.32 (d, J=1.76 Hz, 1H), 6.13 (d, J=1.68 Hz, 1H), 3.65 (d, J=15.2 Hz, 1H), 2.45 (dt, J=11.20-4.44 Hz, 1H), 2.23-2.25 (m, 1H), 1.94 (m, 1H), 1.74 (m, 1H), 1.63 (dt, J=11.56-4.48 Hz, 1H), 1.51 (s, 6H), 1.46-1.42 (m, 2H), 1.31 (s, 3H), 1.23 (m, 1H), 1.14 (s, 6H), 1.02 (s, 5H), 0.78 (t, J=7.36 Hz, 3H). LC-MS (ESI+): 468.3 (M+H⁺); R.T.: 4.695 min. (HPLC Method C).

### Example 53. (6aR,10aR)-1-hydroxy-6,6-dimethyl-3-(2-methylpentan-2-yl)-N-(1-(trifluoromethyl)cyclopropyl)-6a,7,10,10a-tetrahydro-6H-benzo[c]chromene-9-carboxamide (Compound 102)

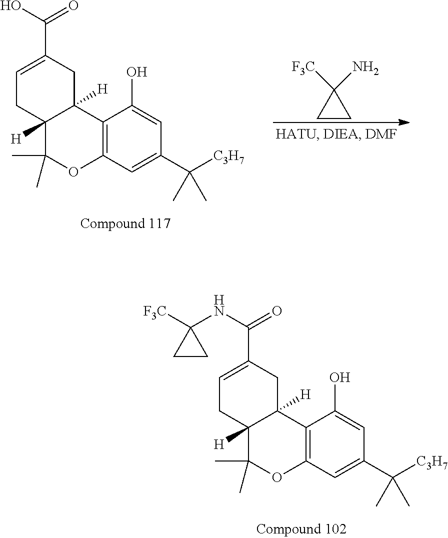

Compound 102 was synthesized according to the “general procedure for amide bond formation with HATU,” with the modification that the reaction was heated at 100° C. under microwave conditions. Yield 18.0% 49 mg). ¹H NMR (400 MHz, CDCl₃) δ 9.23 (s, 1H), 8.48 (s, 1H), 6.56 (m, 1H), 6.31 (d, J=1.84 Hz, 1H), 6.13 (d, J=1.80 Hz, 1H), 3.67 (d, J=15.60 Hz, 1H), 2.43 (dt, J=11.20-4.40 Hz, 1H), 2.33-2.25 (m, 1H), 1.99-1.91 (m, 1H), 1.75-1.67 (m, 1H), 1.63 (t, J=11.56-4.64 Hz, 1H), 1.46-1.41 (m, 2H), 1.30 (s, 3H), 1.22 (m, 2H), 1.01 (s, 6H), 1.01 (s, 7H), 0.78 (t, J=7.40 Hz, 3H). LC-MS (ESI+): 466.2 (M+H⁺); R.T.: 4.301 min. (HPLC Method C).

### Example 54. Synthesis of (6aR,10aR)-1-hydroxy-N-(2-hydroxyethoxy)-6,6-dimethyl-3-(2-methyloctan-2-yl)-6a,7,10,10a-tetrahydro-6H-benzo[c]chromene-9-carboxamide (Compound 54)

### Step a. Synthesis of (6aR,10aR)-N-(2-((tert-butyldimethylsilyl)oxy)ethoxy)-1-hydroxy-6,6-dimethyl-3-(2-methyloctan-2-yl)-6a,7,10,10a-tetrahydro-6H-benzo[c]chromene-9-carboxamide

The intermediate of step a. was prepared using the “general procedure for amide bond formation with HATU,” by using O-(2-((tert-butyldimethylsilyl)oxy)ethyl)hydroxylamine as amine source (prepared from TBDMS protection of 2-(aminooxy)ethan-1-ol) in 81.0% yield (232 mg). LC-MS (ESI+): 474.4 (M+H⁺); R.T.: 2.061 min. (HPLC Method E).

### Step b. Synthesis of (6aR,10aR)-1-hydroxy-N-(2-hydroxyethoxy)-6,6-dimethyl-3-(2-methyloctan-2-yl)-6a,7,10,10a-tetrahydro-6H-benzo[c]chromene-9-carboxamide (Compound 54)

TBDMS deprotection was performed analogously to Compound 106 to afford the title compound in 56.6% yield (102 mg). ¹H NMR (300 MHz, CDCl₃) δ 11.06 (s, 1H), 9.24 (s, 1H), 6.45 (s, 1H), 6.31 (s, 1H), 6.12 (s, 1H), 4.73 (t, J=5.4 Hz, 1H), 3.79 (t, J=4.30 Hz, 2H), 3.64 (d, J=17.8 Hz, 1H), 3.53 (dd, J=9.9, 4.5 Hz, 2H), 2.47 (t, J=7.98 Hz, 1H), 2.28 (d, J=17.0 Hz, 1H), 1.94 (t, J=16.3 Hz, 1H), 1.78-1.58 (m, 2H), 1.49-1.399 (m, 2H), 1.31 (s, 3H), 1.14 (s, 12H), 1.01 (s, 5H), 0.81 (s, 3H). LC-MS (ESI+): 460.3 (M+H⁺); R.T.: 4.01 min. (HPLC Method C).

### Example 55. Synthesis of (6aR,10aR)-N-(azetidin-3-yl)-1-hydroxy-6,6-dimethyl-3-(2-methyloctan-2-yl)-6a,7,10,10a-tetrahydro-6H-benzo[c]chromene-9-carboxamide (Compound 103)

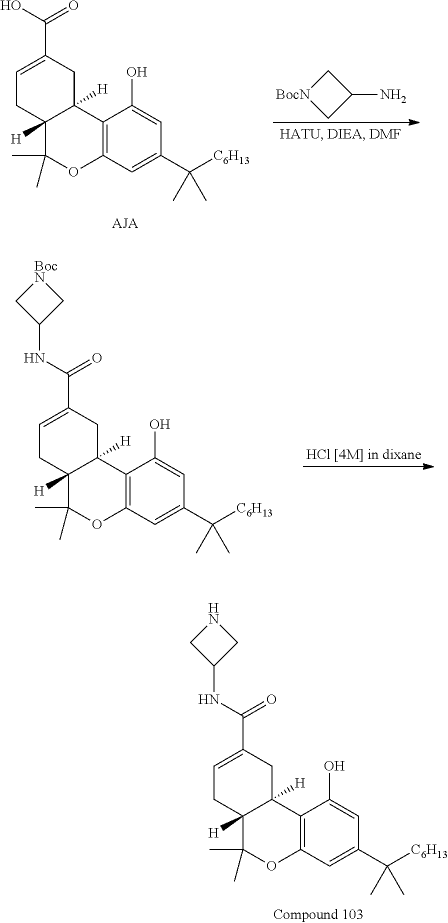

### Step a. Tert-butyl 3-((6aR,10aR)-1-hydroxy-6,6-dimethyl-3-(2-methyloctan-2-yl)-6a,7,10,10a-tetrahydro-6H-benzo[c]chromene-9-carboxamido)azetidine-1-carboxylate (Boc protected Compound 103)

The intermediate of step a. was prepared using the “general procedure for amide bond formation with HATU.” Yield 66.9% (298 mg). ¹H NMR (300 MHz, CDCl₃) δ 6.78 (d, J=3.09 Hz, 1H), 6.36 (s, 1H), 6.33 (s, 1H), 6.18 (d, J=7.05 Hz, 1H), 4.69 (dd, J=12.63-5.94 Hz, 1H), 4.26 (q, J=8.46 Hz, 1H), 3.75 (m, 3H), 2.80 (s, 1H), 2.70 (dt, J=10.41-4.20 Hz, 1H), 2.39 (m, 1H), 2.06-1.93 (m, 2H), 1.82 (dt, J=11.48-4.25 Hz, 1H), 1.59 (s, 2H), 1.54-1.46 (m, 2H), 1.44 (s, 9H), 1.40 (s, 3H), 1.20 (s, 12H), 1.12 (s, 3H), 1.05 (bs, 2H), 0.84 (t, J=6.93 Hz, 3H). LC-MS (ESI+): 555.4 (M+H⁺); R.T.: 4.905 min. (HPLC Method C).

### Step b. (6aR,10aR)-N-(azetidin-3-yl)-1-hydroxy-6,6-dimethyl-3-(2-methyloctan-2-yl)-6a,7,10,10a-tetrahydro-6H-benzo[c]chromene-9-carboxamide (Compound 103)

The intermediate from step a. (0.278 g, 0.50 mmol, 1 eq) was dissolved in HCl (4M) in dioxane (5 mL) and stirred for 3 hours at room temperature. The reaction was concentrated under reduced pressure and the residue (white solid) was suspended in heptane and concentrated again. This process was repeated three times. The residue was purified by reverse phase column (MAP4BIC; from 39% [Aqueous phase]-61% [Organic phase] to 11% [Aqueous phase]-89% [Organic phase]; Aqueous phase: 25 mM NH₄HCO₃; Organic phase: ACN:MeOH 1:1). The fractions containing desired compound were collected and concentrated under reduced pressure. The residue was re-dissolved in anhydrous ACN and concentrated under reduced pressure at 65° C. to yield Compound 103 as a white solid (5.0 mg, 2.9%). ¹H NMR (400 MHz, CDCl₃) δ 6.40 (d, J=2.09 Hz, 1H), 6.29 (s, 1H), 6.18 (s, 1H), 4.16 (bs, 2H), 3.79 (t, J=11.21 Hz, 1H), 3.70 (dd, J=11.41-2.65 Hz, 1H), 3.61 (m, 1H), 3.49 (bs, 1H), 2.61 (dt, J=11.32-4.11 Hz), 2.36 (dt, J=14.93-4.89 Hz, 1H), 2.04-1.94 (m, 2H) 1.73 (dt, J=11.58-3.86 Hz, 1H), 1.41-1.37 (m, 2H), 1.33 (s, 3H), 1.26-1.167 (m, 2H) 1.11 (s, 13H), 0.99 (m, 2H), 0.84 (t, J=7.16 Hz, 3H). LC-MS (ESI+): 455.3 (M+H⁺); R.T.: 3.335 min. (HPLC Method C).

### Example 56. Synthesis of ((6aR,10aR)-1-hydroxy-6,6-dimethyl-3-(2-methyloctan-2-yl)-6a,7,10,10a-tetrahydro-6H-benzo[c]chromen-9-yl)(piperazin-1-yl)methanone (Compound 104)

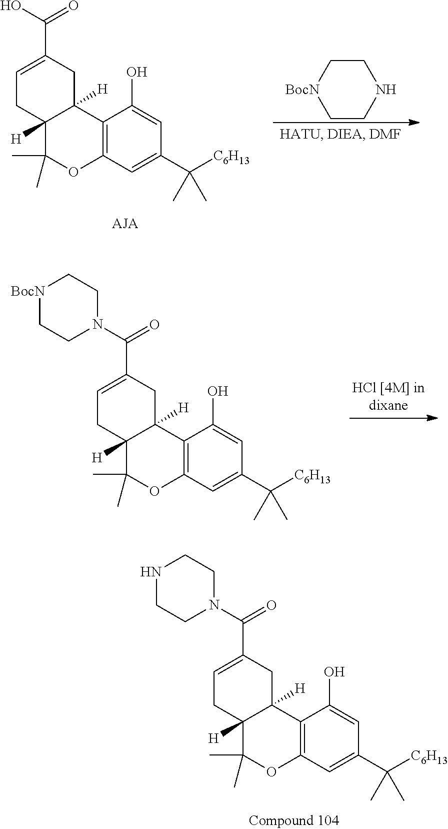

### Step a. ((6aR,10aR)-1-hydroxy-6,6-dimethyl-3-(2-methyloctan-2-yl)-6a,7,10,10a-tetrahydro-6H-benzo[c]chromen-9-yl)(piperazin-1-yl)methanone (Boc protected Compound 104)

The intermediate of step a. was prepared using the “general procedure for amide bond formation with HATU.” Yield 60.7% (345 mg). ¹H NMR (300 MHz, CDCl₃) δ 6.48 (s, 1H), 6.33 (d, J=3.84 Hz, 2H), 5.85 (d, J=3.63 Hz, 1H), 3.74 (dd, J=16.3-3.33 Hz, 1H), 3.56 (bs, 4H), 3.42 (bs, 4H), 2.75 (m, 1H), 2.35-2.27 (m, 1H), 2.04-1.82 (m, 3H), 1.61 (s, 1H), 1.47 (s, 11H), 1.39 (s, 3H), 1.19 (s, 12H), 1.09 (s, 3H), 1.05 (bs, 2H), 0.84 (t, J=6.96 Hz, 3H). LC-MS (ESI+): 513.1 (M+H⁺); R.T.: 1.915 min. (HPLC Method E).

### Step b. ((6aR,10aR)-1-hydroxy-6,6-dimethyl-3-(2-methyloctan-2-yl)-6a,7,10,10a-tetrahydro-6H-benzo[c]chromen-9-yl)(piperazin-1-yl)methanone (Compound 104)

The intermediate of step a. was dissolved in HCl [4M] in dioxane (2.64 mL), the mixture was stirred 14 hours at room temperature. The solvent was removed under reduced pressure, the white solid was suspended in heptane and concentrated again, repeat 3 times, to get Compound 104 as a white solid (0.103 g, 83.2%). ¹H NMR (400 MHz, DMSO-d₆) δ 9.25 (s, 1H), 9.21 (s, 2H), 6.32 (d, J=1.76 Hz, 1H), 6.13 (d, J=1.76 Hz, 1H), 5.86 (d, J=4.36 Hz, 1H), 3.69 (m, 4H), 3.52 (dd, J=17.64-2.68 Hz, 1H), 3.07 (t, J=4.52 Hz, 4H), 2.56 (dt, J=11.12-4.64 Hz, 1H), 2.29-2.2 (m, 1H), 1.95-1.79 (m, 2H), 1.73 (dt, J=11.52-4.40 Hz, 1H), 1.47-1.43 (m, 2H), 1.32 (s, 3H), 1.17 (m, 6H), 1.13 (s, 7H), 1.03 (s, 3H), 1.00 (bs, 2H), 0.81 (t, J=7.04 Hz, 3H). LC-MS (ESI+): 469.3 (M+H⁺); R.T.: 3.076 min. (HPLC Method C).

### Example 57. Synthesis of (6aR,10aR)-N,1-dihydroxy-6,6-dimethyl-3-(2-methyloctan-2-yl)-6a,7,10,10a-tetrahydro-6H-benzo[c]chromene-9-carboxamide (Compound 105)

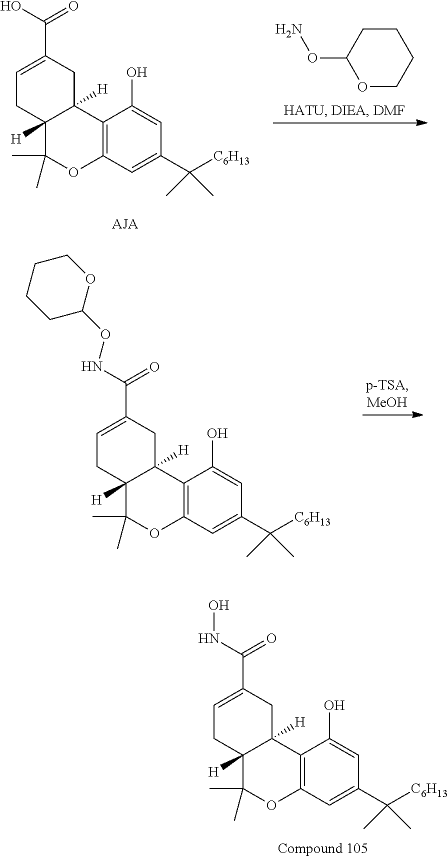

### Step a. (6aR,10aR)-N,1-dihydroxy-6,6-dimethyl-3-(2-methyloctan-2-yl)-6a,7,10,10a-tetrahydro-6H-benzo[c]chromene-9-carboxamide (protected Compound 105)

The intermediate of step a. was prepared using the “general procedure for amide bond formation with HATU.” Yield 50.1% (250 mg). LC-MS (ESI+): 416.2 (M+H⁺-84); R.T.: 1.789 min. (HPLC Method E).

### Step b. (6aR,10aR)-N,1-dihydroxy-6,6-dimethyl-3-(2-methyloctan-2-yl)-6a,7,10,10a-tetrahydro-6H-benzo[c]chromene-9-carboxamide (Compound 105)

p-Toluenesulfonic acid (16 mg, 0.084 mmol, 0.2 eq) was added portion wise to a stirred solution of the intermediate from step a. (210 mg, 0.42 mmol) in MeOH (3.6 mL). The reaction mixture was stirred 30 minutes at room temperature. An aqueous solution of NaHCO₃ (2 M) was added to pH=4-5. The product was extracted with EtOAc (3×5 mL). The combined organic layers were dried over anhydrous MgSO₄, filtered and concentrated under reduced pressure to afford the title compound as a white solid (16 mg, 10%). ¹H NMR (300 MHz, DMSO-d6) δ 10.50 (s, 1H), 9.23 (s, 1H), 8.67 (s, 1H), 6.36 (s, 1H), 6.31 (s, 1H), 6.12 (s, 1H), 3.65 (d, J=17.46 Hz, 1H), 2.46 (m, 1H), 2.26 (s, J=17.46 Hz, 1H), 1.92 (t, J=15.57 Hz, 1H), 1.76-1.58 (m, 2H), 1.44 (bs, 2H), 1.30 (s, 3H), 1.14 (s, 12H), 1.01 (s, 5H), 0.81 (t, J=6.24 Hz, 3H). LC-MS (ESI+): 416.2 (M+H⁺); R.T.: 4.264 min. (HPLC Method C).

### Example 58. Synthesis of (6aR,10aR)-N-cyano-1-hydroxy-6,6-dimethyl-3-(2-methyloctan-2-yl)-6a,7,10,10a-tetrahydro-6H-benzo[c]chromene-9-carboxamide (Compound 106)

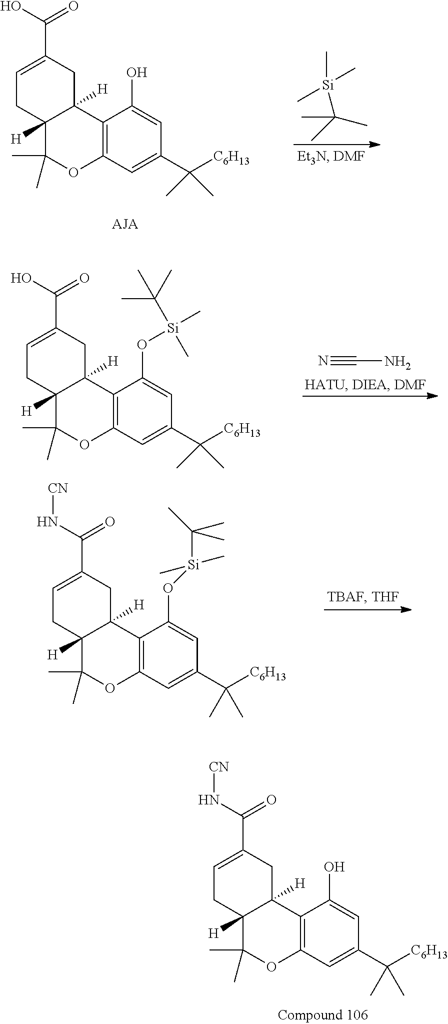

Compound 106 was prepared by phenyl hydroxyl group protection via tert-butyldimethylsilyl (TBDMS) ether followed by amide bond formation and deprotection of the TBDMS group as described below.

### Step a. (6aR,10aR)-1-((tert-butyldimethylsilyl)oxy)-6,6-dimethyl-3-(2-methyloctan-2-yl)-6a,7,10,10a-tetrahydro-6H-benzo[c]chromene-9-carboxylic acid

Triethylamine (8.7 mL, 62.4 mmol, 5 eq) and tert-butyldimethylsilyl chloride (3.76 g, 24.9 mmol, 2.0 eq) were added to a solution of (6aR,10aR)-1-hydroxy-6,6-dimethyl-3-(2-methyloctan-2-yl)-6a,7,10,10a-tetrahydro-6H-benzo[c]chromene-9-carboxylic acid (5.0 g, 12.48 mmol, 1.0 eq) in DMF (38 mL) under nitrogen atmosphere. The reaction mixture was stirred at room temperature for 20 h. Then additional triethylamine (4.3 mL, 31.2 mmol, 2.5 eq) and tert-Butyldimethylsilyl chloride (1.9 r, 12.6 mmol, 1.0 eq) were added and the reaction mixture was stirred for 3 hours. The mixture was diluted with EtOAc and washed with a saturated aqueous solution of NaHCO₃, the organic layer was dried over anhydrous MgSO₄, filtered and concentrated under reduced pressure. The residue was purified by chromatography on silica gel (Heptane: EtOAc; from 100:0 to 80:20). The desired fractions were collected and concentrated under reduced pressure to afford the desired product as yellow foam. (4.62 g; 71.9%). ¹H NMR (300 MHz, CDCl₃) δ 7.13 (s, 1H), 6.39 (d, J=12.1 Hz, 2H), 3.86 (d, J=17.8 Hz, 1H), 2.55 (td, J=11.0, 3.8 Hz, 1H), 2.44 (d, J=18.2 Hz, 1H), 2.12-1.76 (m, 3H), 1.50 (dd, J=9.8, 6.1 Hz, 2H), 1.41 (s, 3H), 1.20 (s, 12H), 1.11 (s, 3H), 1.05 (s, 2H), 0.98 (s, 10H), 0.84 (t, J=6.5 Hz, 3H), 0.27 (s, 3H), 0.14 (s, 3H). LC-MS (ESI+): 515.3 (M+H⁺); R.T.: 2.327 min. (HPLC Method E).

### Step b. (6aR,10aR)-1-((tert-butyldimethylsilyl)oxy)-N-cyano-6,6-dimethyl-3-(2-methyloctan-2-yl)-6a,7,10,10a-tetrahydro-6H-benzo[c]chromene-9-carboxamide

DIEA (0.423 mL, 2.4 mmol, 2.5 eq) and HATU (0.391 g, 1.02 mmol, 1.05 eq) were added to a solution of the intermediate from step. a (0.5 g, 0.971 mmol) in dry DMF (9.7 mL) under nitrogen atmosphere. The mixture was stirred for 5 minutes. Then, cyanamide (0.082 g, 1.942 mmol, 2.0 eq) was added and the reaction mixture was stirred at rt for 6 hours. The solution was diluted with EtOAc and washed with a saturated aqueous solution of NaHCO₃ (×3) and a saturated aqueous solution of NH₄Cl (×3). The organic layer was dried over anhydrous MgSO₄, filtered and concentrated under reduced pressure to get yellow oil. The crude reaction was used for next step without further purification.

### Step c. (6aR,10aR)-N-cyano-1-hydroxy-6,6-dimethyl-3-(2-methyloctan-2-yl)-6a,7,10,10a-tetrahydro-6H-benzo[c]chromene-9-carboxamide (Compound 106)

A solution of tetrabutylammonium fluoride in THE [1 M] (1.45 mL, 1.45 mmol, 1.5 eq) was added dropwise to a stirred solution of the intermediate from step b. (0.971 mmol) in THE (2.9 mL). The reaction mixture was stirred 3 hours at room temperature. The solution was diluted with EtOAc and washed with an aqueous solution of NaHCO₃, the organic layer was dried over anhydrous MgSO₄, filtered and concentrated under reduced pressure. The product was purified on silica gel (Heptane: EtOAc; from 100:0 to 70:30), the desired fractions were collected and concentrated under reduced pressure to yield the title compound as a white solid (0.167 g, 40.5%). H NMR (300 MHz, DMSO-d₆) δ 11.47 (s, 1H), 9.28 (s, 1H), 6.84 (s, 1H), 6.31 (s, 1H), 6.13 (s, 1H) 3.77 (d, J=17.64 Hz, 1H), 2.36 (t, J=21.17 Hz, 1H), 2.05 (t, J=16.95 Hz, 1H), 1.811-1.60 (m, 2H), 1.45 (bs, 2H), 1.31 (s, 3H), 1.14 (s, 12H), 1.02 (s, 5H), 0.81 (s, 3H). LC-MS (ESI+): 425.3 (M+H⁺); R.T.: 4.281 min. (HPLC Method C).

### Example 59. Synthesis of (6aR,10aR)-1-((tert-butyldimethylsilyl)oxy)-N-methoxy-6,6-dimethyl-3-(2-methyloctan-2-yl)-6a,7,10,10a-tetrahydro-6H-benzo[c]chromene-9-carboxamide (Compound 107)

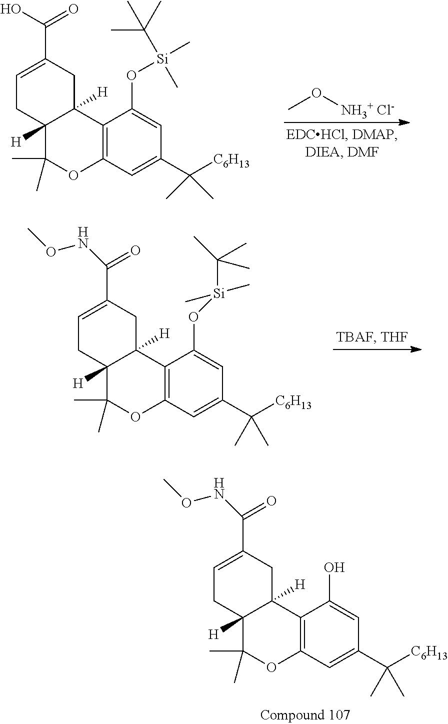

The title compound was prepared analogously to the synthesis of Compound 106.

### Step a. (6aR,10aR)-1-((tert-butyldimethylsilyl)oxy)-N-methoxy-6,6-dimethyl-3-(2-methyloctan-2-yl)-6a,7,10,10a-tetrahydro-6H-benzo[c]chromene-9-carboxamide

Yield 66.1% (359 mg). ¹H NMR (300 MHz, CDCl₃) δ 8.24 (s, 1H), 6.81 (s, 1H), 6.42 (s, 1H), 6.36 (s, 1H), 3.82 (s, 3H), 3.65 (d, J=16.0 Hz, 1H), 2.56 (dt, J=9.4, 3.6 Hz 1H), 2.39 (d, J=17.1 Hz, 1H), 2.02-1-77 (m, 3H), 1.57 (s, 3H), 1.52-1.44 (m, 2H), 1.39 (s, 3H), 1.19 (s, 13H), 1.09 (s, 3H), 0.99 (s, 12H), 0.84 (t, J=6.3 Hz), 0.26 (s, 3H), 0.12 (s, 3H). LC-MS (ESI+): 544.4 (M+H⁺); R.T.: 2.297 min. (HPLC Method E).

### Step b. (6aR,10aR)-1-((tert-butyldimethylsilyl)oxy)-N-methoxy-6,6-dimethyl-3-(2-methyloctan-2-yl)-6a,7,10,10a-tetrahydro-6H-benzo[c]chromene-9-carboxamide (Compound 107)

Yield 66.1% (359 mg). ¹H NMR (300 MHz, CDCl₃) δ 11.0 (s, 1H), 9.26 (s, 1H), 6.42 (s, 1H), 6.31 (s, 1H), 6.12 (s, 1H), 3.66 (d, J=9.3 Hz, 1H), 3.59 (s, 3H), 2.45 (t, J=3.4 Hz, 1H), 2.28 (d, J=16.7 Hz, 1H), 1.93 (t, J=16.0 Hz, 1H), 1.73-1.58 (m, 2H), 1.50-1.40 (m, 2H), 1.30 (s, 3H), 1.14 (s, 12H), 1.01 (s, 5H), 0.81 (s, 3H). LC-MS (ESI+): 430.3 (M+H⁺); R.T.: 4.198 min. (HPLC Method C).

### Example 60. Synthesis of (6aR,10aR)-1-hydroxy-6,6-dimethyl-3-(2-methyloctan-2-yl)-N-(methylsulfonyl)-6a,7,10,10a-tetrahydro-6H-benzo[c]chromene-9-carboxamide (Compound 108)

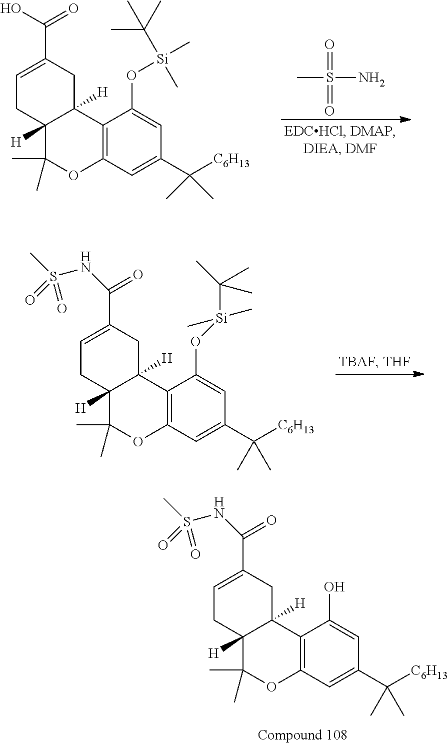

The title compound was prepared analogously to the synthesis of Compound 106.

### Step a. (6aR,10aR)-1-((tert-butyldimethylsilyl)oxy)-6,6-dimethyl-3-(2-methyloctan-2-yl)-N-(methylsulfonyl)-6a,7,10,10a-tetrahydro-6H-benzo[c]chromene-9-carboxamide

Yield 56.5% (260 mg). ¹H NMR (300 MHz, CDCl₃) δ 7.88 (s, 1H), 6.94 (s, 1H), 6.42 (s, 1H) 6.37 (s, 1H) 3.77 (d, J=16.6 Hz, 1H), 2.92 (s, 3H), 2.60-2-39 (m, 2H), 2.21-1-77 (m, 3H), 1.40 (s, 3H), 1.20 (s, 13H), 1.11 (s, 3H), 1.05 (bs, 1H), 0.98 (s, 1 OH), 0.84 (t, J=6.2 Hz, 3H), 0.27 (s, 3H), 0.13 (s, 3H). LC-MS (ESI+): 592.4 (M+H⁺); R.T.: 1.932 min. (HPLC Method E).

### Step b. (6aR,10aR)-1-hydroxy-6,6-dimethyl-3-(2-methyloctan-2-yl)-N-(methylsulfonyl)-6a,7,10,10a-tetrahydro-6H-benzo[c]chromene-9-carboxamide (Compound 108)

Yield 12.9% (27 mg). ¹H NMR (300 MHz, CDCl₃) δ 11.48 (s, 1H), 9.26 (s, 1H), 6.87 (s, 1H), 6.32 (s, 1H), 6.13 (s, H), 3.73 (d, J=16.3 Hz, 1H), 2.40 (t, J=19.8 Hz, 1s), 2.01 (t, J=15.6 Hz, 1H), 1.78-1-59 (m, 2H), 1.50-1-40 (m, 2H), 1.32 (s, 3H), 1.14 (s, 13H), 1.02 (s, 5H), 0.81 (s, 3H). LC-MS (ESI+): 478.2 (M+H⁺); R.T.: 4.165 min. (HPLC Method C).

### Example 61. Synthesis of (6aR,10aR)-N-cyclobutyl-1-hydroxy-6,6-dimethyl-3-(2-methyloctan-2-yl)-6a,7,10,10a-tetrahydro-6H-benzo[c]chromene-9-carboxamide (Compound 44)

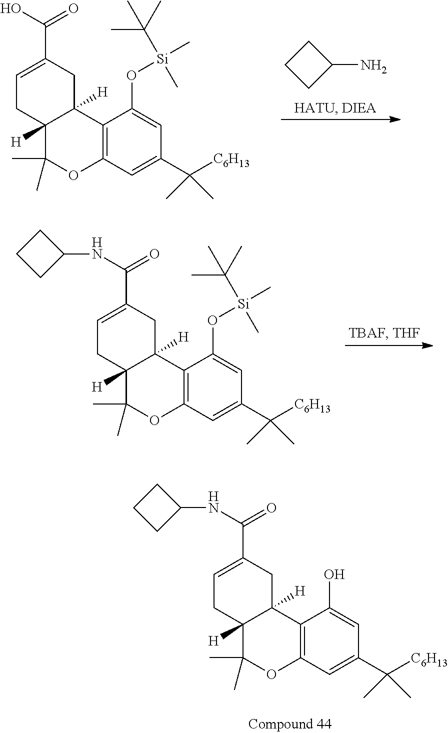

The title compound was prepared analogously to the synthesis of Compound 106.

### Step a. (6aR,10aR)-1-((tert-butyldimethylsilyl)oxy)-N-cyclobutyl-6,6-dimethyl-3-(2-methyloctan-2-yl)-6a,7,10,10a-tetrahydro-6H-benzo[c]chromene-9-carboxamide

The intermediate of step a. was made as a crude product and was used without further purification.

### Step b. (6aR,10aR)-N-cyclobutyl-1-hydroxy-6,6-dimethyl-3-(2-methyloctan-2-yl)-6a,7,10,10a-tetrahydro-6H-benzo[c]chromene-9-carboxamide (Compound 44)

Yield 43% (190 mg). ¹H NMR (300 MHz, CDCl₃) δ 9.21 (s, 1H), 7.87 (d, J=7.2 Hz, 2H), 6.47 (s, 1H), 6.31 (s, 1H) 6.13 (s, 1H), 4.25 (q, J=7.7 Hz, 1H), 3.64 (d, J=16.7 Hz, 1H), 2.44 (t, J=10.4 Hz, 1H), 2.28 (d, J=18 Hz, 1H), 2.1 (bs, 2H), 1.95 (m, 3H), 1.73 (m, 1H), 1.60 (m, 3H), 1.44 (m, 2H), 1.31 (s, 3H), 1.1 (s, 12H), 1.0 (s, 5H), 0.81 (s, 3H). LC-MS (ESI+): 454.3 (M+H⁺); R.T.: 4.433 min. (HPLC Method C).

### Example 62. (6aR,10aR)-6,6-dimethyl-3-(2-methylhexan-2-yl)-1-(pivaloyloxy)-6a,7,10,10a-tetrahydro-6H-benzo[c]chromene-9-carboxylic acid (Compound 109)

### Step a. 2-(3,5-dimethoxyphenyl)-2-methylpropanenitrile

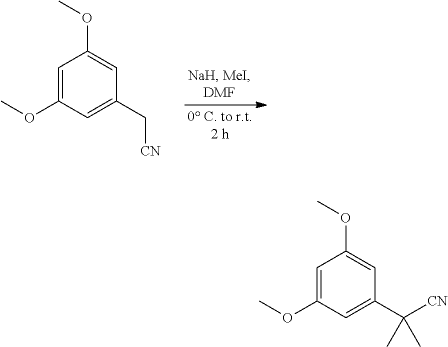

To a stirred suspension of sodium hydride (60% dispersion in mineral oil) (6.77 g, 169.3 mmol, 3 eq) in dry DMF (120 mL) at 0° C. was added dropwise a solution of 2-(3,5-dimethoxyphenyl)acetonitrile (10.0 g, 56.4 mmol, 1 eq) and iodomethane (10.5 mL, 169.3 mmol, 3 eq) in dry DMF (50 mL). The reaction mixture was stirred at 0° C. for 30 minutes and at room temperature for 90 minutes. The reaction mixture was quenched with a saturated aqueous solution NH₄Cl (50 mL). The desired compound was extracted with diethyl ether (3×20 mL). The combined organic layers were washed with water and brine, dried over anhydrous MgSO₄, filtered and concentrated under reduced pressure, the resulted oil was diluted with toluene and concentrated under reduced pressure (repeated 3 times). The crude product was purified by chromatography on silica gel (Heptane/EtOAc; from 100:0 to 90:10), to give the desired compound as a colorless oil (9.2 g, 79.9%). ¹H NMR (300 MHz, DMSO-d₆) δ 6.62 (s, 2H), 6.48 (s, 1H), 3.77 (s, 6H), 1.66 (s, 6H). LC-MS (ESI+): 206.1 (M+H⁺); R.T.: 1.327 min. (HPLC Method E).

### Step b. 2-(3,5-Dimethoxyphenyl)-2-methylpropanal

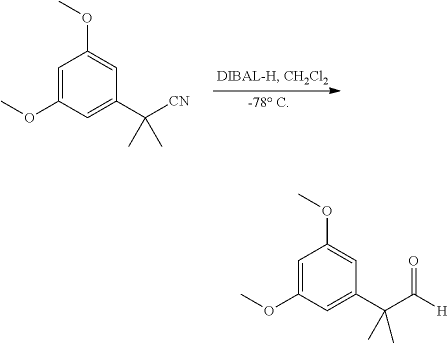

A solution of DIBAL ([1M] in toluene) (160.9 mL, 160.9 mmol, 2.5 eq) was added dropwise to a stirred solution of 2-(3,5-Dimethoxy-phenyl)-2-methyl-propionitrile (13.2 g, 64.36 mmol, 1 eq) in dry CH₂Cl₂ (320 mL) under nitrogen at −78° C. The reaction mixture was stirred at −78° C. for 1 h. An aqueous solution of potassium sodium tartrate (10% solution in water) was added dropwise and the reaction mixture was warmed to room temperature and stirred vigorously overnight. The solid was filtered through celite and rinsed with CH₂Cl₂, the product was extracted with CH₂Cl₂. The combined organic layers were washed with brine and water, dried over anhydrous MgSO₄, filtered and the solvent was removed under reduced pressure. The product was purified by chromatography on silica gel (heptane/EtOAc, from 100:0 to 80:20). The desired fractions were collected and concentrated to afford the title product as a colorless oil. (11.6 g, 86.5%). ¹H NMR (300 MHz, CDCl₃) δ 9.46 (s, 1H), 6.40 (s, 3H), 3.79 (s, 6H), 1.43 (s, 6H). LC-MS (ESI+): 209.1 (M+H⁺); R.T.: 1.388 min. (HPLC Method E).

### Step c. (Z)-1,3-dimethoxy-5-(2-methylhex-3-en-2-yl)benzene

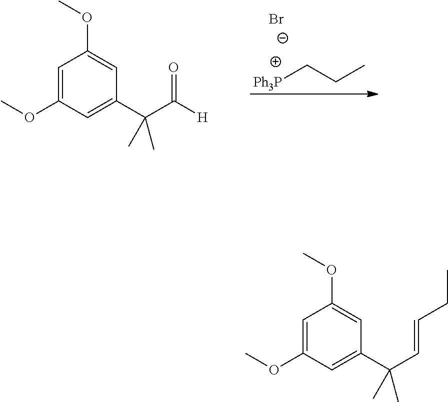

Triphenyl(propyl)phosphonium bromide (8.60 g, 22.33 mmol, 1.2 eq) was dissolved in dry THE (30 mL) and cooled at 0° C., LiHMDS ([1 M] in THF) (46.5 mL, 46.5 mmol, 2.5 eq) was added dropwise and the solution was stirred at room temperature for 1 hour. Then, a solution of 2-(3,5-Dimethoxy-phenyl)-2-methyl-propinal (3.87 g, 18.60 mmol, 1 eq) in dry THE (26 mL) was added dropwise. The resulted mixture was stirred at room temperature for 2 days. The reaction was quenched with an aqueous solution of HCl (10%). The product was isolated by extraction with EtOAc (3×25 mL). The combined organic layers were dried over MgSO₄, filtered and concentrated under reduced pressure. The product was purified by chromatography on silica gel (Heptane/EtOAc; from 100:0 to 90:10) to afford the desired compound as a colorless oil. (3.03 g, 69.4%). ¹H NMR (300 MHz, DMSO-d₆) δ 6.46 (s, 2H), 6.31 (s, 1H), 5.57 (d, J=11.37 Hz, 1H), 5.23 (m, 1H), 3.70 (s, 6H), 1.59 (p, J=7.17 Hz, 2H) 1.33 (s, 6H), 0.69 (t, J=7.23 Hz, 3H). LC-MS (ESI+): 235.1 (M+H⁺); R.T.: 1.758 min. (HPLC Method E).

### Step d. 1,3-dimethoxy-5-(2-methylhexan-2-yl)benzene

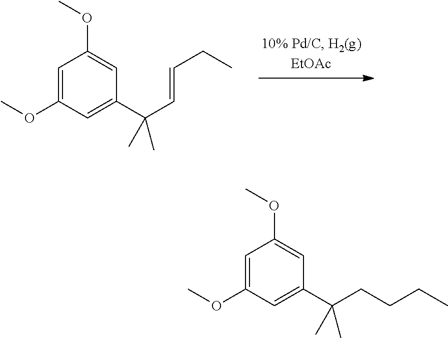 [SMILES: CC/C=C/C(C)(C)c1cc(OC)cc(OC)c1 ; CCCCC(C)(C)c1cc(OC)cc(OC)c1]

(Z)-1,3-dimethoxy-5-(2-methylhex-3-en-2-yl)benzene (3.0 g, 12.80 mmol, 1 eq) was dissolved in EtOAc (39 mL), Pd on C (10% wet) (0.30 g) was added and the mixture was purge with vacuum and H2 (g). The reaction mixture was stirred at room temperature for 18 hours, under H2 (gas) atmosphere, at 1 atm of pressure. The mixture was filtered by passing through a plug of Celite and rinsed with EtOAc, the solvent was removed under reduced pressure and the resulted oil was used without further purification. (2.94 g. 97.2%). ¹H NMR (300 MHz, DMSO-d₆) δ 6.41 (s, 2H), 6.31 (s, 1H) 3.71 (s, 6H), 1.56-1.49 (m, 2H), 1.20 (s, 8H), 0.98 (m, 2H), 0.79 (t, J=6.99 Hz, 3H). LC-MS (ESI+): 237.2 (M+H⁺); R.T.: 1.845 min. (HPLC Method E).

### Step e. 5-(2-methylhexan-2-yl)benzene-1,3-diol

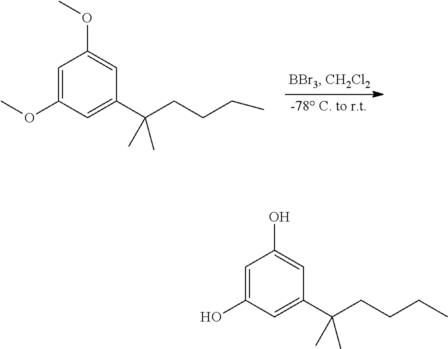 [SMILES: CCCCC(C)(C)c1cc(OC)cc(OC)c1 ; CCCCC(C)(C)c1cc(O)cc(O)c1]

Boron tribromide (1.5 mL, 15.93 mmol, 2.2 eq) was added dropwise (30 minutes) to a stirred solution od 1,3-dimethoxy-5-(2-methylhexan-2-yl)benzene (2.93 g, 12.40 mmol, 1 eq) in dry CH₂Cl₂ (62 mL) under nitrogen atmosphere at −78° C. After the addition, the cooling bath was removed, and the reaction mixture was stirred 1 hour at room temperature. The reaction was quenched by addition of ice while cooling with an ice-water bath. CH₂Cl₂ was added to dissolve the yellow solid, the organic layer was separated, and the product was extracted from aqueous layer with CH₂Cl₂, the combined organic layers were dried over anhydrous MgSO₄, filtered and concentrated under reduced pressure. The product was purified by chromatography on silica gel (Heptane/EtOAc; from 100:0 to 80:20). The desired fractions were collected and concentrated under reduced pressure to afford the product as colourless oil. (2.37 g, 91.8%). ¹H NMR (300 MHz, DMSO-d₆) δ 8.96 (s, 2H), 6.14 (s, 2H), 6.00 (s, 1H), 1.49-1.41 (m, 2H), 1.14 (s, 8H), 0.97 (m, 2H), 0.79 (t, J=6.87 Hz, 3H). LC-MS (ESI+): 209.1 (M+H⁺); R.T.: 1.365 min. (HPLC Method E).

### Step f. (6aR,10aR)-6,6,9-trimethyl-3-(2-methylhexan-2-yl)-6a,7,10,10a-tetrahydro-6H-benzo[c]chromen-1-ol

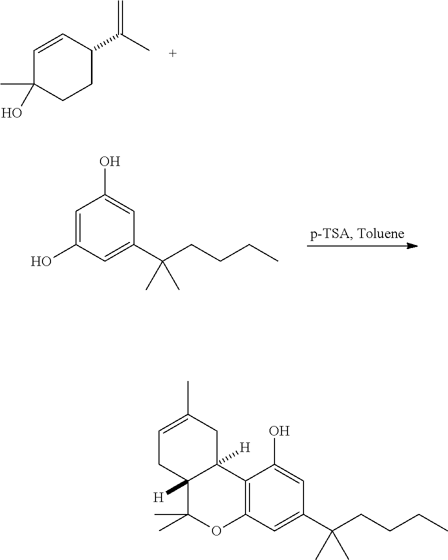 [SMILES: C=C(C)[C@H]1C=CC(C)(O)CC1 ; CCCCC(C)(C)c1cc(O)cc(O)c1 ; CCCCC(C)(C)c1cc(O)c2c(c1)OC(C)(C)[C@@H]1CC=C(C)C[C@@H]21]

A solution of para-menthane-3,8-diol (PMD) (1.90 g, 12.52 mmol, 1.1 eq) in toluene (5 mL) was added dropwise to a stirred solution of the intermediate from step e. (2.37 g, 11.38 mmol, 1 eq) and p-toluenesulfonic acid (p-TSA) (0.433 g, 2.28 mmol, 0.2 eq in toluene (29.0 mL). The mixture was stirred at room temperature for 1 h. Then the reaction mixture was stirred at 70-80° C. under partial vacuum with a Dean-Stark trap for 6 hours. The organic layer was washed with water and brine, dried over anhydrous MgSO₄, filtered and concentrated under reduced pressure to yield a crude that was purified by chromatography on silica gel (Heptane/EtOAc; from 100:0 to 80:20), the desired fractions were collected and concentrated under reduced pressure to give a yellow oil. (3.33 g, 85.5%). ¹H NMR (300 MHz, DMSO-d6) δ 9.13 (s, 1H), 6.29 (s, 1H), 6.11 (s, 1H), 5.39 (s, 1H), 3.21 (m, 1H), 2.10-1.98 (m, 1H), 1.84-1.70 (m, 1H), 1.64 (s, 4H), 1.58 (s, 1H), 1.47-1.40 (m, 2H), 1.28 (s, 3H), 1.14 (s, 8H), 1.00 (s, 5H), 0.80 (t, J=6.12 Hz, 3H). LC-MS (ESI+): 343.2 (M+H⁺); R.T.: 2.019 min. (HPLC Method E).

### Step g. (6aR,10aR)-6,6,9-trimethyl-3-(2-methylhexan-2-yl)-6a,7,10,10a-tetrahydro-6H-benzo[c]chromen-1-01

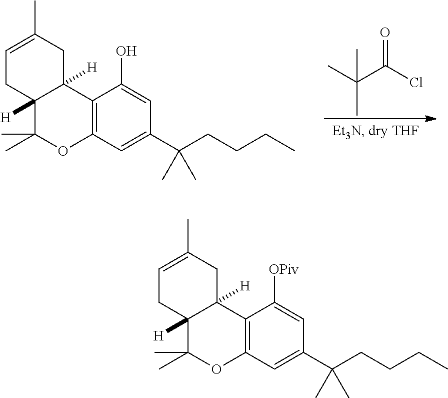

Triethylamine (9.85 mL, 97.31 mmol, 10 eq) and pivaloyl chloride (7.18 mL, 58.39 mmol, 6 eq) were added to a solution of (6aR,10aR)-6,6,9-trimethyl-3-(2-methylhexan-2-yl)-6a,7,10,10a-tetrahydro-6H-benzo[c]chromen-1-ol (intermediate from step f.) (3.33 g, 9.83 mmol, 1 eq) in dry THE (29 mL) under N₂ (g) atmosphere. The mixture reaction was stirred overnight. Then, additional triethylamine (4.92 mL, 48.65 mmol, 5 eq) and pivaloyl chloride (3.59 mL, 29.19 mmol, 3.0 eq) were added, and the reaction mixture was stirred at rt for 4 hours. The solution was diluted with EtOAc and washed with a saturated aqueous solution of NaHCO₃ and brine. The organic layer was dried over anhydrous MgSO₄, filtered and concentrated under reduced pressure. The product was purified by chromatography on silica gel (Heptane/EtOAc; from 100:0 to 90:10). The desired fractions were collected and concentrated under reduced pressure to give a colourless oil. (2.28 g, 54.9%). ¹H NMR (300 MHz, DMSO-d₆) δ 6.59 (d, J=1.74 Hz, 1H), 6.43 (d, J=1.77 Hz, 1H), 5.41 (bs, 1H), 2.69 (dd, J=15.99-3.81 Hz, 1H), 2.40 (dt, J=11.04-4.38 Hz, 1H), 2.10 (d, J=15.87 Hz, 1H), 1.86-1.68 (m, 2H), 1.63 (s, 3H), 1.51-1.46 (m, 2H), 1.32 (s, 3H), 1.29 (s, 9H), 1.20 (s, 1H), 1.17 (s, 8H), 1.11 (s, 1H), 1.01 (s, 4H), 0.80 (t, J=7.38 Hz, 3H). LC-MS (ESI+): 427.3 (M+H⁺); R.T.: 2.371 min. (HPLC Method E).

### Step h. (6aR,10aR)-6,6-dimethyl-3-(2-methylhexan-2-yl)-1-(pivaloyloxy)-6a,7,10,10a-tetrahydro-6H-benzo[c]chromene-9-carboxylic acid

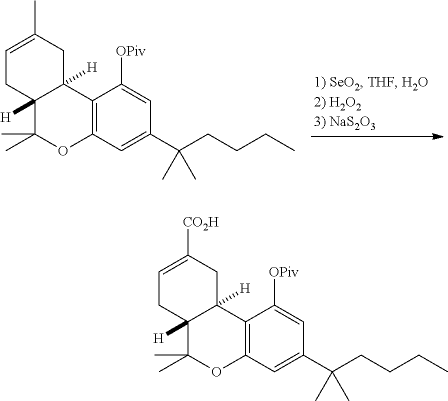

Selenium dioxide (0.740 g, 6.67 mmol, 1.25 eq) was added to a stirred solution of (6aR,10aR)-6,6,9-trimethyl-3-(2-methylhexan-2-yl)-6a,7,10,10a-tetrahydro-6H-benzo[c]chromen-1-ol (intermediate from step g.) (2.28 g, 5.34 mmol, 1 eq) in THE (16 mL) and water (19 μL, 1.07 mmol, 0.2 eq). The reaction mixture was stirred at 60° C. for 18 hours. The solution was cooled to 0° C., then a solution of hydrogen peroxide 30% in water (495 μL, 16.00 mmol, 3 eq) was added slowly and the reaction mixture was stirred at room temperature for 16 hours. The reaction was quenched with a 20 wt % sodium thiosulfate. The solution was filtered through a pad of Celite then was diluted with EtOAc and washed with brine. The organic layer was dried over anhydrous MgSO₄, filtered and concentrated under reduced pressure. The product was purified by chromatography on silica gel (Heptane: EtOAc; from 100:0 to 80:20) to yield the product as a light yellow solid (0.186 g, 7.64%). ¹H NMR (300 MHz, DMSO-d₆) δ 12.20 (bs, 1H), 6.88 (s, 1H), 6.60 (d, J=1.71 Hz, 1H), 6.45 (d, J=1.71 Hz, 1H), 3.25 (m, 1H), 2.40-2.32 (m, 2H), 2.07-1.95 (m, 1H), 1.84-1.69 (m, 2H), 1.50-1.44 (m, 2H), 1.34 (s, 3H), 1.20 (s, 9H), 1.18 (s, 8H), 1.03 (s, 5H), 0.80 (t, J=7.32 Hz, 3H). LC-MS (ESI+): 457.3 (M+H⁺); R.T.: 1.622 min. (HPLC Method E).

### Step i. (6aR,10aR)-6,6-dimethyl-3-(2-methylhexan-2-yl)-1-(pivaloyloxy)-6a,7,10,10a-tetrahydro-6H-benzo[c]chromene-9-carboxylic acid (Compound 109)

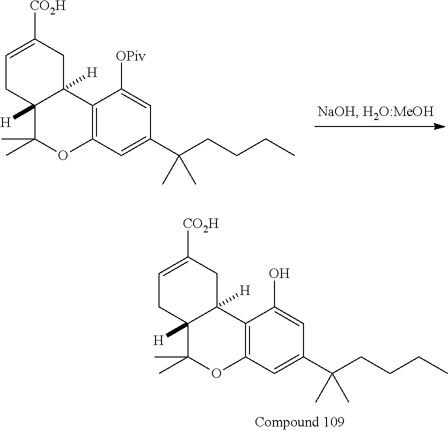

(6aR,10aR)-6,6-dimethyl-3-(2-methylhexan-2-yl)-1-(pivaloyloxy)-6a,7,10,10a-tetrahydro-6H-benzo[c]chromene-9-carboxylic acid (intermediate from step h.) (64 mg, 0.14 mmol, 1 eq) was dissolved into a mixture MeOH:H₂O (1:1) (0.42 mL) and cooled at 0° C., then sodium methoxide 25% in methanol (0.16 mL, 0.70 mmol, 5 eq) was added and the mixture reaction was stirred at room temperature for 16 hours. The solution was acidified to pH=5 with an aqueous solution of [1 M] HCl, the product was extracted with EtOAc. The combined organic layers were dried over anhydrous MgSO₄, filtered and concentrated under reduced pressure. The product was purified by flash column chromatography on silica gel (Heptane: EtOAc; from 100:0 to 70:30). The fractions contained desired product were collected and concentrated under reduced pressure to yield Compound 109 as a white solid (0.033 g, 63.4%). ¹H NMR (300 MHz, DMSO-d₆) d. 12.12 (s, 1H), 9.21 (s, 1H), 6.88 (s, 1H), 6.32 (d, J=1.47 Hz, 1H), 6.13 (d, J=1.47 Hz, 1H), 3.75 (d, J=15.84 Hz, 1H), 2.43-2.27 (m, 1H), 2.00 (t, J=18.33 Hz, 1H), 1.66 (dt, J=11.45-4.17 Hz, 2H), 1.49-1.43 (m, 2H), 1.31 (s, 3H), 1.14 (s, 9H), 1.02 (s, 5H), 0.80 (t, J=7.35 Hz, 3H). LC-MS (ESI+): 373.2 (M+H⁺); R.T.: 3.982 min. (HPLC Method C).

### Example 63. Synthesis of 6aR,10aR)-1-hydroxy-6,6-dimethyl-3-(2-methylhexan-2-yl)-6a,7,10,10a-tetrahydro-6H-benzo[c]chromene-9-carboxylic acid (Compound 110)

Compound 110 was synthesized analogously to Compound 109 using ethyltriphenylphosphonium bromide instead of triphenyl(propyl)phosphonium bromide.

### Step a. (Z)-1,3-dimethoxy-5-(2-methylpent-3-en-2-yl)benzene

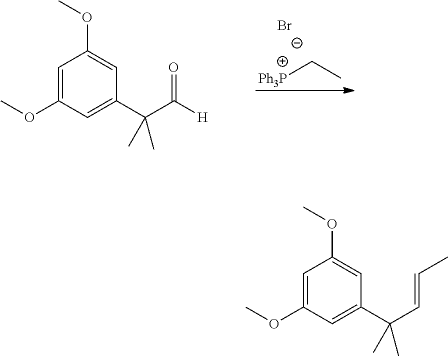

Yield 69.8% (2.86 g). ¹H NMR (300 MHz, DMSO-d₆) δ 6.46 (s, 2H), 6.31 (s, 1H), 5.62 (d, J=10.89 Hz, 1H), 5.43-5.32 (m, 1H), 3.70 (s, 6H), 1.34 (s, 6H), 1.21 (d, J=6.78 Hz, 3H). LC-MS (ESI+): 221.1 (M+H⁺); R.T.: 1.678 min. (HPLC Method E).

### Step b. 1,3-dimethoxy-5-(2-methylpentan-2-yl)benzene

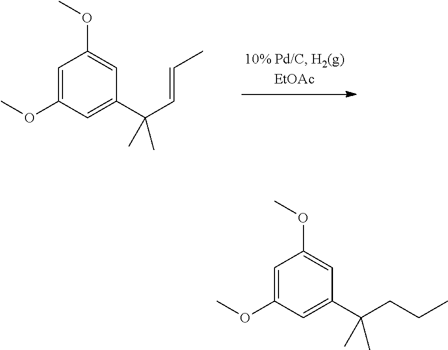 [SMILES: C/C=C/C(C)(C)c1cc(OC)cc(OC)c1 ; CCCC(C)(C)c1cc(OC)cc(OC)c1]

Yield 56.3% (1.63 g). ¹H NMR (300 MHz, CDCl₃) δ 6.42 (s, 1H), 6.31 (s, 1H), 3.71 (s, 6H), 1.51 (m, 2H), 1.20 (s, 6H), 0.99 (q, J=7.62 Hz, 2H), 0.77 (t, J=6.63 Hz, 3H). LC-MS (ESI+): 223.1 (M+H⁺); R.T.: 1.775 min. (HPLC Method E).

### Step c. 5-(2-methylpentan-2-yl)benzene-1,3-diol

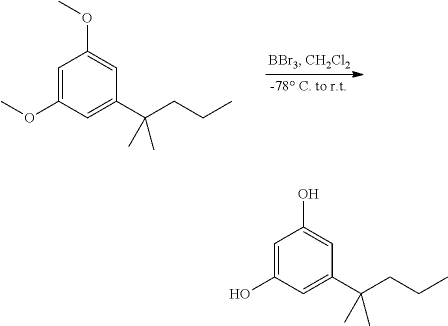 [SMILES: CCCC(C)(C)c1cc(OC)cc(OC)c1 ; CCCC(C)(C)c1cc(O)cc(O)c1]

Yield 90.3% (1.27 g). ¹H NMR (300 MHz, DMSO-d₆) δ 8.96 (s, 2H), 6.14 (s, 2H), 6.00 (s, 1H), 1.47-1.41 (m, 2H), 1.14 (s, 6H), 1.01 (q, J=7.02 Hz, 2H), 0.78 (t, J=6.63 Hz, 3H). LC-MS (ESI+): 195.1 (M+H⁺); R.T.: 1.275 min. (HPLC Method E).

### Step d. (6aR,10aR)-6,6,9-trimethyl-3-(2-methylpentan-2-yl)-6a,7,10,10a-tetrahydro-6H-benzo[c]chromen-1-ol

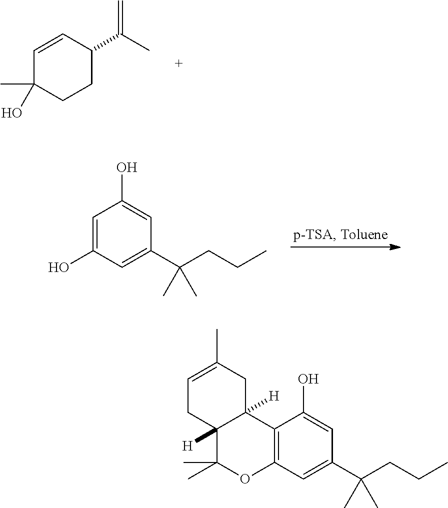

Yield 87.2% (1.87 g). ¹H NMR (300 MHz, DMSO-d₆) δ 9.13 (s, 1H), 6.29 (s, 1H), 6.11 (s, 1H), 5.39 (s, 1H), 3.21 (m, 1H), 2.10-1.98 (m, 1H), 1.84-1.70 (m, 1H), 1.64 (s, 4H), 1.58 (s, 1H), 1.47-1.40 (m, 2H), 1.28 (s, 3H), 1.14 (s, 1H), 1.00 (s, 5H), 0.78 (t, J=6.45 Hz, 3H). LC-MS (ESI+): 329.2 (M+H⁺); R.T.: 1.964 min. (HPLC Method E).

### Step e. (6aR,10aR)-6,6,9-trimethyl-3-(2-methylpentan-2-yl)-6a,7,10,10a-tetrahydro-6H-benzo[c]chromen-1-yl pivalate

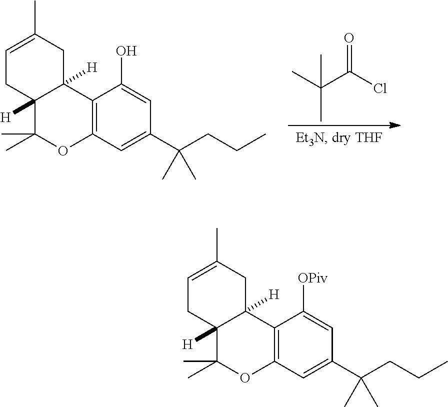

Yield 88.0% (2.07 g). ¹H NMR (300 MHz, DMSO-d₆) δ 6.59 (d, J=1.77 Hz, 1H), 6.43 (d, J=1.80 Hz, 1H), 5.41 (bs, 1H), 2.69 (dd, J=15.63-3.96 Hz, 1H), 2.40 (dt, J=11.55-4.32 Hz, 1H), 2.10 (d, J=14.70 Hz, 1H), 1.86-1.68 (m, 2H), 1.63 (s, 3H), 1.49-1.44 (m, 2H), 1.32 (s, 3H), 1.30 (s, 9H), 1.20 (s, 1H), 1.18 (s, 6H), 1.11 (s, 1H), 1.01 (s, 4H), 0.78 (t, J=7.29 Hz, 3H). LC-MS (ESI+): 413.3 (M+H⁺); R.T.: 2.27 min. (HPLC Method E).

### Step f. (6aR,10aR)-6,6-dimethyl-3-(2-methylpentan-2-yl)-1-(pivaloyloxy)-6a,7,10,10a-tetrahydro-6H-benzo[c]chromene-9-carboxylic acid

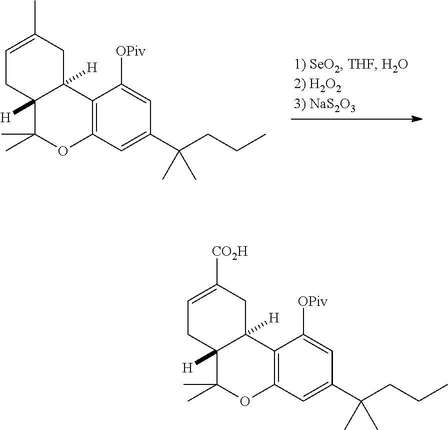

Yield 17.2% (380 mg). ¹H NMR (300 MHz, DMSO-d₆) δ 12.21 (bs, 1H), 6.88 (s, 1H), 6.60 (d, J=1.77 Hz, 1H), 6.45 (d, J=1.74 Hz, 1H), 3.25 (m, 1H), 2.40-2.31 (m, 2H), 2.07-1.95 (m, 1H), 1.84-1.69 (m, 2H), 1.50-1.44 (m, 2H), 1.34 (s, 3H), 1.20 (s, 9H), 1.18 (s, 6H), 1.03 (s, 5H), 0.78 (t, J=7.35 Hz, 3H). LC-MS (ESI+): 443.2 (M+H⁺); R.T.: 1.487 min. (HPLC Method E).

### Step g. (6aR,10aR)-1-hydroxy-6,6-dimethyl-3-(2-methylpentan-2-yl)-6a,7,10,10a-tetrahydro-6H-benzo[c]chromene-9-carboxylic acid (Compound 110)

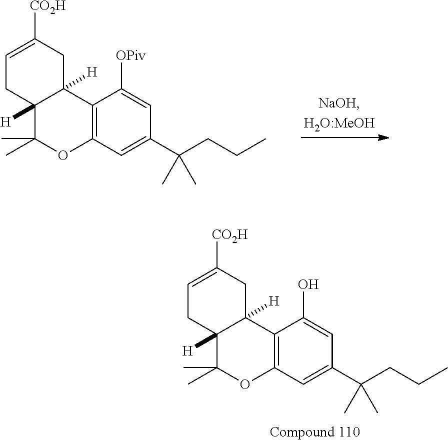

Yield 50.4% (80 mg). ¹H NMR (300 MHz, CDCl₃) δ 7.15 (d, J=4.41 Hz, 1H), 6.39 (d, J=1.50 Hz, 1H), 6.23 (d, J=1.35 Hz, 1H), 3.82 (dd, J=16.89-2.37 Hz, 1H), 2.67 (dt, J=11.01-4.62 Hz, 1H), 2.44 (m, 1H), 2.9-1.93 (m, 2H), 1.84 (dt, J=11.52-4.23 Hz, 1H), 1.51-1.45 (m, 2H), 1.41 (s, 3H), 1.20 (s, 7H), 1.13 (s, 3H), 1.10-0.99 (m, 2H), 0.80 (t, J=7.29 Hz, 3H). LC-MS (ESI+): 359.2 (M+H⁺); R.T.: 3.825 min. (HPLC Method C).

### Example 64. Synthesis of (6aR,10aR)-3-(7-carboxy-2-methylheptan-2-yl)-1-hydroxy-6,6-dimethyl-6a,7,10,10a-tetrahydro-6H-benzo[c]chromene-9-carboxylic acid (Compound 111)

Compound 111 was prepared analogously to the synthesis of Compound 109 using (4-carboxybutyl)triphenylphosphonium bromide (synthesized according to the reference procedure provided in J. Am. Chem. Soc., 1970, 92 (11), pp 3429-3433) and 2-(3,5-dimethoxyphenyl)-2-methylpropanal (synthesized according to step b. in the preparation of Compound 109) as starting materials.

### Step a. (Z)-7-(3,5-dimethoxyphenyl)-7-methyloct-5-enoic acid

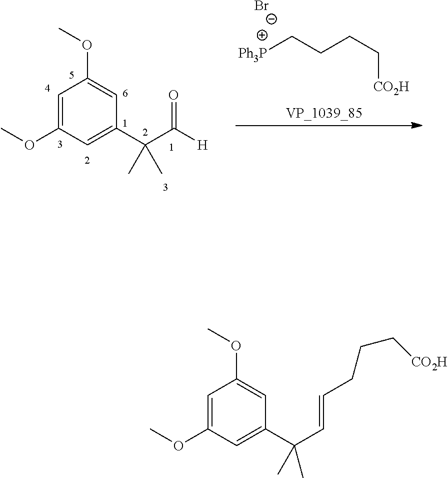

Yield 69.4% (11.2 g). ¹H NMR (300 MHz, CDCl₃) δ 6.53 (s, 2H), 6.29 (s, 1H), 5.68 (d, J=11.4 Hz, 1H), 5.47-5.10 (m, 1H), 3.78 (s, 6H), 2.10 (t, J=7.5 Hz, 2H), 1.69 (dd, J=14.4, 7.2 Hz, 2H), 1.55-1.44 (m, 2H), 1.39 (s, 6H). LC-MS (ESI+): 293.2 (M+H⁺); R.T.: 1.461 min. (HPLC Method E).

### Step b. 7-(3,5-dimethoxyphenyl)-7-methyloctanoic acid

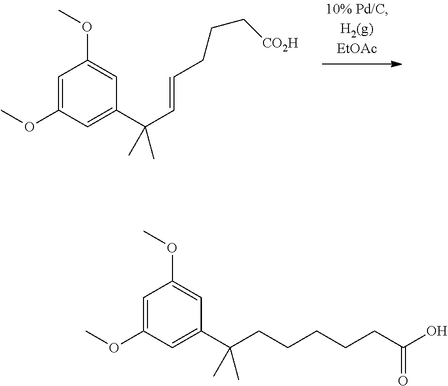 [SMILES: COc1cc(OC)cc(C(C)(C)/C=C/CCCC(=O)O)c1 ; COc1cc(OC)cc(C(C)(C)CCCCCC(=O)O)c1]

Yield 93% (10.5 g). LC-MS (ESI+): 295.2 (M+H⁺); R.T.: 1.490 min. (HPLC Method E).

### Step c. 7-(3,5-dihydroxyphenyl)-7-methyloctanoic acid

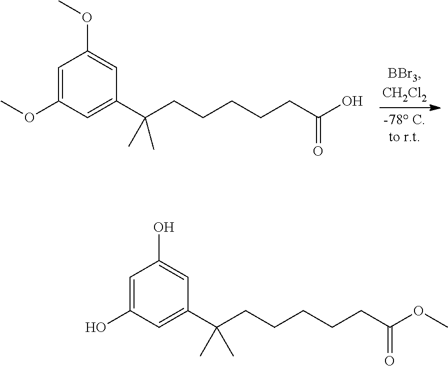 [SMILES: COc1cc(OC)cc(C(C)(C)CCCCCC(=O)O)c1 ; COC(=O)CCCCCC(C)(C)c1cc(O)cc(O)c1]

Yield 73.5% (9.4 g). ¹H NMR (300 MHz, CDCl₃) δ 6.37 (s, 2H), 6.20 (s, 1H), 5.99 (s, 2H), 3.66 (s, 3H), 2.24 (t, J=7.3 Hz, 2H), 1.74-1.31 (m, 4H), 1.20 (d, J=13.5 Hz, 8H), 1.04 (s, 2H). LC-MS (ESI+): 281.2 (M+H⁺); R.T.: 1.269 min. (HPLC Method E).

### Step d. Methyl 7-((6aR,10aR)-1-hydroxy-6,6,9-trimethyl-6a,7,10,10a-tetrahydro-6H-benzo[c]chromen-3-yl)-7-methyloctanoate

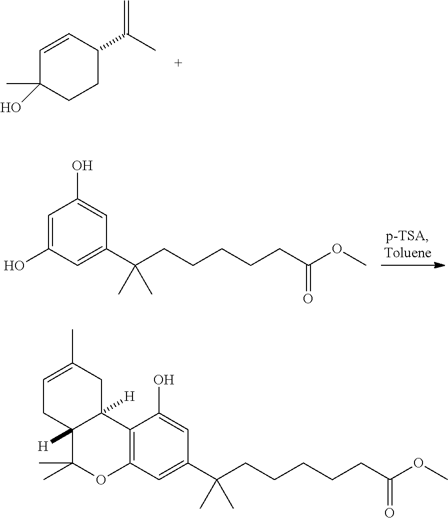

Yield 52.4% (7.3 g). ¹H NMR (300 MHz, CDCl₃) δ 6.37 (s, 1H), 6.22 (s, 1H), 5.42 (s, 1H), 4.91 (s, 1H), 3.65 (s, 2H), 3.20 (dd, J=16.1, 4.5 Hz, 1H), 2.71 (dd, J=21.0, 6.6 Hz, 1H), 2.24 (t, J=7.4 Hz, 2H), 2.15 (d, J=12.7 Hz, 1H), 1.97-1.76 (m, 3H), 1.70 (s, 3H), 1.54-1.45 (m, 3H), 1.38 (s, 3H), 1.25 (s, 2H), 1.20 (s, 8H), 1.11 (s, 5H). LC-MS (ESI+): 415.3 (M+H⁺); R.T.: 1.90 min. (HPLC Method E).

### Step e. Methyl 7-((6aR,10aR)-6,6,9-trimethyl-1-(pivaloyloxy)-6a,7,10,10a-tetrahydro-6H-benzo[c]chromen-3-yl)octanoate

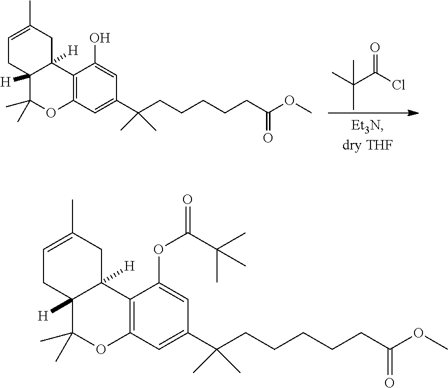

Yield 41.9% (4.5 g). ¹H NMR (300 MHz, CDCl₃) δ 6.66 (s, 1H), 6.40 (s, 1H), 5.41 (s, 1H), 3.64 (s, 3H), 2.76 (d, J=16.8 Hz, 1H), 2.54 (m, 1H), 2.24 (t, J=7.4 Hz, 2H), 2.13 (m, 1H), 1.99-1.72 (m, 3H), 1.67 (s, 3H), 1.63-1.42 (m, 5H), 1.37 (s, 11H), 1.22 (s, 9H), 1.11 (s, 4H). LC-MS (ESI+): 516.4 (M+H₂O+); R.T.: 2.144 min. (HPLC Method E).

### Step f. (6aR,10aR)-3-(8-methoxy-2-methyl-8-oxooctan-2-yl)-6,6-dimethyl-1-(pivaloyloxy)-6a,7,10,10a-tetrahydro-6H-benzo[c]chromene-9-carboxylic acid

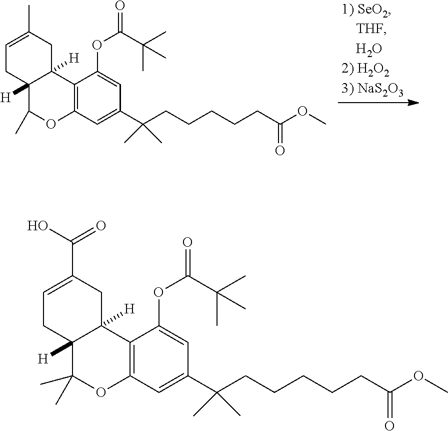

Yield 22.4% (178 mg). LC-MS (ESI+): 529.3 (M+H⁺) 546.3 (H₂O+); R.T.: 1.833 min. (HPLC Method E).

### Step g. (6aR,10aR)-3-(7-carboxy-2-methylheptan-2-yl)-1-hydroxy-6,6-dimethyl-6a,7,10,10a-tetrahydro-6H-benzo[c]chromene-9-carboxylic acid (Compound 111)

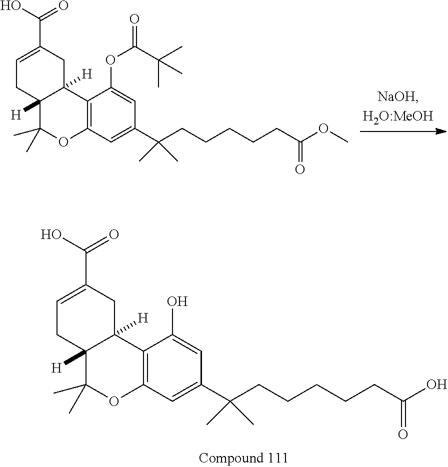

The intermediate from step f. (189 mg, 0.357 mmol, 1 eq) was dissolved into a mixture MeOH:H₂O (1:1) and cooled at 0° C., then NaOH (0.143 g, 3.57 mmol, 10 eq) was added and the mixture reaction was stirred at room temperature for overnight. The solution was acidified to pH=3 with an aqueous solution [1 M] of HCl, the product was extracted with CH₂Cl₂. The combined organic layers were dried over anhydrous MgSO₄, filtered and concentrated under reduced pressure. The residue was purified by flash column chromatography on silica gel (CH₂Cl₂: EtOAc; from 100:0 to 50:50). The desired fractions were collected and concentrated under reduced pressure to afford the title compound in 35.1% yield (54 mg). ¹H NMR (300 MHz, DMSO-d₆) δ 12.05 (s, 2H), 9.23 (s, 1H), 6.88 (s, 1H), 6.32 (s, 1H), 6.14 (s, 1H), 3.74 (d, J=17.3 Hz, 1H), 2.45-2.28 (m, 2H), 2.13 (t, J=6.7 Hz, 2H), 2.06-1.89 (m, 1H), 1.66 (t, J=12.7 Hz, 2H), 1.41 (d, J=8.1 Hz, 4H), 1.31 (s, 3H), 1.15 (s, 8H), 1.03 (s, 5H). LC-MS (ESI+): 431.3 (M+H⁺); R.T.: 3.299 min. (HPLC Method C).

### Example 65. Synthesis of (6aR,10aR)-1-hydroxy-3-(8-hydroxy-2-methyloctan-2-yl)-6,6-dimethyl-6a,7,10,10a-tetrahydro-6H-benzo[c]chromene-9-carboxylic acid (Compound 57)

### Step a. (6aR,10aR)-3-(8-hydroxy-2-methyloctan-2-yl)-6,6,9-trimethyl-6a,7,10,10a-tetrahydro-6H-benzo[c]chromen-1-ol

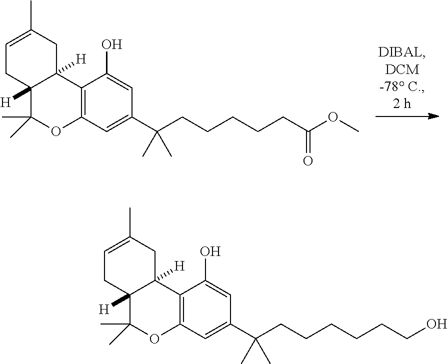

To a solution of the intermediate of step f. in the synthesis of Compound 111 (6.37 g, 15.36 mmol, 1 eq.) in dry CH₂Cl₂ (46 mL) under nitrogen atmosphere at −78° C. was added DIBAL ([1 M] solution in toluene) (38.4 mL, 38.4 mmol, 2.5 eq). The reaction mixture was stirred at −78° C. for 1 hour. Then an aqueous solution of Na/K tartrate (10%) was added and the mixture was warmed to room temperature and stirred vigorously overnight. The solid was filtered through celite and rinsed with CH₂Cl₂. The filtrate was extracted with CH₂Cl₂ and the combined organic layer was washed with brine and water, dried over anhydrous MgSO₄, filtered and condensed in vacuum. The residue was purified by chromatography on silica gel (Heptane: EtOAc, from 100:0 to 80:20). The desired fractions were collected and concentrated under reduced 5 pressure to afford the title compound as a colourless oil (5.4 g, 90.1%). ¹H NMR (300 MHz, CDCl₃) δ 6.37 (s, 1H), 6.24 (s, 1H), 5.42 (d, J=2.3 Hz, 1H), 5.19 (s, 1H), 3.63 (t, J=5.4 Hz, 2H), 3.20 (dd, J=16.2, 3.3 Hz, 1H), 2.69 (td, J=10.5, 4.3 Hz, 1H), 2.15 (d, J=12.2 Hz, 1H), 1.95-1.77 (m, 3H), 1.70 (s, 3H), 1.61 (s, 1H), 1.54-1.44 (m, 4H), 1.38 (s, 3H), 1.36-1.22 (m, 4H), 1.20 (s, 6H), 1.11 (s, 5H). LC-MS (ESI+): 397.3 (M+H⁺); R.T.: 1.777 min. (HPLC Method E).

### Step b. 7-methyl-7-((6aR,10aR)-6,6,9-trimethyl-1-(pivaloyloxy)-6a,7,10,10a-tetrahydro-6H-benzo[c]chromen-3-yl)octyl pivalate

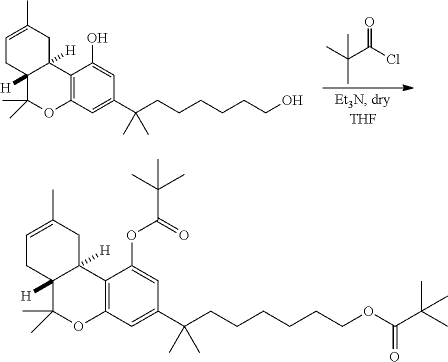

Yield 41.9% (4.5 g). ¹H NMR (300 MHz, CDCl₃) δ 6.66 (s, 1H), 6.40 (s, 1H), 5.41 (s, 1H), 4.00 (t, J=6.2 Hz, 2H), 2.76 (d, J=16.8 Hz, 1H), 2.55 (m, 1H), 2.12 (m, 3H), 1.97-1.74 (m, 3H), 1.67 (s, 5H), 1.63-1.44 (m, 4H), 1.37 (s, 11H), 1.29-1.14 (m, 21H), 1.11 (s, 4H). LC-MS (ESI+): 555.4 (M+H⁺) 572.4 (M+Na⁺); R.T.: 2.59 min. (HPLC Method E).

### Step c. (6aR,10aR)-6,6-dimethyl-3-(2-methyl-8-(pivaloyloxy)octan-2-yl)-1-(pivaloyloxy)-6a,7,10,10a-tetrahydro-6H-benzo[c]chromene-9-carboxylic acid

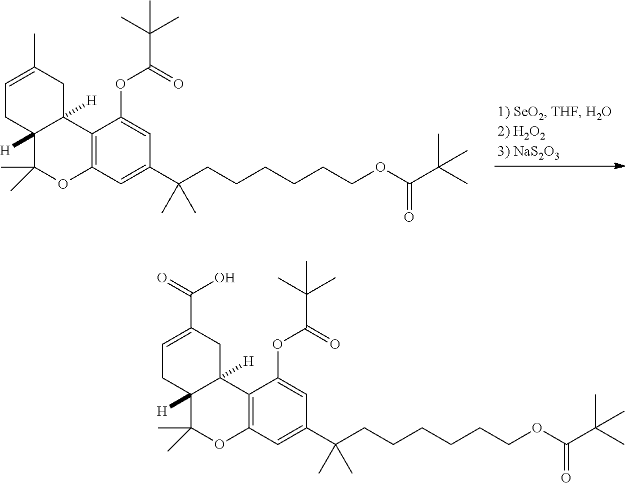

Yield 13.0% (640 mg). ¹H NMR (300 MHz, CDCl₃) δ 7.14 (s, 1H), 6.68 (s, 1H), 6.44 (s, 1H), 4.01 (t, J=5.9 Hz, 2H), 3.42 (d, J=17.2 Hz, 1H), 2.49 (dd, J=26.4, 15.4 Hz, 2H), 2.22-1.65 (m, 3H), 1.51 (d, J=13.0 Hz, 3H), 1.44-1.02 (m, 38H). LC-MS (ESI+): 585.4 (M+H⁺) 602.4 (NH₄+); R.T.: 1.824 min. (HPLC Method E).

### Step d. (6aR,10aR)-1-hydroxy-3-(8-hydroxy-2-methyloctan-2-yl)-6,6-dimethyl-6a,7,10,10a-tetrahydro-6H-benzo[c]chromene-9-carboxylic acid (Compound 57)

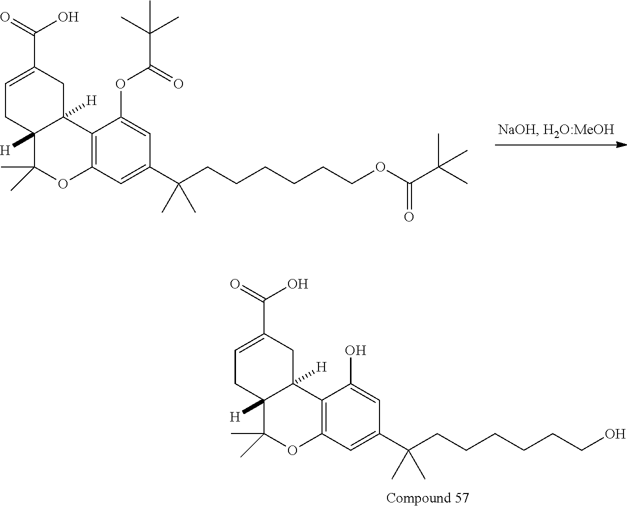

Yield 50.4% (80 mg). ¹H NMR (300 MHz, DMSO-d₆) δ 12.16 (s, 1H), 9.22 (s, 1H), 6.87 (s, 1H), 6.32 (s, 1H), 6.13 (s, 1H), 4.28 (s, 1H), 3.75 (d, J=17.4 Hz, 2H), 2.45-2.24 (m, 1H), 2.11-1.87 (m, 1H), 1.66 (t, J=11.5 Hz, 2H), 1.50-1.41 (m, 2H), 1.31 (s, 4H), 1.15 (s, 14H), 1.02 (s, 4H). LC-MS (ESI+): 417.3 (M+H⁺); R.T.: 3.401 min. (HPLC Method C).

### Example 66. Synthesis of (6aR,10aR)-6,6-dimethyl-3-(2-methyloctan-2-yl)-6a,7,10,10a-tetrahydro-6H-benzo[c]chromene-9-carboxylic acid (Compound 21)

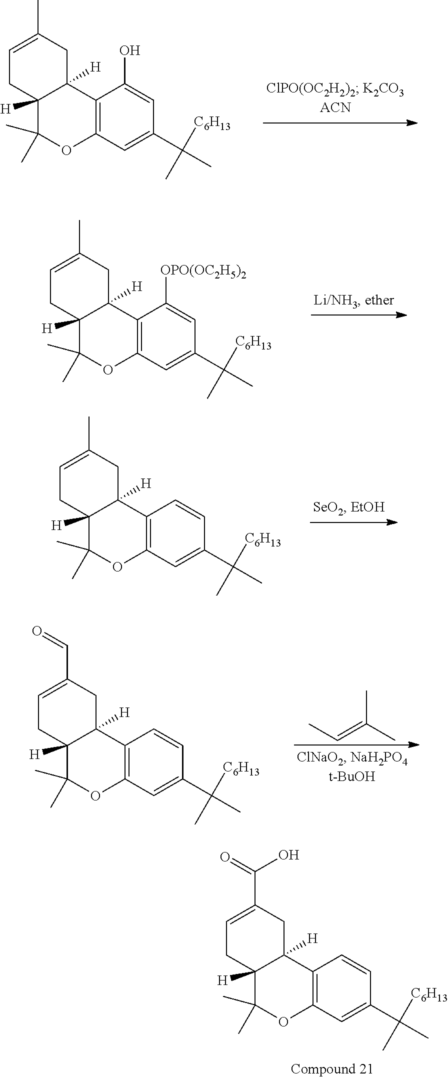

### Step a. Diethyl ((6aR,10aR)-6,6,9-trimethyl-3-(2-methyloctan-2-yl)-6a,7,10,10a-tetrahydro-6H-benzo[c]chromen-1-yl) phosphate

The starting material was prepared analogously to the intermediate from step f. of Compound 109. To a solution of the starting material (6.55 g, 16.7 mmol, 1.0 eq) in dry acetonitrile (53 mL) was added K₂CO3 (20.3 g, 146.7 mmol, 8.3 eq) followed by diethylchlorophosphate (3.8 g, 26.5 mmol, 1.5 eq), under N₂ atmosphere. The reaction mixture was stirred at 90° C. for 1 h. The residue was poured in water and diethyl ether. The aqueous layer was separated and extracted with diethyl ether. The combined organic layers were dried over anhydrous MgSO₄, filtered and concentrated under reduced pressure. The crude was purified by flash column chromatography on silica gel (heptane/EtOAc; from 100:0 to 90:10), the desired fractions were collected and concentrated under reduced pressure to afford the title compound as a yellow oil (8.8 g, 98.3%). ¹H NMR (300 MHz, CDCl₃) δ 6.83 (s, 1H), 6.61 (s, 1H), 5.42 (s, 1H), 4.31-4.00 (m, 4H), 3.05 (d, J=17.6 Hz, 1H), 2.80 (s, 1H), 2.12 (s, 1H), 1.97-1.74 (m, 3H), 1.69 (s, 3H), 1.61-1.44 (m, 5H), 1.44-0.91 (m, 30H), 0.83 (d, J=6.1 Hz, 3H). LC-MS, R.T.: 4.005 min. (HPLC Method E).

### Step b. (6aR,10aR)-6,6,9-trimethyl-3-(2-methyloctan-2-yl)-6a,7,10,10a-tetrahydro-6H-benzo[c]chromene

A solution of the intermediate from step a. (7.0 g, 13.8 mmol) in dry ether was added to liquid ammonia (300 mL). The reaction was stirred vigorously and small pieces of Li (0.16 g) were added until blue color persisted for more >5 min. Excess Li was then decomposed by addition of NH₄Cl and NH₃ was allowed to evaporate using a stream of nitrogen. The residue was partitioned between water and ether. The aqueous layer was separated and extracted with diethyl ether. The combined organic layers were washed with water, dried over anhydrous MgSO₄, filtered and concentrated under reduced pressure. The product was purified by chromatography on silica gel (100% heptane), the desired fractions were collected and concentrated under reduced pressure to afford the title compound as colorless oil (2.53 g, 51.7%). ¹H NMR (300 MHz, CDCl₃) δ 7.12 (d, J=7.9 Hz, 1H), 6.84 (d, J=7.7 Hz, 1H), 6.76 (s, 1H), 5.45 (s, 1H), 2.78-2.51 (m, 2H), 2.16 (d, J=15.7 Hz, 1H), 2.07-1.61 (m, 6H), 1.54 (d, J=9.0 Hz, 3H), 1.40 (s, 3H), 1.17 (dd, J=31.0, 21.9 Hz, 15H), 0.84 (s, 4H). GS/MS (EI+): M+354.4; R.T.: 13.26 min. (HPLC Method GC/MS20 MB).

### Step c. (6aR,10aR)-6,6-dimethyl-3-(2-methyloctan-2-yl)-6a,7,10,10a-tetrahydro-6H-benzo[c]chromene-9-carbaldehyde

To a solution of of the intermediate from step b. (2.53 gr, 7.13 mmol) in ethanol (30 mL) was added dropwise a solution of Se₂O (1.9 g, 17.12 mmol) in EtOH/H₂O (30 mL/3 mL) over 0.5 h at room temperature. The reaction was then refluxed overnight. The reaction mixture was cooled, filtered through a celite pad, and washed with MeOH. The filtrate was concentrated, and the residue was dissolved in diethyl ether, washed with water and aqueous saturated NaHCO₃ solution. The organic layer was dried over anhydrous MgSO₄, filtered and concentrated under reduced pressure. The crude was purified by chromatography on silica gel (heptane: EtOAc; from 100:0 to 95:5) to afford the title compound as a colourless oil. (0.74 g; 28.1%). ¹H NMR (300 MHz, CDCl₃) δ 9.52 (s, 1H), 7.20 (d, J=8.0 Hz, 1H), 6.87 (d, J=5.5 Hz, 2H), 6.76 (s, 1H), 3.20 (dd, J=17.2, 3.8 Hz, 1H), 2.71-2.48 (m, 2H), 2.15 (dd, J=18.5, 12.3 Hz, 1H), 2.05-1.91 (m, 1H), 1.85 (td, J=11.5, 4.8 Hz, 1H), 1.55 (t, J=9.3 Hz, 3H), 1.45 (s, 3H), 1.25 (s, 7H), 1.20 (s, 8H), 1.06 (bs, 1H), 0.83 (t, J=6.2 Hz, 3H). LC-MS (ESI+): 369.2, R.T.: 2.094 min. (HPLC Method E.).

### Step d. (6aR,10aR)-6,6-dimethyl-3-(2-methyloctan-2-yl)-6a,7,10,10a-tetrahydro-6H-benzo[c]chromene-9-carboxylic acid (Compound 21)

A round-bottom flask with a condenser and nitrogen inlet was charged with the intermediate of step c. (0.73 gr, 1.98 mmol), t-BuOH (30 mL) and 2-methyl-2-butene (30 mL). To this was added a solution of NaClO₂ and KH₂PO₄ in water (2.06 g, 2.23 gr, 20.0 mL) over 0.5 h. The biphasic mixture was vigorously stirred for 2.5 hours. The reaction mixture was concentrated, and the residue was partitioned between water and ether. The aqueous layer was separated and extracted with diethyl ether (3×40 mL). The combined organics layers were washed with [1 M] HCl and water, dried over MgSO₄, filtered and concentrated under reduced pressure. The residue was purified by chromatography on silica gel (heptane: EtOAc; from 100:0 to 85:15). The desired fractions were collected and concentrated under reduced pressure to afford a white solid (0.4 g, 52.5 ¹H NMR (300 MHz, CDCl₃) δ 7.20 (d, J=7.6 Hz, 2H), 6.88 (dd, J=8.1, 1.8 Hz, 1H), 6.77 (d, J=1.8 Hz, 1H), 3.22 (dd, J=16.1, 4.6 Hz, 1H), 2.67 (td, J=11.2, 5.1 Hz, 1H), 2.56-2.38 (m, 1H), 2.22-1.94 (m, 2H), 1.79 (td, J=11.5, 4.9 Hz, 1H), 1.62-1.48 (m, 2H), 1.43 (s, 3H), 1.22 (d, J=19.0 Hz, 17H), 1.13-0.96 (m, 2H), 0.84 (t, J=6.6 Hz, 3H). %). LC-MS (ESI+): 385.3 (M+H⁺); R.T.: 4.722 min. (HPLC Method C). UV purity: 99%.

### Example 67. Synthesis of (6aR,10aR)-1-hydroxy-6,6-dimethyl-3-(2-methyloctan-2-yl)-6a,7,10,10a-tetrahydro-6H-benzo[c]chromene-9-carbonitrile (Compound 112)

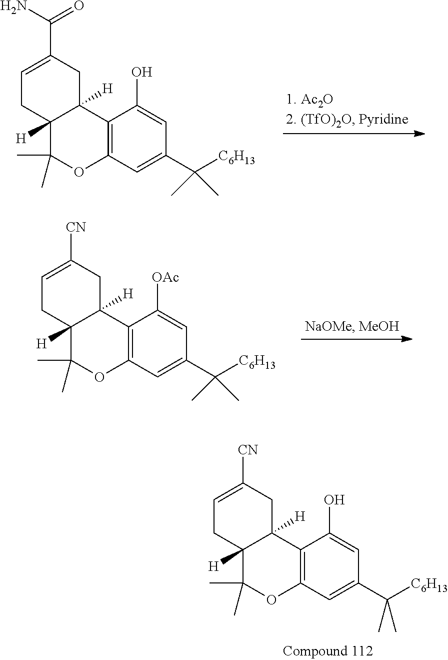

### Step a. (6aR,10aR)-9-cyano-6,6-dimethyl-3-(2-methyloctan-2-yl)-6a,7,10,10a-tetrahydro-6H-benzo[c]chromen-1-yl acetate

Acetic anhydride (1.63 mL, 17.30 mmol, 2.0 eq) was added to a stirred solution of (6aR,10aR)-1-hydroxy-6,6-dimethyl-3-(2-methyloctan-2-yl)-6a,7,10,10a-tetrahydro-6H-benzo[c]chromene-9-carboxamide (3.457 g, 8.65 mmol, 1 eq, Compound 4) in pyridine (26 mL). The mixture was stirred at room temperature for 1 hour. The solution was diluted with EtOAc and washed with an aqueous solution of HCl [1M], and brine, the organic layer was dried over anhydrous MgSO₄, filtered and concentrated under reduced pressure. The residue was dissolved in pyridine (26 mL) and cooled at 0° C., then trifluoromethanesulfonic anhydride (2.33 mL, 13.84 mmol, 1.6 eq) was added dropwise. The reaction mixture was warmed to rt and stirred for 1 h. The mixture was diluted with EtOAc and washed with an aqueous solution of HCl [1 M], and brine, the organic layer was dried over anhydrous MgSO₄, filtered and concentrated under reduced pressure. The residue was purified by chromatography on silica gel (Heptane: EtOAc; from 100:0 to 90:10) to yield the title compound as an oil (1.77 g; 48.3%). ¹H NMR (300 MHz, DMSO-d₆) δ 6.82 (s, 1H), 6.58 (d, J=8.73 Hz, 2H), 2.97 (d, J=16.92 Hz, 1H), 2.56 (m, 1H), 2.39 (d, J=17.91 Hz, 1H), 2.29 (s, 3H), 2.14-1.98 (m, 2H), 1.76 (t, J=9.60 Hz, 1H), 1.48 (m, 2H), 1.33 (s, 3H), 1.17 (s, 12H), 1.02 (s, 5H), 0.81 (t, J=5.73 Hz, 3H). LC-MS (ESI+): 424.3 (M+H⁺); R.T.: 1.986 min. (HPLC Method E).

### Step b. (6aR,10aR)-1-hydroxy-6,6-dimethyl-3-(2-methyloctan-2-yl)-6a,7,10,10a-tetrahydro-6H-benzo[c]chromene-9-carbonitrile (Compound 112)

Sodium methoxide 25% in methanol (0.08 mL, 0.354 mmol, 1.5 eq) was added dropwise to a solution of the intermediate from step a. (0.1 g, 0.236 mmol, 1 eq) in MeOH (0.63 mL). The reaction mixture was stirred at room temperature for 1 hour. The solution was diluted with water, the pH was adjusted to 7 with an aqueous solution of [1M] HCl, and the mixture was extracted with EtOAc. The combined organic layers were dried over anhydrous MgSO₄, filtered and concentrated under reduced pressure. The residue was purified by chromatography on silica gel (Heptane/EtOAc; from 100:0 to 70:30) to yield the title compound as a white solid (68 mg, 75.5%). ¹H NMR (300 MHz, DMSO-d₆) δ 9.36 (s, 1H), 6.79 (s, 1H), 6.33 (s, 1H), 6.14 (s, 1H), 3.52 (d, J=16.68 Hz, 1H), 2.57 (m, 1H), 2.38 (d, J=18.45 Hz, 1H), 2.07-1.90 (m, 2H), 1.71 (t, J=10.53 Hz, 1H), 1.45 (m, 2H), 1.30 (s, 3H), 1.13 (s, 12H), 1.01 (s, 5H), 0.81 (t, J=5.82 Hz, 3H). LC-MS (ESI+): 382.2 (M+H⁺); R.T.: 4.599 min. (HPLC Method C).

### Example 68. Synthesis of (6aR,10aR)-2,4-dichloro-1-hydroxy-6,6-dimethyl-3-(2-methyloctan-2-yl)-6a,7,10,10a-tetrahydro-6H-benzo[c]chromene-9-carboxylic acid (Compound 113)

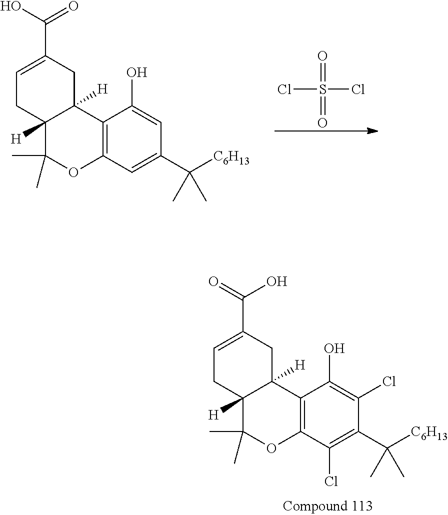

Sulfuryl chloride (0.50 mL, 6.24 mmol, 2.5 eq) was added dropwise a solution of JBT-101 (1.0 g, 2.497 mmol, 1 eq) in CH₂Cl₂ (4.5 mL) at 0° C. The mixture was stirred at 0° C. for 1 hour. The reaction was quenched with an aqueous solution of NaOH [1 M] (10 mL), the mixture was stirred for 15 min. The mixture was diluted with DCM and acidified to pH=3 with HCl [1 M]. The organic layer was separated, and the aqueous layer was extracted with DCM (2×20 mL). The combined organic layers were dried over anhydrous MgSO₄, filtered and concentrated under reduced pressure. The residue was purified by chromatography on silica gel (Heptane: EtOAc; from 100:0 to 70:30) to yield the tile compound as a yellow oil (0.173 g, 7.1%). ¹H NMR (300 MHz, CDCl₃) δ 7.15 (m, 1H), 6.33 (s, 1H), 3.78 (d, J=13.08 Hz, 1H), 2.73 (dt, J=8.43-3.45 Hz, 1H), 2.50-2.42 (m, 1H), 2.10-1.84 (m, 5H), 1.68 (d, J=1.56 Hz, 6H), 1.50 (s, 3H), 1.21 (m, 7H), 1.11 (s, 5H), 0.85 (t, J=5.22 Hz, 3H). LC-MS (ESI+): 468.9 (M+), 470.9 (M+2+); R.T.: 5.437 min. (HPLC Method C).

### Example 69. Affinity for CB₁ and CB₂ Receptors as Determined by a Radioligand Binding Assay

The binding affinity (% inhibition, Ki) of compounds of the invention for the CB₁ and CB₂ receptors was determined by a competitive radioligand binding assay, the results of which are provided in Table 2 and Table 3. Exemplary methods for the determination of binding affinity for a cannabinoid receptor by competitive radioligand binding can be found in the literature, for example, in Reggio P. H., et al. The bioactive conformation of aminoalkylindoles at the cannabinoid CB₁ and CB₂ receptors: insights gained from (E)- and (Z)-naphthylidene indenes. J Med Chem. 41(26): 5177-5187 (1998); and Munro S., et al. Molecular characterization of a peripheral receptor for cannabinoids. Nature 365:61-65 (1993).

CB₁ Radioligand Binding Assay: A compound of the invention (0.5 μM in 1% DMSO) was incubated with Chem-1 cells expressing human recombinant CB₁ receptor in buffer (50 mM HEPES, pH 7.4, 5 mM MgCl₂, 1 mM CaCl₂, 0.2% BSA) for 90 minutes at 37° C. in the presence of 2.0 nM [H³] SR141716A (CB₁ radioligand). % Inhibition was determined as a function of radioligand binding to the CB₁ receptor.

CB₂ Radioligand Binding Assay: A compound of the invention (0.05 μM in 1% DMSO) was incubated with CHO-K1 cells expressing human recombinant CB₂ receptor in buffer (20 mM HEPES, pH 7.0, 0.5% BSA) for 90 minutes at 37° C. in the presence of 2.4 nM [H³] WIN-55,212-2 (CB₂ radioligand). % Inhibition was determined as a function of radioligand binding to the CB₁ receptor.

TABLE 2

| | CB₁ Binding | CB₂ Binding | Fold |
|---|---|---|---|
| | at 0.5 μM % | at 0.05 μM % | selectivity |
| Compound | Inhibition | Inhibition | (CB₁/CB₂) |
| AJA | −1% | 0% | n/a |
| | (Ki = >3000 nM) | (Ki = 633 nM) | |
| 1 | 10% | 4% | n/a |
| | (Ki = >3000 nM) | (Ki = 1307 nM) | |
| 2 | 9% | 0% | n/a |
| 3 | 13% | 8% | n/a |
| 4 | 70% | 57% | n/a |
| 5 | 49% | 57% | 12.6 |
| | (Ki = 350 nM) | (Ki = 28 nM) | |
| 6 | 64% | 98% | 12 |
| | (Ki = 45 nM) | (Ki = 3.9 nM) | |
| 7 | 21% | 11% | n/a |
| 9 | −8% | 18% | n/a |
| 10 | 7% | 11% | n/a |
| 13 | 49% | 68% | 34 |
| | (Ki = 420 nM) | (Ki = 12.4 nM) | |
| 14 | 25% | 47% | 22 |
| | (Ki = 1230 nM) | (Ki = 56 nM) | |
| 15 | 16% | 54% | 18.3 |
| | (Ki = 660 nM) | (Ki = 36 nM) | |
| 16 | 84% | 57% | 4 |
| | (Ki = 94 nM) | (Ki = 26 nM) | |
| 17 | 23% | 8% | n/a |
| 18 | 5% | 26% | n/a |
| 19 | 111% | 115% | 4 |
| | (Ki = 6.8 nM) | (Ki = 1.9 nM) | |
| 20 | −10% | 9% | n/a |
| 22 | 107% | 92% | n/a |
| 24 | 44% | 60% | 35 |
| | (Ki = 1933 nM) | (Ki = 55 nM) | |
| 25 | 29% | 36% | 4.33 |
| | (Ki = 316 nM) | (Ki = 73 nM) | |
| 26 | 9% | 2% | n/a |
| 27 | 9% | 7% | n/a |
| 28 | 74% | 77% | 22 |
| | (Ki = 699 nM) | (Ki = 32 nM) | |
| 29 | 65% | 79% | n/a |
| 30 | 19% | −2% | n/a |
| 31 | 12% | 20% | n/a |
| | (Ki = >3000 nM) | (Ki = 2038 nM) | |
| 34 | (Ki = >3000 nM) | (Ki = >3000 nM) | n/a |
| 35 | (Ki = >3000 nM) | (Ki = >3000 nM) | n/a |
| 54 | (Ki = 353 nM) | (Ki = 11 nM) | 32 |
| 55 | (Ki = 619 nM) | (Ki = 33 nM) | 19 |
| 56 | (Ki = 708 nM) | (Ki = 41 nM) | 17 |

TABLE 3

| | | | Fold Selectivity |
|---|---|---|---|
| Compound | Ki CB1 (nM) | Ki CB2 (nM) | (CB1/CB2) |
| 21 | >5000 | >3000 | na |
| 41 | 27.4 | 4.3 | 6.4 |
| 42 | 59.5 | 175.0 | 0.3 |
| 43 | 891 | 75.6 | 11.8 |
| 44 | 33.5 | 2.4 | 14.0 |
| 45 | 144.0 | 10.1 | 14.3 |
| 46 | 90.3 | 22.9 | 3.9 |
| 48 | 233.0 | 9.2 | 25.3 |
| 57 | 162 | 56.3 | 2.9 |
| 88 | 5.4 | 1.0 | 5.7 |
| 89 | 62.0 | 4.3 | 14.4 |
| 90 | 10.0 | 2.5 | 4.0 |
| 91 | 34.8 | 3.2 | 10.9 |
| 92 | 45.7 | 3.8 | 12.0 |
| 93 | 243 | 31.9 | 7.6 |
| 94 | 18.8 | 12.6 | 1.5 |
| 95 | 31.1 | 3.2 | 9.7 |
| 96 | 25.0 | 1.3 | 19.2 |
| 97 | 780 | 72.0 | 10.8 |
| 98 | 320.0 | 10.0 | 32.0 |
| 99 | 8.9 | 2.0 | 4.5 |
| 100 | 72.3 | 7.2 | 10.0 |
| 101 | >5000 | 84.0 | >60 |
| 102 | 860 | 51.0 | 16.9 |
| 103 | 270 | 51.0 | 5.3 |
| 104 | 260 | 30.0 | 8.7 |
| 105 | 9.2 | 24.1 | 0.4 |
| 106 | 803 | 810 | 1.0 |
| 107 | 36.2 | 4.9 | 7.4 |
| 108 | >3000 | 302.0 | >9.9 |
| 109 | >3000 | >3000 | na |
| 110 | >3000 | >3000 | na |
| 111 | >3000 | >3000 | na |
| 112 | 1.0 | 0.7 | 1.4 |
| 113 | >3000 | >3000 | n/a |

### Example 70. CB₁- and CB₂-Mediated Activity as Determined by Cyclic Adenosine Monophosphate (cAMP) Assays

cAMP Assay #1 (Table 4)

Compounds of the invention were assayed in an adenylylcyclase assay to determine agonist activity on the CB₁ and CB₂ receptors, the results of which are provided in Table 4. Exemplary methods for the adenylylcyclase assay can be found in the literature, for example, in Rhee, M-H., et al. Cannabinol Derivatives: Binding to Cannabinoid Receptors and Inhibition of Adenylylcyclase. J Med Chem. 40: 3228-3233 (1997).

CB₁ Activity as determined by Adenylylcyclase Assay: CB₁ receptor agonist activity was measured by measuring cAMP production. A compound of the invention was incubated with CHO cells expressing human recombinant CB₁ for 20 minutes at 37° C. CB₁ activity (expressed as EC₅₀) was determined as a function of the agonist effect observed in the same assay using a positive control for CB₁ activation (positive control: 10 nM CP 55940).

CB₂ Activity as determined by Adenylylcyclase Assay: CB₂ receptor agonist activity was measured by measuring cAMP production. A compound of the invention was incubated with CHO cells expressing human recombinant CB₂ for 10 minutes at 37° C. CB₂ activity (expressed as EC₅₀) was determined as a function of the agonist effect observed in the same assay using a positive control for CB₂ activation (positive control: 100 nM WIN 55212-2).

Determination of EC₅₀ values: The EC₅₀ values (concentration producing a half-maximal response) were determined by non-linear regression analysis of the concentration-response curves generated with mean replicate values using Hill equation curve fitting: Y=D+[(A−D)/(1+(C/C₅₀)^{nH})], where Y=response, A=left asymptote of the curve, D=right asymptote of the curve, C=compound concentration, and C₅₀=EC₅₀, and nH=slope factor. This analysis was performed using software developed at Cerep (Hill software) and validated by comparison with data generated by the commercial software SigmaPlot® 4.0 for Windows® (© 1997 by SPSS Inc.).

TABLE 4

| | | |
|---|---|---|
| | CB₁ Activity EC₅₀ | CB₂ Activity EC₅₀ |
| Compound | (cAMP) | (cAMP) |

| | | | | |
|---|---|---|---|---|
| AJA | 4.6 | nM | 21 | nM |
| 5 | <3 | nM | 3.3 | nM |
| 6 | <3 | nM | <3 | nM |
| 13 | 4.6 | nM | 3.1 | nM |
| 14 | 10 | nM | 9.7 | nM |
| 15 | 68 | nM | 7.5 | nM |
| 24 | 13 | nM | 2.4 | nM |
| 25 | 16 | nM | 11 | nM |
| 28 | 8.2 | nM | 59 | nM |
| 29 | 6.8 | nM | 2.3 | nM |
| 31 | >100 | nM | 53 | nM |
| 34 | >300 | nM | >300 | nM |
| 35 | >300 | nM | >300 | nM |
| 54 | 1.2 | nM | 1.2 | nM |
| 55 | 45 | nM | 5.2 | nM |
| 56 | 3.6 | nM | 8.7 | nM |

cAMP Assay #2: Hit Hunter@ (Table 5)

Compounds of the invention were assayed in the Hit Hunter® cAMP assay to determine Gi-coupled agonist activity on the CB₁ and CB₂ receptors, the results of which are provided in Table 5.

The Hit Hunter® cAMP assay monitors the activation of a GPCR via Gi and Cs secondary messenger signaling in a homogenous, non-imaging assay format using a technology developed by DiscoverX called Enzyme Fragment Complementation (EFC) with β-galactosidase (β-Gal) as the functional reporter. The enzyme is split into two inactive complementary portions: EA for Enzyme Acceptor and ED for Enzyme Donor. ED is fused to cAMP and in the assay competes with cAMP generated by cells for binding to a cAMP-specific antibody. Active β-Gal is formed by complementation of exogenous EA to any unbound ED cAMP. Active enzymes can then convert a chemiluminescent substrate, generating an output signal detectable on a standard microplate reader.

cAMP Hunter cell lines were expanded from freezer stocks according to standard procedures. Cells were seeded in a total volume of 20 μL into white walled, 384-well microplates and incubated at 37° C. for the appropriate time prior to testing. cAMP modulation was determined using the DiscoverX Hit Hunter cAMP XS+ assay. For Gi agonist activity determination, cells were incubated with sample in the presence of EC80 forskolin to induce response (20 μM and 25 μM in the CB₁ and CB₂ assays, respectively). Media was aspirated from cells and replaced with 15 μL 2:1 HBSS/10 mM HEPES:cAMP XS+Ab reagent.

Intermediate dilution of sample stocks was performed to generate 4× sample in assay buffer. 5 μL of 4× compound was added to cells and incubated at 37° C. or room temperature for 30 or 60 minutes. Final vehicle concentration was 1%. Assay signal was generated through incubation with 20 μL cAMP XS+ED/CL lysis cocktail for one hour at room temperature. Microplates were read following signal generation with a PerkinElmer Envision™ instrument for chemiluminescent signal detection. Compound activity was analyzed using CBIS data analysis suite (Chem Innovation, CA). For agonist assays, percentage activity was calculated using the following formula: % Activity=100%×(1−(mean RLU of test sample−mean RLU of Max control ligand)/(mean RLU of vehicle control−mean RLU of Max control ligand). Control ligand was the non-selective CB₁/CB₂ agonist CP55,940.

TABLE 5

| | CB1, EC50 | CB2, EC50 | CB2 |
|---|---|---|---|
| Compound | (nM) | (nM) | fold selectivity |
| AJA | 77.1 | 7.0 | 11.0 |
| 5 | 32.2 | 3.9 | 8.2 |
| 6 | 4.7 | 0.8 | 6.0 |
| 13 | 66.2 | 2.5 | 26.5 |
| 14 | 470.0 | 4.1 | 115.2 |
| 15 | 448.4 | 2.7 | 164.1 |
| 16 | >3000 | 2.7 | >1000 |
| 24 | 222.6 | 3.5 | 63.5 |
| 25 | 359.6 | 9.0 | 40.0 |
| 29 | 9.6 | 1.0 | 9.6 |
| 31 | 635.7 | 68.4 | 9.3 |
| 41 | 6.3 | 1.5 | 4.3 |
| 45 | 273.5 | 12.0 | 22.9 |
| 46 | 263.2 | 8.4 | 31.4 |
| 48 | >3000 | 7.0 | >428 |
| 55 | 174.7 | 5.7 | 30.5 |
| 56 | 250.4 | 8.4 | 30.0 |
| 57 | 92.1 | 39.7 | 2.3 |
| 89 | 43.6 | 1.3 | 34.6 |
| 91 | 6.9 | 1.5 | 4.5 |
| 92 | 28.8 | 1.6 | 17.7 |
| 94 | 7.9 | 6.3 | 1.3 |
| 95 | 13.2 | 1.2 | 11.0 |
| 99 | 19.1 | 1.3 | 14.9 |
| 100 | 106.6 | 2.1 | 49.7 |
| 105 | 11.4 | 11.2 | 1.0 |
| 109 | 1224.5 | 148.3 | 8.3 |
| 110 | >3000 | 211.3 | >14 |

### Example 71. CB₁- and CB₂-Mediated Activity as Determined by β-Arrestin Assay

Compounds of the invention were assayed in the PathHunter® β-Arrestin assay to determine agonist activity on the CB₁ and CB₂ receptors, the results of which are provided in Table 6.

The PathHunter® β-Arrestin assay monitors the activation of a GPCR in a homogenous, non-imaging assay format using a technology developed by DiscoverX called Enzyme Fragment Complementation (EFC) with β-galactosidase (3-Gal) as the functional reporter. The enzyme is split into two inactive complementary portions (EA for Enzyme Acceptor and PK for ProLink) expressed as fusion proteins in the cell. EA is fused to β-Arrestin and PK is fused to the GPCR of interest.

PathHunter cell lines were expanded from freezer stocks according to standard procedures. Cells were seeded in a total volume of 20 μL into white walled, 384-well microplates and incubated at 37° C. for the appropriate time prior to testing. For agonist activity determination, cells were incubated with sample to induce response. Intermediate dilution of sample stocks was performed to generate 5× sample in assay buffer. 5 μL of 5× sample was added to cells and incubated at 37° C. or room temperature for 90 to 180 minutes. Vehicle concentration was 1%. Assay signal was generated through a single addition of 12.5 or 15 μL (50% v/v) of PathHunter Detection reagent cocktail, followed by a one hour incubation at room temperature. Microplates were read following signal generation with a PerkinElmer Envision™ instrument for chemiluminescent signal detection. Compound activity was analyzed using CBIS data analysis suite (ChemInnovation, CA). For agonist assays, percentage activity was calculated using the following formula: % Activity=100%×(mean RLU of test sample− mean RLU of vehicle control)/(mean MAX control ligand− mean RLU of vehicle control).

TABLE 6

| | | |
|---|---|---|
| | CB₁ Activity | CB₂ Activity |
| Compound | (β-Arrestin) | (β-Arrestin) |

| | | | | |
|---|---|---|---|---|
| AJA | 1244 | nM | 11 | nM |
| 1 | >3000 | nM | 58 | nM |
| 2 | >3000 | nM | >3000 | nM |
| 3 | >3000 | nM | >3000 | nM |
| 5 | 620 | nM | 520 | nM |
| 6 | 160 | nM | 210 | nM |
| 13 | 787 | nM | 807 | nM |
| 14 | >3000 | nM | 8 | nM |
| 15 | >3000 | nM | >3000 | nM |
| 16 | >3000 | nM | 5 | nM |
| 20 | >3000 | nM | >3000 | nM |
| 21 | 2669.0 | nM | >1000 | nM |
| 22 | 42 | nM | 4 | nM |
| 24 | >3000 | nM | 2 | nM |
| 25 | >3000 | nM | 10 | nM |
| 26 | >3000 | nM | >3000 | nM |
| 27 | >3000 | nM | >3000 | nM |
| 28 | 654 | nM | 1.7 | nM |
| 29 | 47 | nM | 3 | nM |
| 30 | >3000 | nM | >3000 | nM |
| 31 | >3000 | nM | 109 | nM |
| 34 | >3000 | nM | >3000 | nM |
| 35 | >3000 | nM | >3000 | nM |

| | | |
|---|---|---|
| 40 | >10000 | >1000 |

| | | | | |
|---|---|---|---|---|
| 41 | 37.8 | nM | 1.8 | nM |
| 44 | 11.1 | nM | 1.6 | nM |
| 45 | 4978.1 | nM | 6.0 | nM |
| 46 | 6002.8 | nM | 3.6 | nM |
| 48 | >3000 | nM | 4.4 | nM |
| 54 | 13 | nM | 4 | nM |
| 55 | >3000 | nM | 6.9 | nM |
| 56 | >3000 | nM | 6.9 | nM |
| 57 | 289.5 | nM | 22.0 | nM |
| 88 | 5.9 | nM | 1.7 | nM |
| 89 | 33.0 | nM | 1.9 | nM |
| 90 | 127.8 | nM | 2.7 | nM |
| 91 | 20.9 | nM | 1.7 | nM |
| 92 | 17.3 | nM | 1.4 | nM |
| 93 | >3000 | nM | 3.1 | nM |
| 94 | 94.9 | nM | 4.3 | nM |
| 95 | 23.9 | nM | 1.3 | nM |
| 96 | 12.8 | nM | 3.0 | nM |
| 97 | >3000 | nM | 17.9 | nM |
| 99 | 32.5 | nM | 1.2 | nM |
| 100 | 256.6 | nM | 1.2 | nM |
| 102 | >3000 | nM | 30.5 | nM |
| 103 | 1713.8 | nM | 9.5 | nM |
| 104 | 4445.3 | nM | 5.0 | nM |
| 105 | 75.6 | nM | 3.7 | nM |
| 107 | 49.4 | nM | 2.9 | nM |
| 108 | 1431.1 | nM | 29.5 | nM |
| 109 | >3000 | nM | 58.1 | nM |
| 110 | >3000 | nM | 37.4 | nM |
| 112 | 2.0 | nM | 1.4 | nM |

### Example 72. Biological Activity as Determined by an Inflammation Bioassay in Human Peripheral Blood Mononuclear Cells (PBMCs)

Compounds of the invention were assayed for their effect on inflammatory cytokine release from lipopolysaccharides (LPS)-induced PBMCs, isolated from a human blood sample, the results of which are provided in Table 7 as fold change. Exemplary methods for quantifying secretion of inflammatory cytokines in PBMCs are known in the art, for example, as described in Hailer et al., Infection and Immunity. 68(2):752-759 (2000); and Merlini et al., Frontiers in Immunology. 7:614 (2016); each of which is incorporated herein by reference with respect to methods for evaluating cytokine release in PBMCs.

In brief, human blood samples from healthy volunteers were collected and PBMCs were isolated. PBMCs were cultured and each compound was assayed in triplicate at a final concentration of 10 μM for 2 hours. 1 μg/ml dexamethasone (DEX) served as a positive control. LPS was added at a final concentration of 0.1 μg/ml and further incubated for 24 hours. At the end of the incubation, the supernatants were collected and the levels of a panel of secreted cytokines were measured by a Human Magnetic Luminex® assay (R&D). The results in Table 7 are presented as a fold change from LPS-treated PBMCs. Reduced levels are expressed by negative values. OOR denotes values that are out of the standard curve range for the specific cytokine and thus, could not be accurately measured. Cytotoxicity was also determined. Compounds with cytotoxicity ≥50% was considered cytotoxic. Baseline cytotoxicity of untreated PBMCs was 17% and all compounds presented in Table 7 induced cytotoxicity below 25%.

TABLE 7

| | | | | | | | IL-12 | | | |
|---|---|---|---|---|---|---|---|---|---|---|
| Compound | MCP-1 | IL-1β | IL-8 | IL-6 | IL-4 | L-31 | (p70) | IL17A | IL-10 | TNFα |
| DEX | −1.7 | −3.8 | −1.0 | −3.9 | −1.5 | −1.6 | −4.5 | −1.5 | −2.0 | −6.0 |
| 15 | −213.2 | −5.9 | −2.4 | −107.7 | −2.7 | OOR | OOR | −3.1 | −299.9 | −75.9 |
| 89 | −143.8 | −4.8 | −4.2 | −237.0 | −3.8 | OOR | OOR | −14.0 | −960.1 | −35.6 |
| 91 | −1.4 | −1.4 | −1.3 | −2.2 | −1.6 | −2.1 | −1.7 | 1.2 | −2.3 | −1.2 |
| 99 | −3.3 | 1.2 | −1.0 | −2.6 | −1.4 | −1.1 | −1.6 | 1.2 | −7.0 | −1.4 |
| 109 | −5.0 | −3.3 | −1.0 | −1.4 | −1.5 | −1.5 | −1.6 | 1.2 | −1.3 | −1.3 |
| 110 | −3.0 | −1.5 | −1.0 | −1.2 | −1.3 | −1.3 | −1.1 | 1.3 | −1.0 | −1.0 |

### Example 73: Biological Activity as Determined by a Phenotypic Screen in the BioMap Diversity Early Screen Platform

Compounds of the invention were assayed for their effects on 148 biomarkers of inflammation, immune modulation and tissue remodeling in the BioMAP® Diversity PLUS® (DiscoverX) platform designed to model aspects of human diseases in vitro (Kunkel 2004, Berg 2006, Melton 2013). Example results are provided in Table 8. The BioMap platform consists of 12 systems of human primary cells: 30: venular endothelial cells stimulated with IL-β, TNFα and INFγ; 4H: venular endothelial cells stimulated with IL-4 and histamine; LPS: PBMCs co-cultured with venular endothelial cells and stimulated with TLR4 ligand; SAg: PBMCs co-cultured with venular endothelial cells and stimulated with TCR ligands; BT: PBMCs co-cultured with B cells and stimulated with α-IGM and TCR ligands; BF4T: bronchial epithelial cells co-cultured with dermal fibroblast and stimulated with TNFα and IL-4; BE3C: bronchial epithelial cells stimulated with IL1-β, TNF and INFγ; CASM3C: coronary artery smooth muscle cells stimulated with IL1-β, TNF and INFγ; HDF3CGF: dermal fibroblasts stimulated with IL1-β, TNFα, INFγ, EGF, bFGF and PDGF-BB; KF3CT: keratinocytes co-cultured with dermal fibroblasts and stimulated with IL-1, TNFα, INFγ and TGFβ; MyoF: lung fibroblasts stimulated with TNFα and TGFβ; /Mphg: venular endothelial cells co-cultured with macrophages and stimulated with TLR2 ligand.

The example compounds (3.3 μM) in Table 8 induced increases or decreases in the levels of the indicated 20 biomarkers, which are at least 1.5-fold change from vehicle control and are outside the significance prediction envelop. All compounds presented in Table 8 were not cytotoxic at the concentration tested.

TABLE 8

| | | Immune modulation, cell surface and tissue remodeling |
|---|---|---|
| Compound | Anti-proliferation | biomarkers |
| 25 | Dermal fibroblasts | Reduced VCAM-1, PAI-I, IL-10, and M-CSF; increased MMP-1 |
| 29 | Dermal fibroblasts | Reduced Eotaxin-3, IL-8, E-selectin, P-selectin, VCAM, EGFR, |
| | | IL-1α, IP-10, and PAI-I |
| 31 | | Reduced Eotaxin-3 and IP-10; increased MMP-1 |
| 44 | | Reduced MCP-1, P-selectin, and IL-1α |
| 55 | Dermal fibroblasts | |
| 91 | | Reduced Eotaxin-3, P-selectin, CD69, and EGFR, IL-10 |
| 92 | | Reduced P-selectin |
| 93 | | Reduced Eotaxin-3, P-selectin, CD69, and M-CSF; increased IL-6 |
| 94 | | Reduced MCP-1 and CD69; increased IL-1α |
| 95 | | Reduced IL-8, P-selectin, CD69, VCAM-1, Thrombomodulin, and |
| | | IL-1α |
| 100 | Dermal fibroblasts | Reduced Eotaxin-3, P-selectin, and CD69, |

### Example 74. Pharmacokinetic Parameters of Compounds

Male C57BL/6 mice were administered an intravenous (IV) dose of 1 mg/kg of the indicated compound. Pharmacokinetic parameters were determined by standard methods, the results of which are provided in Table 9.

TABLE 9

| Dose | Dose | PK | | Compound | | | | |
|---|---|---|---|---|---|---|---|---|
| (mg/kg) | route | parameters | | 15 | 29 | AJA | 55 | 56 |
| 1 | IV | CL | L/hr/kg | 0.635 | 0.280 | 0.400 | 0.421 | 0.522 |
| | N = 9 | Vₛₛ | L/kg | 1.37 | 0.430 | 0.75 | 1.42 | 1.20 |
| | | T_{1/2} | hr | 1.74 | 4.89 | 1.41 | 3.99 | 2.39 |
| | | C₀ | ng/mL | 1835 | 17492 | 1753 | 3060 | 2791 |
| | | AUCₗₐₛₜ | hr*ng/mL | 1508 | 3550 | 2421 | 2359 | 1790 |
| | | AUC_{INF} | hr*ng/mL | 1574 | 3568 | 2509 | 2375 | 1915 |
| | | AUC_{0-24 hr} | hr*ng/mL | 1720 | 3550 | 2766 | 2359 | 2080 |
| | | MRT_{INF} | hr | 2.16 | 1.53 | 1.89 | 3.38 | 2.30 |

Pharmacokinetic parameters were determined by standard methods, the results of which are provided in Table 10.

TABLE 10

| Dose | Dose | PK | | Compound | | | | |
|---|---|---|---|---|---|---|---|---|
| (mg/kg) | route | parameters | | 15 | 29 | AJA | 55 | 56 |
| 10 | PO | Tₘₐₓ | hr | 4.00 | 6.00 | 1 | 1 | 1 |
| | N = 9 | Cₘₐₓ | ng/mL | 779 | 203 | 3857 | 882 | 902 |
| | | T_{1/2} | hr | 2.65 | 4.21 | 2.19 | 2.72 | 2.63 |
| | | T_{lag} | hr | 0.00 | 0.00 | 0 | 0 | 0 |
| | | AUCₗₐₛₜ | hr*ng/mL | 5872 | 1400 | 17753 | 7168 | 4415 |
| | | AUC_{INF} | hr*ng/mL | 5886 | 1422 | 17759 | 7205 | 4426 |
| | | AUC_{0-24 hr} | hr*ng/mL | 5872 | 1400 | 17753 | 7168 | 4415 |
| | | MRT_{INF} | hr | 5.07 | 6.20 | 3.42 | 6.40 | 4.89 |
| | | F | % | 37.4 | 3.98 | 70.79 | 30.33 | 23.11 |

Male C57BL/6 mice were administered an oral (P0) dose of 10 mg/kg of the indicated compound. The brain/plasma ratios of the indicated compounds were determined by standard methods, the results of which are provided in Table 11.

TABLE 11

| | Dose | | | |
|---|---|---|---|---|
| Dose | route | | | |
| (mg/kg) | PO | Time | | |
| 10 | N = 9 | points | Compound 15 | Compound 29 |

| Concentration (ng/g) | 4 | h | 107 | 1.72 |
|---|---|---|---|---|
| | 10 | h | 50.4 | 7.93 |
| | 24 | h | 0 | 0 |
| Brain/Plasma | 4 | h | 0.139 | 0.00637 |
| | 10 | h | 0.331 | 0.106 |
| | 24 | h | 0 | 0 |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A compound described by formula (I): 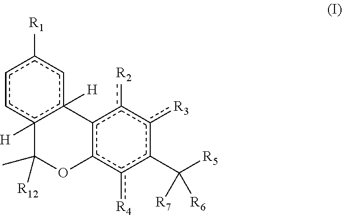
wherein each dashed line is optionally a double bond;
R₁ is optionally substituted carboxyl, optionally substituted amide, optionally substituted thioester, optionally substituted thioamide, optionally substituted sulfonamide, an optionally substituted alkyl, or cyano;
R₂ is H, O, Cl, F, NH₂, hydroxyl, or optionally substituted alkoxy;
R₃ and R₄ are each independently H, O, Cl, or F;
R₅ is optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl or optionally substituted C1-C20 alkoxy;
R₆ and R₇ are each independently H, —CH₃, —CF₃, or —CH₂OH; and
R₁₂ is —CH₃ or —CH₂OH,
or a pharmaceutically acceptable salt thereof;
and wherein the compound described by formula (I) is not ajulemic acid (AJA):

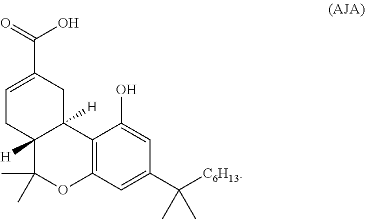

2. The compound of claim 1, wherein the compound is described by formula (II): 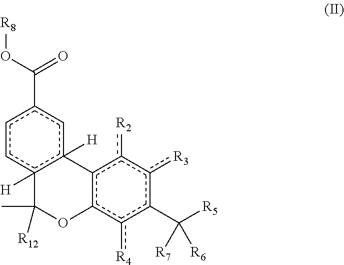
wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino,
or a pharmaceutically acceptable salt thereof.

3. The compound of claim 2, wherein the compound is described by formula (I-1):

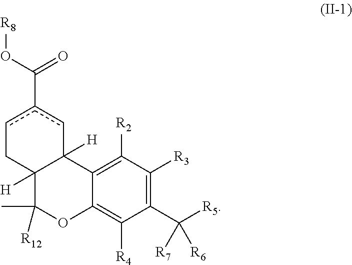

4. The compound of claim 3, wherein the compound is described by formula (II-2):

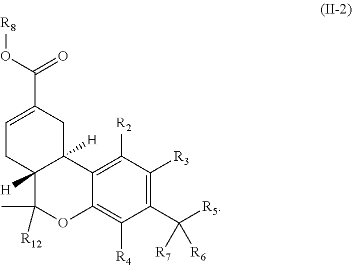

5. The compound of claim 3, wherein the compound is described by formula (II-3):

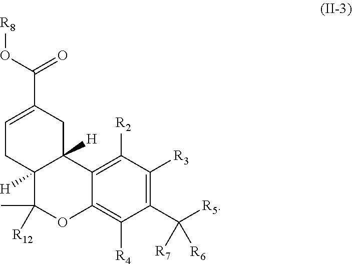

6. The compound of claim 3, wherein the compound is described by formula (II-4):

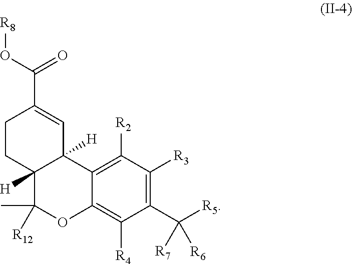

7. The compound of claim 2, wherein the compound is described by formula (II-5):

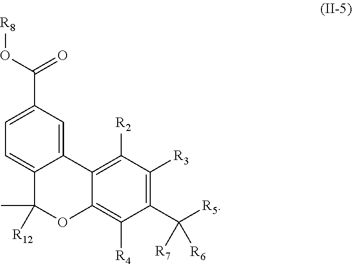

8. The compound of any one of claims 3-7, wherein R₂ is H, OH, Cl, F, NH₂, or methoxy.

9. The compound of any one of claims 3-8, wherein R₃ and R₄ are each independently H, Cl, or F.

10. The compound of claim 2, wherein the compound is described by formula (II-6):

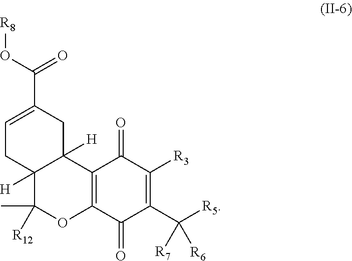

11. The compound of claim 10, wherein the compound is described by formula (II-7):

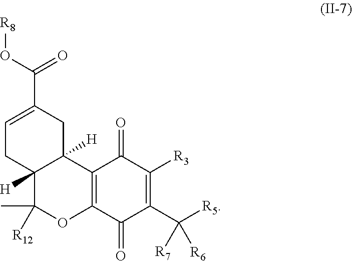

12. The compound of claim 10 or 11, wherein R₃ is H, Cl, or F.

13. The compound of claim 2, wherein the compound is described by formula (II-8):

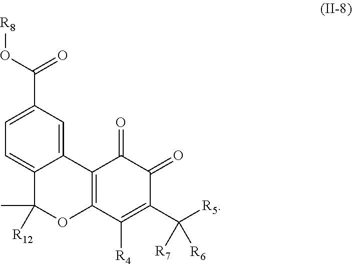

14. The compound of claim 13, wherein R₄ is H, Cl, or F.

15. The compound of claim 1, wherein the compound is described by formula (III): 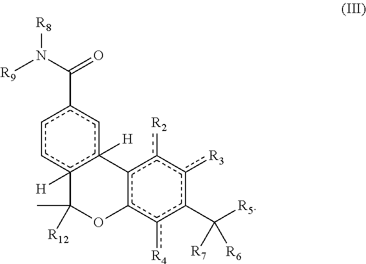
wherein R₈ and R₉ are each independently H, OH, optionally substituted amine, optionally substituted C1-C20 alkyl, optionally substituted C1-C20 alkenyl, optionally substituted C1-C20 alkynyl, optionally substituted C5-C15 aryl, optionally substituted C1-C20 alkoxy, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or
R₈ and R₉ form an optionally substituted C3-C20 heterocyclyl;
or a pharmaceutically acceptable salt thereof.

16. The compound of claim 15, wherein the compound is described by formula (III-1):

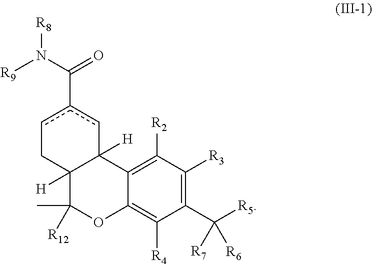

17. The compound of claim 16, wherein the compound is described by formula (III-2):

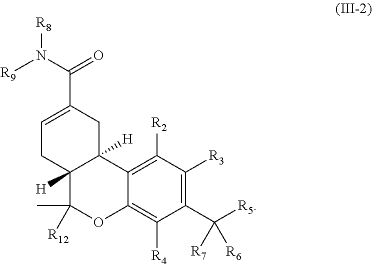

18. The compound of claim 16, wherein the compound is described by formula (III-3):

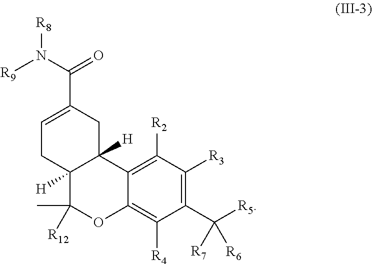

19. The compound of claim 16, wherein the compound is described by formula (III-4):

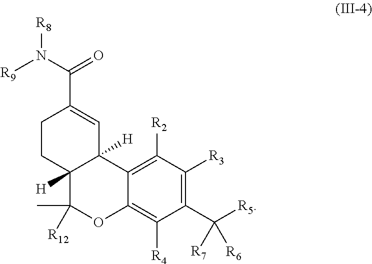

20. The compound of claim 15, wherein the compound is described by formula (III-5):

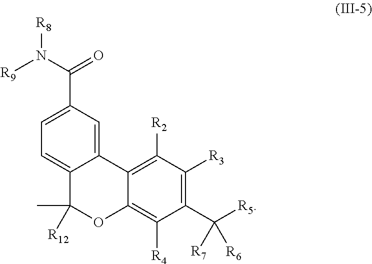

21. The compound of any one of claims 16-20, wherein R₂ is H, OH, Cl, F, NH₂, or methoxy.

22. The compound of any one of claims 16-21, wherein R₃ and R₄ are each independently H, Cl, or F.

23. The compound of claim 15, wherein the compound is described by formula (III-6):

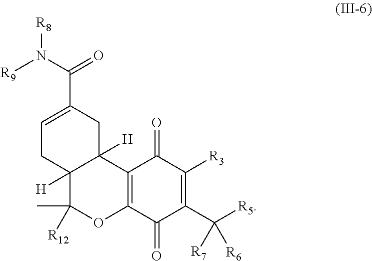

24. The compound of claim 23, wherein the compound is described by formula (III-7):

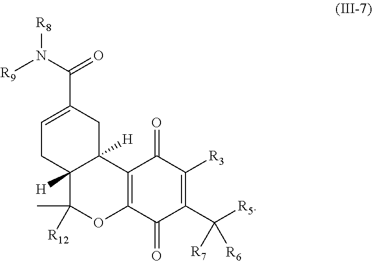

25. The compound of claim 23 or 24, wherein R₃ is H, CI, or F.

26. The compound of claim 15, wherein the compound is described by formula (III-8):

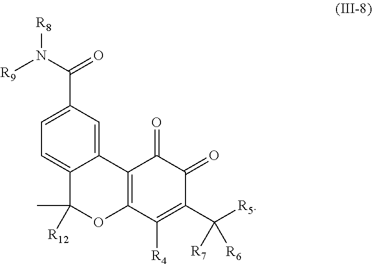

27. The compound of claim 26, wherein R₄ is H, Cl, or F.

28. The compound of any one of claims 15-27, wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino.

29. The compound of any one of claims 15-28, wherein R₈ is H or C1-C4 alkyl.

30. The compound of claim 29, wherein R₈ is H.

31. The compound of claim 29, wherein R₈ is CH₃.

32. The compound of any one of claims 15-27, wherein R₈ and R₉ form an optionally substituted C3-C20 heterocyclyl.

33. The compound of claim 32, wherein the optionally substituted C3-C20 heterocyclyl is selected from:

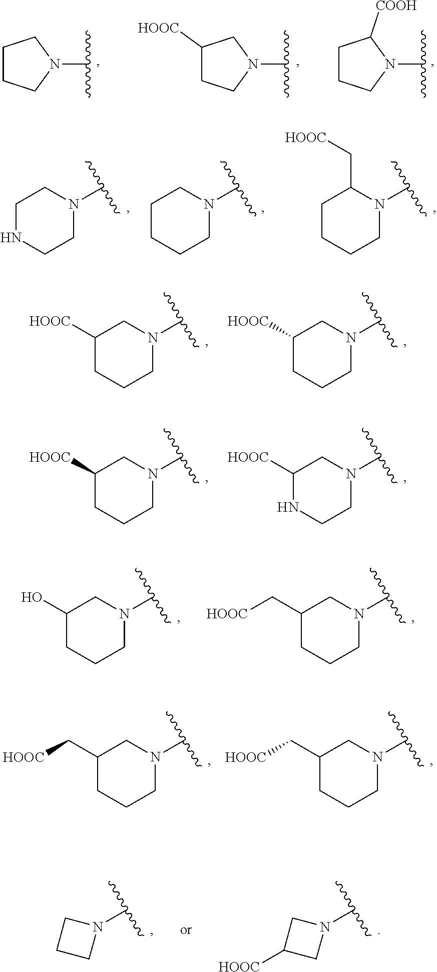

34. The compound of claim 1, wherein the compound is described by formula (IV): 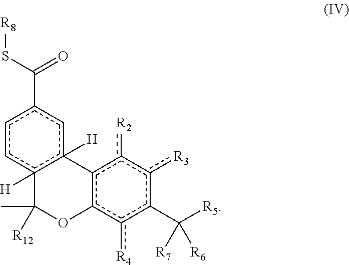
wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino,
or a pharmaceutically acceptable salt thereof.

35. The compound of claim 34, wherein the compound is described by formula (IV-1):

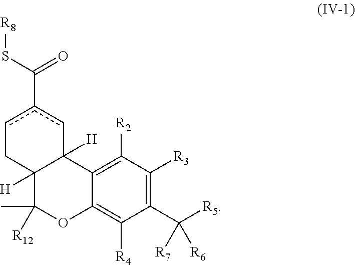

36. The compound of claim 35, wherein the compound is described by formula (IV-2):

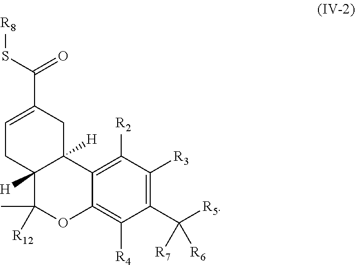

37. The compound of claim 35, wherein the compound is described by formula (IV-3):

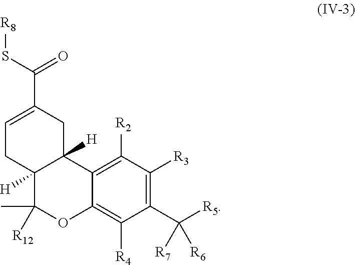

38. The compound of claim 35, wherein the compound is described by formula (IV-4):

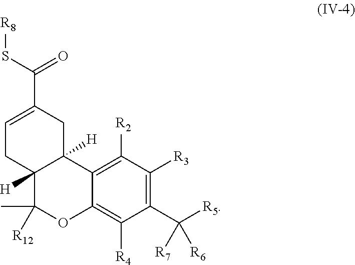

39. The compound of claim 34, wherein the compound is described by formula (IV-5):

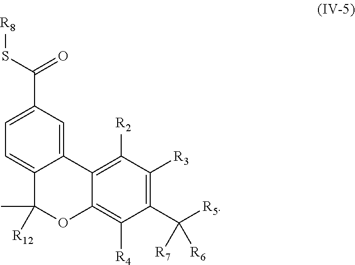

40. The compound of any one of claims 34-39, wherein R₂ is H, OH, Cl, F, NH₂, or methoxy.

41. The compound of any one of claims 34-40, wherein R₃ and R₄ are each independently H, Cl, or F.

42. The compound of claim 34, wherein the compound is described by formula (IV-6):

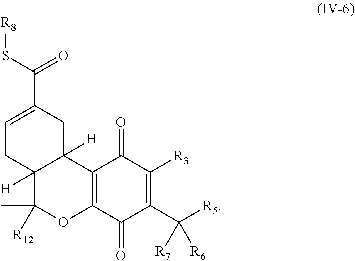

43. The compound of claim 42, wherein the compound is described by formula (IV-7):

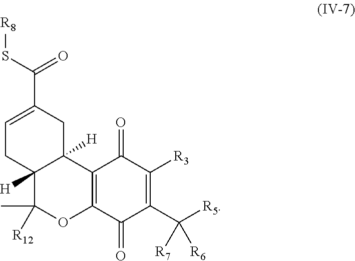

44. The compound of claim 42 or 43, wherein R₃ is H, Cl, or F.

45. The compound of claim 34, wherein the compound is described by formula (IV-8):

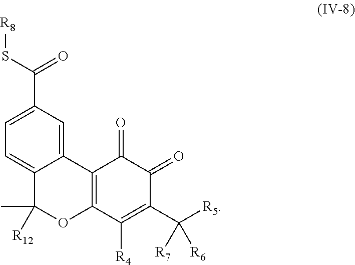

46. The compound of claim 45, wherein R₄ is H, Cl, or F.

47. The compound of claim 1, wherein the compound is described by formula (V): 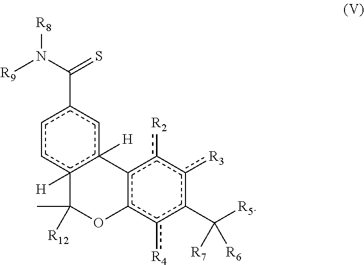
wherein R₈ and R₉ are each independently H, OH, optionally substituted amine, optionally substituted C1-C20 alkyl, optionally substituted C1-C20 alkenyl, optionally substituted C1-C20 alkynyl, optionally substituted C1-C20 alkoxy, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or
R₈ and R₉ form an optionally substituted C3-C20 heterocyclyl;
or a pharmaceutically acceptable salt thereof.

48. The compound of claim 47, wherein the compound is described by formula (V-1):

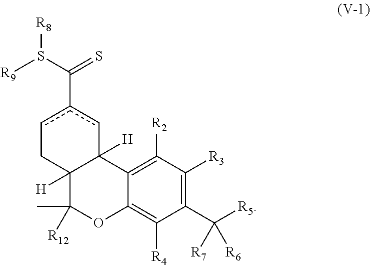

49. The compound of claim 48, wherein the compound is described by formula (V-2):

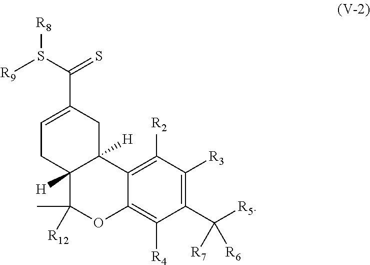

50. The compound of claim 48, wherein the compound is described by formula (V-3):

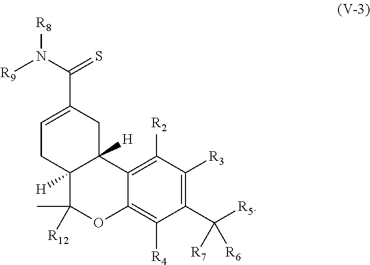

51. The compound of claim 48, wherein the compound is described by formula (V-4):

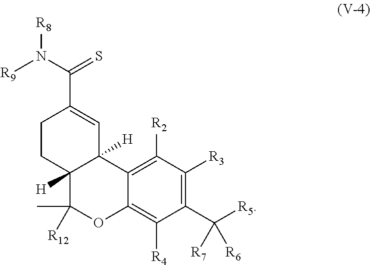

52. The compound of claim 47, wherein the compound is described by formula (V-5):

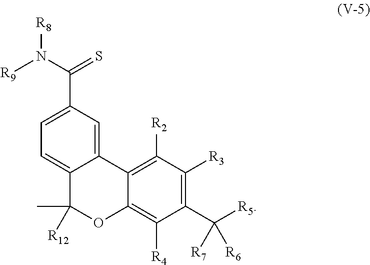

53. The compound of any one of claims 48-52, wherein R₂ is H, OH, Cl, F, NH₂, or methoxy.

54. The compound of any one of claims 48-52, wherein R₃ and R₄ are each independently H, Cl, or F.

55. The compound of claim 47, wherein the compound is described by formula (V-6):

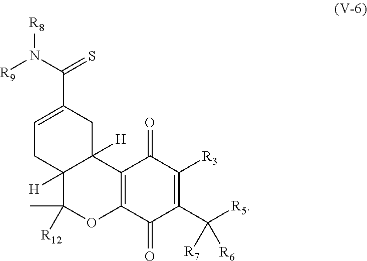

56. The compound of claim 55, wherein the compound is described by formula (V-7):

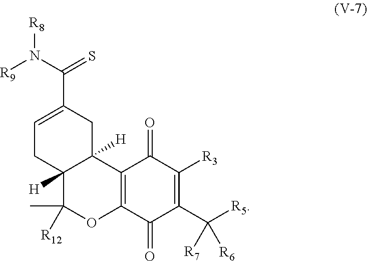

57. The compound of claim 55 or 56, wherein R₃ is H, Cl, or F.

58. The compound of claim 47, wherein the compound is described by formula (V-8):

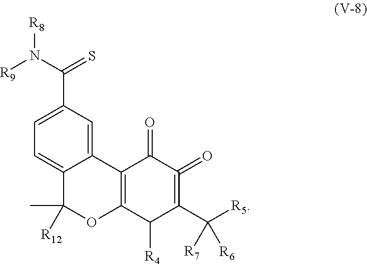

59. The compound of claim 58, wherein R₄ is H, Cl, or F.

60. The compound of any one of claims 47-59, wherein R₈ is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino.

61. The compound of any one of claims 47-60, wherein R₈ is H or C1-C4 alkyl.

62. The compound of claim 61, wherein R₈ is H.

63. The compound of claim 61, wherein R₈ is CH₃.

64. The compound of any one of claims 47-59, wherein R₈ and R₉ form an optionally substituted C3-C20 heterocyclyl.

65. The compound of claim 64, wherein the optionally substituted C3-C20 heterocyclyl is selected from:

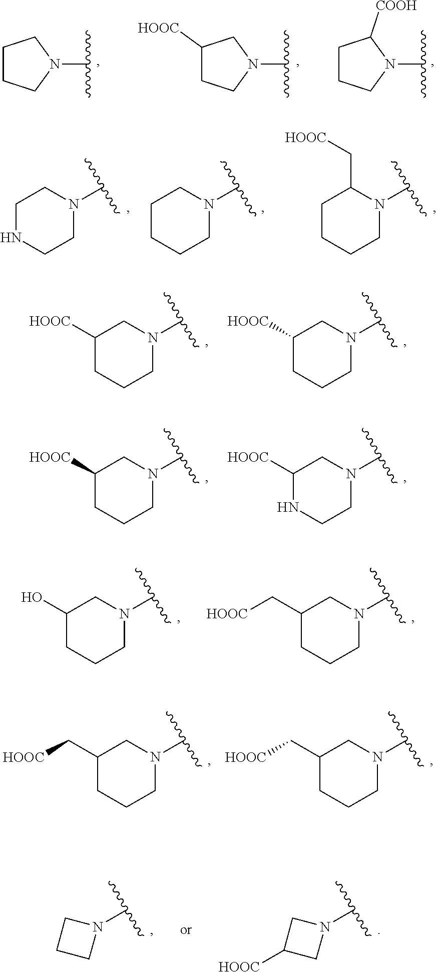

66. The compound of claim 1, wherein the compound is described by formula (VI): 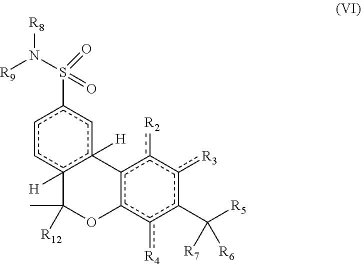
wherein R₈ and R₉ are each independently H, OH, optionally substituted amine, optionally substituted C1-C20 alkyl, optionally substituted C1-C20 alkenyl, optionally substituted C1-C20 alkynyl, optionally substituted C5-C15 aryl, optionally substituted C1-C20 alkoxy, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or
R₈ and R₉ form an optionally substituted C3-C20 heterocyclyl;
or a pharmaceutically acceptable salt thereof.

67. The compound of claim 66, wherein the compound is described by formula (VI-1):

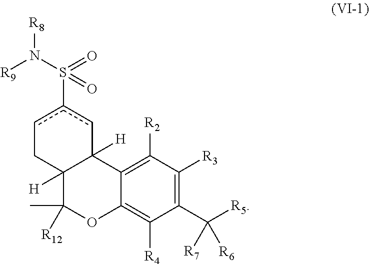

68. The compound of claim 67, wherein the compound is described by formula (VI-2):

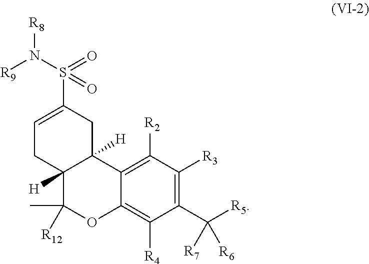

69. The compound of claim 67, wherein the compound is described by formula (VI-3):

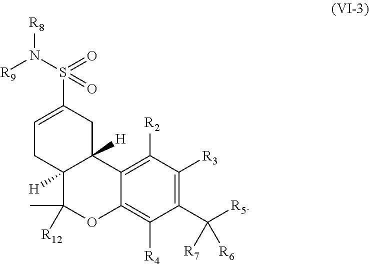

70. The compound of claim 67, wherein the compound is described by formula (VI-4):

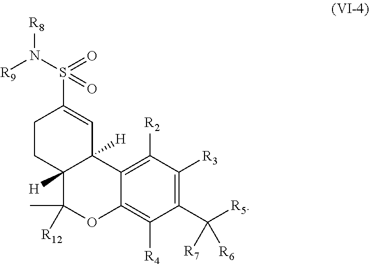

71. The compound of claim 66, wherein the compound is described by formula (VI-5):

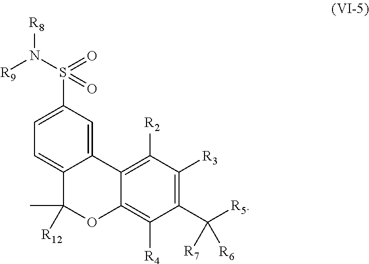

72. The compound of any one of claims 67-71, wherein R₂ is H, OH, Cl, F, NH₂, or methoxy.

73. The compound of any one of claims 67-72, wherein R₃ and R₄ are each independently H, Cl, or F.

74. The compound of claim 66, wherein the compound is described by formula (VI-6):

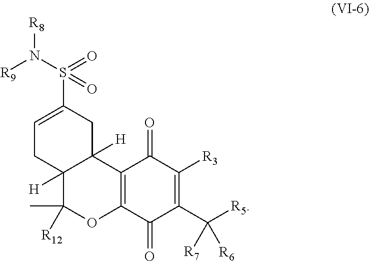

75. The compound of claim 74, wherein the compound is described by formula (VI-7):

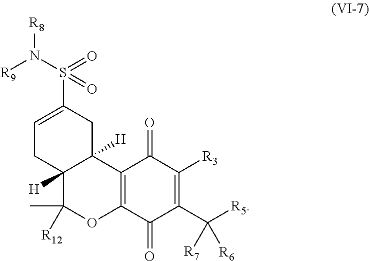

76. The compound of claim 74 or 75, wherein R₃ is H, Cl, or F.

77. The compound of claim 66, wherein the compound is described by formula (VI-8):

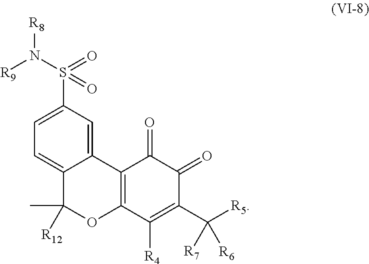

78. The compound of claim 77, wherein R₄ is H, CI, or F.

79. The compound of any one of claims 66-78, wherein Re is H, optionally substituted C1-C20 alkyl, optionally substituted C1-C20 alkenyl, optionally substituted C1-C20 alkynyl, optionally substituted C5-C15 aryl, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino.

80. The compound of any one of claims 66-79, wherein Re is H or C1-C4 alkyl.

81. The compound of claim 80, wherein Re is H.

82. The compound of claim 80, wherein Re is CH₃.

83. The compound of any one of claims 66-78, wherein R₈ and R₉ form an optionally substituted C3-C20 heterocyclyl.

84. The compound of claim 83, wherein the optionally substituted C3-C20 heterocyclyl is selected from:

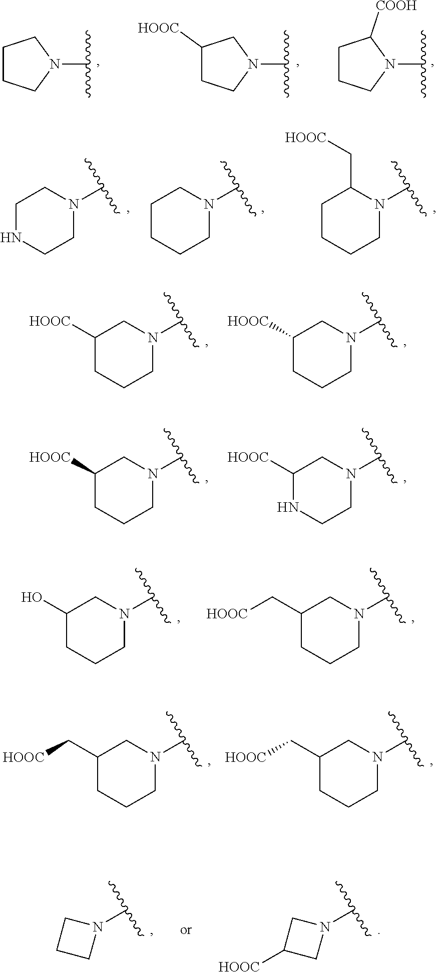

85. The compound of any one of claims 2-84, wherein R₈ is selected from:

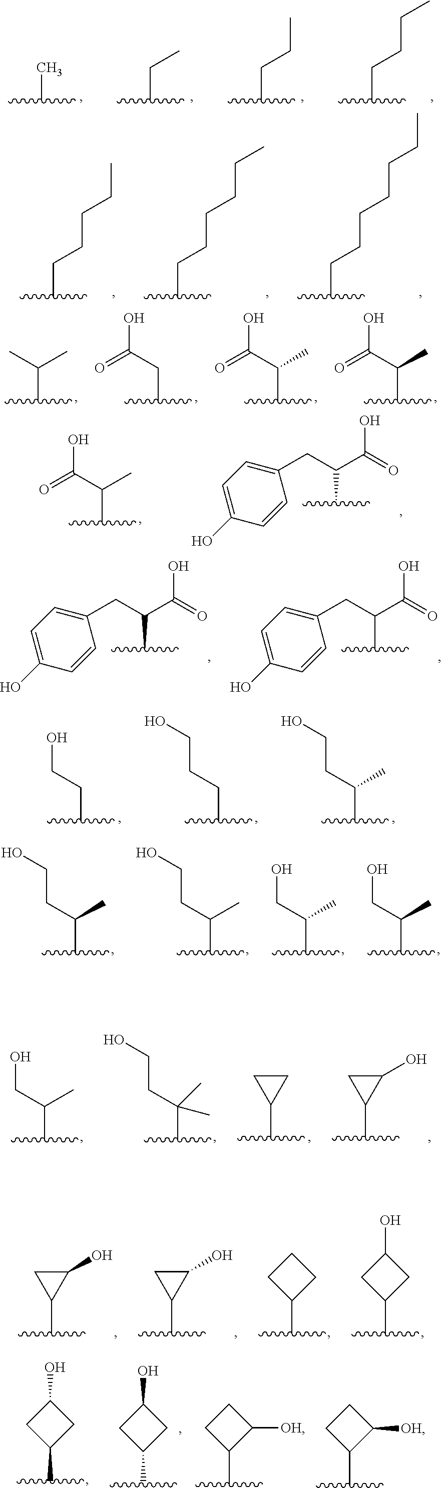

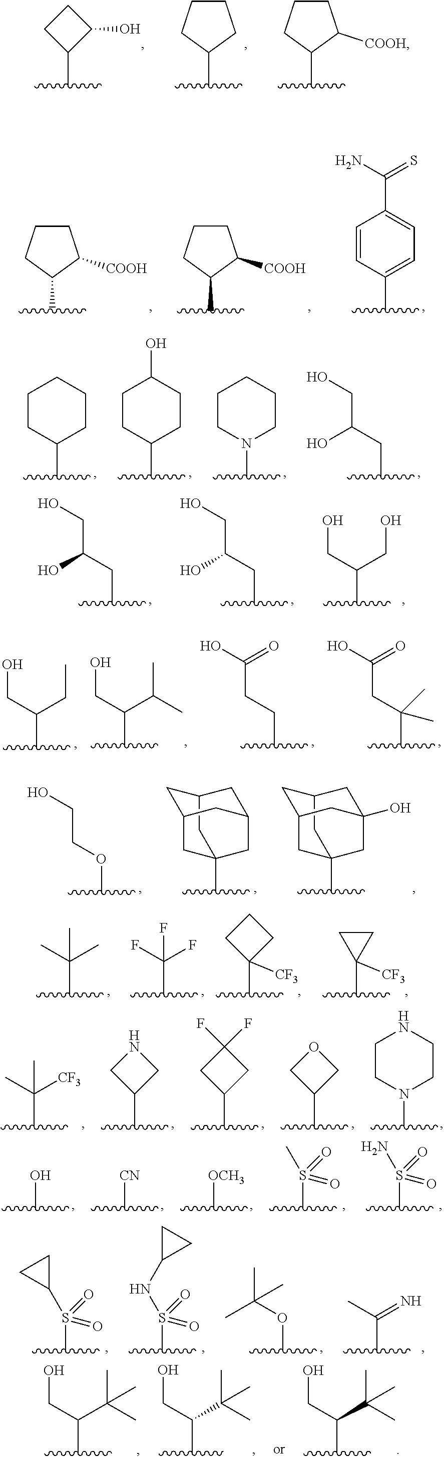

86. The compound of claim 1, wherein the compound is described by formula (VII): 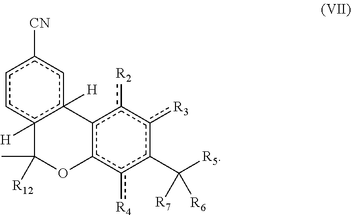
or a pharmaceutically acceptable salt thereof.

87. The compound of claim 86, wherein the compound is described by formula (VII-1):

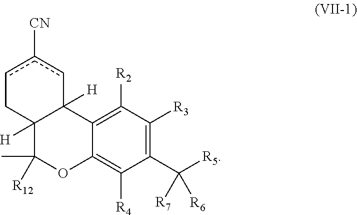

88. The compound of claim 87, wherein the compound is described by formula (VII-2):

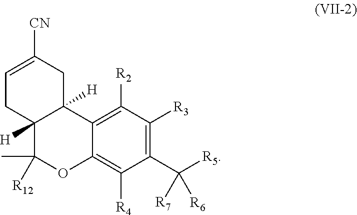

89. The compound of claim 87, wherein the compound is described by formula (VII-3):

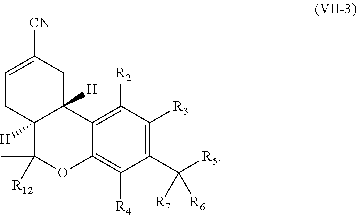

90. The compound of claim 87, wherein the compound is described by formula (VII-4):

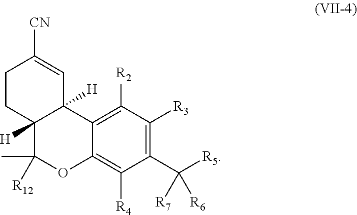

91. The compound of claim 86, wherein the compound is described by formula (VII-5):

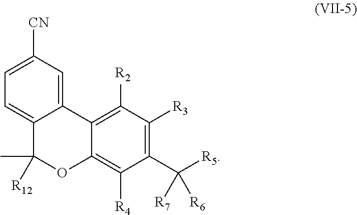

92. The compound of any one of claims 87-91, wherein R₂ is H, OH, Cl, F, NH₂, or methoxy.

93. The compound of any one of claims 87-92, wherein R₃ and R₄ are each independently H, Cl, or F.

94. The compound of claim 86, wherein the compound is described by formula (VII-6):

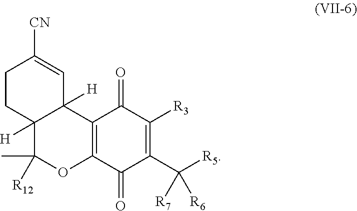

95. The compound of claim 94, wherein the compound is described by formula (VII-7):

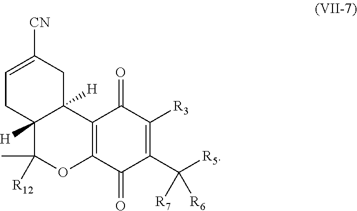

96. The compound of claim 94 or 95, wherein R₃ is H, Cl, or F.

97. The compound of claim 86, wherein the compound is described by formula (VII-8):

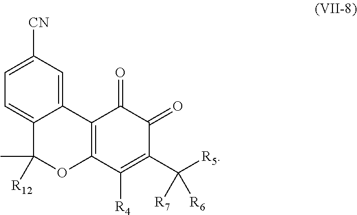

98. The compound of claim 97, wherein R₄ is H, Cl, or F.

99. The compound of claim 1, wherein the compound is described by formula (VIII): 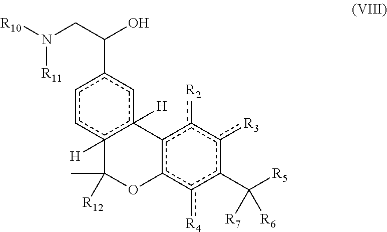
wherein R₁₀ and R₁₁ are each independently H, OH, optionally substituted amine, optionally substituted C1-C20 alkyl, optionally substituted C1-C20 alkenyl, optionally substituted C1-C20 alkynyl, optionally substituted C5-C15 aryl, optionally substituted C1-C20 alkoxy, optionally substituted C2-C15 heteroaryl, optionally substituted C3-C20 cycloalkyl, optionally substituted C1-C20 heteroalkyl, optionally substituted C3-C20 heterocyclyl, optionally substituted C6-C35 alkaryl, optionally substituted C6-C35 heteroalkaryl, optionally substituted sulfonyl, or optionally substituted imino; or
R₁₀ and R₁₁ form an optionally substituted C3-C20 heterocyclyl;
or a pharmaceutically acceptable salt thereof.

100. The compound of claim 99, wherein the compound is described by formula (VIII-1):

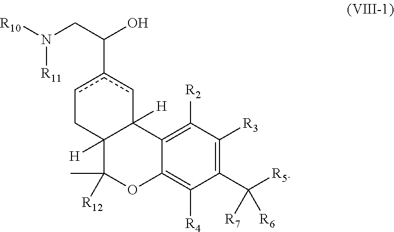

101. The compound of claim 100, wherein the compound is described by formula (VIII-2):

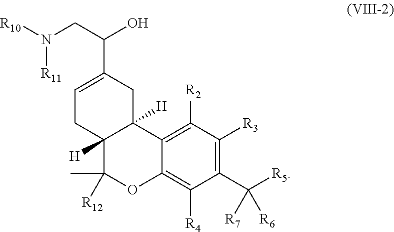

102. The compound of claim 100, wherein the compound is described by formula (VIII-3):

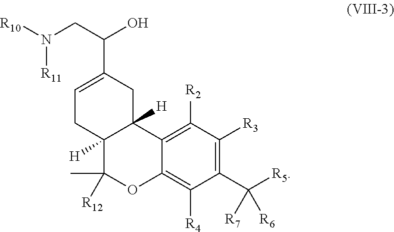

103. The compound of claim 100, wherein the compound is described by formula (VIII-4):

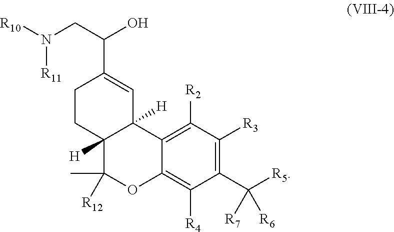

104. The compound of claim 99, wherein the compound is described by formula (VIII-5):

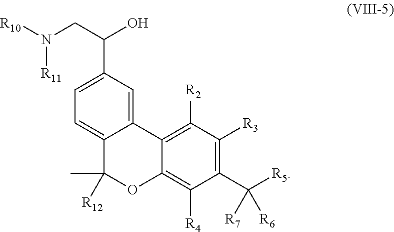

105. The compound of any one of claims 100-104, wherein R₂ is H, OH, Cl, F, NH₂, or methoxy.

106. The compound of any one of claims 100-105, wherein R₃ and R₄ are each independently H, Cl, or F.

107. The compound of claim 99, wherein the compound is described by formula (VIII-6):

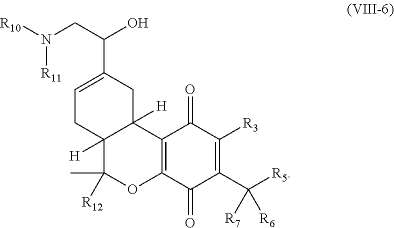

108. The compound of claim 107, wherein the compound is described by formula (VIII-7):

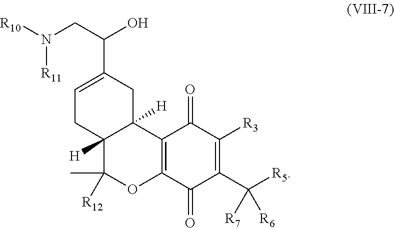

109. The compound of claim 107 or 108, wherein R₃ is H, Cl, or F.

110. The compound of claim 99, wherein the compound is described by formula (VIII-8):

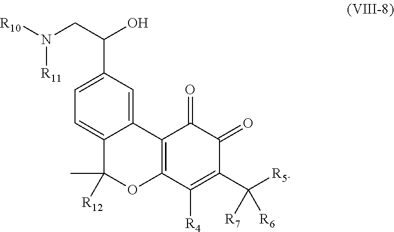

111. The compound of claim 110, wherein R₄ is H, Cl, or F.

112. The compound of any one of claims 99-111, wherein R₁₀ and R₁₁ are each independently selected from H or any one of:

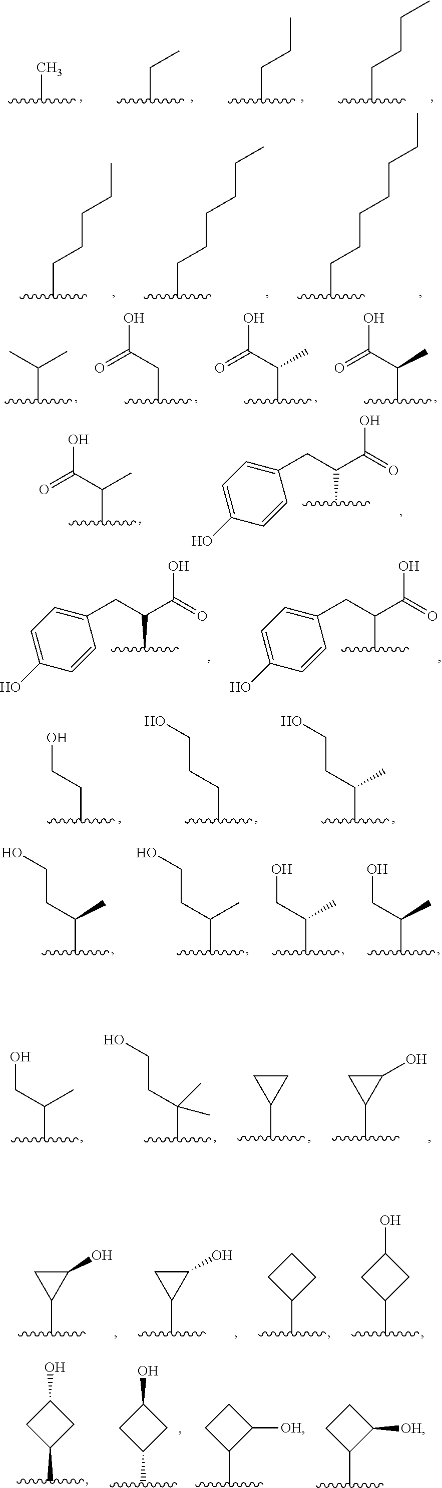

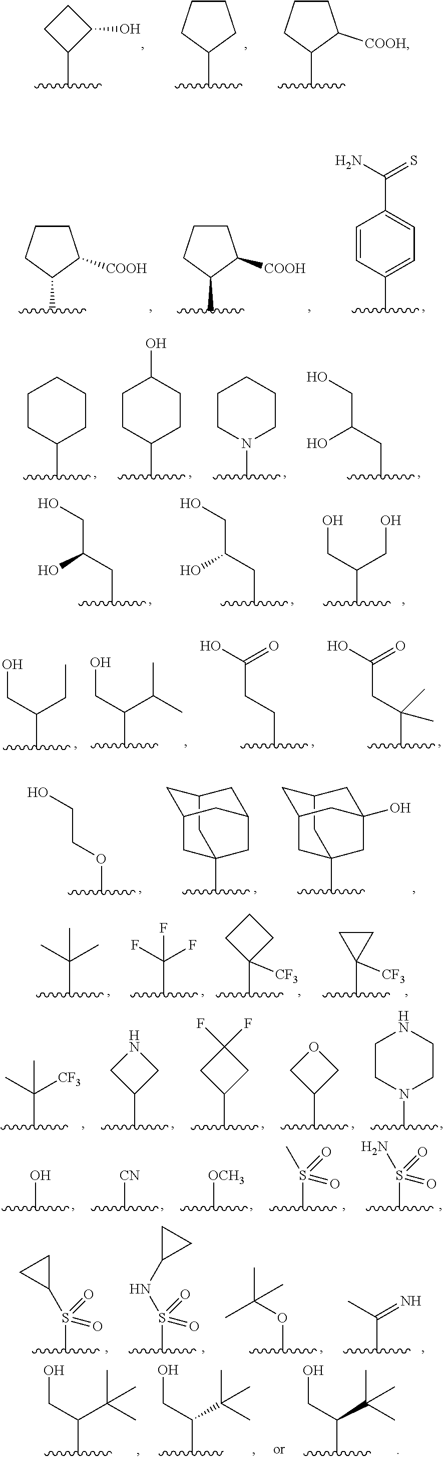

113. The compound of any one of claims 99-112, wherein R is H.

114. The compound of any one of claims 99-112, wherein R is —CH₃.

115. The compound of any one of claims 99-111, wherein R₁₀ and R₁₁ form an optionally substituted C3-C20 heterocyclyl.

116. The compound of claim 115, wherein the optionally substituted C3-C20 heterocyclyl is selected from:

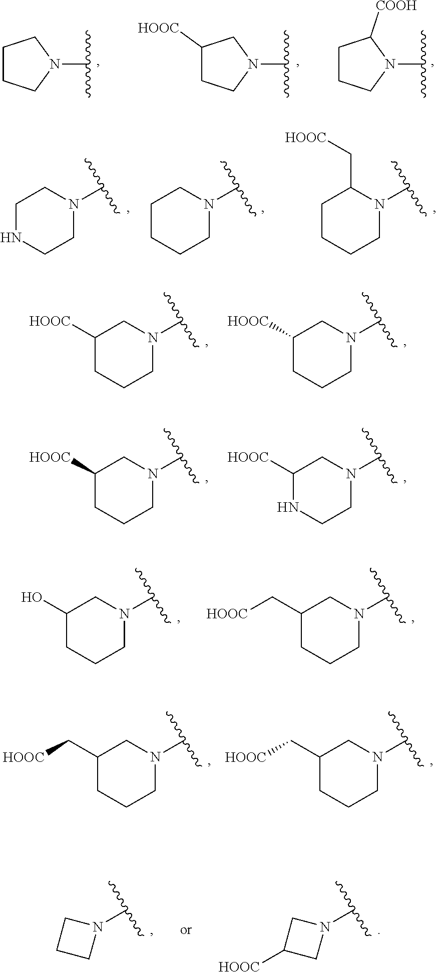

117. The compound of any one of claims 1-116, wherein R₅ is selected from:

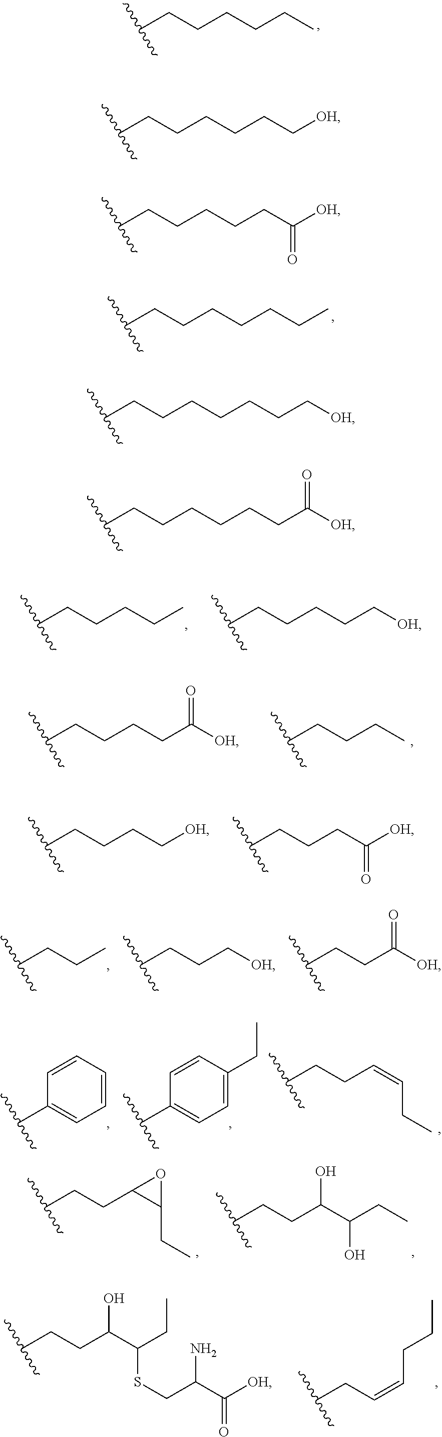

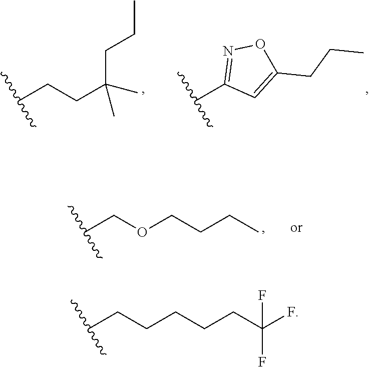

118. The compound of any one of claims 1-117, wherein R₂ is H.

119. The compound of any one of claims 1-117, wherein R₂ is OH.

120. The compound of any one of claims 1-117, wherein R₂ is methoxy.

121. The compound of any one of claims 1-117, wherein R₂ is C1.

122. The compound of any one of claims 1-117, wherein R₂ is F.

123. The compound of any one of claims 1-117, wherein R₂ is NH₂.

124. The compound of any one of claims 1-123, wherein R₃ is H.

125. The compound of any one of claims 1-123, wherein R₃ is C1.

126. The compound of any one of claims 1-123, wherein R₃ is F.

127. The compound of any one of claims 1-126, wherein R₄ is H.

128. The compound of any one of claims 1-126, wherein R₄ is C1.

129. The compound of any one of claims 1-126, wherein R₄ is F.

130. The compound of any one of claims 1-129, wherein R₆ is —CH₃.

131. The compound of any one of claims 1-129, wherein R₆ is —CH₂OH.

132. The compound of any one of claims 1-129, wherein R₆ is —CF₃.

133. The compound of any one of claims 1-132, wherein R₇ is —CH₃.

134. The compound of any one of claims 1-132, wherein R₇ is —CH₂OH.

135. The compound of any one of claims 1-133, wherein R₇ is —CF₃.

136. The compound of any one of claims 1-135, wherein R₁₂ is —CH₃.

137. The compound of any one of claims 1-135, wherein R₁₂ is —CH₂OH.

138. The compound of claim 1, wherein the compound is any one of compounds 1-144.

139. A pharmaceutical composition comprising the compound of any claims 1-138, or a salt thereof, and a pharmaceutically acceptable excipient.

140. A method of treating an inflammatory disease in a subject in need thereof, the method comprising administering to the subject a pharmaceutical composition of claim 139 in an amount sufficient to treat the condition.

141. The method of claim 140, wherein said inflammatory disease is selected from the group consisting of dermatomyositis, systemic lupus erythematosus, acquired immune deficiency syndrome (AIDS), multiple sclerosis, rheumatoid arthritis, psoriasis, diabetes, cancer, asthma, atopic dermatitis, an autoimmune thyroid disorders, ulcerative colitis, Crohn's disease, stroke, ischemia, a neurodegenerative disease, amyotrophic lateral sclerosis (ALS), chronic traumatic encephalopathy (CTE), chronic inflammatory demyelinating polyneuropathy, an autoimmune inner ear disease, uveitis, iritis, and peritonitis.

142. A method of treating a fibrotic disease in a subject in need thereof, the method comprising administering to the subject the pharmaceutical composition of claim 139 in an amount sufficient to treat the condition.

143. The method of claim 142, wherein said fibrotic disease is selected from the group consisting of cystic fibrosis, scleroderma, liver cirrhosis, interstitial pulmonary fibrosis, idiopathic pulmonary fibrosis, Dupuytren's contracture, keloids, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, or organ fibrosis.
